(19) United States
(12) Patent Application Publication
(10) Pub. No.: US 2025/0326713 A1
(43) Pub. Date: Oct. 23, 2025

(54) INHIBITORS OF MsbA AS ANTIBIOTICS, PHARMACEUTICAL COMPOSITIONS, AND USES THEREOF

(71) Applicant: Merck Sharp & Dohme LLC, Rahway, NJ (US)

(72) Inventors: Izzat Raheem, Doylestown, PA (US); Carl J. Balibar, Richboro, PA (US); Jason W. Skudlarek, Audubon, PA (US); Marc A. Labroli, Moorestown, NJ (US); Helen Mitchell, Richboro, PA (US); Ling Tong, Warren, NJ (US); Ashley Forster, Gilbertsville, PA (US); David N. Hunter, Hatfield, PA (US); Sarah W. Li, Audubon, PA (US); Hao Wang, Furlong, PA (US); Chengwei Wu, Maple Glen, PA (US); Alexei Buevich, Highland Park, NJ (US); Li-Kang Zhang, Edison, NJ (US); Kerim Babaoglu, Sudbury, MA (US); Zhe Wu, Palo Alto, CA (US); Anthony W. Shaw, Harleysville, PA (US); Andrew J. Cooke, Doylestown, PA (US)

(73) Assignee: Merck Sharp & Dohme LLC, Rahway, NJ (US)

(21) Appl. No.: 18/866,785

(22) PCT Filed: May 15, 2023
(86) PCT No.: PCT/US2023/022200

§ 371 Date: Nov. 18, 2024

Related U.S. Application Data
(60) Provisional application No. 63/344,373, filed on May 20, 2022.
(60) Provisional application No. 63/437,858, filed on Jan. 9, 2023.

(51) Int. Cl. C07D 205/04; A61K 31/196; A61K 31/341; A61K 31/381; A61K 31/397; A61K 31/415; A61K 31/4155; A61K 31/4192; A61K 31/422; A61K 31/426; A61K 31/427; A61K 31/4427; A61K 31/4439; A61K 31/5377; A61P 31/04; C07C 311/21; C07D 231/18; C07D 265/30; C07D 277/36; C07D 307/16; C07D 333/34; C07D 401/12; C07D 401/14; C07D 403/12; C07D 403/14; C07D 405/14; C07D 409/12; C07D 409/14; C07D 413/12; C07D 413/14; C07D 417/12

(52) U.S. Cl. CPC C07D 205/04; A61K 31/196; A61K 31/341; A61K 31/381; A61K 31/397; A61K 31/415; A61K 31/4155; A61K 31/4192; A61K 31/422; A61K 31/426; A61K 31/427; A61K 31/4427; A61K 31/4439; A61K 31/5377; A61P 31/04; C07C 311/21; C07D 231/18; C07D 265/30; C07D 277/36; C07D 307/16; C07D 333/34; C07D 401/12; C07D 401/14; C07D 403/12; C07D 403/14; C07D 405/14; C07D 409/12; C07D 409/14; C07D 413/12; C07D 413/14; C07D 417/12; C07C 2601/04

(57) ABSTRACT

The present disclosure is directed to certain functionalized dual substituted arene derivatives (e.g., arenesulfonamide, arenesulfamide, areneoxalamide and areneamide derivatives) of Formula I; and pharmaceutically acceptable salts thereof, wherein X¹, X², Y, Z, G, R¹, R², R³, R⁴, and R⁵ are as defined herein, which are potent inhibitors of MsbA and may be useful in the treatment of infections caused by any multi-drug resistant (MDR) Gram-negative bacteria. The disclosure is also directed to pharmaceutical compositions comprising these compounds and the use of these compounds and compositions in the treatment of infections in which MsbA is involved.

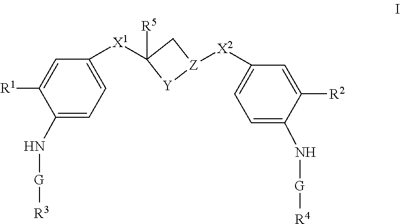

### BACKGROUND OF THE DISCLOSURE

New drugs are needed to treat gram-negative bacterial infections. These bacteria are protected by an outer membrane which prevents many antibiotics from reaching their cellular targets. Over the past several years, the incidence of nosocomial infections caused by multi-drug resistant (MDR) Gram-negative bacteria has risen dramatically. More troublingly, this increase in MDR pathogens amongst hospital-acquired infections has left clinicians with very few treatment options. Standard of care for hospital-acquired MDR Gram-negative bacteria revolves around empiric treatment for suspected agent(s) causing infection. Without a definitive culture and antibiogram data or in treating a polymicrobial infection, the Infectious Disease Society of America recommends treatment regimens including carbapenems, fluoroquinolones, and oxazolidinones, modified based on local isolate epidemiology. Of particular concern is the increasing prevalence of carbapenem-resistant Acinetobacter baumannii (CRAB) amongst ICU patients, particularly those requiring mechanical ventilation. This has led to the growing use of older and more toxic antibacterials (e.g., colistin) and even bacteriophage therapy. Colistin, a polymyxin, is an antibacterial that had fallen out of use with the advent of less toxic advanced carbapenems and cephalosporins. However, the increase in CRAB has driven the use of highly nephrotoxic colistin in this very vulnerable patient population. Incidence of drug-resistant A. baumannii have been rising resulting in the designation of CRAB as a high priority public health threat by both the World Health Organization and the Centers for Disease Control and Prevention. In 2017 in the United States there were an estimated 8500 cases of CRAB resulting in 700 deaths and an attributable $281M in excess healthcare costs. Worldwide, >60% of A. baumannii clinical isolates are drug-resistant, and that resistance can exceed 90% in some regions. Mortality rates for A. baumannii-mediated HAP and BSI approach 60%. Successful development of novel agents to combat MDR Gram-negatives is desperately needed. The high incidence of CRAB points to the need to combat infection by exploiting novel targets beyond those of the agents in common clinical use (e.g., b-lactams, tetracyclines, fluoroquinolones). Novel chemical matter and targets may also mitigate some of the toxicities seen with older agents (e.g., colistin). The outer membrane (OM) of Gram-negative bacteria is an asymmetric lipid bilayer consisting of phospholipids on the inner leaflet and lipopolysaccharides (LPS) on the outer leaflet. The presence of the OM and the properties of LPS contribute to the robust permeability barrier function of the OM. LPS biosynthesis begins on the cytoplasmic face of the inner membrane (IM). After assembly of the core LPS molecule, MsbA flips it to the periplasmic face of the IM (Voss, B. J. & Trent, M. S. LPS Transport: Flipping Out over MsbA. Curr Biol (2018). 28: R30-R3). MsbA is an ABC transporter that acts as the “flippase” on the IM and is not the target of any approved antibacterial agents. MsbA is encoded by an essential gene and LPS is its only known substrate. The Lpt machine then transports LPS across the aqueous periplasm and into the OM. Inhibiting MsbA could be an adventitious way to combat infection by any Gram-negative.

MsbA is biochemically well-behaved and plays an essential role in lipopolysaccharide (LPS) biogenesis in Gram-negative bacteria which is why it has long been used as a model ABC transporter; Thelot, F. A. et al. Science doi: 10.1126/science.abi9009 (doi: doi.org/10.1101/2021.05.25.445681); Zhang, G. et al. PNAS, Vol. 115, No. 26, 2018, 6834-6839 (www.pnas.org/cgi/doi/10.1073/pnas. 1804670115). Inhibition of MsbA leads to accumulation of LPS intermediates in the inner membrane, which is toxic and leads to cell death, highlighting the potential of MsbA as a target for development of novel antibiotics against multidrug-resistant pathogens.

While progress has been made in understanding the detailed mechanism of MsbA-driven LPS flipping, investigation on small molecule inhibition of MsbA has lagged behind, hindering the discovery of antibiotics to block LPS transport and outer membrane biogenesis; Thelot, F., et al. Curr Opin Struct Biol 63, 26-33 (2020). The current disclosure describes novel, narrow-spectrum, antibacterial compounds that act through inhibition of the genetically essential flippase MsbA.

### SUMMARY OF THE DISCLOSURE

The present disclosure is directed to certain functionalized dual substituted arene derivatives (e.g., arenesulfonamide, arenesulfamide, areneoxalamide and areneamide derivatives) joined by a cyclic or heterocyclic linker, which are collectively or individually referred to herein as “compound(s) of the disclosure” or “compounds of Formula I”, as described herein. Applicant has found, surprisingly and advantageously, that the compounds of Formula I, exhibit excellent MsbA inhibitory activity. The compounds of the disclosure may be useful as an antibacterial in the treatment or prevention of infections caused by any multi-drug resistant (MDR) Gram-negative bacteria. The disclosure is also directed to pharmaceutical compositions comprising a compound of the disclosure and to methods for the use of such compounds and compositions for the treatments described herein.

### DETAILED DESCRIPTION OF THE DISCLOSURE

For each of the following embodiments, any variable not explicitly defined in the embodiment is as defined in Formula (I). In each of the embodiments described herein, each variable is selected independently of the other unless otherwise noted.

In one embodiment, the compounds of the disclosure have the structural Formula I:

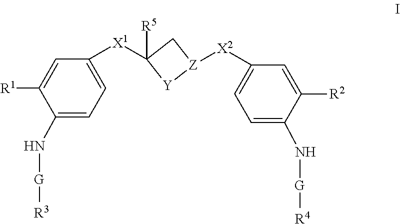

or a pharmaceutically acceptable salt thereof, wherein:
X¹ and X² are independently selected from —O—, —CH₂—, —CH₂O—, —OCH₂—, —NHCH₂—, and —CH₂NH—;
Y is selected from —CHRⁱ—, —O—, and —NH—;
Z is selected from —N—, —CH₂, —CH₂—CH₂, and CH₂—CH₂—CH₂;
R¹ and R² are independently selected from —(CH₂)ₙC(O)OR, —C(O) NHSO₂C₁₋₆alkyl, —SO₂OH, —SO₂OCl, and tetrazolyl;
R is selected from H and C₁₋₆alkyl;
Rⱼ is selected from H, C₁₋₆alkyl, C₆₋₁₀aryl, C₃₋₁₀heterocycloalkyl, and C₃₋₁₀heteroaryl, said alkyl, aryl, heterocycloalkyl and heteroaryl optionally substituted with 1 to 3 groups of R^{a}; each G is independently selected from —S(O₂)—, —C(O)C(O)NH—, and —C(O)—;
R³ and R⁴ are independently selected from —C₁₋₆alkyl, —(CH₂)₁₋₄C(O), C₆₋₁₀aryl, C₃-10 heterocycloalkyl, and C₃₋₁₀heteroaryl, said aryl, heterocycloalkyl and heteroaryl optionally substituted with 1 to 3 groups of R^{a};
R⁵ is selected from hydrogen and C₁₋₆alkyl;
R^{a} is selected from C₁₋₆alkyl, —OC₁₋₃haloalkyl, phenyl, —Ophenyl, and pyridyl, said alkyl, phenyl and pyridyl optionally substituted with 1 to 3 groups of R^{b};
R^{b} is selected from OH phenyl, and halogen; and
n is 0, 1, 2, or 3.

In one embodiment of the compounds of structural Formula I,
X¹ and X² are independently selected from —O—, —CH₂—, —CH₂O—, —OCH₂—, —NHCH₂—, and —CH₂NH—;
Y is selected from —CHRⁱ—, —O—, and —NH—;
Z is selected from —N—, —CH₂, —CH₂—CH₂, and CH₂—CH₂—CH₂;
R¹ and R² are independently selected from —(CH₂)ₙC(O)OR, —C(O) NHSO₂C₁₋₆alkyl, —SO₂OH, —SO₂OCl, and tetrazolyl;
R is selected from H and C₁₋₆alkyl;
R^{j} is selected from H, C₁₋₆alkyl, C₆₋₁₀aryl, C₃₋₁₀heterocycloalkyl, and C₄₋₁₀heteroaryl, said alkyl, aryl, heterocycloalkyl and heteroaryl optionally substituted with 1 to 3 groups of R^{a}; each G is independently selected from —S(O₂)—, —C(O)C(O) NH—, and —C(O)—;

R³ and R⁴ are independently selected from —C₁₋₆alkyl, —(CH₂)₁₋₄C(O), C₆₋₁₀aryl, C₃₋₁₀ heterocycloalkyl, and C₄₋₁₀heteroaryl, said aryl, heterocycloalkyl and heteroaryl optionally substituted with 1 to 3 groups of R^{a};
R⁵ is selected from hydrogen and C₁₋₆alkyl;
R^{a} is selected from C₁₋₆alkyl, —OC₁₋₃haloalkyl, phenyl, —Ophenyl, and pyridyl, said alkyl, phenyl and pyridyl optionally substituted with 1 to 3 groups of R^{b};
R^{b} is selected from OH phenyl, and halogen; and
n is 0, 1, 2, or 3, or a pharmaceutically acceptable salt thereof.

An embodiment of Formula I is realized when R is H.

An embodiment of Formula I is realized when R is C₁₋₆alkyl. A subembodiment of this aspect of the disclosure is realized when R is CH₃.

An embodiment of Formula I is realized when X¹ is —O—.

An embodiment of Formula I is realized when X¹ is —CH₂—.

Another embodiment of Formula I is realized when X¹ is —CH₂O—.

Another embodiment of Formula I is realized when X¹ is —OCH₂—.

Another embodiment of Formula I is realized when X¹ is —NHCH₂—.

Another embodiment of Formula I is realized when X¹ is —CH₂NH—.

Still another embodiment of Formula I is realized when X² is —O—.

Still another embodiment of Formula I is realized when X² is —CH₂—.

Another embodiment of Formula I is realized when X² is —CH₂O—.

Another embodiment of Formula I is realized when X² is —OCH₂—.

Another embodiment of Formula I is realized when X² is —NHCH₂—.

Still another embodiment of Formula I is realized when X² is —CH₂NH—.

Another embodiment of Formula I is realized when X¹ and X², respectively are selected from 1)—O— and CH₂; 2)—OCH₂— and —CH₂O—and 3) both are —NHCH₂—.

Yet another embodiment of Formula I is realized when X¹ and X² are —O— and CH₂, respectively.

Another embodiment of Formula I is realized when X¹ and X² are —OCH₂— and —CH₂O—, respectively.

Another embodiment of Formula I is realized when X¹ and X² are both —NHCH₂—.

Another embodiment of Formula I is realized when Y is —CH₂—.

Another embodiment of Formula I is realized when Y is —CH(C₁₋₆ alkyl), said alkyl, optionally substituted with 1 to 3 groups of R^{a}.

Another embodiment of Formula I is realized when Y is —CH(C₆₋₁₀ aryl), aryl, optionally substituted with 1 to 3 groups of R^{a}.

Another embodiment of Formula I is realized when Y is —CH(C₃₋₁₀ heterocycloalkyl), said heterocycloalkyl optionally substituted with 1 to 3 groups of R^{a}.

Another embodiment of Formula I is realized when Y is —CH(C₃₋₁₀ heteroaryl), said heteroaryl optionally substituted with 1 to 3 groups of R^{a}.

Another embodiment of Formula I is realized when Y is —O—.

Another embodiment of Formula I is realized when Y is —NH—.

Yet another embodiment of Formula I is realized when X¹ and X² are —O— and CH₂, respectively and Y is —CH₂—.

Another embodiment of Formula I is realized when X¹ and X² are —OCH₂— and —CH₂O—, respectively and Y is —O—.

Another embodiment of Formula I is realized when X¹ and X² are both —NHCH₂— and Y is CH₂.

Another embodiment of Formula I is realized when Z is —N—. Another embodiment of Formula I is realized when Z is —N—then Y is not —O—.

Another embodiment of Formula I is realized when Z is —CH₂.

Another embodiment of Formula I is realized when Z is —CH₂—CH₂. A subembodiments of the disclosure of Formula I is realized when the carbon atoms where Z is CH₂—CH₂ combine together with the atoms in the ring containing Y and Z to form a five membered ring. A subembodiment of this aspect of the disclosure is realized when the atoms in the five membered ring, including Y, are all carbon. A further subembodiment of the disclosure where the atoms in the five membered ring, including Y, are all carbon is realized when a bicyclic ring structure is formed.

Another embodiment of Formula I is realized when Z is CH₂—CH₂—CH₂. A subembodiment of the disclosure of Formula I is realized when the carbon atoms where Z is CH₂—CH₂—CH₂ combine together with the atoms in the ring containing Y and Z to form a six membered ring. A subembodiment of this aspect of the disclosure is realized when the atoms in the six membered ring, including Y, are all carbon. A further subembodiment of the disclosure this aspect of the disclosure is realized when a bridged ring structure is formed.

Another embodiment of Formula I is realized when Y is —O—and Z is C₁₋₆alkyl.

Another embodiment of Formula I is realized when Y is —O— or —NH— and Z is selected from —CH₂—CH₂ and CH₂—CH₂—CH₂.

Yet another embodiment of Formula I is realized when X¹ and X² are —O—and CH₂, respectively, Y is —CH₂— and Z is N.

Another embodiment of Formula I is realized when X¹ and X² are —OCH₂— and —CH₂O—, respectively, Y is —O—and Z is —CH₂—CH₂.

Another embodiment of Formula I is realized when X¹ and X² are both —NHCH₂—, Y is CH₂ and Z is CH₂.

Still another embodiment of Formula I is realized when R¹ is —(CH₂)ₙC(O)OR. An aspect of this embodiment is realized when R¹ is —CH₂C(O) OH. An aspect of this embodiment is realized when R¹ is —C(O)OCH₃ or —C(O)OCH₂CH₃. Another aspect of this embodiment is realized when R¹ is —C(O)OH.

Another embodiment of Formula I is realized when R¹ is —C(O) NHSO₂C₁₋₆alkyl. An aspect of this embodiment is realized when R¹ is selected from —C(O) NHSO₂CH₃, and —C(O) NHSO₂CH₂CH₃.

Still another embodiment of Formula I is realized when R² is —(CH₂)ₙC(O)OR. An aspect of this embodiment is realized when R² is —CH₂C(O) OH. An aspect of this embodiment is realized when R² is —C(O)OCH₃ or —C(O)OCH₂CH₃. Another aspect of this embodiment is realized when R² is —C(O)OH.

Another embodiment of Formula I is realized when R² is —C(O) NHSO₂C₁₋₆alkyl. An aspect of this embodiment is realized when R² is selected from —C(O) NHSO₂CH₃, and —C(O)NHSO₂CH₂CH₃.

Another embodiment of Formula I is realized when R² is —SO₂OH.

Another embodiment of Formula I is realized when R² is tetrazolyl.

Another embodiment of Formula I is realized when R¹ and R² are both —C(O)OH.

Another embodiment of Formula I is realized when R¹ and R² are both —C(O)OCH₃.

Yet another embodiment of Formula I is realized when X¹ and X² are —O—and CH₂, respectively, Y is —CH₂—, Z is N and R¹ and R² are both —C(O)OH or —C(O) OCH₃.

Another embodiment of Formula I is realized when X¹ and X² are —OCH₂— and —CH₂O—, respectively, Y is —O—, Z is —CH₂—CH₂, and R¹ and R² are both —C(O)OH or —C(O)OCH₃.

Another embodiment of Formula I is realized when X¹ and X² are both —NHCH₂—, Y is CH₂, Z is CH₂, and R¹ and R² are both —C(O)OH or —C(O)OCH₃.

Still another embodiment of Formula I is realized when G is —S(O₂)—. A subembodiment of this aspect of the disclosure is realized when G is —S(O₂)—, X¹ and X² are —O—and CH₂, respectively, and Y is —CH₂— or —O—. Another embodiment of Formula I is realized when G is —S(O₂)—, X¹ and X² are —OCH₂— and —CH₂O—, respectively and Y is —O—. Another subembodiment of this aspect of the disclosure is realized when G is —S(O₂)—, X¹ and X² are both —NHCH₂— and Y is CH₂. Another subembodiment of this aspect of the disclosure is realized when G is —S(0) 2, X¹ and X² are —O—and CH₂, respectively, Y is —CH₂— and Z is N. Another subembodiment of this aspect of the disclosure is realized when G is —S(O₂)—, X¹ and X² are —OCH₂— and —CH₂O—, respectively, Y is —O—and Z is —CH₂—CH₂. Another subembodiment of this aspect of the disclosure is realized when G is —S(O₂)—, X¹ and X² are both —NHCH₂—, Y is CH₂ and Z is CH₂. Yet another subembodiment of this aspect of the disclosure is realized when G is —S(O₂)—, X¹ and X² are —O—and CH₂, respectively, Y is —CH₂—, Z is N and R¹ and R² are both —C(O)OH or —C(O)OCH₃. Another subembodiment of this aspect of the disclosure is realized when G is —S(O₂)—, X¹ and X² are —OCH₂— and —CH₂O—, respectively, Y is —O—, Z is —CH₂—CH₂, and R¹ and R² are both —C(O)OH or —C(O)OCH₃. Another subembodiment of this aspect of the disclosure is realized when G is —S(O₂)—, X¹ and X² are both —NHCH₂—, Y is CH₂, Z is CH₂, and R¹ and R² are both —C(O)OH or —C(O)OCH₃. Another subembodiment of this disclosure is realized when G is —S(O₂)—, X¹ and X² are —O—and CH₂, respectively, Y is —CH₂—, Z is N and R¹ and R² are both —C(O) OH or —C(O)OCH₃.

Another embodiment of Formula I is realized when G is —C(O)C(O) NH—.

Still another embodiment of Formula I is realized when G is —C(O)—.

Another embodiment of Formula I is realized when R³ is —C₁₋₆alkyl.

Another embodiment of Formula I is realized when R³ is optionally substituted C₆₋₁₀aryl. A subembodiment of this aspect of the disclosure is realized when the optionally substituted C₆-10aryl of R³ is phenyl.

Another embodiment of Formula I is realized when R³ is optionally substituted C₃₋₁₀heterocycloalkyl. A subembodiment of this aspect of the disclosure is realized when the optionally substituted C₃₋₁₀heterocycloalkyl of R³ is selected from optionally substituted azetidinyl, oxazolidinyl, or morpholinyl.

Another embodiment of Formula I is realized when R³ is optionally substituted C₃₋₁₀heteroaryl. A subembodiment of this aspect of the disclosure is realized when the optionally substituted heteroaryl of R³ is selected from optionally substituted pyrazolyl, triazolyl, thienyl, thiazolyl, and oxazolyl. A subembodiment of this aspect of the disclosure is realized when the optionally substituted heteroaryl of R³ is pyrazolyl. A subembodiment of this aspect of the disclosure is realized when the optionally substituted heteroaryl of R³ is thiazolyl. Another embodiment of Formula I is realized when R⁴ is —C₁₋₆alkyl.

Another embodiment of Formula I is realized when R⁴ is optionally substituted C₆₋₁₀aryl. A subembodiment of this aspect of the disclosure is realized when the optionally substituted C₆-10aryl of R⁴ is phenyl.

Another embodiment of Formula I is realized when R⁴ is optionally substituted C₃₋₁₀heterocycloalkyl. A subembodiment of this aspect of the disclosure is realized when the optionally substituted C₃₋₁₀heterocycloalkyl of R⁴ is selected from azetidinyl, oxazolidinyl, or morpholinyl.

Another embodiment of Formula I is realized when R⁴ is optionally substituted C₄₋₁₀ heteroaryl. A subembodiment of this aspect of the disclosure is realized when the optionally substituted heteroaryl of R⁴ is selected from pyrazolyl, pyridyl, triazolyl, thienyl, thiazolyl, and oxazolyl. A subembodiment of this aspect of the disclosure is realized when the optionally substituted heteroaryl of R⁴ is pyrazolyl. A subembodiment of this aspect of the disclosure is realized when the optionally substituted heteroaryl of R⁴ is thiazolyl.

Another embodiment of Formula I is realized when R³ and R⁴ are both optionally substituted C₆₋₁₀aryl, C₃₋₁₀heteroaryl or C₃₋₁₀heterocycloalkyl. A subembodiment of this aspect of the disclosure is realized when the aryl, heteroaryl, and heterocycloalkyl are selected from optionally substituted phenyl, morpholinyl, azetidinyl, oxazolidinyl, pyrazolyl, triazolyl, thienyl, thiazolyl, and oxazolyl. Another subembodiment of Formula I is realized when R³ and R⁴ are both optionally substituted pyrazolyl. Another subembodiment of Formula I is realized when R³ and R⁴ are both optionally substituted morpholinyl. Another subembodiment of Formula I is realized when R³ and R⁴ are both optionally substituted pyridyl. Another subembodiment of Formula I is realized when R³ and R⁴ are both optionally substituted triazolyl.

Another subembodiment of this aspect of Formula I is realized when one of R³ and R⁴ is pyrazolyl and the other is selected from optionally substituted phenyl, morpholinyl, azetidinyl, oxazolidinyl, pyrazolyl, triazolyl, thienyl, thiazolyl, and oxazolyl.

Another embodiment of Formula I is realized when one of R³ and R⁴ is C₁-6alkyl and the other is selected from C₁₋₆alkyl and optionally substituted phenyl, morpholinyl, azetidinyl, oxazolidinyl, pyrazolyl, triazolyl, thienyl, thiazolyl, and oxazolyl. Another embodiment of Formula I is realized when both R³ and R⁴ are C₁₋₆alkyl.

Another embodiment of Formula I is realized when at least one of R³ and R⁴ is —(CH₂) 1-4C(O)—.

Another embodiment of Formula I is realized when R⁵ is hydrogen. Still another embodiment of Formula I is realized when R⁵ is C₁-6alkyl. A subembodiment of this aspect of the disclosure is realized when R⁵ is CH₃.

Another embodiment of Formula I is realized when both R^{a} and R^{b} are phenyl optionally substituted with halogen. A subembodiment of this aspect of the disclosure is realized when both R^{a} and R^{b} are phenyl, said phenyls independently substituted with 1 to 3 groups of fluorine or chlorine. Another embodiment of Formula I is realized when one of R^{a} and R^{b} is optionally substituted phenyl and the other is selected from C₁₆alkyl and optionally substituted-Ophenyl, and pyridyl.

In another embodiment, the compounds of Formula I or a pharmaceutically acceptable salt thereof is realized by structural Formula II:

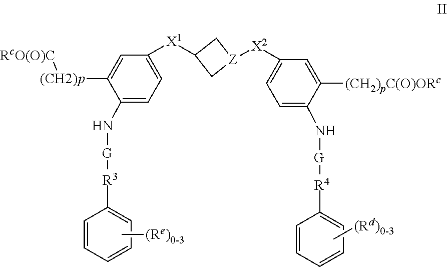

wherein X¹, X², Z, G, R³, and R⁴ are as originally described, each R^{e} is independently selected from hydrogen, CH₃, and CH₂CH₃, p is 0 or 1, and R^{c} and R^{d} when present are independently selected from chlorine and fluorine.

Another embodiment of Formula II is realized when R^{c} is hydrogen.

Another embodiment of Formula II is realized when Z is N and R^{c} and R^{d} when present are independently selected from chlorine and fluorine.

Another embodiment of Formula II is realized when Z is —CH₂— and R^{c} and R^{d} when present are independently selected from chlorine and fluorine.

Another embodiment of Formula II is realized when G is —S(O₂)—.

Another embodiment of Formula II is realized when G is —C(O)C(O) NH—.

Another embodiment of Formula II is realized when X¹ and X² are —O—and CH₂, respectively and R^{c} and R^{d} when present are independently selected from chlorine and fluorine.

Another embodiment of Formula II is realized when X¹ and X² are —OCH₂— and —NHCH₂—,respectively and R^{c} and R^{d} when present are independently selected from chlorine and fluorine. A subembodiment of this aspect of the disclosure is realized when Z is N and R^{c} and R^{d}, when present, are independently selected from chlorine and fluorine. Another subembodiment of this aspect of the disclosure is realized when Z is CH₂ and R^{c} and R^{d}, when present, are independently selected from chlorine and fluorine.

Another embodiment of Formula II is realized when X¹ and X² are —CH₂O—and —CH₂—, respectively and R^{c} and R^{d} when present are independently selected from chlorine and fluorine. A subembodiment of this aspect of the disclosure is realized when Z is N and R^{c} and R^{d}, when present, are independently selected from chlorine and fluorine. Another subembodiment of this aspect of the disclosure is realized when Z is CH₂ and R^{c} and R^{d}, when present, are independently selected from chlorine and fluorine.

Another embodiment of Formula II is realized when X¹ and X² are both —NHCH₂— and R^{e} and R^{d} when present are independently selected from chlorine and fluorine. A subembodiment of this aspect of the disclosure is realized when Z is N and R^{c} and R^{d}, when present, are independently selected from chlorine and fluorine. Another subembodiment of this aspect of the disclosure is realized when Z is CH₂ and R^{c} and R^{d}, when present, are independently selected from chlorine and fluorine.

Another embodiment of Formula II is realized when X¹ and X² is CH₂NH and —NHCH₂—, respectively and R^{c} and R^{d} when present are independently selected from chlorine and fluorine. A subembodiment of this aspect of the disclosure is realized when Z is N and R^{c} and R^{d}, when present, are independently selected from chlorine and fluorine. Another subembodiment of this aspect of the disclosure is realized when Z is CH₂ and R^{c} and R^{d}, when present, are independently selected from chlorine and fluorine.

Another embodiment of Formula II is realized when R³ and R⁴ are both optionally substituted C₆₋₁₀aryl, C₃₋₁₀heteroaryl or C₃₋₁₀heterocycloalkyl. A subembodiment of this aspect of the disclosure is realized when the aryl, heteroaryl, and heterocycloalkyl are selected from optionally substituted phenyl, morpholinyl, azetidinyl, oxazolidinyl, pyrazolyl, triazolyl, thienyl, thiazolyl, and oxazolyl. Another subembodiment of Formula II is realized when R³ and R⁴ are both optionally substituted pyrazolyl. Another subembodiment of Formula II is realized when R³ and R⁴ are both optionally substituted morpholinyl. Another subembodiment of Formula II is realized when R³ and R⁴ are both optionally substituted pyridyl. Another subembodiment of Formula II is realized when R³ and R⁴ are both optionally substituted triazolyl.

Another subembodiment of this aspect of Formula II is realized when one of R³ and R⁴ is pyrazolyl and the other is selected from optionally substituted phenyl, morpholinyl, azetidinyl, oxazolidinyl, pyrazolyl, triazolyl, thienyl, thiazolyl, and oxazolyl.

Another embodiment of Formula II is realized when one of R³ and R⁴ is C₁-6alkyl and the other is selected from C₁₋₆alkyl or optionally substituted phenyl, morpholinyl, azetidinyl, oxazolidinyl, pyrazolyl, triazolyl, thienyl, thiazolyl, and oxazolyl. Another embodiment of Formula II is realized when both R³ and R+ are C₁₋₆alkyl.

Another embodiment of the disclosure of Formula I or a pharmaceutically acceptable salt thereof is represented by structural Formula III:

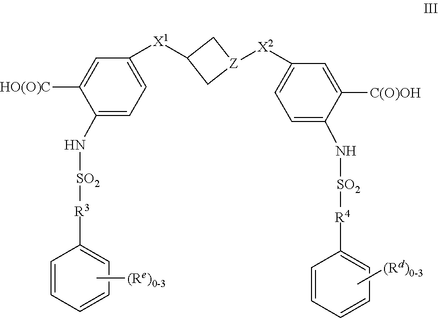

wherein X¹, X², Z, R³, and R⁴ are as originally described and R^{c} and R^{d}, when present, are independently selected from chlorine and fluorine.

Another embodiment of Formula III is realized when Z is N and R^{c} and R^{d}, when present, are independently selected from chlorine and fluorine.

Another embodiment of Formula III is realized when Z is CH₂ and R^{c} and R^{d}, when present, are independently selected from chlorine and fluorine.

Another embodiment of Formula III is realized when X¹ and X² are —O—and CH₂, respectively and R^{c} and R^{d} when present are independently selected from chlorine and fluorine. A subembodiment of this aspect of the disclosure is realized when Z is N and R^{c} and R^{d}, when present, are independently selected from chlorine and fluorine. Another subembodiment of this aspect of the disclosure is realized when Z is CH₂ and R^{c} and R^{d}, when present, are independently selected from chlorine and fluorine.

Another embodiment of Formula III is realized when X¹ and X² are —OCH₂— and —NHCH₂—, respectively and R^{c} and R^{d} when present are independently selected from chlorine and fluorine. A subembodiment of this aspect of the disclosure is realized when Z is N and R^{c} and R^{d}, when present, are independently selected from chlorine and fluorine. Another subembodiment of this aspect of the disclosure is realized when Z is CH₂ and R^{c} and R^{d}, when present, are independently selected from chlorine and fluorine.

Another embodiment of Formula III is realized when X¹ and X² are —CH₂O—and —CH₂—, respectively and R^{c} and R^{d} when present are independently selected from chlorine and fluorine. A subembodiment of this aspect of the disclosure is realized when Z is N and R^{c} and R^{d}, when present, are independently selected from chlorine and fluorine. Another subembodiment of this aspect of the disclosure is realized when Z is CH₂ and R^{c} and R^{d}, when present, are independently selected from chlorine and fluorine.

Another embodiment of Formula III is realized when X¹ and X² are both —NHCH₂— and R^{e} and R^{d} when present are independently selected from chlorine and fluorine. A subembodiment of this aspect of the disclosure is realized when Z is N and R^{c} and R^{d}, when present, are independently selected from chlorine and fluorine. Another subembodiment of this aspect of the disclosure is realized when Z is CH₂ and R^{c} and R^{d}, when present, are independently selected from chlorine and fluorine.

Another embodiment of Formula III is realized when X¹ and X² is CH₂NH and —NHCH₂—, respectively and R^{c} and R^{d} when present are independently selected from chlorine and fluorine. A subembodiment of this aspect of the disclosure is realized when Z is N and R^{c} and R^{d}, when present, are independently selected from chlorine and fluorine. Another subembodiment of this aspect of the disclosure is realized when Z is CH₂ and R^{c} and R^{d}, when present, are independently selected from chlorine and fluorine.

Another embodiment of Formula III is realized when R³ and R⁴ are both optionally substituted C₆₋₁₀aryl, C₃₋₁₀heteroaryl or C₃₋₁₀heterocycloalkyl. A subembodiment of this aspect of the disclosure is realized when the aryl, heteroaryl, and heterocycloalkyl are selected from optionally substituted phenyl, morpholinyl, azetidinyl, oxazolidinyl, pyrazolyl, triazolyl, thienyl, thiazolyl, and oxazolyl. Another subembodiment of Formula III is realized when R³ and R⁴ are both optionally substituted pyrazolyl. Another subembodiment of Formula III is realized when R³ and R⁴ are both optionally substituted morpholinyl. Another subembodiment of Formula III is realized when R³ and R⁴ are both optionally substituted pyridyl. Another subembodiment of Formula III is realized when R³ and R⁴ are both optionally substituted triazolyl.

Another subembodiment of this aspect of Formula III is realized when one of R³ and R⁴ is pyrazolyl and the other is selected from optionally substituted phenyl, morpholinyl, azetidinyl, oxazolidinyl, pyrazolyl, triazolyl, thienyl, thiazolyl, and oxazolyl.

Another embodiment of Formula III is realized when one of R³ and R⁴ is C₁-6alkyl and the other is selected from C₁₋₆alkyl or optionally substituted phenyl, morpholinyl, azetidinyl, oxazolidinyl, pyrazolyl, triazolyl, thienyl, thiazolyl, and oxazolyl. Another embodiment of Formula III is realized when both R³ and R⁴ are C₁₋₆alkyl.

In each of the preceding embodiments and alternative embodiments described above and herein, pharmaceutically acceptable salts of each embodiment are also contemplated.

In another embodiment, the compounds of the disclosure include those identified herein as Examples below, and pharmaceutically acceptable salts thereof.

In another embodiment, the present disclosure provides pharmaceutical compositions comprising a pharmaceutically acceptable carrier and a compound of the disclosure or a pharmaceutically acceptable salt thereof.

In another embodiment, the present disclosure provides a method of treating bacterial infections caused by multi-drug resistant (MDR) Gram-negative bacteria, said method comprising administering to a subject (e.g., mammal, person, or patient) in need of such treatment an effective amount of a compound of the disclosure, or a pharmaceutically acceptable salt thereof, or pharmaceutically acceptable composition thereof. Examples of Gram-negative infections include those caused by Pseudomonas, Klebsiella, Proteus, Salmonella, Providencia, Escherichia, Morganella, Aeromonas, and Citrobacter.

Another embodiment provides the use of a compound of the disclosure, or a pharmaceutically acceptable salt thereof, and a pharmaceutically acceptable carrier, for the manufacture of a medicament for the treatment of infections caused by MDR gram-negative bacteria. The disclosure may also encompass the use of a compound of the disclosure, or a pharmaceutically acceptable salt thereof, and a pharmaceutically acceptable carrier, in therapy.

Another embodiment provides for medicaments or pharmaceutical compositions which may be useful for treating bacterial infections in which MsbA is involved which comprise a compound of the disclosure, or a pharmaceutically acceptable salt thereof, and a pharmaceutically acceptable carrier.

Another embodiment provides for the use of a compound of the disclosure which may be useful for treating MDR gram-negative bacterial infections in which MsbA is involved. Another embodiment provides a method for the manufacture of a medicament or a composition which may be useful for treating MDR gram-negative bacterial infections in which MsbA is involved, comprising combining a compound of the disclosure with one or more pharmaceutically acceptable carriers.

The compounds of the disclosure may contain one or more asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this disclosure. Unless a specific stereochemistry is indicated, the present disclosure is meant to encompass all such isomeric forms of these compounds.

The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diastereomeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

In the compounds of the disclosure, the atoms may exhibit their natural isotopic abundances, or one or more of the atoms may be artificially enriched in a particular isotope having the same atomic number, but an atomic mass or mass number different from the atomic mass or mass number predominantly found in nature. The present disclosure is meant to include all suitable isotopic variations of the compounds of generic Formula I. For example, different isotopic forms of hydrogen (H) include protium (1H) and deuterium (2H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula I can be prepared without undue experimentation by conventional techniques well known to those skilled in the art or by processes analogous to those described in the Schemes and Examples herein using appropriate isotopically-enriched reagents and/or intermediates.

When a compound of the disclosure is capable of forming tautomers, all such tautomeric forms are also included within the scope of the present disclosure. For example, compounds including carbonyl —CH₂C(O)— groups (keto forms) may undergo tautomerism to form hydroxyl —CH—C(OH)— groups (enol forms). Both keto and enol forms, where present, are included within the scope of the present disclosure.

When any variable (e.g. R⁵, etc.) occurs more than one time in any constituent, its definition on each occurrence is independent at every other occurrence. Also, combinations of substituents and variables are permissible only if such combinations result in stable compounds. Lines drawn into the ring systems from substituents represent that the indicated bond may be attached to any of the substitutable ring atoms. If the ring system is bicyclic, it is intended that the bond be attached to any of the suitable atoms on either ring of the bicyclic moiety.

It is understood that one or more silicon (Si) atoms can be incorporated into the compounds of the instant disclosure in place of one or more carbon atoms by one of ordinary skill in the art to provide compounds that are chemically stable and that can be readily synthesized by techniques known in the art from readily available starting materials. Carbon and silicon differ in their covalent radius leading to differences in bond distance and the steric arrangement when comparing analogous C-element and Si-element bonds. These differences lead to subtle changes in the size and shape of silicon-containing compounds when compared to carbon. One of ordinary skill in the art would understand that size and shape differences can lead to subtle or dramatic changes in potency, solubility, lack of off-target activity, packaging properties, and so on. (Diass, J. O. et al. Organometallics (2006) 5:1188-1198; Showell, G. A. et al. Bioorganic & Medicinal Chemistry Letters (2006) 16:2555-2558).

It is understood that substituents and substitution patterns on the compounds of the instant disclosure can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” should be understood as meaning that the group in question is either unsubstituted or may be substituted with one or more substituents.

“(C₁-Cₙ)Alkyl” means an aliphatic hydrocarbon group, which may be straight or branched, comprising 1 to n carbon atoms. Thus, for example, “(C₁-C₆)alkyl” means an aliphatic hydrocarbon group, which may be straight or branched, comprising 1 to 6 carbon atoms. Similarly, for example, “(C₁-C₃)alkyl” means an aliphatic hydrocarbon group, which may be straight or branched, comprising 1 to 3 carbon atoms. Branched means that one or more lower alkyl groups such as methyl, ethyl or propyl, are attached to a linear alkyl chain. Non-limiting examples of alkyl groups include methyl, ethyl, n-propyl, isopropyl, n-butyl, i-butyl, and t-butyl.

“Haloalkyl” means an alkyl as defined above wherein one or more hydrogen atoms on the alkyl is replaced by a halogen atom. As appreciated by those of skill in the art, “halo” or “halogen” as used herein is intended to include chloro (Cl), fluoro (F), bromo (Br) and iodo (I). Chloro (C₁) and fluoro (F) halogens are generally preferred.

“Halogen” (or “halo”) means fluorine (F), chlorine (Cl), bromine (Br), or iodine (I). Preferred are fluorine, chlorine and bromine.

“Alkyl” means an aliphatic hydrocarbon group, which may be straight or branched, comprising 1 to 10 carbon atoms. “Lower alkyl” means a straight or branched alkyl group comprising 1 to 4 carbon atoms. Branched means that one or more lower alkyl groups such as methyl, ethyl or propyl, are attached to a linear alkyl chain. Non-limiting examples of suitable alkyl groups include methyl (Me), ethyl (Et), n-propyl, isopropyl, n-butyl, i-butyl, and t-butyl. “Aryl” means an aromatic monocyclic or multicyclic ring system comprising 6 to 14 carbon atoms, preferably 6 to 10 carbon atoms. Non-limiting examples of suitable aryl groups include phenyl and naphthyl. “Monocyclic aryl” means phenyl.

“Heteroaryl” means an aromatic monocyclic or multicyclic ring system comprising 4 to 14 ring atoms, preferably 5 to 10 ring atoms, in which one or more of the ring atoms is an element other than carbon, for example nitrogen, oxygen or sulfur, alone or in combination.

Preferred heteroaryls contain 5 to 6 ring atoms. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. “Heteroaryl” may also include a heteroaryl as defined above fused to an aryl as defined above. Non-limiting examples of suitable heteroaryls include pyridyl, pyrazinyl, furanyl, thienyl (which alternatively may be referred to as thiophenyl), pyrimidinyl, pyridone (including N-substituted pyridones), isoxazolyl, isothiazolyl, oxazolyl, oxadiazolyl, thiazolyl, thiadiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a|pyridinyl, imidazo|2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like. The term “monocyclic heteroaryl” refers to monocyclic versions of heteroaryl as described above and includes 4- to 7-membered monocyclic heteroaryl groups comprising from 1 to 4 ring heteroatoms, said ring heteroatoms being independently selected from the group consisting of N, O, and S, and oxides thereof. The point of attachment to the parent moiety is to any available ring carbon or ring heteroatom. Non-limiting examples of monocyclic heteroaryl moieties include pyridyl, pyrazinyl, furanyl, thienyl, pyrimidinyl, pyridazinyl, pyridone, thiazolyl, isothiazolyl, oxazolyl, oxadiazolyl, isoxazolyl, pyrazolyl, furazanyl, pyrrolyl, pyrazolyl, triazolyl, thiadiazolyl (e.g., 1,2,4-thiadiazolyl), imidazolyl, and triazinyl (e.g., 1,2,4-triazinyl), and oxides thereof.

“Cycloalkyl” means a non-aromatic monocyclic or multicyclic ring system comprising 3 to 10 carbon atoms, preferably 3 to 6 carbon atoms. The cycloalkyl can be optionally substituted with one or more substituents, which may be the same or different, as described herein. Monocyclic cycloalkyl refers to monocyclic versions of the cycloalkyl moieties described herein. Non-limiting examples of suitable monocyclic cycloalkyls include cyclopropyl, cyclopentyl, cyclohexyl, cycloheptyl and the like. Non-limiting examples of multicyclic cycloalkyls include [1.1.1]-bicyclo pentane, 1-decalinyl, norbornyl, adamantyl and the like.

“Heterocycloalkyl” (or “heterocyclyl”) means a non-aromatic saturated monocyclic or multicyclic ring system comprising 3 to 10 ring atoms, preferably 5 to 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain 5 to 6 ring atoms. The prefix aza, oxa or thia before the heterocyclyl root name means that at least a nitrogen, oxygen or sulfur atom respectively is present as a ring atom. Any-NH in a heterocyclyl ring may exist protected such as, for example, as an —N(Boc), —N(CBz), —N(Tos) group and the like; such protections are also considered part of this disclosure. The heterocyclyl can be optionally substituted by one or more substituents, which may be the same or different, as described herein. The nitrogen or sulfur atom of the heterocyclyl can be optionally oxidized to the corresponding N-oxide, S-oxide or S,S-dioxide. Thus, the term “oxide,” when it appears in a definition of a variable in a general structure described herein, refers to the corresponding N-oxide, S-oxide, or S,S-dioxide. “Heterocyclyl” also includes rings wherein ═O replaces two available hydrogens on the same carbon atom (i.e., heterocyclyl includes rings having a carbonyl group in the ring). Such ═O groups may be referred to herein as “oxo.” An example of such a moiety is pyrrolidinone (or pyrrolidone):

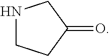

As used herein, the term “monocyclic heterocycloalkyl” refers to monocyclic versions of the heterocycloalkyl moieties described herein and include a 4- to 7-membered monocyclic heterocycloalkyl groups comprising from 1 to 4 ring heteroatoms, said ring heteroatoms being independently selected from the group consisting of N, N-oxide, O, S, S-oxide, S(O), and S(O) 2. The point of attachment to the parent moiety is to any available ring carbon or ring heteroatom. Non-limiting examples of monocyclic heterocycloalkyl groups include azetidinyl, piperidyl, oxetanyl, pyrrolyl, piperazinyl, morpholinyl, thiomorpholinyl, thiazolidinyl, 1,4-dioxanyl, tetrahydrofuranyl (also referred to herein as oxolanyl), tetrahydrothiophenyl, beta lactam, gamma lactam, delta lactam, beta lactone, gamma lactone, delta lactone, and pyrrolidinone, and oxides thereof. Non-limiting examples of lower alkyl-substituted oxetanyl include the moiety:

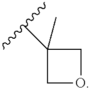 [SMILES: *C1(C)COC1]

It should be noted that in hetero-atom containing ring systems of this disclosure, there are no hydroxyl groups on carbon atoms adjacent to a N, O or S, as well as there are no N or S groups on carbon adjacent to another heteroatom.

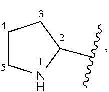 [SMILES: *C1CCCN1]

there is no-OH attached directly to carbons marked 2 and 5.

Any of the foregoing functional groups may be unsubstituted or substituted as described herein. The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound′ or “stable structure” is meant a compound that is sufficiently robust to survive isolation to a useful degree of purity from a reaction mixture, and formulation into an efficacious therapeutic agent.

The term “optionally substituted” means optional substitution of an available hydrogen atom of the relevant moiety with the specified groups, radicals or moieties.

When a variable appears more than once in a group, e.g., R⁶ in —N(R⁶)₂, or a variable appears more than once in a structure presented herein, the variables can be the same or different.

The line-, as a bond generally indicates a mixture of, or either of, the possible isomers, e.g., containing (R)— and(S)-stereochemical configuration. For example:

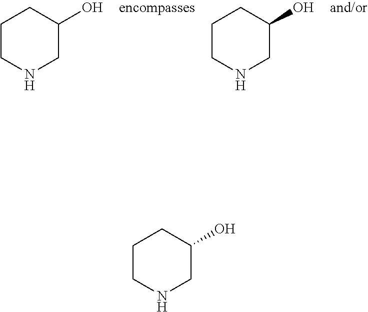 [SMILES: OC1CCCNC1 ; O[C@@H]1CCCNC1 ; O[C@H]1CCCNC1]

Furthermore, unwedged-bolded or unwedged-hashed lines are used in structures containing multiple stereocenters in order to depict relative configuration where it is known. For example:

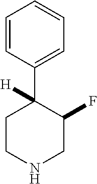 [SMILES: F[C@H]1CNCC[C@@H]1c1ccccc1]

means that the fluorine and hydrogen atoms are on the same face of the piperidine ring, but represents a mixture of, or one of, the possible isomers at right

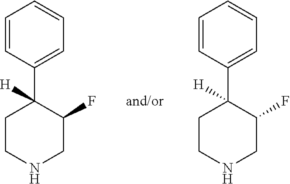 [SMILES: F[C@H]1CNCC[C@@H]1c1ccccc1 ; F[C@@H]1CNCC[C@H]1c1ccccc1]

whereas:

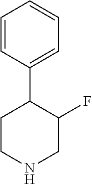 [SMILES: FC1CNCCC1c1ccccc1]

represents a mixture of, or one of, the possible isomers at right

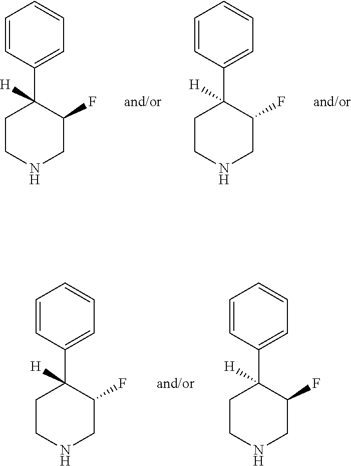 [SMILES: F[C@H]1CNCC[C@@H]1c1ccccc1 ; F[C@@H]1CNCC[C@H]1c1ccccc1 ; F[C@@H]1CNCC[C@@H]1c1ccccc1 ; F[C@H]1CNCC[C@H]1c1ccccc1]

In all cases, compound name(s) accompany the structure drawn and are intended to capture each of the stereochemical permutations that are possible for a given structural isomer based on the synthetic operations employed in its preparation. Lists of discrete stereoisomers that are conjoined using or indicate that the presented compound (e.g. ‘Example number’) was isolated as a single stereoisomer, and that the identity of that stereoisomer corresponds to one of the possible configurations listed. Lists of discrete stereoisomers that are conjoined using and indicate that the presented compound was isolated as a racemic mixture or diastereomeric mixture.

A specific absolute configuration is indicated by use of a wedged-bolded or wedged-hashed line. Unless a specific absolute configuration is indicated, the present disclosure is meant to encompass all such stereoisomeric forms of these compounds.

The wavy line, as used herein, indicates a point of attachment to the rest of the compound. Lines drawn into the ring systems, such as, for example:

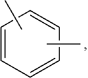

indicate that the indicated line (bond) may be attached to any of the substitutable ring carbon atoms.

In this specification, where there are multiple oxygen and/or sulfur atoms in a ring system, there cannot be any adjacent oxygen and/or sulfur present in said ring system.

As well known in the art, a bond drawn from a particular atom wherein no moiety is depicted at the terminal end of the bond indicates a methyl group bound through that bond to the atom, unless stated otherwise. For example:

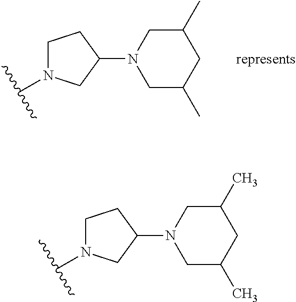 [SMILES: *N1CCC(N2CC(C)CC(C)C2)C1 ; *N1CCC(N2CC(C)CC(C)C2)C1]

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The compounds can be administered in the form of pharmaceutically acceptable salts. The term “pharmaceutically acceptable salt” refers to a salt which possesses the effectiveness of the parent compound and which is not biologically or otherwise undesirable (e.g., is neither toxic nor otherwise deleterious to the recipient thereof). When the compounds of the disclosure contain one or more acidic groups or basic groups, the disclosure includes the corresponding pharmaceutically acceptable salts.

Thus, the compounds of the disclosure that contain acidic groups (e.g., —COOH) can be used according to the disclosure as, for example but not limited to, alkali metal salts, alkaline earth metal salts or as ammonium salts. Examples of such salts include but are not limited to sodium salts, potassium salts, calcium salts, magnesium salts or salts with ammonia or organic amines such as, for example, ethylamine, ethanolamine, triethanolamine or amino acids. Compounds of the disclosure which contain one or more basic groups, i.e., groups which can be protonated, can be used according to the disclosure in the form of their acid addition salts with inorganic or organic acids as, for example but not limited to, salts with hydrogen chloride, hydrogen bromide, phosphoric acid, sulfuric acid, nitric acid, benzenesulfonic acid, methanesulfonic acid, p-toluenesulfonic acid, naphthalenedisulfonic acids, oxalic acid, acetic acid, trifluoroacetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, etc. If the compounds of the disclosure simultaneously contain acidic and basic groups in the molecule the disclosure also includes, in addition to the salt forms mentioned, inner salts or betaines (zwitterions). Salts can be obtained from the compounds of the disclosure by customary methods which are known to the person skilled in the art, for example by combination with an organic or inorganic acid or base in a solvent or dispersant, or by anion exchange or cation exchange from other salts. The present disclosure also includes all salts of the compounds of the disclosure which, owing to low physiological compatibility, are not directly suitable for use in pharmaceuticals but which can be used, for example, as intermediates for chemical reactions or for the preparation of pharmaceutically acceptable salts.

The terms “treating” or “treatment” (of, e.g., a disease, disorder, or conditions or associated symptoms, which together or individually may be referred to as “indications”) as used herein include: inhibiting the disease, disorder or condition, i.e., arresting or reducing the development of the disease or its biological processes or progression or clinical symptoms thereof; or relieving the disease, i.e., causing regression of the disease or its biological processes or progression and/or clinical symptoms thereof. “Treatment” as used herein also refers to control, amelioration, or reduction of risks to the subject afflicted with a disease, disorder or condition in which MsbA is involved. The terms “preventing”, or “prevention” or “prophylaxis” of a disease, disorder or condition as used herein includes: impeding the development or progression of clinical symptoms of the disease, disorder, or condition in a mammal that may be exposed to or predisposed to the disease, disorder or condition but does not yet experience or display symptoms of the disease, and the like.

As would be evident to those skilled in the art, subjects treated by the methods described herein are generally mammals, including humans and non-human animals (e.g., laboratory animals and companion animals), in whom the inhibition of MsbA activity is indicated or desired. The term “therapeutically effective amount” means the amount of the subject compound that will elicit the biological or medical response of a tissue, system, animal or human that is being sought by the researcher, veterinarian, medical doctor or other clinician.

The term “composition” as used herein is intended to encompass a product comprising a compound of the disclosure or a pharmaceutically acceptable salt thereof, together with one or more additional specified ingredients in the specified amounts, as well as any product which results, directly or indirectly, from combination of the specified ingredients in the specified amounts. Such term in relation to a pharmaceutical composition, is intended to encompass a product comprising the active ingredient(s), which include a compound of the disclosure or a pharmaceutically acceptable salt thereof, optionally together with one or more additional active ingredients, and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure, or a pharmaceutically acceptable salt thereof, and a pharmaceutically acceptable carrier. By “pharmaceutically acceptable” it is meant the carrier, diluent or excipient must be compatible with the other ingredients of the formulation and not deleterious to the recipient thereof.

As noted above, additional embodiments of the present disclosure are each directed to a method for the treatment a disease, disorder, or condition, or one or more symptoms thereof (“indications”) in which MsbA is involved and for which the inhibition of MsbA is desired, which method comprises administering to a subject in need of such treatment a therapeutically effective amount of a compound of the disclosure, or a pharmaceutically acceptable salt thereof, or a pharmaceutical composition comprising said compound or salt thereof.

In another embodiment, the present disclosure is directed to a method for the manufacture of a medicament for inhibition of MsbA activity in a subject comprising combining a compound of the present disclosure, or a pharmaceutically acceptable salt thereof, with a pharmaceutical carrier or diluent.

One such embodiment provides a method of treating MRD gram-negative infections in a subject in need thereof, said method comprising administering to a subject in need of such treatment a therapeutically effective amount of a compound of the disclosure, or a pharmaceutically acceptable salt thereof, or a pharmaceutical composition comprising said compound or salt thereof. In one such embodiment, the subject is a human.

The present disclosure includes within its scope prodrugs of the compounds of this disclosure. In general, such prodrugs will be functional derivatives of the compounds of this disclosure which are readily convertible in vivo into the required compound. Thus, in the methods of treatment of the present disclosure, the terms “administration of” or “administering a” compound shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985. Metabolites of these compounds include active species produced upon introduction of compounds of this disclosure into the biological milieu.

The compounds of the present disclosure may be used in combination with one or more other drugs in the treatment, prevention, control, amelioration, or reduction of risk of diseases or conditions for which compounds of the disclosure or the other drugs may have utility, where the combination of the drugs together are safer or more effective than either drug alone. Such other drug(s) may be administered, by a route and in an amount commonly used therefore, contemporaneously or sequentially with a compound of Formula I. When a compound of Formula I is used contemporaneously with one or more other drugs, a pharmaceutical composition in unit dosage form containing such other drugs and the compound of Formula I is preferred. However, the combination therapy may also include therapies in which the compound of Formula I and one or more other drugs are administered on different overlapping schedules. It is also contemplated that when used in combination with one or more other active ingredients, the compounds of the present disclosure and the other active ingredients may be used in lower doses than when each is used singly. Accordingly, the pharmaceutical compositions of the present disclosure include those that contain one or more other active ingredients, in addition to a compound of Formula I.

The present compounds may be used in conjunction with one or more additional therapeutic agents. For example, the compounds of the disclosure can be used in combination with antibiotic agents for the treatment of infections known or suspected to be caused by A. baumannii or polymicrobial infections where A. baumannii is a known or suspected etiological agent. Examples of antibiotics that can be combined with the compounds of this disclosure include, but are not limited to, penicillins (e.g., phenoxymethylpenicillin, dicloxacillin, amoxicillin with clavulanic acid, ampicillin, nafcillin, oxacillin, penicillin V, penicillin G, and other known penicillins), cephalosporins (e.g., cefaclor, cefazolin, cefadroxil, cephalexin, cefuroxime, cefixime, cefoxitin, ceftriaxone, ceftibuten, cefepime, and other known cephalosporins), carbapenems (e.g., ertapenem, doripenem, imipenem/cilastatin, meropenem, and other known carbapenems), tetracyclines (e.g., doxycycline, minocycline, sarecycline, tigecycline, and other know tetracyclines), macrolides (e.g., erythromycin, clarithromycin, azithromycin, fidaxomicin, roxithromycin, and other know macrolides), lincosamides (e.g., clindamycin, lincomycin, and other known lincosamides), fluoroquinolones (e.g., ciprofloxacin, ofloxacin, levofloxacin, moxifloxacin, and other known fluoroquinolones), sulfonamides (e.g., sulfamethoxazole with trimethoprim, sulfasalazine, sulfacetamide, sulfadiazine silver, and other known sulfonamides), aminoglycosides (e.g., amikacin, gentamicin, kanamycin, neomycin, netilmicin, tobramycin, paromomycin, streptomycin, spectinomycin, and other known aminoglycosides), glycopeptides (e.g., vancomycin, oritavancin, telavancin, and other known glycopeptides), or polypeptides (e.g., colistin, polymyxin B and other known polypetides). When co-administered with any of these antibiotics or others, the combination of the compound of the disclosure and the antibiotic can provide a synergistic effect. The terms “synergistic effect” and “synergy” indicate that the effect produced when two or more drugs are co-administered is greater than would be predicted based on the effect produced when the compounds are administered individually.

The above combinations include combinations of a compound of the present disclosure not only with one other active compound, but also with two or more other active compounds. Likewise, compounds of the present disclosure may be used in combination with other drugs that are used in the prevention, treatment, control, amelioration, or reduction of risk of the diseases or conditions for which compounds of the present disclosure are useful. Such other drugs may be administered, by a route and in an amount commonly used therefore, contemporaneously or sequentially with a compound of the present disclosure. When a compound of the present disclosure is used contemporaneously with one or more other drugs, a pharmaceutical composition containing such other drugs in addition to the compound of the present disclosure is preferred. Accordingly, the pharmaceutical compositions of the present disclosure include those that also contain one or more other active ingredients, in addition to a compound of the present disclosure.

The weight ratio of the compound of the present disclosure to the other active ingredient(s) may be varied and will depend upon the effective dose of each ingredient. Generally, an effective dose of each will be used. Thus, for example, when a compound of the present disclosure is combined with another agent, the weight ratio of the compound of the present disclosure to the other agent will generally range from about 1000:1 to about 1:1000, or from about 200:1 to about 1:200. Combinations of a compound of the present disclosure and other active ingredients will generally also be within the aforementioned range, but in each case, an effective dose of each active ingredient should be used.

In such combinations the compound of the present disclosure and other active agents may be administered separately or in conjunction. In addition, the administration of one element may be prior to, concurrent to, or subsequent to the administration of other agent(s), and via the same or different routes of administration.

The compounds of the present disclosure may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, buccal or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals the compounds of the disclosure are effective for use in humans.

The pharmaceutical compositions for the administration of the compounds of this disclosure may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformLy and intimately bringing the active ingredient into association with a liquid carrier or a finely divided solid carrier or both, and then, if necessary, shaping the product into the desired formulation. In the pharmaceutical composition the active compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, solutions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets may be uncoated, or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and U.S. Pat. No. 4,265,874 to form osmotic therapeutic tablets for control release. Oral tablets may also be formulated for immediate release, such as fast melt tablets or wafers, rapid dissolve tablets or fast dissolve films.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanthin and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanthin, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

The compounds of the present disclosure may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions and the like, containing the compounds of the present disclosure are employed. Similarly, transdermal patches may also be used for topical administration.

The pharmaceutical composition and method of the present disclosure may further comprise other therapeutically active compounds as noted herein which are usually applied in the treatment of the above-mentioned pathological conditions.

In the treatment, prevention, control, amelioration, or reduction of risk of conditions which require inhibition of MsbA activity an appropriate dosage level will generally be about 0.01 to 500 mg per kg patient body weight per day which can be administered in single or multiple doses. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions may be provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day or may be administered once or twice per day. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Methods for preparing the compounds of this disclosure are illustrated in the following Schemes and Examples. Starting materials are made according to procedures known in the art or as illustrated herein.

### Methods For Making the Compounds of Formula (I):

The Compounds of Formula (I) may be prepared from known or readily prepared starting materials, following methods known to one skilled in the art of organic synthesis. Representative methods useful for making the Compounds of Formula (I) are set forth in the Examples below. Alternative synthetic pathways and analogous structures will be apparent to those skilled in the art of organic synthesis.

One skilled in the art of organic synthesis will recognize that the synthesis of multicyclic and/or heterocyclic cores contained in Compounds of Formula (I) may require protection of certain functional groups (i.e., derivatization for the purpose of chemical compatibility with a particular reaction condition). Suitable protecting groups for the various functional groups of these Compounds and methods for their installation and removal are well known in the art of organic chemistry. A summary of many of these methods can be found in Greene et al., Protective Groups in Organic Synthesis, Wiley-Interscience, New York, (1999).

One skilled in the art of organic synthesis will also recognize that one route for the synthesis of the multicyclic heterocycle cores of the Compounds of Formula (I) may be more desirable depending on the choice of appendage substituents.

Additionally, one skilled in the art will recognize that in some cases the order of reactions may differ from that presented herein to avoid functional group incompatibilities and thus adjust the synthetic route accordingly.

The preparation of multicyclic intermediates useful for making the multicyclic and/or heterocyclic cores of the Compounds of Formula (I) have been described in the literature and in compendia such as “Comprehensive Heterocyclic Chemistry” editions I, II and III, published by Elsevier and edited by A. R. Katritzky & R. JK Taylor. Manipulation of the required substitution patterns have also been described in the available chemical literature as summarized in compendia such as “Comprehensive Organic Chemistry” published by Elsevier and edited by

DH R. Barton and W. D. Ollis; “Comprehensive Organic Functional Group Transformations” edited by edited by A. R. Katritzky & R. JK Taylor and “Comprehensive Organic Transformation” published by Wiley-CVH and edited by R. C. Larock.

The starting materials used and the intermediates prepared using the methods set forth in the Examples below may be isolated and purified if desired using conventional techniques, including but not limited to filtration, distillation, crystallization, chromatography and alike. Such materials can be characterized using conventional means, including physical constants and spectral data.

One skilled in the art will be aware of standard formulation techniques as set forth in the open literature as well as in textbooks such as Zheng, “Formulation and Analytical Development for Low-dose Oral Drug Products,” Wiley, 2009, ISBN.

### General Schemes for Representative Compounds and Intermediates of Interest:

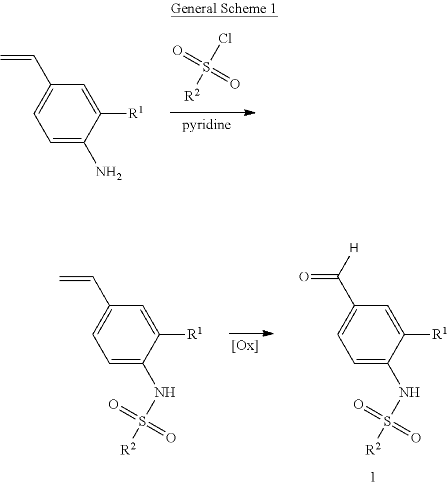

Aldehyde intermediates of the general structure 1 in the present disclosure can be accomplished in two steps starting from appropriately functionalized 4-vinyl-anilines or-aminoheterocycles. The first step involves the reaction of the appropriately functionalized 4-vinyl-anilines or-aminoheterocycles with a sulfonyl chloride in pyridine or an organic solvent with an appropriate base. Styrene oxidation can be accomplished with a variety of homogeneous or heterogeneous catalysts to provide the desired aldehyde.

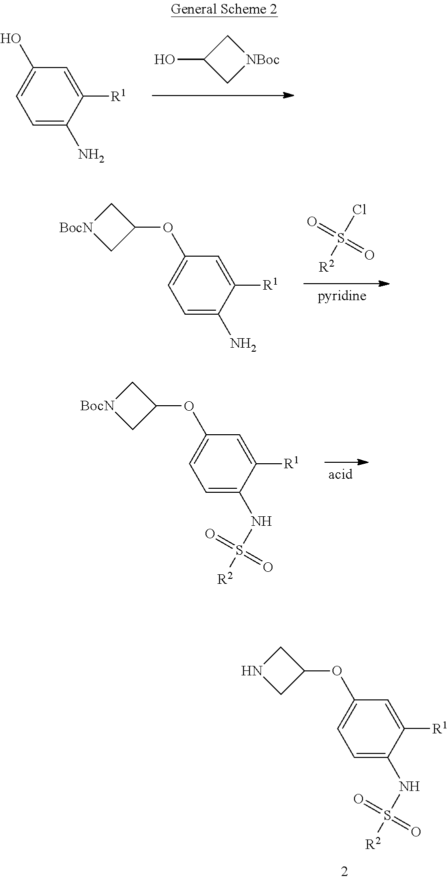

Azetidine intermediates of the general structure 2 in the present disclosure can be prepared in three steps starting from appropriately functionalized 4-aminophenol. The first step involves the reaction of an alcohol with the appropriately functionalized 4-aminophenol under Mitsunobu conditions. Reaction of the resulting aniline with a sulfonyl chloride in pyridine or an organic solvent with an appropriate base provides the desired sulfonamide or sulfonamide. Deprotection of Boc-protected amine using acids such as TFA or HCl provides the desired azetidine.

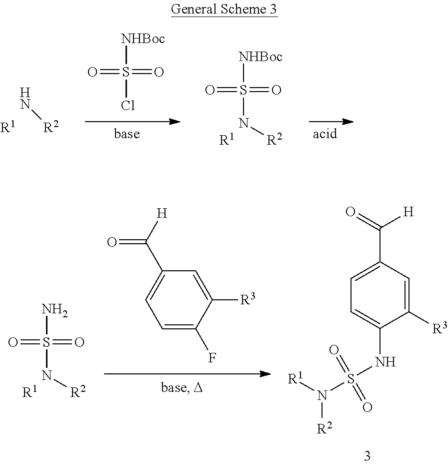

Aldehyde intermediates of the general structure 3 can be prepared in three steps starting from appropriately functionalized primary or secondary amines. The first step involves sulfonylation with tert-butyl chlorosulfonylcarbamate with the appropriately functionalized primary or secondary amines to form the desired sulfamide. Deprotection of the Boc-protecting group is accomplished using acids such as TFA or HCl. Nucleophilic aromatic substation of the deprotected sulfamide with appropriately functioned 4-fluorobenzaldhye furnishes the desired aldehyde.

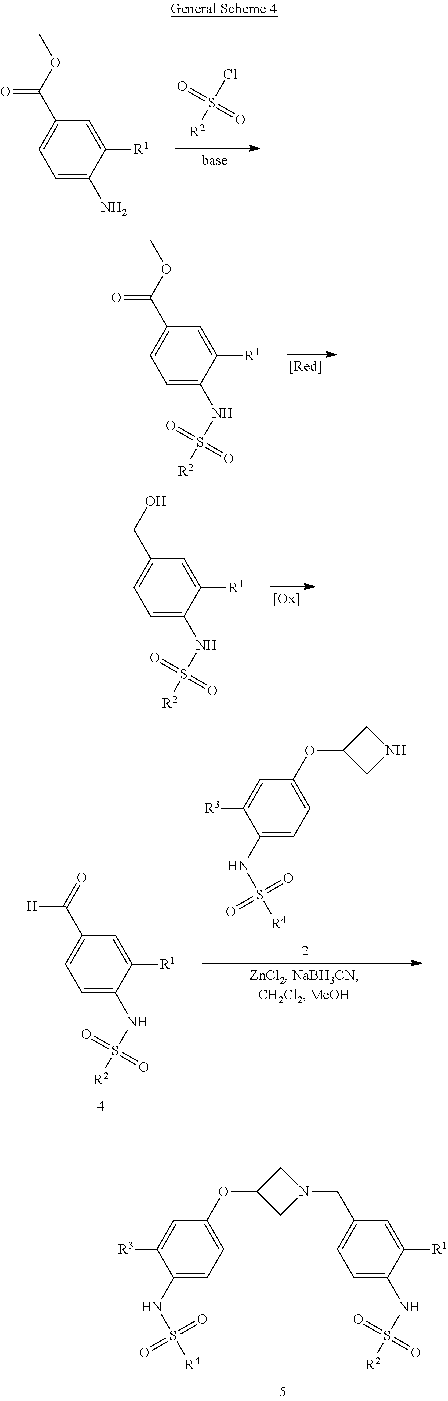

Aldehyde intermediates of the general structure 4 and azetidine-linked intermediates of the general structure 5 in the present disclosure can be prepared in four steps starting from appropriately functionalized anilines or aminoheterocycles. The first step involves the reaction of the appropriately functionalized anilines or aminoheterocycles with a sulfonyl chloride in an organic solvent with an appropriate base to furnish arylsulfonamides. The second step involves reduction of an ester to the corresponding alcohol with a suitable reducing agent such as DIBAL-H in a suitable solvent. The resulting alcohol is oxidized to the desired aldehyde using a suitable oxidizing agent such as MnO2 in a suitable solvent. Aldehydes of the structure 4 can then be reacted with azetidines of the structure 2 under reductive amination conditions to furnish the desired azetidine linked intermediates.

### Preparation of Compounds and Intermediates

The disclosure is illustrated by the following examples. For all of the examples, standard work-up and purification methods known to those skilled in the art can be utilized. Unless otherwise indicated, all temperatures are expressed in° C. (degrees Centigrade). All reactions are conducted at room temperature unless otherwise noted. Synthetic methodologies illustrated herein are intended to exemplify the applicable chemistry through the use of specific examples and are not indicative of the scope of the disclosure.

The following abbreviations are used herein:
OAc acetate
AcOH acetic acid
anh anhydrous
aq aqueous
Bn benzyl
BSI bloodstream infection
Boc or BOC tert-butoxycarbonyl
Bz benzoyl
Cbz Benzyloxycarbonyl
CAMHB cation-adjusted Mueller Hinton broth
calc'd calculated
CDI carbonyl diimidazole
Celite diatomaceous earth
CRAB carbapenem-resistant Acinetobacter baumannii
dba dibenzylidineacetone
DBAD di-tert-butyl azodicarboxylate
DBU 1,8-diazabicyclo(5.4.0) undec-7-ene
DCE dichloroethane
DCM dichloromethane
DIEA or DIPEA N,N-diisopropylethylamine
DMAP 4-dimethylaminopyridine
DMF N, N-dimethylformamide
DMP Dess-Martin periodinane
DMSO dimethyl sulfoxide
DNA deoxyribonucleic acid
EDC 1-Ethyl-3-(3-dimethylaminopropyl) carbodiimide
ESI electrospray ionization
Et ethyl
Et20 diethyl ether
EtOH Ethanol
EtOAc ethyl acetate
Et3N triethylamine
g gram
h hour
HAP hospital-acquired pneumonia
HATU 1-[bis(dimethylamino)methylene]-1H-1,2,3-triazolo[4,5-b]pyridinium 3-oxide hexafluorophosphate
HCl hydrochloric acid
hex hexanes
HMDS hexamethyldisilazane
HPLC high-performance liquid chromatography
IM inner membrane
int intermediate
IPA isopropanol
iPr isopropyl
LC liquid chromatography
LC/MS liquid chromatography mass spectrometry
LPS lipopolysaccharide
MDR Multi-drug resistant
Me Methyl
MeCN acetonitrile
MeOH methanol
mg milligrams
min minutes
MP—(OAc) 3BH MP-triacetoxyborohydride
HL microliters
mL milliliters
mmol millimoles
MS mass spectrometry
NMI N-methylimidazole
NMR nuclear magnetic resonance spectroscopy
OM outer membrane
dppf 1,1′-bis(diphenylphosphino) ferrocene
Pet. Petroleum
Ph phenyl
ppm parts per million
psi pounds per square inch
Py pyridine
Pd palladium
PPh3 triphenylphosphine
RT or rt room temperature
Sat saturated Sat
SFC supercritical fluid chromatography
TBAF tert-butyl ammonium fluoride
TBS or TBDMS tert-butyldimethylsilyl
TBSCl tert-butyldimethylsilyl chloride
t-Bu tert-butyl
TEA triethylamine
TBDPS tert-butyldiphenylsilyl
TBDPSCI tert-butyldiphenylsilyl chloride
TCFH chloro-N,N,N′ N′-tetramethylformamidinium hexafluorophosphate
TMSN3 trimethylsilyl azide
TMSBr trimethylsilyl bromide
TEA triethylamine
TFA trifluoroacetic acid
THF tetrahydrofuran
TLC thin layer chromatography
TMS trimethylsilyl
Tris tris(hydroxymethyl)aminomethane
UPLC ultra-performance liquid chromatography

### General Methods

Solvents, reagents, and intermediates that are commercially available were used as received. Intermediates that are not commercially available were prepared in the manner as described below. ¹H NMR spectra are reported as ppm downfield from Me4Si with number of protons, multiplicities, and coupling constants in Hertz indicated parenthetically. Where LC/MS data are presented, the observed parent ion is given. Flash column chromatography was performed using pre-packed normal phase silica or bulk silica.

### Intermediate 1

### Preparation of Intermediate Compound Int-1

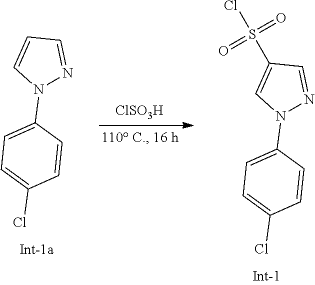

### Step 1:1-(4-chlorophenyl)-1H-pyrazole-4-sulfonyl chloride (Int-1)

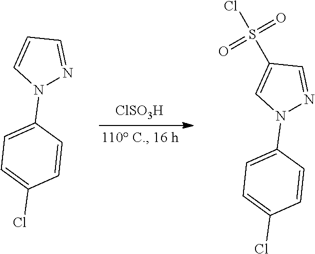

1-(4-Chlorophenyl)-1H-pyrazole (1 g, 5.60 mmol) was added dropwise to chlorosulfuric acid (1.5 mL, 22.40 mmol) while stirring at 0° C. under N₂. The reaction mixture was heated at 110° C. for 16 h. The reaction mixture was cooled to room temperature and poured carefully into ice water (10 mL) and extracted with CH₂Cl₂ (10 mL×3). The combined organic phases were washed with brine (10 mL), dried over anhydrous Na₂SO₄, filtered, and concentrated in vacuo. The residue was purified by flash chromatography on SiO₂ [Isco®, SepaFlash® 12 g column, isocratic eluent of 15% EtOAc/Pet. Ether at 30 mL/min] to provide 1-(4-chlorophenyl)-1H-pyrazole-4-sulfonyl chloride. ¹H NMR (500 MHZ, CDCl₃) ¿ (ppm) 8.51 (s, 1H), 8.18 (s, 1H), 7.68 (d, J=9.0 Hz, 2H), 7.52 (d, J=9.0 Hz, 2H).

The following intermediate of the present disclosure was made using the methods described in Intermediate 1 above, and substituting the appropriate reactants and/or reagents:

| Compound | Structure |
|---|---|
| Int-2 | 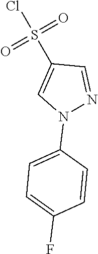 [SMILES: O=S(=O)(Cl)c1cnn(-c2ccc(F)cc2)c1] |

### Intermediate 3

### Preparation of Intermediate Compound Int-3

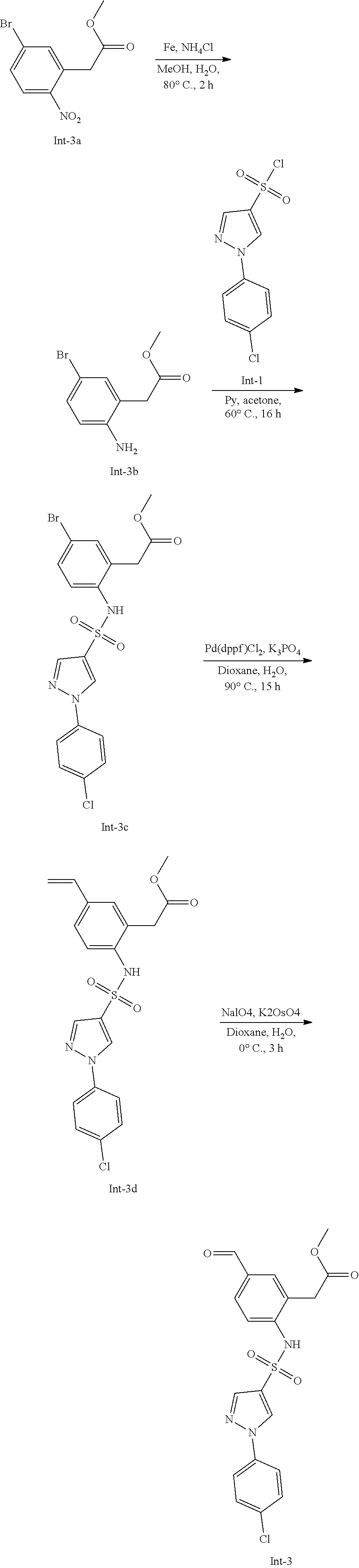 [SMILES: COC(=O)Cc1cc(Br)ccc1[N+](=O)[O-] ; COC(=O)Cc1cc(Br)ccc1N ; O=S(=O)(Cl)c1cnn(-c2ccc(Cl)cc2)c1 ; COC(=O)Cc1cc(Br)ccc1NS(=O)(=O)c1cnn(-c2ccc(Cl)cc2)c1 ; C=Cc1ccc(NS(=O)(=O)c2cnn(-c3ccc(Cl)cc3)c2)c(CC(=O)OC)c1 ; COC(=O)Cc1cc(C=O)ccc1NS(=O)(=O)c1cnn(-c2ccc(Cl)cc2)c1]

### Step 1: methyl 2-(2-amino-5-bromophenyl)acetate (Int-3b)

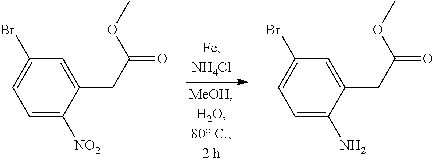

To a solution of methyl 2-(5-bromo-2-nitrophenyl)acetate (1.5 g, 5.47 mmol) in MeOH (20 mL) and water (2 mL) was added NH₄Cl (2.93 g, 54.7 mmol) and iron (3.06 g, 54.7 mmol) at room temperature. The reaction mixture was stirred at 80° C. for 2 h. The mixture was filtered and the solvent was concentrated in vacuo, then diluted with water (20 mL) and extracted with EtOAc (2×20 mL). The combined organic layers were washed with water (20 mL), dried over Na₂SO₄, filtered, and the filtrate was concentrated in vacuo. The resulting residue was purified by flash chromatography on SiO₂ [Isco®; SepaFlash® 12 g column, isocratic eluent of 19% EtOAc/Pet. ether at 30 mL/min] to provide methyl 2-(2-amino-5-bromophenyl)acetate. MS (ESI, m/z): 244.2 [M+H⁺]. ¹H NMR (400 MHZ, CD₃OD) δ (ppm) 7.11-7.16 (m, 2H), 6.67 (d, J=8.4 Hz, 1H), 3.69 (s, 3H), 3.55 (s, 2H).

### Step 2: methyl 2-(5-bromo-2-(1-(4-chlorophenyl)-1H-pyrazole-4-sulfonamido)phenyl)acetate (Int-3c)

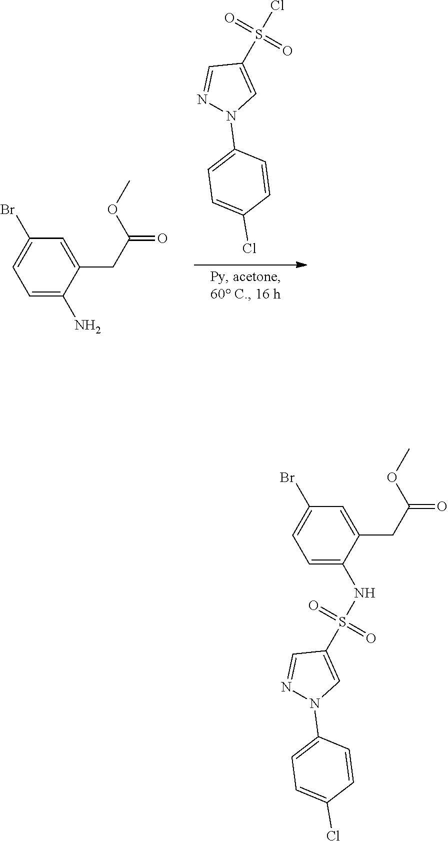 [SMILES: COC(=O)Cc1cc(Br)ccc1N ; O=S(=O)(Cl)c1cnn(-c2ccc(Cl)cc2)c1 ; COC(=O)Cc1cc(Br)ccc1NS(=O)(=O)c1cnn(-c2ccc(Cl)cc2)c1]

To a solution of methyl 2-(2-amino-5-bromophenyl)acetate (Int-3b; 400 mg, 1.639 mmol) in acetone (3 mL) and pyridine (1 mL) was added 1-(4-chlorophenyl)-1H-pyrazole-4-sulfonyl chloride (Int-1; 681 mg, 2.458 mmol) at room temperature. The reaction mixture was stirred at 60° for 16 h. The solvent was removed in vacuo, and the resulting residue was purified by flash chromatography on SiO₂ [Isco®, SepaFlash® 12 g column, eluent of 0 to 20% EtOAc/Pet. ether at 30 mL/min] to provide methyl 2-(5-bromo-2-(1-(4-chlorophenyl)-1H-pyrazole-4-sulfonamido)phenyl)acetate. MS (ESI, m/z): 486.0 [M+H⁺]. ¹H NMR (400 MHZ, CDCl₃) δ (ppm) 8.21 (s, 1H), 8.09 (s, 1H), 7.90 (s, 1H), 7.60 (d, J=9.0 Hz, 2H), 7.45-7.50 (m, 2H), 7.40-7.44 (m, 1H), 7.34-7.39 (m, 2H), 3.73 (s, 3H), 3.49 (s, 2H).

### Step 3: methyl 2-(2-(1-(4-chlorophenyl)-1H-pyrazole-4-sulfonamido)-5-vinylphenyl)acetate (Int-3d)

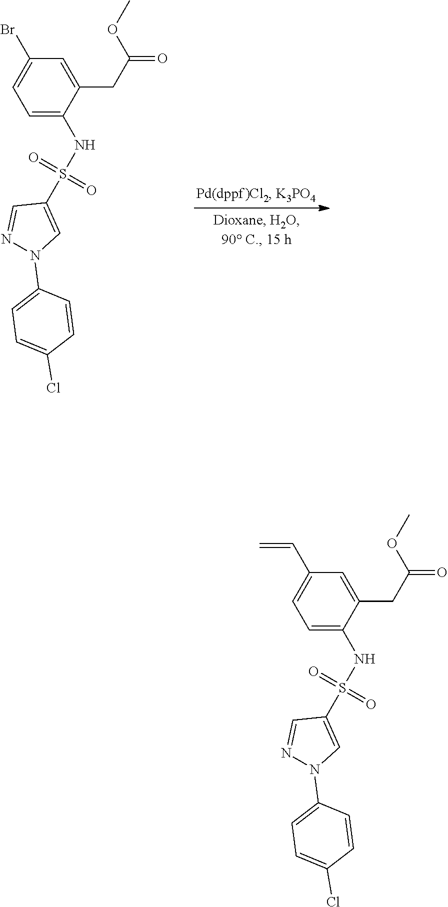 [SMILES: COC(=O)Cc1cc(Br)ccc1NS(=O)(=O)c1cnn(-c2ccc(Cl)cc2)c1 ; C=Cc1ccc(NS(=O)(=O)c2cnn(-c3ccc(Cl)cc3)c2)c(CC(=O)OC)c1]

A mixture of methyl 2-(5-bromo-2-(1-(4-chlorophenyl)-1H-pyrazole-4-sulfonamido)phenyl)acetate (Int-3c; 450 mg, 0.928 mmol), Pd (dppf) Cl2 (67.9 mg, 0.093 mmol), and K3PO4 (591 mg, 2.78 mmol) in 1,4-dioxane (8 mL) and water (0.8 mL) was degassed under N₂ (3×) and the reaction mixture was stirred at 90° C. for 16 h. The reaction mixture was filtered and the filtrate was concentrated in vacuo. The resulting residue was diluted with EtOAc (20 mL) and washed with water (20 mL). The organic layer was separated, dried over Na₂SO₄, filtered, and concentrated in vacuo. The resulting residue was purified by flash chromatography on SiO₂ [Isco®, SepaFlash® 4g column, isocratic eluent of 20% EtOAc/Pet. ether at 30 mL/min] to provide methyl 2-(2-(1-(4-chlorophenyl)-1H-pyrazole-4-sulfonamido)-5-vinylphenyl)acetate. MS (ESI, m/z): 432.1 [M+H⁺]. ¹H NMR (400 MHZ, CDCl₃) δ (ppm) 8.18-8.21 (m, 1H), 8.13 (s, 1H), 7.89 (s, 1H), 7.56-7.61 (m, 2H), 7.44-7.48 (m, 3H), 7.32-7.36 (m, 1H), 7.23 (s, 1H), 6.64 (dd, J=17.4, 10.76 Hz, 1H), 5.71 (d, J=16.8 Hz, 1H), 5.27 (d, J=11.4 Hz, 1H), 3.71-3.73 (m, 3H), 3.51 (s, 2H).

### Step 4: methyl 2-(2-(1-(4-chlorophenyl)-1H-pyrazole-4-sulfonamido)-5-formylphenyl)acetate (Int-3)

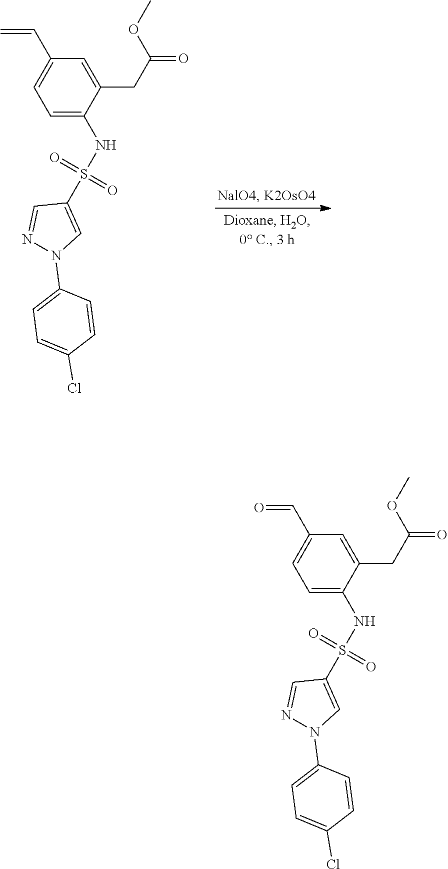 [SMILES: C=Cc1ccc(NS(=O)(=O)c2cnn(-c3ccc(Cl)cc3)c2)c(CC(=O)OC)c1 ; COC(=O)Cc1cc(C=O)ccc1NS(=O)(=O)c1cnn(-c2ccc(Cl)cc2)c1]

To a solution of methyl 2-(2-(1-(4-chlorophenyl)-1H-pyrazole-4-sulfonamido)-5-vinylphenyl)acetate (Int-3d; 130 mg, 0.301 mmol) in dioxane (4 mL) and water (1 mL) was added 2,6-dimethylpyridine (64.5 mg, 0.602 mmol), potassium dioxidodioxoosmium dihydrate (22.18 mg, 0.060 mmol), and sodium periodate (258 mg, 1.204 mmol) at 0° C. in a cooling bath. The cooling bath was removed and the reaction mixture was allowed to warm to room temperature while stirring for 3 h. The reaction mixture was filtered and the filtrate was quenched with aqueous Na2SO₃ (5 mL) and water (10 mL) and extracted with EtOAc (10 mL×3).

The combined organic layers were washed with brine (10 mL), dried over anhydrous Na₂SO₄, filtered, and concentrated in vacuo. The resulting residue was purified by preparative TLC (Pet. ether/EtOAc=1:1) to provide methyl 2-(2-(1-(4-chlorophenyl)-1H-pyrazole-4-sulfonamido)-5-formylphenyl)acetate. MS (ESI) m/z: 434.1 [M+H⁺].

### Intermediate 4

### Preparation of Intermediate Compound Int-4

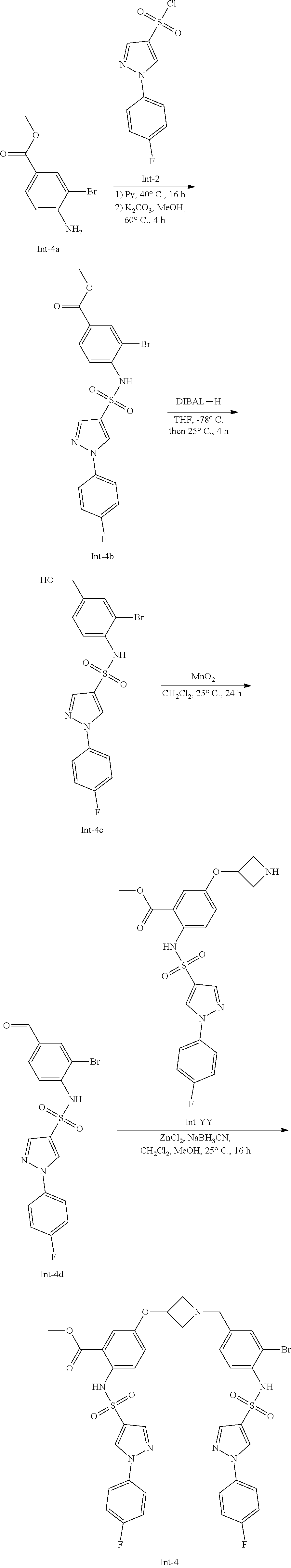

### Step 1: methyl 3-bromo-4-((1-(4-fluorophenyl)-1H-pyrazole)-4-sulfonamido)benzoate (Int-4b)

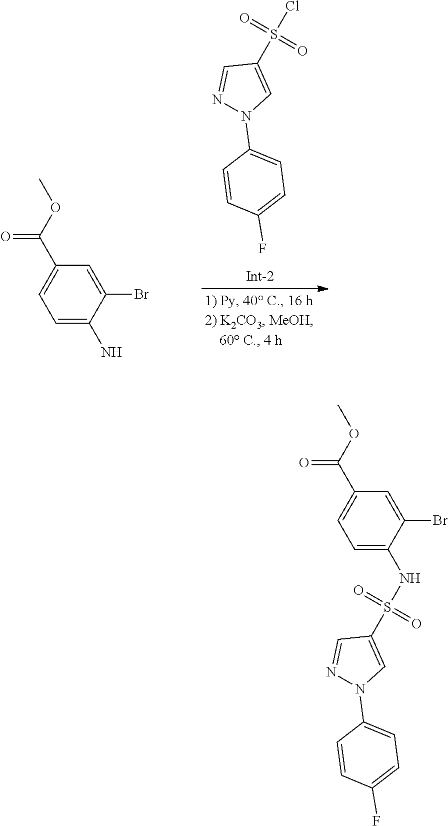 [SMILES: COC(=O)c1ccc(N)c(Br)c1 ; O=S(=O)(Cl)c1cnn(-c2ccc(F)cc2)c1 ; COC(=O)c1ccc(NS(=O)(=O)c2cnn(-c3ccc(F)cc3)c2)c(Br)c1]

To a stirred mixture of methyl 4-amino-3-bromobenzoate (2 g, 8.69 mmol) in pyridine (20 mL) was added 1-(4-fluorophenyl)-1H-pyrazole-4-sulfonyl chloride (Int-2; 3.40 g, 13.04 mmol) and the mixture was heated to 40° C. with stirring for 16 h. The solvent was concentrated in vacuo. The resulting residue was dissolved in MeOH (20 mL), K₂CO₃ (3.60 g, 26.1 mmol) was added, and the mixture was stirred for another 4 h at 60° C. H₂O (50 mL) was added and the mixture was extracted with EtOAc (2×100 mL). The combined organic fractions were washed with brine (50 mL), dried over anhydrous Na₂SO₄, filtered, and concentrated in vacuo. The resulting residue was purified by column chromatography on SiO₂ [Isco®, Silica 40 g column, eluent of 0 to 50% EtOAc/Pet. ether at 30 mL/min] to provide methyl 3-bromo-4-((1-(4-fluorophenyl)-1H-pyrazole)-4-sulfonamido)benzoate. MS (ESI, m/z): 456.0 [M+H⁺].

### Step 2: N-(2-bromo-4-(hydroxymethyl)phenyl)-1-(4-fluorophenyl)-1H-pyrazole-4-sulfonamide (Int-4c)

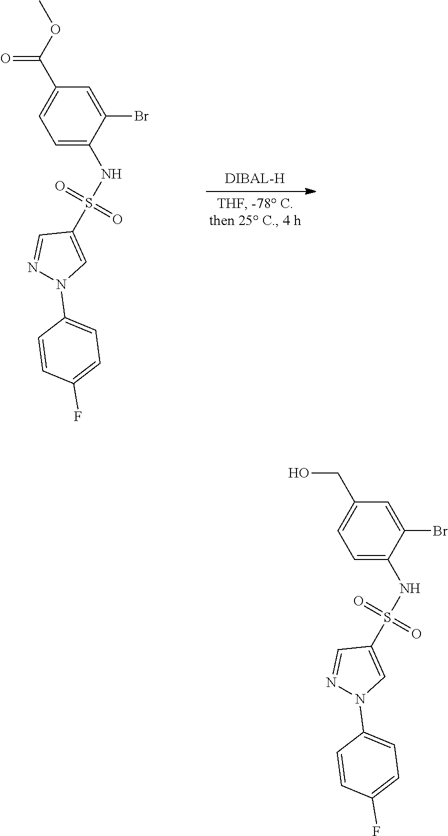 [SMILES: COC(=O)c1ccc(NS(=O)(=O)c2cnn(-c3ccc(F)cc3)c2)c(Br)c1 ; O=S(=O)(Nc1ccc(CO)cc1Br)c1cnn(-c2ccc(F)cc2)c1]

To a stirred mixture of methyl 3-bromo-4-((1-(4-fluorophenyl)-1H-pyrazole)-4-sulfonamido)-benzoate (Int-4b; 2.9 g, 6.38 mmol) in THF (40 mL) at −78° C. was added a 1M THF solution of

DIBAL-H (19.15 mL, 19.15 mmol). The mixture was warmed to room temperature and stirred for 4 h under N₂. Potassium sodium tartrate solution (sat. aq., 20 mL) was added and the mixture was stirred for an additional 30 min. The mixture was extracted with EtOAc (2×100 mL) and the combined organic fractions were dried over any. Na₂SO₄, filtered, and concentrated in vacuo. The resulting residue was purified by column chromatography on SiO₂ [Isco®, Silica 20 g column, eluent of 0 to 40% EtOAc/Pet. ether at 35 mL/min] to provide N-(2-bromo-4-(hydroxymethyl)phenyl)-1-(4-fluorophenyl)-1H-pyrazole-4-sulfonamide. MS (ESI, m/z): 428.0 [M+H⁺].

### Step 3: N-(2-bromo-4-formylphenyl)-1-(4-fluorophenyl)-1H-pyrazole-4-sulfonamide (Int-4d)

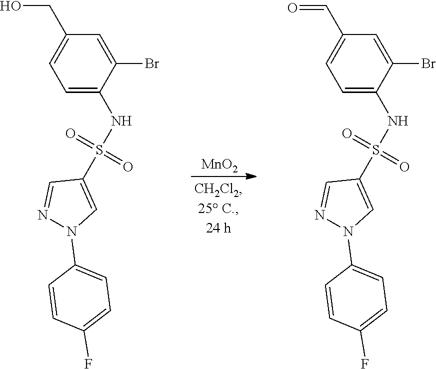

To a stirred mixture of N-(2-bromo-4-(hydroxymethyl)phenyl)-1-(4-fluorophenyl)-1H-pyrazole-4-sulfonamide (Int-4c; 2 g, 4.69 mmol) in CH₂Cl2 (20 mL) was added manganese (IV) oxide (8.16 g, 94 mmol) and the mixture was stirred at 25° C. for 24 h. The mixture was filtered and the filter cake was washed with CH₂Cl₂ (40 mL). The filtrate was concentrated in vacuo to provide N-(2-bromo-4-formylphenyl)-1-(4-fluorophenyl)-1H-pyrazole-4-sulfonamide, which was used in the subsequent step without further purification. MS (ESI, m/z): 426.1 [M+H⁺]. Step 4: methyl 5-((1-(3-bromo-4-((1-(4-fluorophenyl)-1H-pyrazole)-4-sulfonamido)-benzyl) azetidin-3-yl)oxy)-2-((1-(4-fluorophenyl)-1H-pyrazole)-4-sulfonamido)benzoate (Int-4)

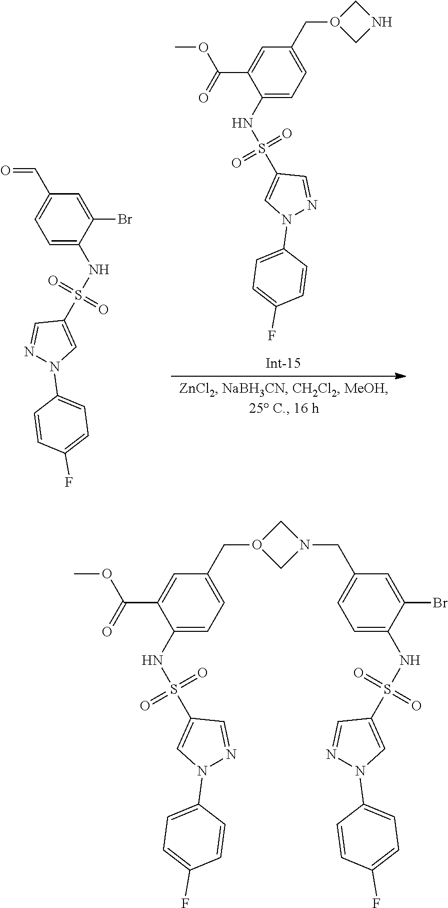

To a stirred mixture of zinc (II) chloride (305 mg, 2.240 mmol), methyl 5-(azetidin-3-yloxy)-2-((1-(4-fluorophenyl)-1H-pyrazole)-4-sulfonamido)benzoate (Int-15; 500 mg, 1.120 mmol), and N-(2-bromo-4-formylphenyl)-1-(4-fluorophenyl)-1H-pyrazole-4-sulfonamide (Int-4d; 475 mg, 1.120 mmol) in CH₂Cl₂ (8 mL) and MeOH (2 mL) was added TEA (0.311 mL, 2.240 mmol) and the mixture was stirred at 25° C. for 10 min. NaBH₃CN (211 mg, 3.36 mmol) was added and the mixture was stirred at 25° C. for 16 h. The solution was concentrated in vacuo, and the resulting residue was purified by column chromatography on SiO₂ [Isco®, Silica 20 g column, eluent of 0 to 40% EtOAc/Pet. ether at 35 mL/min| to provide methyl 5-((1-(3-bromo-4-((1-(4-fluorophenyl)-1H-pyrazole)-4-sulfonamido)-benzyl) azetidin-3-yl)oxy)-2-((1-(4-fluorophenyl)-1H-pyrazole)-4-sulfonamido)benzoate. MS (ESI, m/z): 856.0 [M+H⁺].

### Intermediate 8

### Preparation of Intermediate Compound Int-8

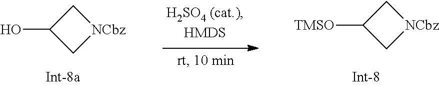

### Step 1: benzyl 3-((trimethylsilyl)oxy) azetidine-1-carboxylate (Int-8)

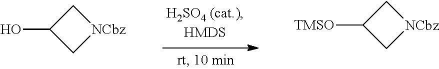

To a solution of benzyl 3-hydroxyazetidine-1-carboxylate (2 g, 9.65 mmol) in HMDS (2.023 mL, 9.65 mmol) was added concentrated H₂SO₄ (10.3 μL, 0.193 mmol). The reaction mixture was stirred at ambient temperature for 10 min. To the resulting solution was added brine (30 mL) and the mixture was extracted with EtOAc (30 mL). The organic fraction was washed with brine (30 mL), dried over anh. Na₂SO₄, filtered, and concentrated in vacuo. The resulting residue was purified by flash chromatography on SiO₂ [Isco®, Agela® 4 g column, eluent of 0 to 1% EtOAc/Pet. ether gradient at 30 mL/min]provide benzyl 3-((trimethylsilyl)oxy) azetidine-1-carboxylate. ¹H NMR (400 MHZ, CDCl₃) δ ppm 7.38-7.29 (m, 5H), 5.09 (s, 2H), 4.56 (tt, J=4.8, 6.8 Hz, 1H), 4.18 (dd, J=6.8, 9.6 Hz, 2H), 3.87 (dd, J=4.4, 10.0 Hz, 2H), 0.11 (s, 9H).

### Intermediate 9

### Preparation of Intermediate Compound Int-9

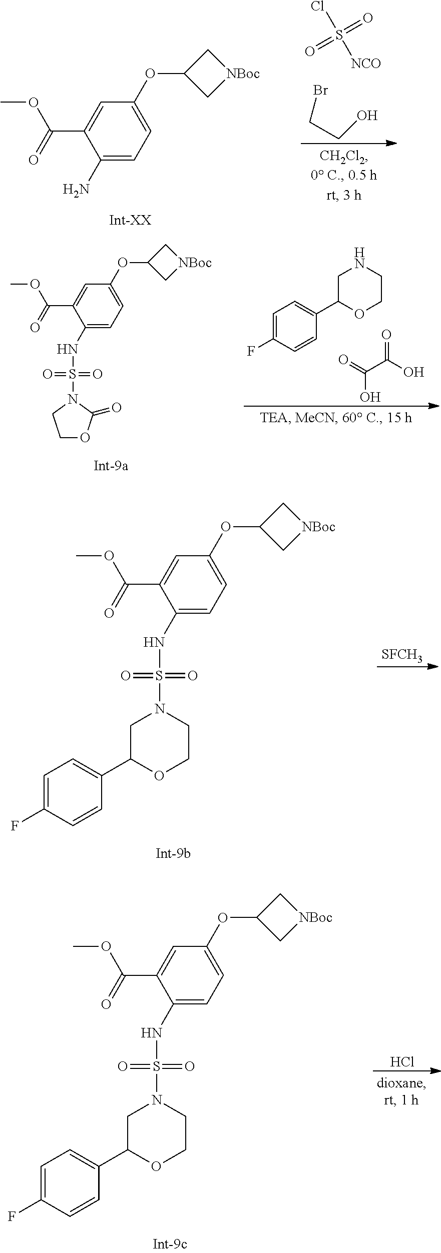

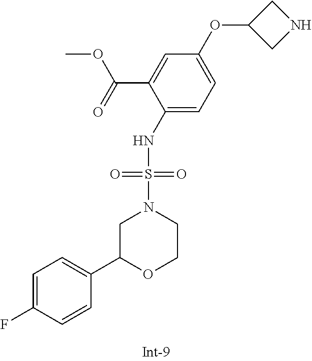 [SMILES: COC(=O)c1cc(OC2CNC2)ccc1NS(=O)(=O)N1CCOC(c2ccc(F)cc2)C1]

### Step 1: tert-butyl 3-(3-(methoxycarbonyl)-4-((2-oxooxazolidine)-3-sulfonamido) phenoxy) azetidine-1-carboxylate (Int-9a)

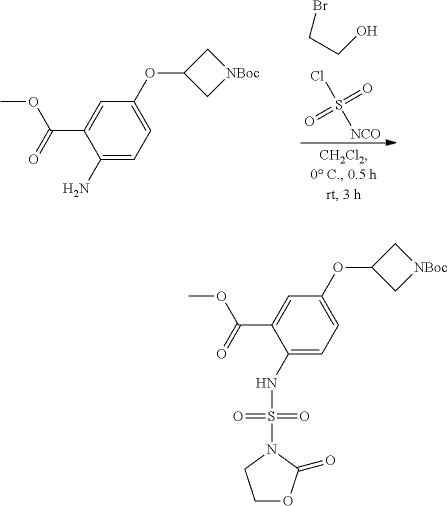

Chlorosulfonyl isocyanate (0.810 mL, 9.33 mmol) was dissolved in CH₂Cl₂ (20 mL) and then cooled to 0° C. A solution of 2-bromoethan-1-ol (1.166 g, 9.33 mmol) in CH₂Cl₂ (10 mL) was added dropwise under argon. After stirring for 30 min, a solution of tert-butyl 3-(4-amino-3-(methoxycarbonyl) phenoxy) azetidine-1-carboxylate (3.01 g, 9.33 mmol) and TEA (3.90 mL, 28.0 mmol) in CH₂Cl₂ (10 mL) were slowly added. The reaction mixture was slowly warmed to room temperature and stirred for 3 h. Sat. aq. NH₄Cl (100 mL) was added and the mixture was extracted with EtOAc (100 mL). The organic fraction was washed with brine (100 mL), dried over anh. Na₂SO₄, filtered, and concentrated in vacuo. The resulting residue was washed with a mixture of EtOAc/hexanes (2:1, 30 mL) to provide tert-butyl 3-(3-(methoxycarbonyl)-4-((2-oxooxazolidine)-3-sulfonamido)-phenoxy)-azetidine-1-carboxylate, which was used in the subsequent step without further purification. MS (ESI, m/z): 472.2 [M+H⁺]. ¹H NMR (500 MHZ, CDCl₃) δ ppm 10.80 (br s, 1H), 7.69 (d, J=9.0 Hz, 1H), 7.33 (d, J=3.0 Hz, 1H), 6.99 (dd, J=3.0, 9.0 Hz, 1H), 4.88 (tt, J=4.0, 6.5 Hz, 1H), 4.36-4.29 (m, 4H), 4.07-3.94 (m, 7H), 1.45 (s, 9H).

### Step 2: tert-butyl 3-(4-((2-(4-fluorophenyl) morpholine)-4-sulfonamido)-3-(methoxycarbonyl) phenoxy) azetidine-1-carboxylate (Int-9b)

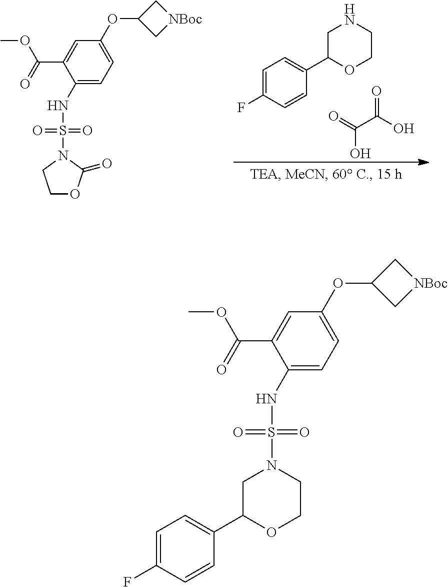

To a stirred solution of 2-(4-fluorophenyl) morpholine (1.015 g, 5.60 mmol)) in MeCN (10 mL) was added tert-butyl 3-(3-(methoxycarbonyl)-4-((2-oxooxazolidine)-3-sulfonamido) phenoxy) azetidine-1-carboxylate (Int-9a; 2.4 g, 5.09 mmol) and TEA (2.129 mL, 15.27 mmol). The reaction mixture was warmed to 60° C. and stirred for 15 h. Then H₂O (30 mL) was added and the mixture was extracted with EtOAc (30 mL). The organic fraction was washed with brine (30 mL), dried over anh. Na₂SO₄, filtered, and concentrated in vacuo. The resulting residue was purified by flash chromatography on SiO₂ [Isco®, Agela® 12 g column, eluent of 0 to 23% EtOAc/Pet. ether gradient at 30 mL/min] to provide tert-butyl 3-(4-((2-(4-fluorophenyl)-morpholine)-4-sulfonamido)-3-(methoxycarbonyl) phenoxy) azetidine-1-carboxylate. MS (ESI, m/z): 588.2 [M+Na⁺]. ¹H NMR (400 MHZ, CDCl₃) δ ppm 10.15 (s, 1H), 7.66 (d, J=9.2 Hz, 1H), 7.34-7.26 (m, 3H), 7.06-6.97 (m, 3H), 4.90-4.84 (m, 1H), 4.41 (dd, J=2.4, 10.4 Hz, 1H), 4.31 (dd, J=6.4, 9.6 Hz, 2H), 4.06-3.92 (m, 6H), 3.74-3.58 (m, 3H), 3.00 (dt, J=3.2, 12.0H_{7., 1}H), 2.77 (dd, J=10.4, 12.0 H₇, 1H), 1.45 (s, 9H).

### Step 3: tert-butyl 3-(4-((2-(4-fluorophenyl) morpholine)-4-sulfonamido)-3-(methoxycarbonyl) phenoxy) azetidine-1-carboxylate and tert-butyl 3-(4-((2-(4-fluorophenyl) morpholine)-4-sulfonamido)-3-(methoxycarbonyl) phenoxy) azetidine-1-carboxylate (Int-9c and Int-9d (enantiomers))

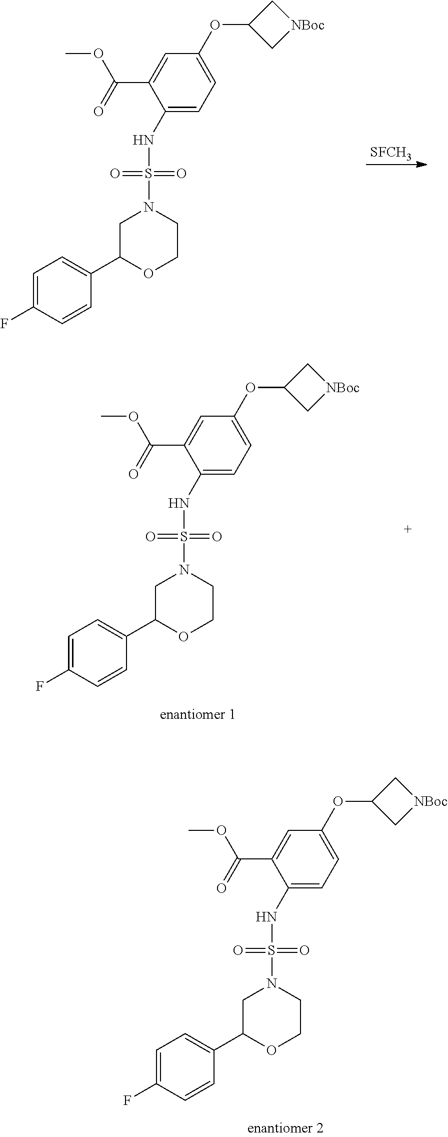 [SMILES: COC(=O)c1cc(OC2CN(C(=O)OC(C)(C)C)C2)ccc1NS(=O)(=O)N1CCOC(c2ccc(F)cc2)C1 ; "SFCH3" ; COC(=O)c1cc(OC2CN(C(=O)OC(C)(C)C)C2)ccc1NS(=O)(=O)N1CCOC(c2ccc(F)cc2)C1 ; COC(=O)c1cc(OC2CN(C(=O)OC(C)(C)C)C2)ccc1NS(=O)(=O)N1CCOC(c2ccc(F)cc2)C1]

The racemic mixture of enantiomers of tert-butyl 3-(4-((2-(4-fluorophenyl) morpholine)-4-sulfonamido)-3-(methoxycarbonyl) phenoxy) azetidine-1-carboxylate (Int-9b, racemic; 900 mg, 1.591 mmol) was separated by chiral Supercritical Fluid chromatography [Column DAICEL CHIRALPAK OJ-H (250 mm*30 mm, 5 μm), conditions 0.1% NH3-H₂O in EtOH using a 30 to 100% gradient] to provide the first eluting peak (P1, Rt=2.9 min) tert-butyl 3-(4-((2-(4-fluorophenyl) morpholine)-4-sulfonamido)-3-(methoxycarbonyl) phenoxy) azetidine-1-carboxylate (Int-9c, enantiomer 1) and the second eluting peak (P2, Rt=3.3 min) tert-butyl 3-(4-((2-(4-fluorophenyl) morpholine)-4-sulfonamido)-3-(methoxycarbonyl) phenoxy) azetidine-1-carboxylate (Int-9d, enantiomer 2).

### Step 4: methyl 5-(azetidin-3-yloxy)-2-((2-(4-fluorophenyl) morpholine)-4-sulfonamido)benzoate (Int-9)

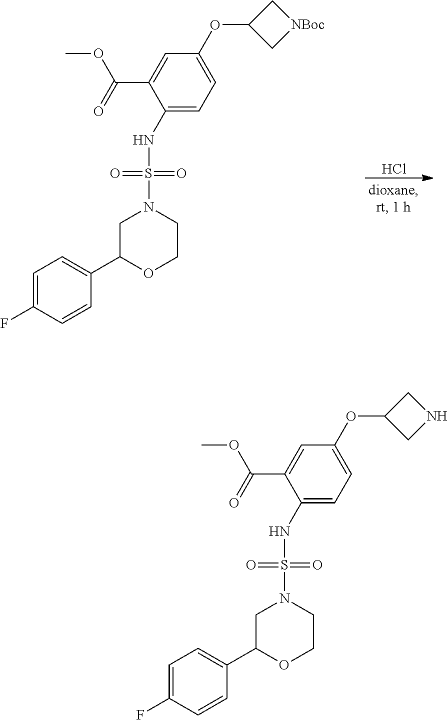 [SMILES: COC(=O)c1cc(OC2CN(C(=O)OC(C)(C)C)C2)ccc1NS(=O)(=O)N1CCOC(c2ccc(F)cc2)C1 ; Cl ; COC(=O)c1cc(OC2CNC2)ccc1NS(=O)(=O)N1CCOC(c2ccc(F)cc2)C1]

A mixture of tert-butyl 3-(4-((2-(4-fluorophenyl) morpholine)-4-sulfonamido)-3-(methoxycarbonyl) phenoxy) azetidine-1-carboxylate (Int-9c; 150 mg, 0.265 mmol) in HCl (4 mL, 4N in 1,4-dioxane) was stirred at room temperature for 1 h. The reaction mixture was concentrated in vacuo to provide methyl 5-(azetidin-3-yloxy)-2-((2-(4-fluorophenyl)-morpholine)-4-sulfonamido)benzoate, which was used in the subsequent step without further purification. MS (ESI, m/z): 466.2 [M+H⁺].

### Intermediate 10

### Preparation of Intermediate Compound Int-10

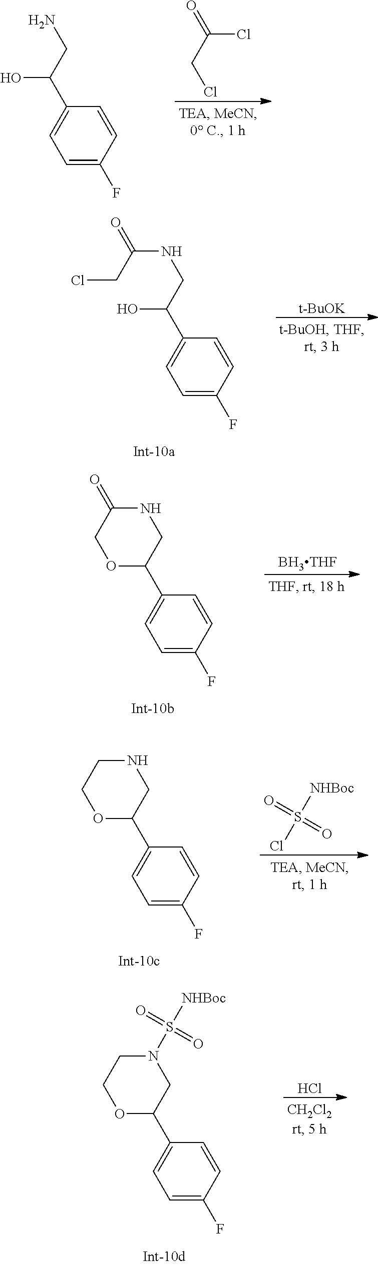

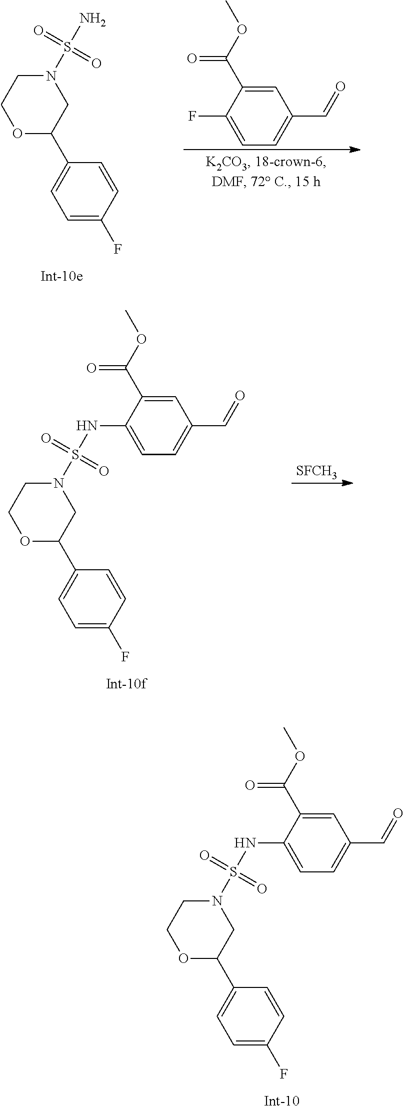 [SMILES: NS(=O)(=O)N1CCOC(c2ccc(F)cc2)C1 ; COC(=O)c1cc(C=O)ccc1F ; COC(=O)c1cc(C=O)ccc1NS(=O)(=O)N1CCOC(c2ccc(F)cc2)C1 ; "SFCH3" ; COC(=O)c1cc(C=O)ccc1NS(=O)(=O)N1CCOC(c2ccc(F)cc2)C1]

### Step 1:2-chloro-N-(2-(4-fluorophenyl)-2-hydroxyethyl) acetamide (Int-10a)

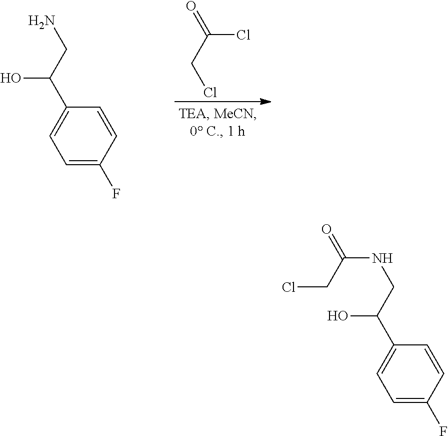 [SMILES: NCC(O)c1ccc(F)cc1 ; O=C(Cl)CCl ; O=C(CCl)NCC(O)c1ccc(F)cc1]

2-Chloroacetyl chloride (0.519 mL, 6.52 mmol) was added to a stirred mixture of 2-amino-1-(4-fluorophenyl) ethan-1-ol HCl salt (1.25 g, 6.52 mmol) and TEA (2.73 mL, 19.57 mmol) in MeCN (20 mL) at 0-5° C. The mixture was stirred at 0° C. for 1 h. H₂O (60 mL) was added and the mixture was extracted with EtOAc (60 mL). The organic fraction was washed with brine (60 mL), dried over anh. Na₂SO₄, filtered, and concentrated in vacuo to provide 2-chloro-N-(2-(4-fluorophenyl)-2-hydroxyethyl) acetamide, which was used in the subsequent step without further purification.

### Step 2:6-(4-fluorophenyl) morpholin-3-one (Int-10b)

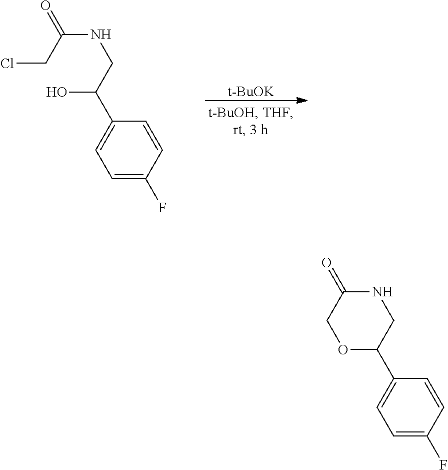 [SMILES: "t-BuOK" ; O=C(CCl)NCC(O)c1ccc(F)cc1 ; O=C1COC(c2ccc(F)cc2)CN1]

Potassium tert-butoxide (1.574 g, 14.03 mmol) was added to a stirred mixture of 2-chloro-N-(2-(4-fluorophenyl)-2-hydroxyethyl) acetamide (Int-10a; 1.3 g, 5.61 mmol) in t-BuOH (30 mL) at 0-5° C. The mixture was stirred at room temperature for 3 h. Sat. aq. NH₄Cl (100 mL) was added and the mixture was extracted with EtOAc (100 mL). The organic fraction was washed with brine (100 mL), dried over anh. Na₂SO₄, filtered, and concentrated in vacuo. The residue was purified by flash chromatography on SiO₂ [Isco®, Agela® 8 g column, eluent of 0 to 53% EtOAc/Pet. ether gradient at 30 mL/min] to provide 6-(4-fluorophenyl) morpholin-3-one. MS (ESI, m/z): 196.3 [M+H⁺]. ¹H NMR (400 MHZ, CDCl₃) δ ppm 7.38-7.32 (m, 2H), 7.19-7.04 (m, 3H), 4.73 (dd, J=4.4, 8.8 Hz, 1H), 4.45-4.39 (m, 1H), 4.37-4.31 (m, 1H), 3.53-3.44 (m, 2H).

### Step 3:2-(4-fluorophenyl) morpholine (Int-10c)

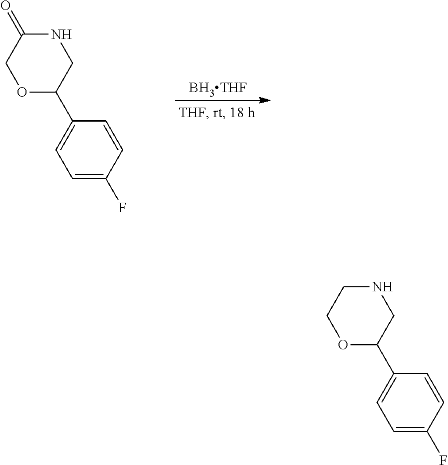 [SMILES: O=C1COC(c2ccc(F)cc2)CN1 ; Fc1ccc(C2CNCCO2)cc1 ; "BH3·THF"]

A solution of BH3. THF (1 M in THF, 3.89 mL, 3.89 mmol) was added to a stirred mixture of 6-(4-fluorophenyl) morpholin-3-one (Int-10b; 380 mg, 1.95 mmol) in THF (10 mL). The mixture was stirred at room temperature for 18 h. The solvent was removed in vacuo to provide 2-(4-fluorophenyl) morpholine, which was used in the subsequent step without further purification. MS (ESI, m/z): 182.2 [M+H⁺].

### Step 4: tert-butyl ((2-(4-fluorophenyl) morpholino) sulfonyl) carbamate (Int-10d)

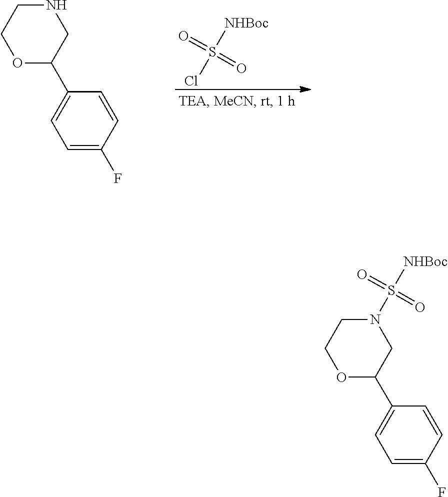 [SMILES: Fc1ccc(C2CNCCO2)cc1 ; CC(C)(C)OC(=O)NS(=O)(=O)Cl ; CC(C)(C)OC(=O)NS(=O)(=O)N1CCOC(c2ccc(F)cc2)C1]

To a solution of tert-butyl(chlorosulfonyl) carbamate (500 mg, 2.318 mmol) and 2-(4-fluorophenyl) morpholine (Int-10c; 350 mg, 1.931 mmol) in MeCN (10 mL) was added TEA (0.808 mL, 5.79 mmol) at room temperature. The mixture was stirred at room temperature for 1 h. Then reaction mixture was concentrated in vacuo, and the resulting residue was purified by flash chromatography on SiO₂ [Isco®, Agela® 4 g column, eluent of 0 to 23% EtOAc/Pet. ether gradient at 30 mL/min] to provide tert-butyl ((2-(4-fluorophenyl)-morpholino)-sulfonyl)-carbamate. ¹H NMR (400 MHZ, CDCl₃) δ ppm 7.38-7.31 (m, 2H), 7.05 (br t, J=8.8 Hz, 3H), 4.55 (dd, J=2.4, 10.4 Hz, 1H), 4.11-4.08 (m, 1H), 3.86-3.78 (m, 2H), 3.70 (br d, J=12.4 Hz, 1H), 3.22 (dt, J=3.6, 12.0 Hz, 1H), 2.95 (dd, J=10.4, 12.4 Hz, 1H), 1.48 (s, 9H).

### Step 5:2-(4-fluorophenyl) morpholine-4-sulfonamide (Int-10e)

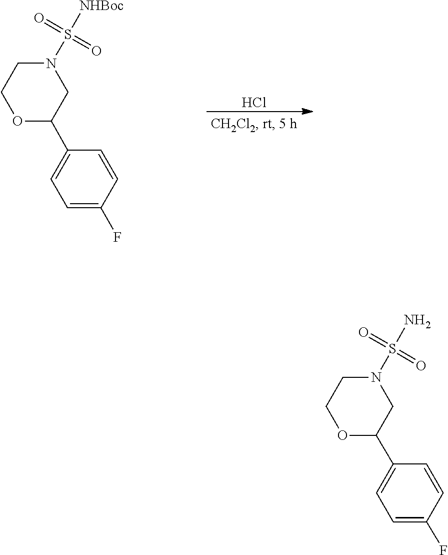 [SMILES: CC(C)(C)OC(=O)NS(=O)(=O)N1CCOC(c2ccc(F)cc2)C1 ; Cl ; NS(=O)(=O)N1CCOC(c2ccc(F)cc2)C1]

A solution of tert-butyl ((2-(4-fluorophenyl) morpholino) sulfonyl) carbamate (Int-10d; 500 mg, 1.387 mmol) in CH₂Cl₂ (5 mL) and HCl (10 mL, 4N in EtOAc) was stirred at room temperature for 5 h. The reaction mixture was concentrated in vacuo to provide 2-(4-fluorophenyl) morpholine-4-sulfonamide, which was used in the subsequent step without further purification. MS (ESI, m/z): 261.2 [M+H⁺].

### Step 6: methyl 2-((2-(4-fluorophenyl) morpholine)-4-sulfonamido)-5-formylbenzoate (Int-10f)

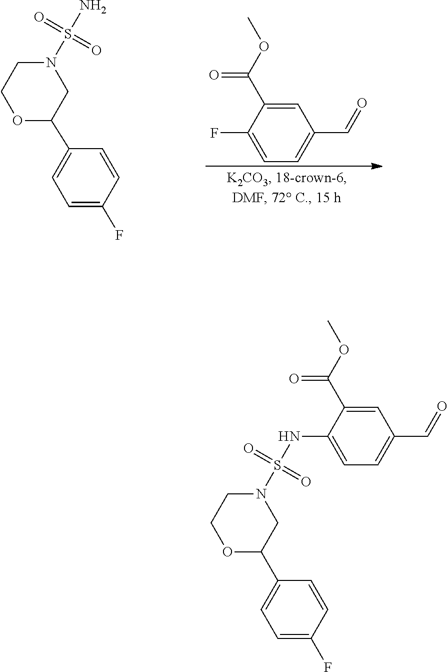 [SMILES: NS(=O)(=O)N1CCOC(c2ccc(F)cc2)C1 ; COC(=O)c1cc(C=O)ccc1F ; COC(=O)c1cc(C=O)ccc1NS(=O)(=O)N1CCOC(c2ccc(F)cc2)C1]

A mixture of 2-(4-fluorophenyl) morpholine-4-sulfonamide (Int-10e; 350 mg, 1.345 mmol), methyl 2-fluoro-5-formylbenzoate (490 mg, 2.69 mmol), 18-crown-6 (355 mg, 1.345 mmol) and K₂CO₃ (372 mg, 2.69 mmol) in DMF (5 mL) was stirred at 72° C. for 15 h. The reaction mixture was filtered and the solvent was concentrated in vacuo. The resulting residue was purified by reverse phase HPLC chromatography [Gilson 281, Phenomenex Synergi C18 column (150*30 mm, 4 μm), eluent of Mobile Phase A: H₂O (0.05% HCl) and Mobile Phase B: MeCN @ 220 nm] to provide racemic methyl 2-((2-(4-fluorophenyl) morpholine)-4-sulfonamido)-5-formylbenzoate. MS (ESI, m/z): 423.1 [M+H′]. ¹H NMR (400 MHZ, CDCl₃) δ ppm 10.96 (s, 1H), 9.94 (s, 1H), 8.56 (d, J=2.0 Hz, 1H), 8.03 (dd, J=2.0, 8.4 Hz, 1H), 7.81 (d, J=8.4 Hz, 1H), 7.30 (dd, J=5.6, 8.4 Hz, 2H), 7.04 (t, J=8.4 Hz, 2H), 4.48 (dd, J=2.4, 10.4 Hz, 1H), 4.08 (dd, J=2.4, 9.2 Hz, 1H), 3.98 (s, 3H), 3.80-3.66 (m, 3H), 3.09 (dt, J=3.6, 11.6 Hz, 1H), 2.84 (dd, J=10.4, 12.0 Hz, 1H).

### Step 7: methyl 2-((2-(4-fluorophenyl) morpholine)-4-sulfonamido)-5-formylbenzoate (Int-10)

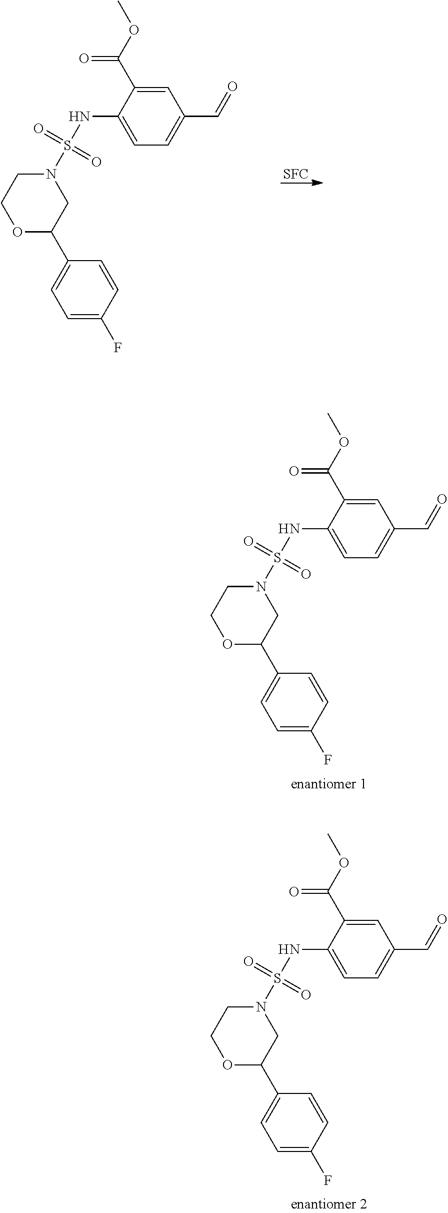 [SMILES: COC(=O)c1cc(C=O)ccc1NS(=O)(=O)N1CCOC(c2ccc(F)cc2)C1 ; "SFC" ; COC(=O)c1cc(C=O)ccc1NS(=O)(=O)N1CCOC(c2ccc(F)cc2)C1 ; COC(=O)c1cc(C=O)ccc1NS(=O)(=O)N1CCOC(c2ccc(F)cc2)C1]

The racemic mixture of enantiomers of methyl 2-((2-(4-fluorophenyl) morpholine)-4-sulfonamido)-5-formylbenzoate (Int-10e, racemic; 950 mg, 2.249 mmol) was separated by Chiral Supercritical Fluid chromatography [Column DAICEL CHIRALPAK OJ-H (250 mm*30 mm, 5 μm), conditions 0.1% NH3·H₂O in IPA using a 40 to 100% gradient] to provide the first eluting peak (P1, Rt=1.62 min)methyl 2-((2-(4-fluorophenyl)-morpholine)-4-sulfonamido)-5-formylbenzoate (Int-10, enantiomer 1) and the second eluting peak (P2, Rt=2.05 min)methyl 2-((2-(4-fluorophenyl) morpholine)-4-sulfonamido)-5-formylbenzoate (Int-10g, enantiomer 2).

### Intermediate 11

### Preparation of Intermediate Compound Int-11

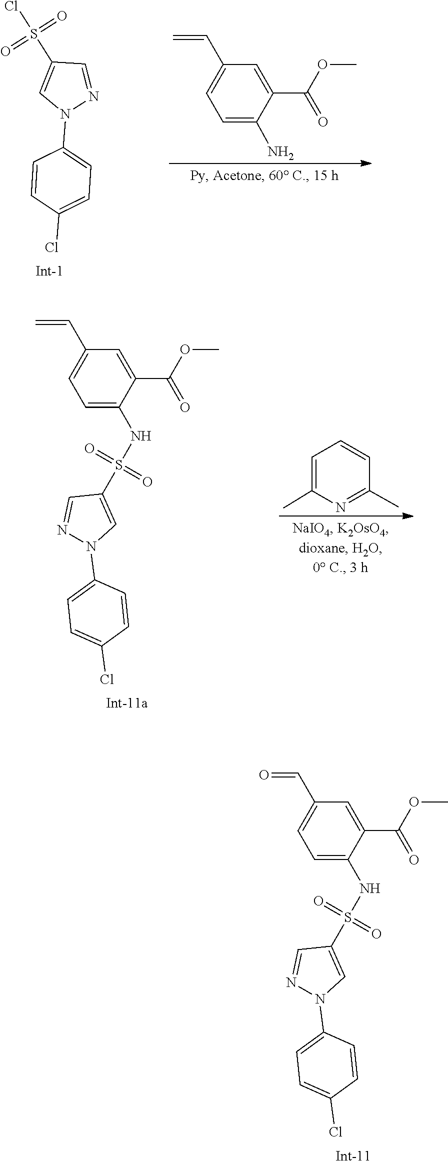

### Step 1: methyl 2-(1-(4-chlorophenyl)-1H-pyrazole-4-sulfonamido)-5-vinylbenzoate (Int-11a)

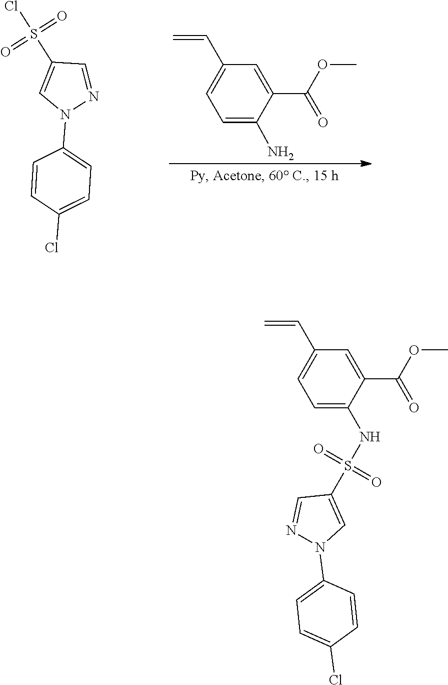 [SMILES: O=S(=O)(Cl)c1cnn(-c2ccc(Cl)cc2)c1 ; C=Cc1ccc(N)c(C(=O)OC)c1 ; C=Cc1ccc(NS(=O)(=O)c2cnn(-c3ccc(Cl)cc3)c2)c(C(=O)OC)c1]

To a solution of methyl 2-amino-5-vinylbenzoate (4 g, 22.57 mmol) in MeCN (50 mL) and pyridine (10 mL) was added 1-(4-chlorophenyl)-1H-pyrazole-4-sulfonyl chloride (Int-1; 6.57 g, 23.70 mmol) and DMAP (0.276 g, 2.257 mmol). The reaction mixture was then warmed to 55° C. for 16 h. The reaction mixture was concentrated in vacuo and the resulting residue was purified by flash chromatography on SiO₂ [Isco®, SepaFlash 40 g column, eluent of 14% EtOAc/Pet. ether gradient at 40 mL/min] to provide methyl 2-(1-(4-chlorophenyl)-1H-pyrazole-4-sulfonamido)-5-vinylbenzoate. MS (ESI, m/z): 418.0 [M+H⁺]. ¹H NMR (400 MHZ, CDCl₃) δ 10.66 (s, 1H), 8.25 (s, 1H), 7.98 (d, J-2.0 Hz, 1H), 7.89 (s, 1H), 7.76 (d, J=8.6 Hz, 1H), 7.61-7.52 (m, 3H), 7.48-7.39 (m, 2H), 6.63 (dd, J=11.0, 17.6 Hz, 1H), 5.71 (d, J=17.6 Hz, 1H), 5.27 (d, J=11.0 Hz, 1H), 3.90 (s, 3H).

### Step 2: methyl 2-(1-(4-chlorophenyl)-1H-pyrazole-4-sulfonamido)-5-formylbenzoate (Int-11)

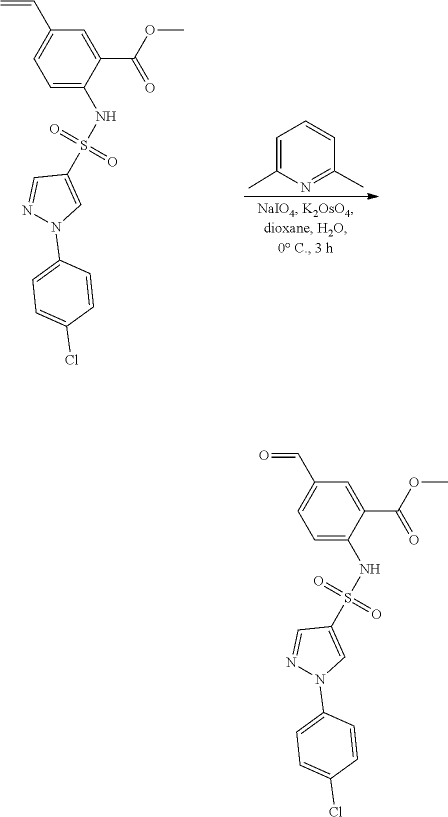

To a solution of methyl 2-(1-(4-chlorophenyl)-1H-pyrazole-4-sulfonamido)-5-vinyl-benzoate (Int-11a; 3.7 g, 8.85 mmol) in 1,4-dioxane (120 mL) and H₂O (30 mL) was added 2,6-dimethylpyridine (2.063 mL, 17.71 mmol), sodium periodate (7.58 g, 35.4 mmol) and potassium osmate (VI) dihydrate (0.652 g, 1.771 mmol) at 0° C. The reaction mixture was warmed to room temperature, and then stirred for 16 h. The reaction mixture was filtered, quenched with sat. aq. Na₂SO₃ (200 mL) and H₂O (100 mL), and extracted with EtOAc (3×100 mL). The combined organic phases were washed with brine (300 mL), dried over anh. Na₂SO₄, filtered, and concentrated in vacuo. The resulting residue was purified by flash chromatography on SiO₂ [Isco®, Agela® 12 g column, eluent of 20% EtOAc/Pet. ether gradient at 35 mL/min] to provide methyl 2-(1-(4-chlorophenyl)-1/-pyrazole-4-sulfonamido)-5-formylbenzoate. This material was then further washed with EtOAc/Pet.ether (1:5, 40 mL), concentrated in vacuo, and dried under vacuum to provide the final product. MS (ESI, m/z): 419.9 [M+H⁺]. ¹H NMR (500 MHZ, CDCl₃) δ=11.25 (s, 1H), 9.92 (s, 1H), 8.53 (d, J-2.0 Hz, 1H), 8.37 (s, 1H), 8.03 (d, J-8.6 Hz, 1H), 8.00 (s, 1H), 7.92 (d, J=8.5 Hz, 1H), 7.62-7.57 (m, 2H), 7.48-7.44 (m, 2H), 3.98 (s, 3H).

The following intemediate of the present disclosure were made using the methods described in Intermediate 11 above, and substituting the appropriate reactants and/or reagents:

| Intermediate | Structure |
|---|---|
| Int-12 | 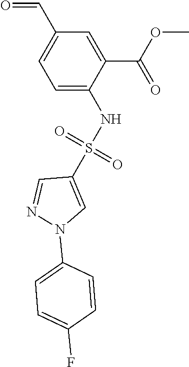 [SMILES: COC(=O)c1cc(C=O)ccc1NS(=O)(=O)c1cnn(-c2ccc(F)cc2)c1] |

### Intermediate 13

### Preparation of Intermediate Compound Int-13

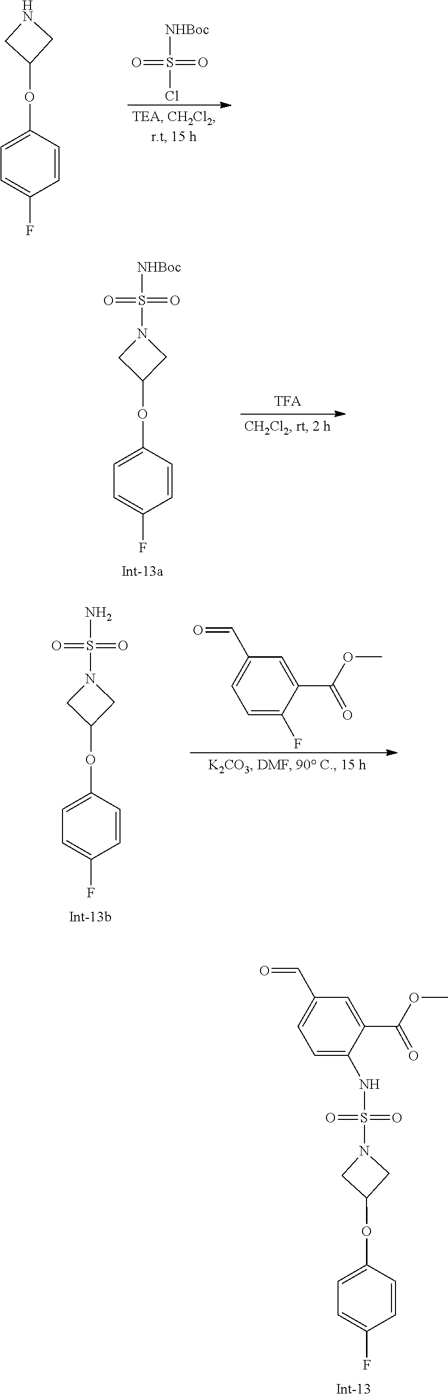 [SMILES: Fc1ccc(OC2CNC2)cc1 ; CC(C)(C)OC(=O)NS(=O)(=O)Cl ; CC(C)(C)OC(=O)NS(=O)(=O)N1CC(Oc2ccc(F)cc2)C1 ; NS(=O)(=O)N1CC(Oc2ccc(F)cc2)C1 ; COC(=O)c1cc(C=O)ccc1F ; COC(=O)c1cc(C=O)ccc1NS(=O)(=O)N1CC(Oc2ccc(F)cc2)C1]

### Step 1: tert-butyl (3-(4-fluorophenoxy) azetidin-1-yl) sulfonylcarbamate (Int-13a)

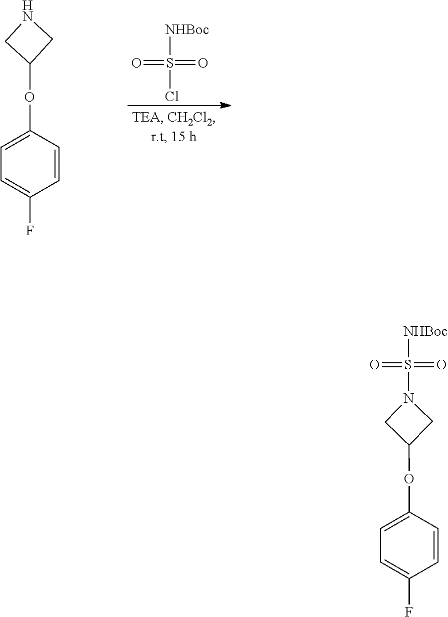 [SMILES: Fc1ccc(OC2CNC2)cc1 ; CC(C)(C)OC(=O)NS(=O)(=O)Cl ; CC(C)(C)OC(=O)NS(=O)(=O)N1CC(Oc2ccc(F)cc2)C1]

To a solution of 3-(4-fluorophenoxy) azetidine (236 mg, 1.412 mmol) and TEA (0.787 mL, 5.65 mmol) in CH₂Cl₂ (5 mL) was added tert-butyl chlorosulfonylcarbamate (365 mg, 1.694 mmol) at room temperature. The reaction mixture was stirred for 15 h, concentrated in vacuo and the resulting residue was purified by reverse phase preparative chromatography [Phenomenex Synergi C18 column (150*21.2 mm, 4 μm), eluent of Mobile Phase A: H₂O (0.1% TFA) and Mobile Phase B: MeCN at 215 nm] to provide tert-butyl (3-(4-fluorophenoxy) azetidin-1-yl) sulfonylcarbamate. MS (ESI) m/z: 291.2 [M+H⁺-56].

### Step 2:3-(4-fluorophenoxy) azetidine-1-sulfonamide (Int-13b)

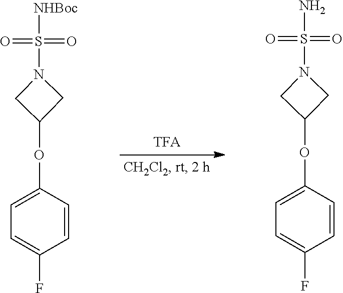 [SMILES: CC(C)(C)OC(=O)NS(=O)(=O)N1CC(Oc2ccc(F)cc2)C1 ; NS(=O)(=O)N1CC(Oc2ccc(F)cc2)C1]

To a solution of tert-butyl (3-(4-fluorophenoxy) azetidin-1-yl) sulfonylcarbamate (Int-13a; 310 mg, 0.895 mmol) in CH₂Cl₂ (6 mL), was added TFA (3 mL, 38.9 mmol) at room temperature. The reaction mixture was stirred for 2 h and concentrated in vacuo to provide the crude 3-(4-fluorophenoxy) azetidine-1-sulfonamide, which was used in the subsequent step without further purification. MS (ESI) m/z: 247.2 [M+H⁺].

### Step 3: methyl 2-(3-(4-fluorophenoxy) azetidine-1-sulfonamido)-5-formylbenzoate (Int-13)

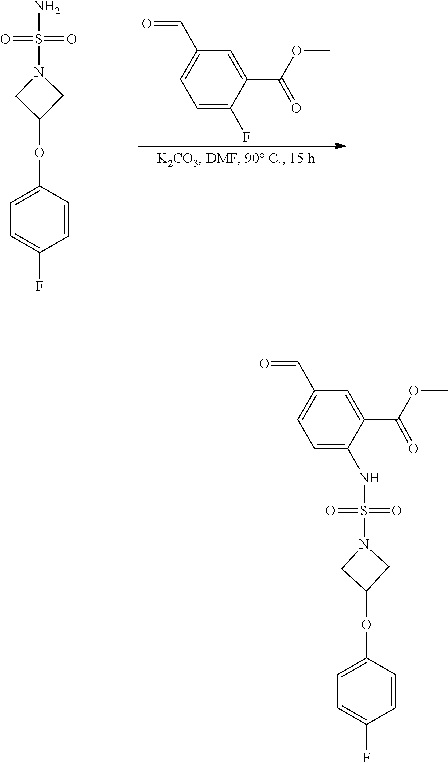 [SMILES: NS(=O)(=O)N1CC(Oc2ccc(F)cc2)C1 ; COC(=O)c1cc(C=O)ccc1F ; COC(=O)c1cc(C=O)ccc1NS(=O)(=O)N1CC(Oc2ccc(F)cc2)C1]

To a solution of methyl 2-fluoro-5-formylbenzoate (37.0 mg, 0.203 mmol) and 3-(4-fluorophenoxy) azetidine-1-sulfonamide (Int-13b; 50 mg, 0.203 mmol) in DMF (1 mL) was added K₂CO₃ (28.1 mg, 0.203 mmol) at room temperature. The reaction mixture was stirred at 90° C. for 15 h, concentrated in vacuo, and the resulting residue was purified by reverse phase preparative chromatography [Phenomenex Synergi C18 column (150*21.2 mm, 4 μm), eluent of Mobile Phase A: H₂O (0.1% TFA) and Mobile Phase B: MeCN at 215 nm] to provide methyl 2-(3-(4-fluorophenoxy) azetidine-1-sulfonamido)-5-formylbenzoate. MS (ESI) m/z: 409.5 [M+H⁺]. ¹H NMR (500 MHZ, CDCl₃) δ ppm 10.94 (s, 1H), 9.95 (s, 1H), 8.56 (d, J=2.0 Hz, 1H), 8.06 (dd, J=2.0, 8.5 Hz, 1H), 7.86 (d, J=8.5 Hz, 1H), 7.01-6.93 (m, 2H), 6.71-6.64 (m, 2H), 4.90-4.84 (m, 1H), 4.31 (dd, J=6.5, 9.5 Hz, 2H), 4.12 (dd, J=5.0, 9.5 Hz, 2H), 3.99 (s, 3H).

### Intermediate 14

### Preparation of Intermediate Compound Int-14

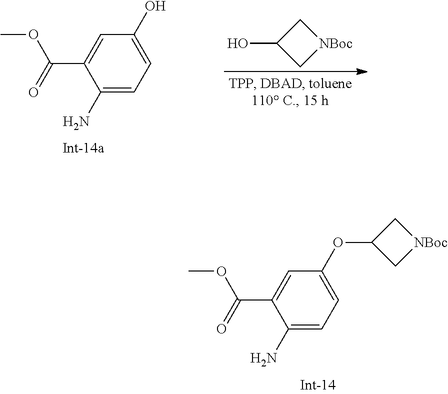 [SMILES: COC(=O)c1cc(O)ccc1N ; CC(C)(C)OC(=O)N1CC(O)C1 ; COC(=O)c1cc(OC2CN(C(=O)OC(C)(C)C)C2)ccc1N]

### Step 1: tert-butyl 3-(4-(1-(4-fluorophenyl)-1H-pyrazole-4-sulfonamido)-3-(methoxy carbonyl) phenoxy) azetidine-1-carboxylate (Int-14)

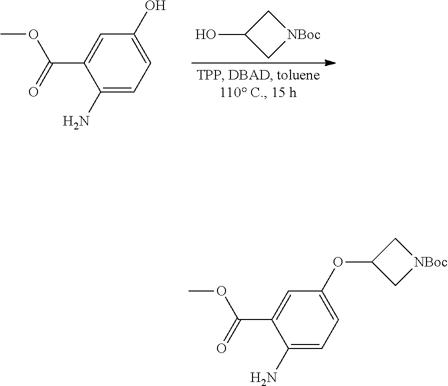 [SMILES: COC(=O)c1cc(O)ccc1N ; CC(C)(C)OC(=O)N1CC(O)C1 ; COC(=O)c1cc(OC2CN(C(=O)OC(C)(C)C)C2)ccc1N]

To a solution of methyl 2-amino-5-hydroxybenzoate (15 g, 90 mmol) in toluene (150 mL) was added tert-butyl 3-hydroxyazetidine-1-carboxylate (15.54 g, 90 mmol), DBAD (26.9 g, 117 mmol) and triphenylphosphine (30.6 g, 117 mmol) at room temperature. The reaction mixture was stirred at 110° C. for 15 h, concentrated in vacuo, and the residue was diluted with H₂O (600 mL), and then extracted with CH₂Cl₂ (200 mL×2). The organic layer was dried over Na₂SO₄, filtered and concentrated under reduced pressure. The resulting residue was purified by flash silica gel chromatography [Isco®, Agela® Flash 120 g Column, eluent of 0 to 24% EtOAc/Pet. ether gradient at 35 mL/min] to provide tert-butyl 3-(4-amino-3-(methoxycarbonyl) phenoxy) azetidine-1-carboxylate. MS (ESI) m/z: 323.3 [M+H⁺]; ¹H NMR (500 MHz, CDCl₃) δ ppm 7.14 (d, J=3.0 Hz, 1H), 6.87 (dd, J=8.5, 2.5 Hz, 1H), 6.64 (d, J=9.0 Hz, 1H), 6.26 (br s, 2H), 4.78-4.85 (m, 1H), 4.26 (dd, J=9.5, 6.5 Hz, 2H), 3.97 (dd, J=9.5, 4.0 Hz, 2H), 3.87 (s, 3H), 1.45 (s, 9H).

### Intermediate 15

### Preparation of Intermediate Compound Int-15

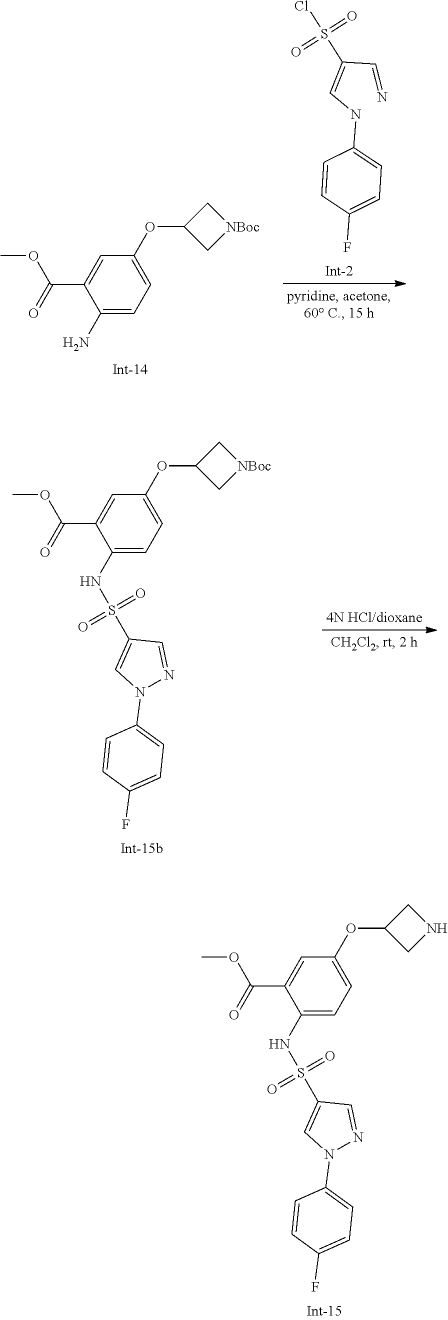 [SMILES: COC(=O)c1cc(OC2CN(C(=O)OC(C)(C)C)C2)ccc1N ; N=C/C(=C\Nc1ccc(F)cc1)S(=O)(=O)Cl ; COC(=O)c1cc(OC2CN(C(=O)OC(C)(C)C)C2)ccc1NS(=O)(=O)c1cnn(-c2ccc(F)cc2)c1 ; COC(=O)c1cc(OC2CNC2)ccc1NS(=O)(=O)c1cnn(-c2ccc(F)cc2)c1]

### Step 1: tert-butyl 3-(4-(1-(4-fluorophenyl)-1H-pyrazole-4-sulfonamido)-3-(methoxy carbonyl) phenoxy) azetidine-1-carboxylate (Int-15b)

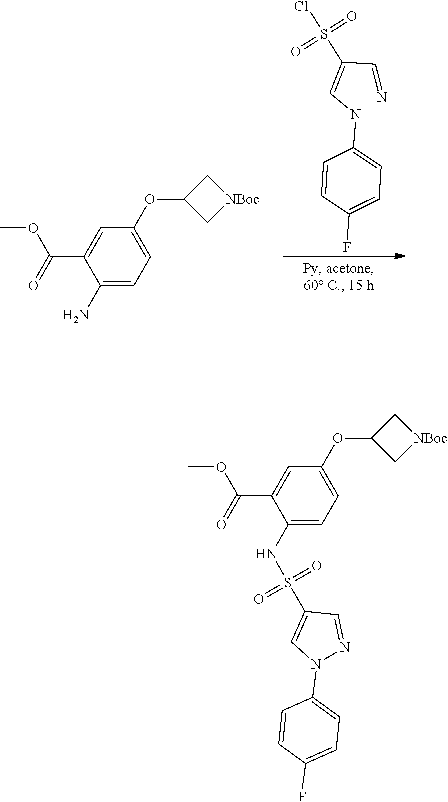 [SMILES: COC(=O)c1cc(OC2CN(C(=O)OC(C)(C)C)C2)ccc1N ; N=C/C(=C\Nc1ccc(F)cc1)S(=O)(=O)Cl ; COC(=O)c1cc(OC2CN(C(=O)OC(C)(C)C)C2)ccc1NS(=O)(=O)c1cnn(-c2ccc(F)cc2)c1]

To a solution of tert-butyl 3-(4-amino-3-(methoxycarbonyl) phenoxy) azetidine-1-carboxylate (Int-14; 4 g, 12.41 mmol) in acetone (15 mL) and pyridine (5 mL) was added 1-(4-fluorophenyl)-1H-pyrazole-4-sulfonyl chloride (3.88 g, 14.89 mmol) at room temperature. The reaction mixture was stirred at 60° C. for 15 h, concentrated in vacuo, and the residue was purified by flash chromatography on SiO₂ [Isco®, SepaFlash® 80 g Column, eluent of 29% EtOAc/Pet. ether isocratic gradient at 30 mL/min) to give tert-butyl 3-(4-(1-(4-fluorophenyl)-1H-pyrazole-4-sulfonamido)-3-(methoxycarbonyl) phenoxy) azetidine-1-carboxylate. MS (ESI) m/z: 547.1 [M+H⁺]; ¹H NMR (400 MHZ, CDCl₃) δ ppm 10.14 (s, 1H), 8.13 (s, 1H), 7.78 (s, 1H), 7.72 (d, J=9.2 Hz, 1H), 7.52-7.59 (m, 2H), 7.23 (s, 1H), 7.12-7.18 (m, 2H), 6.97 (dd, J=9.2, 3.2 Hz, 1H), 4.82 (tt, J=6.4, 4.0 Hz, 1H), 4.26 (dd, J=9.6, 6.4 Hz, 2H), 3.94 (dd, J=10.0, 4.0 Hz, 2H), 3.83 (s, 3H), 1.42 (s, 9H).

### Step 2: methyl 5-(azetidin-3-yloxy)-2-(1-(4-fluorophenyl)-1H-pyrazole-4-sulfonamido)benzoate (Int-15)

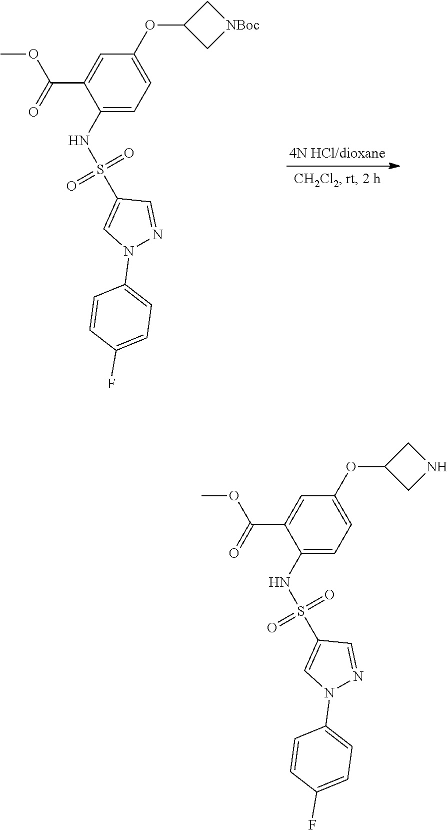 [SMILES: COC(=O)c1cc(OC2CN(C(=O)OC(C)(C)C)C2)ccc1NS(=O)(=O)c1cnn(-c2ccc(F)cc2)c1 ; COC(=O)c1cc(OC2CNC2)ccc1NS(=O)(=O)c1cnn(-c2ccc(F)cc2)c1]

To a solution of tert-butyl 3-(4-(1-(4-fluorophenyl)-1H-pyrazole-4-sulfonamido)-3-(methoxycarbonyl) phenoxy) azetidine-1-carboxylate (Int-15b; 3.3 g, 6.04 mmol) in CH₂Cl₂ (5 mL) was added HCl (4 M in 1,4-dioxane, 5.0 mL, 20 mmol) at room temperature. The reaction mixture was stirred at room temperature for 2 h. The reaction was concentrated to give the crude methyl 5-(azetidin-3-yloxy)-2-(1-(4-fluorophenyl)-1H-pyrazole-4-sulfonamido)benzoate, which was used in the subsequent step without further purification. MS (ESI) m/z: 447.2 [M+H⁺]

The following intermediate of the present disclosure were made using the methods described in Intermediate 15 above, and substituting the appropriate reactants and/or reagents:

| Compound | Structure |
|---|---|
| Int-16 | 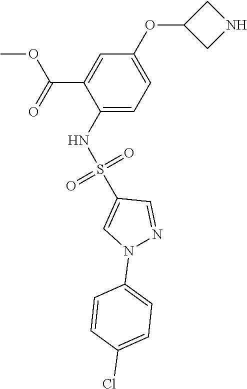 [SMILES: COC(=O)c1cc(OC2CNC2)ccc1NS(=O)(=O)c1cnn(-c2ccc(Cl)cc2)c1] |

### Intermediate 17

### Preparation of Intermediate 17

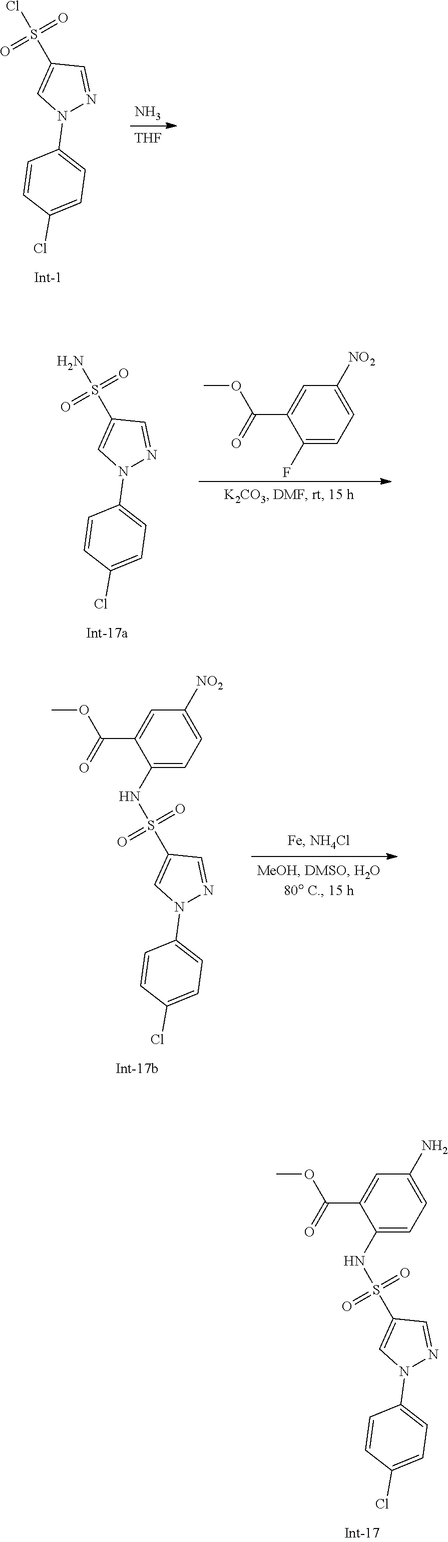

### Step 1:1-(4-chlorophenyl)-1H-pyrazole-4-sulfonamide (Int-17a)

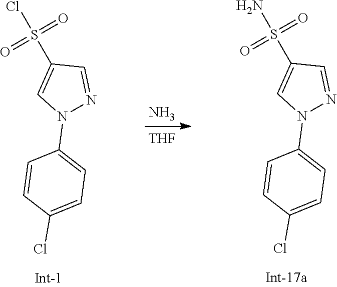

To a stirred solution of 1-(4-chlorophenyl)-1H-pyrazole-4-sulfonyl chloride (2 g, 7.22 mmol) in THF (20 mL) was added aq. NH3 H₂O (10 mL) at 20° C. The reaction mixture was stirred at 20° C. for 15 h, concentrated in vacuo, and the resulting residue was washed with H₂O (3×10 mL) and EtOAc (3×10 mL) and filtered to afford 1-(4-chlorophenyl)-1H-pyrazole-4-sulfonamide. MS (ESI) m/z: 258.0 [M+H⁺]. ¹H NMR (400 MHZ, DMSO-d6) δ=8.98 (s, 1H), 8.02 (s, 1H), 7.92 (d, J=8.6 Hz, 2H), 7.66-7.54 (m, 2H), 7.42 (s, 2H).

### Step 2: methyl 2-((1-(4-chlorophenyl)-1H-pyrazole)-4-sulfonamido)-5-nitrobenzoate (Int-17b)

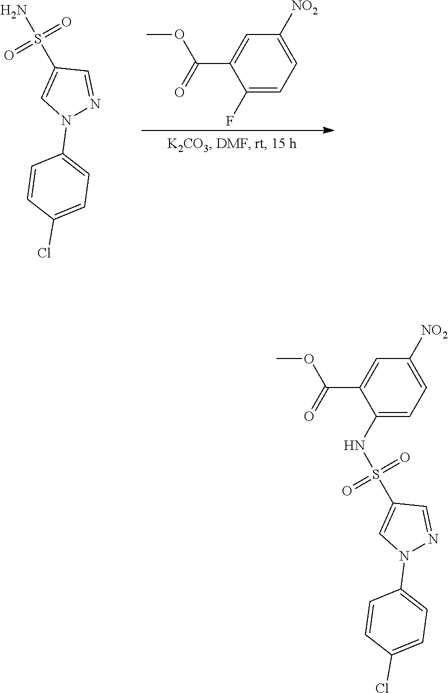 [SMILES: NS(=O)(=O)c1cnn(-c2ccc(Cl)cc2)c1 ; COC(=O)c1cc([N+](=O)[O-])ccc1F ; COC(=O)c1cc([N+](=O)[O-])ccc1NS(=O)(=O)c1cnn(-c2ccc(Cl)cc2)c1]

To a solution of methyl 2-fluoro-5-nitrobenzoate (1.159 g, 5.82 mmol) in DMF (10 mL) were added 1-(4-chlorophenyl)-1H-pyrazole-4-sulfonamide (1.5 g, 5.82 mmol) and K₂CO₃ (1.609 g, 11.64 mmol) at room temperature. The reaction mixture was stirred at room temperature for 15 h, then diluted with H₂O (50 mL) and extracted with EtOAc (3×20 mL). The organic layers were combined, washed with H₂O (20 mL), dried over anh. Na₂SO₄, and filtered. The filtrate was concentrated in vacuo to provide methyl 2-(1-(4-chlorophenyl)-1H-pyrazole-4-sulfonamido)-5-nitrobenzoate, which was used in the subsequent step without further purification. ¹H NMR (400 MHz, DMSO-d6) δ ppm 11.03 (s, 1H), 9.41 (s, 1H), 8.62-8.63 (m, 1H), 8.36-8.40 (m, 1H), 8.33 (s, 1H), 7.90 (d, J=8.0 Hz, 2H), 7.83 (d, J=8.0 Hz, 1H), 7.59 (d, J=8.0 Hz, 2H), 3.90 (s, 3H).

### Step 3: methyl 5-amino-2-((1-(4-chlorophenyl)-1H-pyrazole)-4-sulfonamido)benzoate (Int-17)

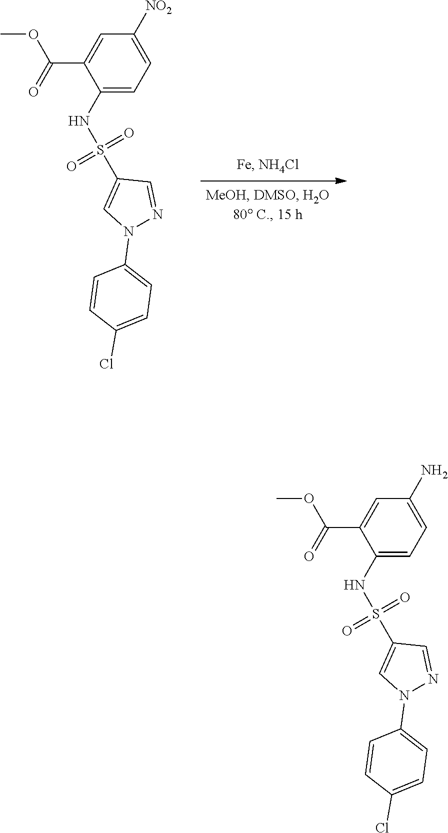 [SMILES: COC(=O)c1cc([N+](=O)[O-])ccc1NS(=O)(=O)c1cnn(-c2ccc(Cl)cc2)c1 ; COC(=O)c1cc(N)ccc1NS(=O)(=O)c1cnn(-c2ccc(Cl)cc2)c1]

To a solution of methyl 2-(1-(4-chlorophenyl)-1H-pyrazole-4-sulfonamido)-5-nitrobenzoate (2.5 g, 5.72 mmol) in MeOH (30 mL), DMSO (50 mL), and H₂O (10 mL) were added iron (3.20 g, 57.2 mmol) and NH₄Cl (3.06 g, 57.2 mmol) at room temperature. The reaction was stirred at 80° C. for 15 h. After cooling to room temperature, the mixture was filtered, and concentrated in vacuo. The resulting residue was diluted with H₂O (100 mL), and extracted with EtOAc (3×40 mL). The organic layers were combined, washed with brine (200 mL), dried over anh. Na₂SO₄, filtered, and the filtrate was concentrated in vacuo. The resulting residue was purified by flash chromatography on SiO₂ eluting with 1:2 mixture of EtOAc: ether give provide 5-amino-2-(1-(4-chlorophenyl)-1H-pyrazole-4-sulfonamido)benzoate. ¹H NMR (400 MHZ, DMSO-d6) δ ppm 9.47 (br s, 1H), 8.95 (s, 1H), 7.94-7.77 (m, 3H), 7.57 (br d, J=8.0 Hz, 2H), 7.17 (br d, J=8.0 Hz, 1H), 6.98 (br s, 1H), 6.76 (br d, J=8.0 Hz, 1H), 5.38 (br s, 2H), 3.66 (s, 3H).

The following intemediate of the present disclosure was made using the methods described in Intermediate 17 above, and substituting the appropriate reactants and/or reagents:

| Compound | Structure |
|---|---|
| Int-18 | 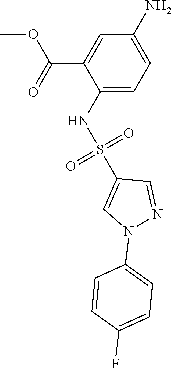 [SMILES: COC(=O)c1cc(N)ccc1NS(=O)(=O)c1cnn(-c2ccc(F)cc2)c1] |

### Intermediate 19

### Preparation of Intermediate 19

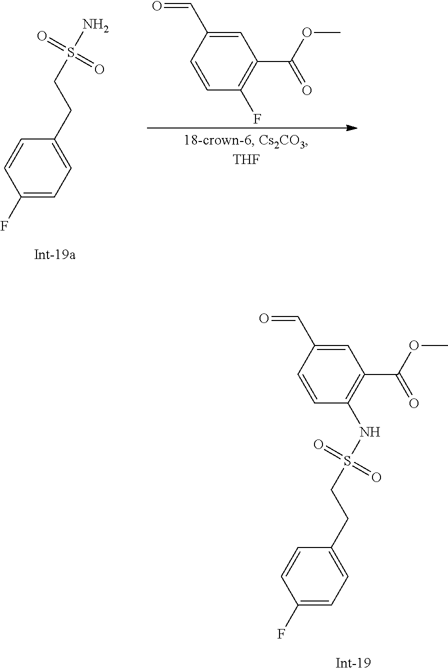 [SMILES: NS(=O)(=O)CCc1ccc(F)cc1 ; COC(=O)c1cc(C=O)ccc1F ; COC(=O)c1cc(C=O)ccc1NS(=O)(=O)CCc1ccc(F)cc1]

### Step 1: methyl 2-((2-(4-fluorophenyl)ethyl) sulfonamido)-5-formylbenzoate (Int-9)

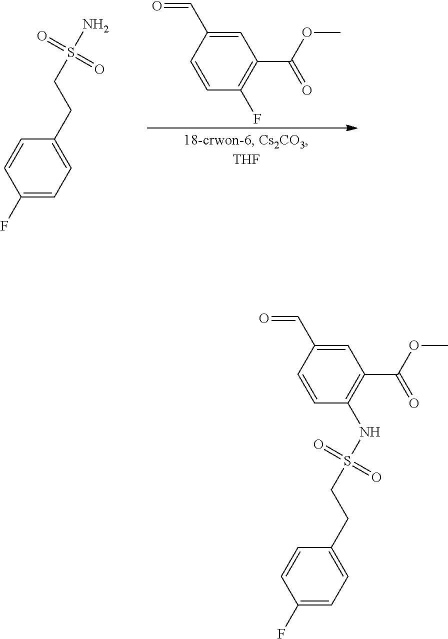 [SMILES: NS(=O)(=O)CCc1ccc(F)cc1 ; COC(=O)c1cc(C=O)ccc1F ; COC(=O)c1cc(C=O)ccc1NS(=O)(=O)CCc1ccc(F)cc1]

To a stirred mixture of 2-(4-fluorophenyl) ethane-1-sulfonamide (50 mg, 0.246 mmol) in THF (1 mL) was added methyl 2-fluoro-5-formylbenzoate (44.8 mg, 0.246 mmol), Cs₂CO₃ (240 mg, 0.738 mmol), and 18-crown-6 (65.0 mg, 0.246 mmol) at 25° C. The reaction mixture was stirred at 80° C. for 16 h, concentrated in vacuo, and the resulting residue was purified by flash chromatography on SiO₂ [Isco®; Silica Flash 20 g Column, eluent of 35 to 100% EtOAc/Pet. ether gradient at 35 mL/min] to provide methyl 2-((2-(4-fluorophenyl)ethyl)-sulfonamido)-5-formylbenzoate. MS (ESI) m/z: 366.0 [M+H⁺]. ¹H NMR (500 MHZ, chloroform-d) δ ppm 3.10-3.20 (m, 2H) 3.46-3.56 (m, 2H) 3.98 (s, 3H) 6.84-6.97 (m, 2H) 7.07 (dd, J=8.54, 5.34 Hz, 2H) 7.87 (d, J=8.70 Hz, 1H) 8.04 (dd, J=8.70, 1.98 Hz, 1H) 8.54 (d, J=1.98 Hz, 1H) 9.94 (s, 1 H).

### Intermediate 20

### Preparation of Intermediate 20

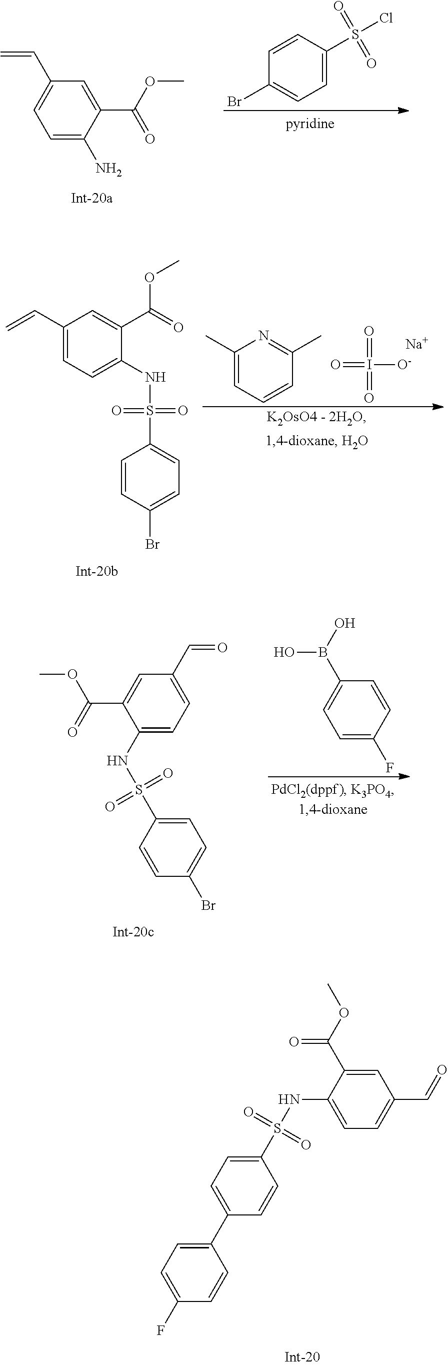

### Step 1: methyl 2-(4-bromophenylsulfonamido)-5-vinylbenzoate (Int-20b)

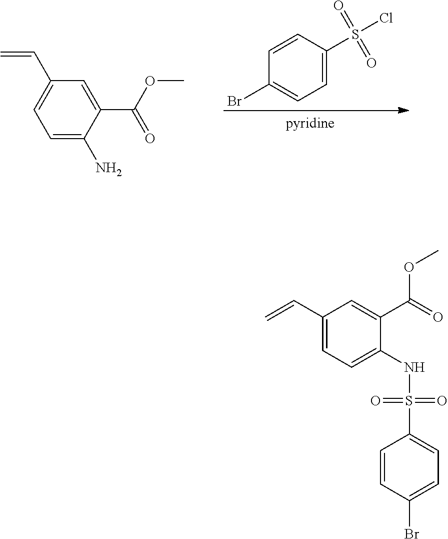 [SMILES: C=Cc1ccc(N)c(C(=O)OC)c1 ; O=S(=O)(Cl)c1ccc(Br)cc1 ; C=Cc1ccc(NS(=O)(=O)c2ccc(Br)cc2)c(C(=O)OC)c1]

To a stirred solution of methyl 2-amino-5-vinylbenzoate (0.5 g, 2.82 mmol) in pyridine (10 ml) was added 4-bromobenzene-1-sulfonyl chloride (1.081 g, 4.23 mmol) at 25° C. The reaction mixture was stirred at 50° C. for 16 h, diluted with H₂O (20 mL), and extracted with EtOAc (2×20 mL). The combined organic layers were dried over anh. Na₂SO₄, filtered, and the filtrate was concentrated in vacuo. The resulting residue was purified by flash chromatography on SiO₂ [Isco®, Agela® Flash 20 g Column, eluent of 10% EtOAc/Pet. ether isocratic gradient at 35 mL/min) to provide methyl 2-(4-bromophenylsulfonamido)-5-vinylbenzoate. MS (ESI) m/z: 395.9, 397.9 [M+H⁺]. ¹H NMR (500 MHZ, CHLOROFORM-d) δ10.49-10.72 (m, 1H), 7.93 (d, J=2.14 Hz, 1H), 7.64-7.73 (m, 3H), 7.50-7.59 (m, 3H), 6.55-6.66 (m, 1H), 5.69 (d, J=17.55 Hz, 1H), 5.26 (d, J=10.99 Hz, 1H), 3.88-3.90 (m, 3H)

### Step 2: methyl 2-(4-bromophenylsulfonamido)-5-formylbenzoate (Int-20c)

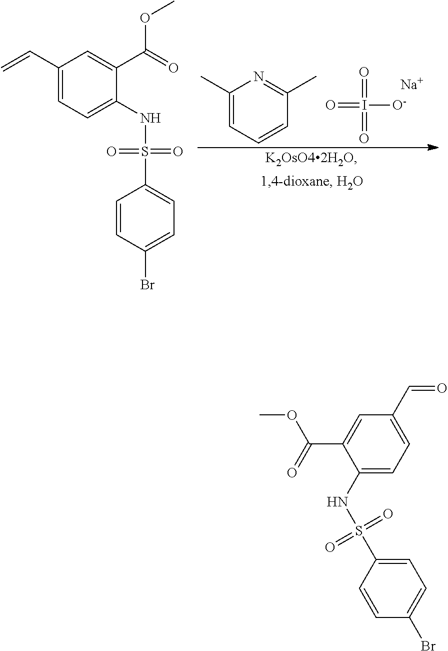

To a solution of methyl 2-(4-bromophenylsulfonamido)-5-vinylbenzoate (100 mg, 0.252 mmol) in 1,4-dioxane (4 mL) and H₂O (1 mL) was added 2,6-dimethylpyridine (0.059 mL, 0.505 mmol), sodium periodate (216 mg, 1.009 mmol), and potassium osmate (VI) dihydrate (9.30 mg, 0.025 mmol) at 0° C. The reaction was allowed to warm to room temperature while stirring for 16 h. The reaction mixture was filtered, quenched with H₂O (10 mL), and extracted with EtOAc (3×10 mL). The combined organic phases were washed with brine (10 mL), dried over anh.

Na₂SO₄, filtered, and concentrated in vacuo. The resulting residue was purified by preparative TLC (EtOAc/Pet. ether 1/3) to provide methyl 2-(4-bromophenylsulfonamido)-5-formylbenzoate. MS (ESI) m/z: 397.9,399.9 [M+H⁺]. ¹H NMR (400 MHZ, CDCl₃) δ 11.18 (br s, 1H), 9.85-9.92 (m, 1H), 8.49 (d, J=1.96 Hz, 1H), 7.92-7.99 (m, 1H), 7.75-7.83 (m, 3H), 7.63 (d, J=8.61 Hz, 2H), 3.97 (s, 3H).

### Step 3: methyl 2-((4′-fluoro-[1,l′-biphenyl])-4-sulfonamido)-5-formylbenzoate (Int-20)

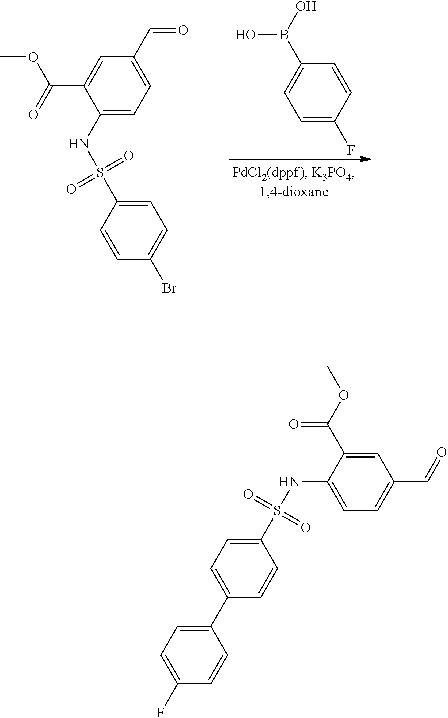 [SMILES: COC(=O)c1cc(C=O)ccc1NS(=O)(=O)c1ccc(Br)cc1 ; OB(O)c1ccc(F)cc1 ; COC(=O)c1cc(C=O)ccc1NS(=O)(=O)c1ccc(-c2ccc(F)cc2)cc1]

To a stirred mixture of methyl 2-((4-bromophenyl) sulfonamido)-5-formylbenzoate (Int-20c; 7 g, 17.58 mmol)) in 1,4-dioxane (140 mL) was added (4-fluorophenyl) boronic acid (2.95 g, 21.09 mmol), PdCl2 (dppf) (1.286 g, 1.758 mmol), and potassium phosphate tribasic (11.19 g, 52.7 mmol) at 25° C. The reaction mixture was stirred at 80° C. for 6 h under N₂. The mixture was cooled to room temperature, diluted with EtOAc (100 mL), washed with H₂O (100 mL), dried over Na₂SO₄, filtered, and the filtrate was evaporated in vacuo. EtOAc (50 mL) was added and the suspension stirred for 10 min. The solvent was removed in vacuo to provide methyl 2-((4′-fluoro-[1,1′-biphenyl])-4-sulfonamido)-5-formylbenzoate, which was used in the subsequent step without further purification. MS (ESI) m/z: 414.2 [M+H⁺]. ¹H NMR (500 MHZ, CDCl₃) δ=11.22 (br s, 1H), 9.89 (s, 1H), 8.49 (s, 1H), 8.09-7.92 (m, 3H), 7.87 (d, J=8.9 Hz, 1H), 7.65 (br d, J=8.2 Hz, 2H), 7.53 (br dd, J=5.3, 8.4 Hz, 2H), 7.15 (t, J=8.5 Hz, 2H), 3.98 (s, 3H).

The following intemediates of the present disclosure were made using the methods described for Intermediate 20 above, and substituting the appropriate reactants and/or reagents:

| Intermediate | Structure |
|---|---|
| Int-21 | 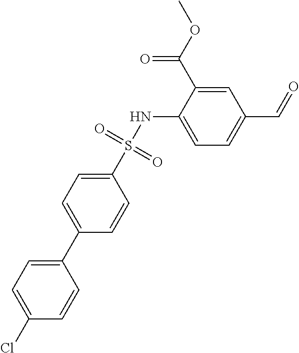 [SMILES: COC(=O)c1cc(C=O)ccc1NS(=O)(=O)c1ccc(-c2ccc(Cl)cc2)cc1] |
| Int-22 | 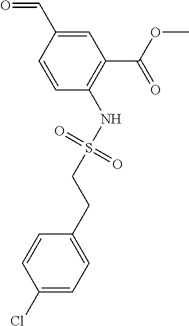 [SMILES: COC(=O)c1cc(C=O)ccc1NS(=O)(=O)CCc1ccc(Cl)cc1] |

### EXAMPLE 1

### Preparation of Compound 1

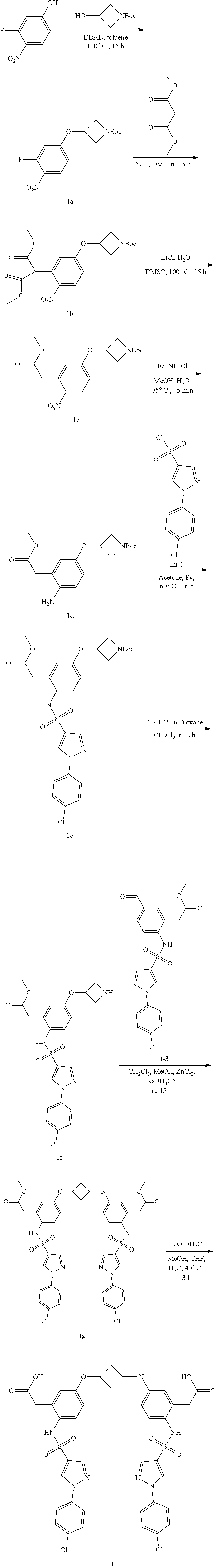

### Step 1: tert-butyl 3-(3-fluoro-4-nitrophenoxy) azetidine-1-carboxylate (1a)

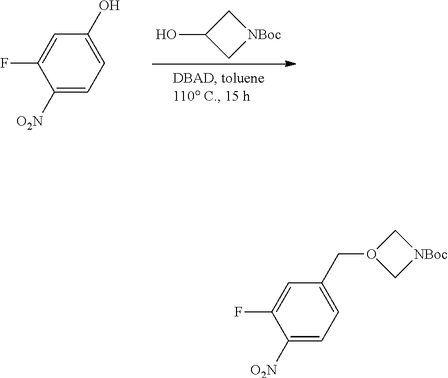

To a solution of 3-fluoro-4-nitrophenol (2 g, 12.73 mmol) in toluene (20 mL) were added t-butyl-3-hydroxyazetidine-1-carboxylate (2.205 g, 12.73 mmol), DBAD (3.81 g, 16.55 mmol), and triphenylphosphine (4.34 g, 16.55 mmol) at room temperature. The resulting reaction mixture was heated to 110° C. for 15 h, concentrated in vacuo, and the resulting residue was purified by flash chromatography on SiO₂ [Isco®; Agela® Flash Column Silica-CS (80 g), Eluent of 0 to 23% EtOAc/Pet.ether @ 35 mL/min] to provide tert-butyl 3-(3-fluoro-4-nitrophenoxy) azetidine-1-carboxylate. ¹H NMR (400 MHZ, CDCl₃) δ (ppm) 8.11 (t, J=8.8 Hz, 1H), 6.58-6.68 (m, 2H), 4.95 (tt, J=6.4, 4.0 Hz, 1H), 4.36 (ddd, J=10.0, 6.4, 1.2 Hz, 2H), 4.03 (dd, J=10.0, 3.2 Hz, 2H), 1.46 (s, 9H).

### Step 2: dimethyl 2-(5-((1-(tert-butoxycarbonyl) azetidin-3-yl)oxy)-2-nitrophenyl) malonate (1b)

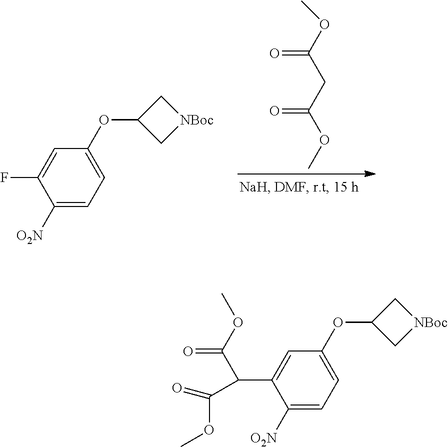

To a stirred solution of dimethyl malonate (0.732 mL, 6.40 mmol) in DMF (10 mL) was slowly added NaH (0.256 g, 6.40 mmol, 60% in mineral oil) at 0° C. The reaction mixture was stirred at 0° C. for 30 min, then a solution of tert-butyl 3-(3-fluoro-4-nitrophenoxy)-azetidine-1-carboxylate (1a; 1.0 g, 3.20 mmol) in DMF (10 mL) was added at 0° C. The reaction was warmed to room temperature and stirred for 15 h. NH4Cl aq. (10 mL) and water (50 mL) were added and the mixture was extracted with EtOAc (30 mL×2). The combined organic fractions were washed with water (30 mL), dried over Na₂SO₄, filtered and concentrated in vacuo. The resulting residue was purified by flash chromatography on SiO₂ [Isco®; Agela® Flash Column Silica-CS (25 g), eluent of 0 to 30.8% EtOAc/Pet. ether gradient @ 40 mL/min] to provide dimethyl 2-(5-((1-(tert-butoxycarbonyl) azetidin-3-yl)oxy)-2-nitrophenyl) malonate. ¹H NMR (400 MHZ, CDCl₃) ¿ (ppm) 8.16 (d, J=9.2 Hz, 1H), 6.87 (d, J=2.8 Hz, 1H), 6.78 (dd, J=9.2, 2.8 Hz, 1H), 5.47 (s, 1H), 4.93-5.01 (m, 1H), 4.34 (dd, J=10.8, 6.4 Hz, 2H), 4.03 (dd, J=10.8, 4.0 Hz, 2H), 3.82 (s, 6H), 1.46 (s, 9H).

### Step 3: tert-butyl 3-(3-(2-methoxy-2-oxoethyl)-4-nitrophenoxy) azetidine-1-carboxylate (1c)

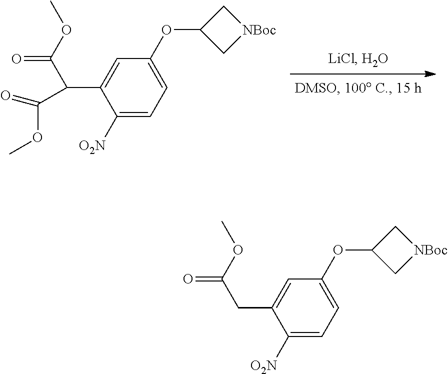 [SMILES: COC(=O)C(C(=O)OC)c1cc(OC2CN(C(=O)OC(C)(C)C)C2)ccc1[N+](=O)[O-] ; COC(=O)Cc1cc(OC2CN(C(=O)OC(C)(C)C)C2)ccc1[N+](=O)[O-]]

To a solution of dimethyl 2-(5-((1-(tert-butoxycarbonyl) azetidin-3-yl)oxy)-2-nitrophenyl) malonate (1b; 650 mg, 1.532 mmol) in DMSO (5 mL) was added lithium chloride (130 mg, 3.06 mmol) and water (0.028 mL, 1.532 mmol) at room temperature. The reaction mixture was stirred at 100° C. for 15 h. Then the reaction mixture was purified directly by reverse phase HPLC chromatography [Gilson 281, YMC-Actus Triart C18 column (100*30 mm*5 μm), eluent of Mobile Phase A: H₂O (0.1% TFA) and Mobile Phase B: MeCN @ 220 nm] to provide tert-butyl 3-(3-(2-methoxy-2-oxoethyl)-4-nitrophenoxy)-azetidine-1-carboxylate. MS (ESI, m/z): 311.2 [M+H⁺-56]. 1H NMR (400 MHZ, CDCl₃) δ (ppm) 8.17-8.22 (m, 1H), 6.67-6.81 (m, 2H), 4.92-4.99 (m, 1H), 4.35 (dd, J=9.2, 6.8 Hz, 2H), 3.99-4.07 (m, 4H), 3.73 (s, 3H), 1.46 (s, 9H).

### Step 4: tert-butyl 3-(4-amino-3-(2-methoxy-2-oxoethyl) phenoxy) azetidine-1-carboxylate (1d)

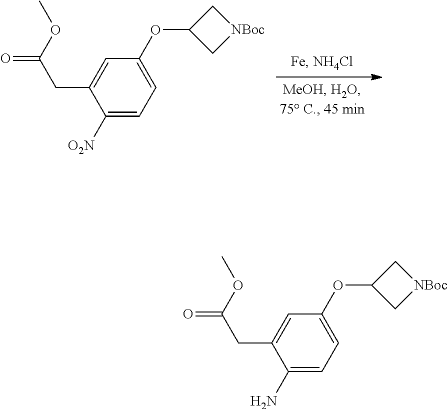 [SMILES: COC(=O)Cc1cc(OC2CN(C(=O)OC(C)(C)C)C2)ccc1[N+](=O)[O-] ; COC(=O)Cc1cc(OC2CN(C(=O)OC(C)(C)C)C2)ccc1N]

To a solution of tert-butyl 3-(3-(2-methoxy-2-oxoethyl)-4-nitrophenoxy) azetidine-1-carboxylate (1c; 280 mg, 0.764 mmol) in MeOH (5 mL) and water (0.5 mL) were added NH₄Cl (409 mg, 7.64 mmol) and iron (427 mg, 7.64 mmol) at room temperature. The reaction mixture was stirred at 75° C. for 45 min, filtered, and the filtrate was concentrated in vacuo. The resulting residue was diluted with water (20 mL) and extracted with EtOAc (20 mL×2). The combined organics were washed with water (20 mL), dried over Na₂SO₄, filtered, and concentrated in vacuo. The resulting residue was purified by flash chromatography on SiO₂ [Isco®; SepaFlash® Silica Flash Column (4 g), isocratic eluent of 30% EtOAc/Pet. ether at 30 mL/min] to provide tert-butyl 3-(4-amino-3-(2-methoxy-2-oxoethyl) phenoxy) azetidine-1-carboxylate. MS (ESI, m/z): 337.3 [M+H⁺]. ¹H NMR (400 MHZ, CDCl₃) δ (ppm) 6.66 (d, J=8.0 Hz, 1H), 6.50-6.58 (m, 2H), 4.79 (tt, J=6.4, 4.0 Hz, 1H), 4.22-4.28 (m, 2H), 3.98 (dd, J=10.0, 4.4 Hz, 2H), 3.71 (s, 3H), 3.54 (s, 2H), 1.45 (s, 9H)

### Step 5: tert-butyl 3-(4-(1-(4-chlorophenyl)-1H-pyrazole-4-sulfonamido)-3-(2-methoxy-2-oxoethyl) phenoxy) azetidine-1-carboxylate (1e)

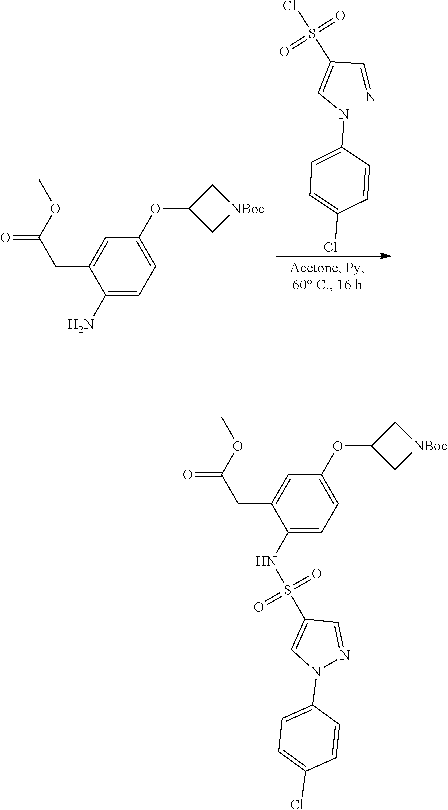 [SMILES: COC(=O)Cc1cc(OC2CN(C(=O)OC(C)(C)C)C2)ccc1N ; N=C/C(=C\Nc1ccc(Cl)cc1)S(=O)(=O)Cl ; COC(=O)Cc1cc(OC2CN(C(=O)OC(C)(C)C)C2)ccc1NS(=O)(=O)c1cnn(-c2ccc(Cl)cc2)c1]

To a solution of tert-butyl 3-(4-amino-3-(2-methoxy-2-oxoethyl) phenoxy) azetidine-1-carboxylate (1d) (120 mg, 0.357 mmol) in acetone (3 mL) and pyridine (0.5 mL) was added 1-(4-chlorophenyl)-1H-pyrazole-4-sulfonyl chloride (Int-1; 148 mg, 0.535 mmol) at room temperature. The reaction was heated to 60° C. and stirred for 16 h. The reaction mixture was concentrated in vacuo and the resulting residue was purified by flash chromatography on SiO₂ [Isco®; SepaFlash® Silica Flash Column (4 g), isocratic eluent of 55% EtOAc/Pet. ether at 30 mL/min] to provide tert-butyl 3-(4-(1-(4-chlorophenyl)-1H-pyrazole-4-sulfonamido)-3-(2-methoxy-2-oxoethyl) phenoxy) azetidine-1-carboxylate. MS (ESI, m/z): 577.2 [M+H⁺].

### Step 6: methyl 2-(5-(azetidin-3-yloxy)-2-(1-(4-chlorophenyl)-1H-pyrazole-4-sulfonamido)phenyl)acetate (If)

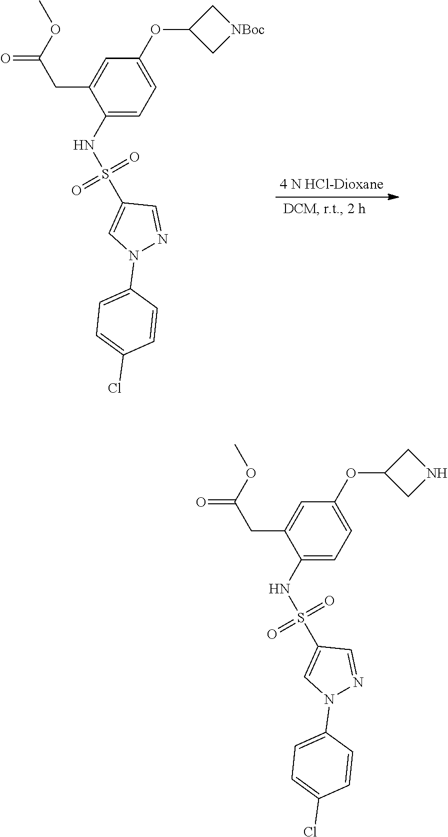 [SMILES: COC(=O)Cc1cc(OC2CN(C(=O)OC(C)(C)C)C2)ccc1NS(=O)(=O)c1cnn(-c2ccc(Cl)cc2)c1 ; COC(=O)Cc1cc(OC2CNC2)ccc1NS(=O)(=O)c1cnn(-c2ccc(Cl)cc2)c1]

To a solution of tert-butyl 3-(4-(1-(4-chlorophenyl)-1H-pyrazole-4-sulfonamido)-3-(2-methoxy-2-oxoethyl) phenoxy) azetidine-1-carboxylate (1e; 120 mg, 0.208 mmol) in CH₂Cl₂ (0.5 mL) was added HCl (4 mL, 16.00 mmol; 4N in dioxane) at room temperature. The reaction mixture was stirred for 2 h, then concentrated in vacuo to provide crude methyl 2-(5-(azetidin-3-yloxy)-2-(1-(4-chlorophenyl)-1H-pyrazole-4-sulfonamido)phenyl)acetate, which was used in the subsequent step without further purification. MS (ESI, m/z): 477.1 [M+H⁺].

### Step 7: methyl 2-(2-(1-(4-chlorophenyl)-1H-pyrazole-4-sulfonamido)-5-((1-(4-(1-(4-chlorophenyl)-1H-pyrazole-4-sulfonamido)-3-(2-methoxy-2-oxoethyl)benzyl) azetidin-3-yl)oxy)phenyl)acetate (1g)

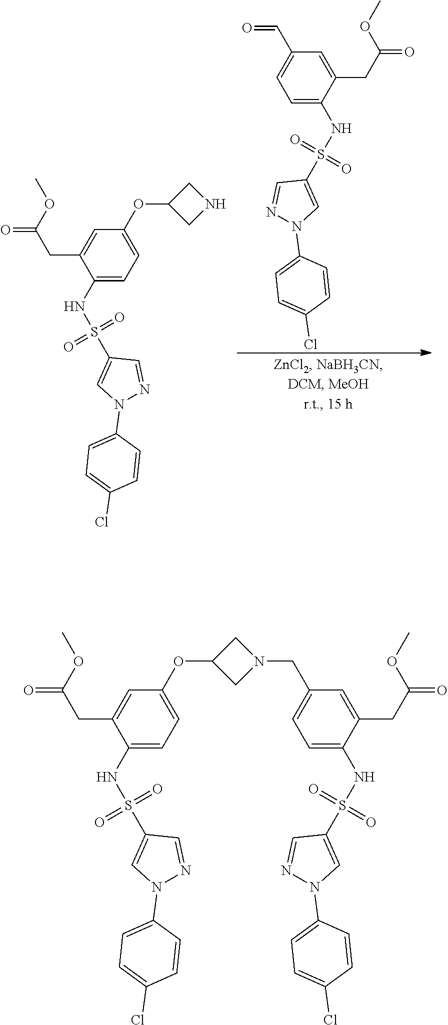 [SMILES: COC(=O)Cc1cc(OC2CNC2)ccc1NS(=O)(=O)c1cnn(-c2ccc(Cl)cc2)c1 ; COC(=O)Cc1cc(C=O)ccc1NS(=O)(=O)c1cnn(-c2ccc(Cl)cc2)c1 ; COC(=O)Cc1cc(CN2CC(Oc3ccc(NS(=O)(=O)c4cnn(-c5ccc(Cl)cc5)c4)c(CC(=O)OC)c3)C2)ccc1NS(=O)(=O)c1cnn(-c2ccc(Cl)cc2)c1]

To a solution of methyl 2-(2-(1-(4-chlorophenyl)-1H-pyrazole-4-sulfonamido)-5-formylphenyl)acetate (1f; 45 mg, 0.104 mmol) in CH₂Cl₂ (1 mL) and MeOH (4 mL) were added methyl 2-(5-(azetidin-3-yloxy)-2-(1-(4-chlorophenyl)-1H-pyrazole-4-sulfonamido)-phenyl)acetate (Int-3; 49.5 mg, 0.104 mmol), zinc (II) chloride (28.3 mg, 0.207 mmol) and NaBH₃CN (19.55 mg, 0.311 mmol) at room temperature. The reaction mixture was stirred at room temperature for 15 h, then concentrated in vacuo to provide crude methyl 2-(2-(1-(4-chlorophenyl)-1H-pyrazole-4-sulfonamido)-5-((1-(4-(1-(4-chlorophenyl)-1H-pyrazole-4-sulfonamido)-3-(2-methoxy-2-oxoethyl)benzyl)-azetidin-3-yl)oxy)phenyl)acetate, which was used in the next step without further purification. LCMS (ESI, m/z): 894.0 [M+H⁺].

### Step 8:2-(5-((1-(3-(carboxymethyl)-4-(1-(4-chlorophenyl)-1H-pyrazole-4-sulfonamido)benzyl) azetidin-3-yl)oxy)-2-(1-(4-chlorophenyl)-1H-pyrazole-4-sulfonamido)phenyl) acetic acid (1)

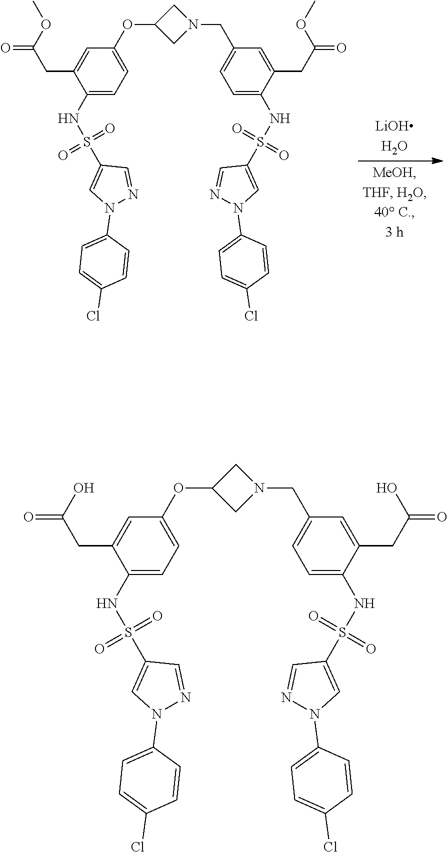 [SMILES: COC(=O)Cc1cc(CN2CC(Oc3ccc(NS(=O)(=O)c4cnn(-c5ccc(Cl)cc5)c4)c(CC(=O)OC)c3)C2)ccc1NS(=O)(=O)c1cnn(-c2ccc(Cl)cc2)c1 ; O=C(O)Cc1cc(CN2CC(Oc3ccc(NS(=O)(=O)c4cnn(-c5ccc(Cl)cc5)c4)c(CC(=O)O)c3)C2)ccc1NS(=O)(=O)c1cnn(-c2ccc(Cl)cc2)c1]

To a solution of methyl 2-(2-(1-(4-chlorophenyl)-1H-pyrazole-4-sulfonamido)-5-((1-(4-(1-(4-chlorophenyl)-1H-pyrazole-4-sulfonamido)-3-(2-methoxy-2-oxoethyl)benzyl) azetidin-3-yl)oxy)phenyl) acetate (1g; 90 mg, 0.101 mmol) in THF (2 mL), MeOH (2 mL) and water (1 mL) was added lithium hydroxide hydrate (42.2 mg, 1.006 mmol) at room temperature. The reaction was heated to 40° C. and stirred for 3 h. The reaction mixture was acidified with 3N HCl to pH=2, concentrated in vacuo, and the resulting residue was purified by reverse phase HPLC chromatography [Gilson 281, YMC-Actus Triart C18 column (100*30 mm*5 μm), eluent of Mobile Phase A: H₂O (0.1% TFA) and Mobile Phase B: MeCN at 220 nm] to provide 2-(5-((1-(3-(carboxymethyl)-4-(1-(4-chlorophenyl)-1H-pyrazole-4-sulfonamido)benzyl) azetidin-3-yl)oxy)-2-(1-(4-chloro-phenyl)-1H-pyrazole-4-sulfonamido)phenyl) acetic acid. MS (ESI, m/z): 864.1 [M−H⁺]. ¹H NMR (500 MHz, CD₃OD) δ (ppm) 8.63 (s, 1H), 8.55 (s, 1H), 7.86 (s, 1H), 7.82 (s, 1H), 7.75-7.79 (m, 4H), 7.50 (t, J=9.0 Hz, 4H), 7.40 (s, 1H), 7.33 (s, 2H), 7.00 (d, J=9.0 Hz, 1H), 6.84 (d, J-2.8 Hz, 1H), 6.70 (dd, J=8.8, 3.0 Hz, 1H), 5.05-5.10 (m, 1H), 4.55 (dd, J=12.0, 6.5 Hz, 2H), 4.39 (s, 2H), 4.19 (dd, J=12.2, 4.0 Hz, 2H), 3.72 (d, J=9.5 Hz, 4H).

### EXAMPLE 2

### Preparation of Compound 2

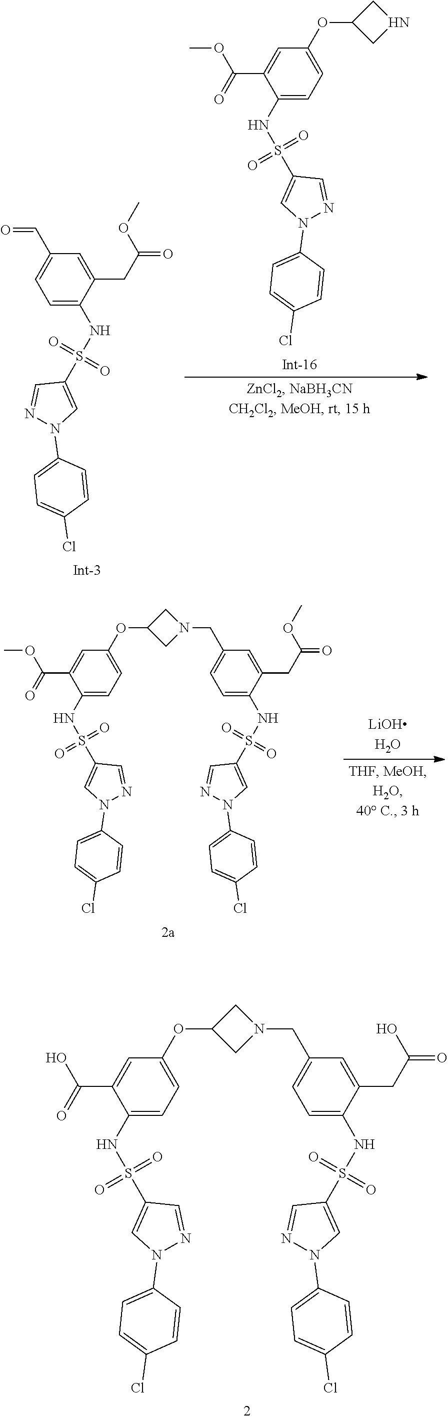 [SMILES: COC(=O)Cc1cc(C=O)ccc1NS(=O)(=O)c1cnn(-c2ccc(Cl)cc2)c1 ; COC(=O)c1cc(OC2CNC2)ccc1NS(=O)(=O)c1cnn(-c2ccc(Cl)cc2)c1 ; COC(=O)Cc1cc(CN2CC(Oc3ccc(NS(=O)(=O)c4cnn(-c5ccc(Cl)cc5)c4)c(C(=O)OC)c3)C2)ccc1NS(=O)(=O)c1cnn(-c2ccc(Cl)cc2)c1 ; O=C(O)Cc1cc(CN2CC(Oc3ccc(NS(=O)(=O)c4cnn(-c5ccc(Cl)cc5)c4)c(C(=O)O)c3)C2)ccc1NS(=O)(=O)c1cnn(-c2ccc(Cl)cc2)c1]

### Step 1: methyl 2-((1-(4-chlorophenyl)-1H-pyrazole)-4-sulfonamido)-5-((1-(4-((1-(4-chlorophenyl)-1H-pyrazole)-4-sulfonamido)-3-(2-methoxy-2-oxoethyl)benzyl) azetidin-3-yl)oxy)benzoate (2a)

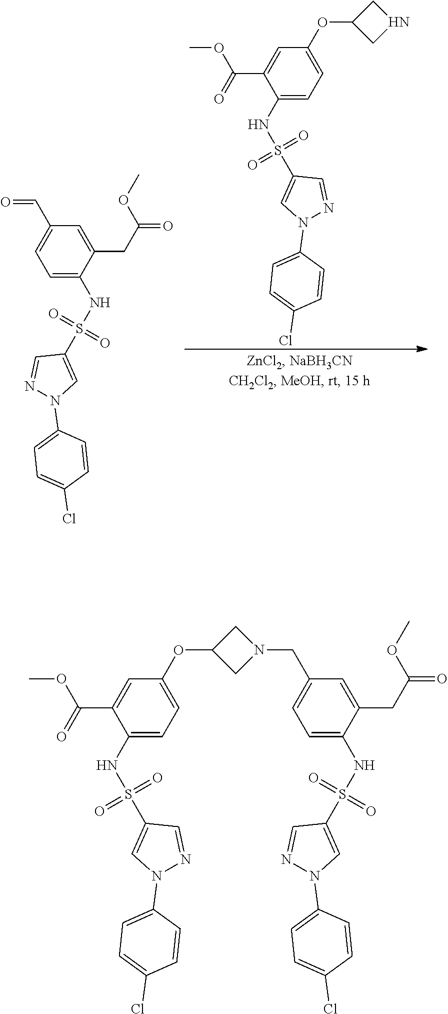 [SMILES: COC(=O)Cc1cc(C=O)ccc1NS(=O)(=O)c1cnn(-c2ccc(Cl)cc2)c1 ; COC(=O)c1cc(OC2CNC2)ccc1NS(=O)(=O)c1cnn(-c2ccc(Cl)cc2)c1 ; COC(=O)Cc1cc(CN2CC(Oc3ccc(NS(=O)(=O)c4cnn(-c5ccc(Cl)cc5)c4)c(C(=O)OC)c3)C2)ccc1NS(=O)(=O)c1cnn(-c2ccc(Cl)cc2)c1]

To a solution of Int-3 (45 mg, 0.104 mmol) in CH₂Cl₂ (2 mL) and MeOH (2 mL) was added methyl 5-(azetidin-3-yloxy)-2-(1-(4-chlorophenyl)-1H-pyrazole-4-sulfonamido)benzoate (Int-16; 48.0 mg, 0.104 mmol), zinc (II) chloride (28.3 mg, 0.207 mmol) and NaBH₃CN (19.55 mg, 0.311 mmol) at room temperature. The reaction mixture was stirred for 15 h and then concentrated in vacuo to provide crude methyl 2-((1-(4-chloro-phenyl)-1H-pyrazole)-4-sulfonamido)-5-((1-(4-((1-(4-chlorophenyl)-1H-pyrazole)-4-sulfonamido)-3-(2-methoxy-2-oxoethyl)benzyl) azetidin-3-yl)oxy)benzoate, which was used in the next step without further purification assuming quantitative conversion. MS (ESI m/z): 880.0 [M+H⁺].

### Step 2:5-((1-(3-(carboxymethyl)-4-((1-(4-chlorophenyl)-1H-pyrazole)-4-sulfonamido)-benzyl) azetidin-3-yl)oxy)-2-((1-(4-chlorophenyl)-1H-pyrazole)-4-sulfonamido)benzoic acid (2)

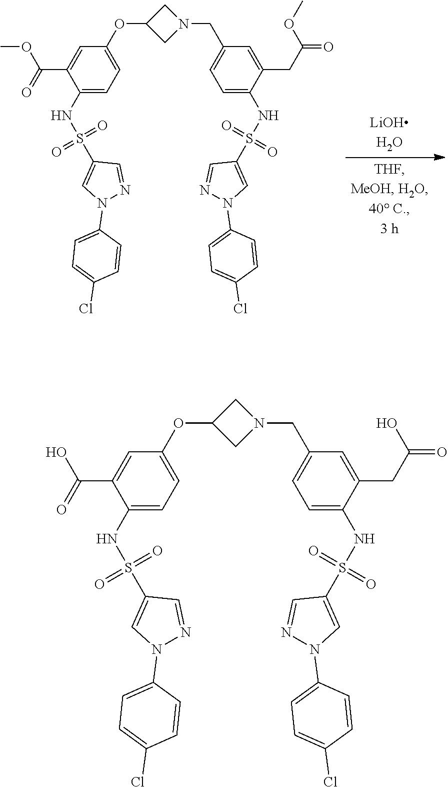 [SMILES: COC(=O)Cc1cc(CN2CC(Oc3ccc(NS(=O)(=O)c4cnn(-c5ccc(Cl)cc5)c4)c(C(=O)OC)c3)C2)ccc1NS(=O)(=O)c1cnn(-c2ccc(Cl)cc2)c1 ; O=C(O)Cc1cc(CN2CC(Oc3ccc(NS(=O)(=O)c4cnn(-c5ccc(Cl)cc5)c4)c(C(=O)O)c3)C2)ccc1NS(=O)(=O)c1cnn(-c2ccc(Cl)cc2)c1]

To a solution of crude methyl 2-(1-(4-chlorophenyl)-1H-pyrazole-4-sulfonamido)-5-((1-(4-(1-(4-chlorophenyl)-1H-pyrazole-4-sulfonamido)-3-(2-methoxy-2-oxoethyl)benzyl)-azetidin-3-yl)oxy)benzoate (2a from Step 1; 0.104 mmol) in THF (2 mL), MeOH (2 mL), and water (1 mL) was added lithium hydroxide monohydrate (23.82 mg, 0.568 mmol) at room temperature. The reaction was stirred at 40° C. for 3 h, then acidified to pH=2 by the addition of 3 N HCl. The solvent was removed in vacuo and the resulting residue was purified by reverse phase HPLC chromatography |Gilson 281, YMC-Actus Triart C18 column (100*30 mm*5 μm), eluent of Mobile Phase A: H₂O (0.1% TFA) and Mobile Phase B: MeCN @ 220 nm] to provide 5-((1-(3-(carboxymethyl)-4-(1-(4-chlorophenyl)-1H-pyrazole-4-sulfonamido)benzyl) azetidin-3-yl)oxy)-2-(1-(4-chlorophenyl)-1H-pyrazole-4-sulfonamido)benzoic acid. MS (ESI m/z): 850.1 [M−H⁺]. ¹H NMR (400 MHZ, CD₃OD) δ ppm 8.71 (s, 1H), 8.56 (br s, 1H), 7.82 (s, 1H), 7.78 (s, 1H), 7.72 (br dd, J=12.4, 8.8 Hz, 5H), 7.38-7.50 (m, 6H), 7.29 (s, 1H), 7.23-7.27 (m, 1H), 7.12 (br d, J=5.8 Hz, 1H), 5.08 (br s, 1H), 4.52-4.60 (m, 2H), 4.39 (s, 2H), 4.20 (br s, 2H), 3.77 (s, 2H).

### EXAMPLE 3

### Preparation of Compound 3

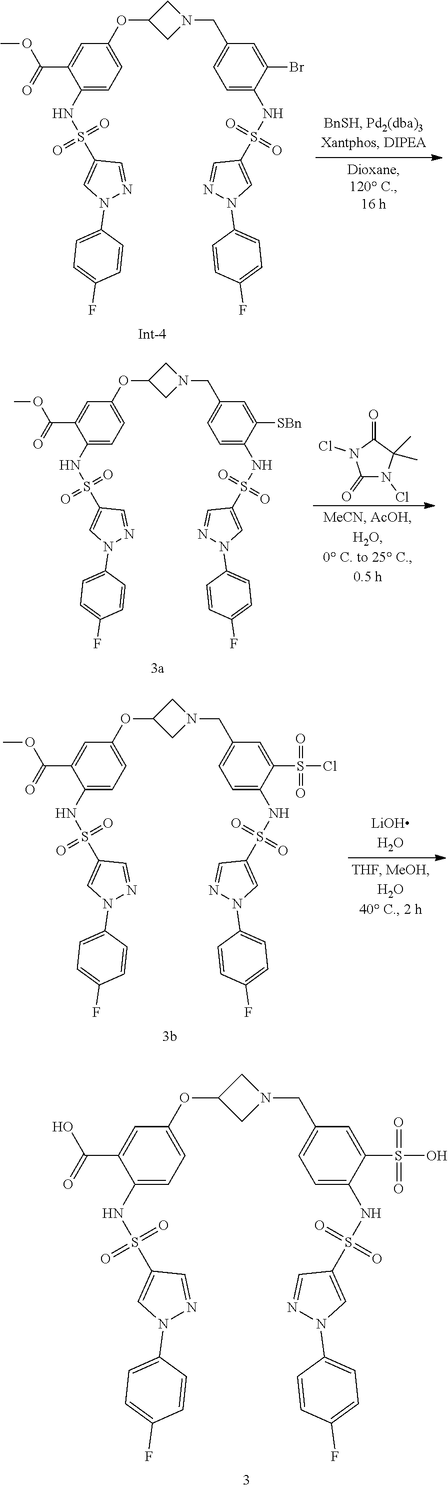

### Step 1: methyl 5-((1-(3-(benzylthio)-4-((1-(4-fluorophenyl)-1H-pyrazole)-4-sulfonamido)benzyl)-azetidin-3-yl)oxy)-2-((1-(4-fluorophenyl)-1H-pyrazole)-4-sulfonamido)benzoate (3a)

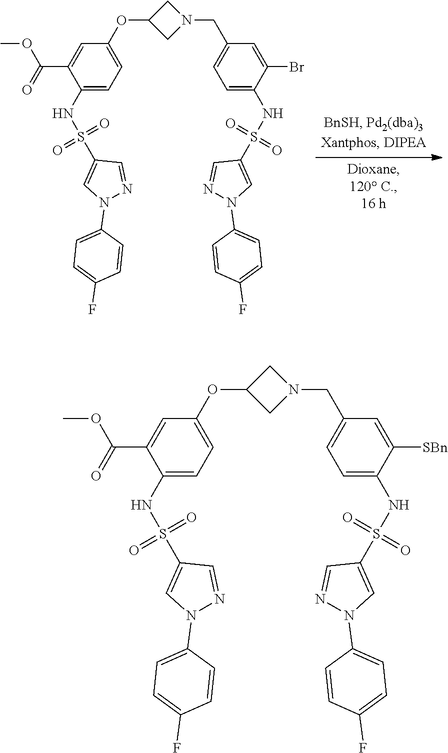

To a stirred mixture of methyl 5-((1-(3-bromo-4-((1-(4-fluorophenyl)-1H-pyrazole)-4-sulfonamido)benzyl) azetidin-3-yl)oxy)-2-((1-(4-fluorophenyl)-1H-pyrazole)-4-sulfon-amido)benzoate (Int-4; 100 mg, 0.117 mmol), benzylthiol (0.014 mL, 0.117 mmol), Pd2 (dba) 3 (10.71 mg, 0.012 mmol), and Xantphos (13.54 mg, 0.023 mmol) in toluene (2 mL) was added DIPEA (0.041 mL, 0.234 mmol). The mixture was heated to 120° C. for 16 h under N₂. Then the solvent was removed in vacuo, and the resulting residue was purified by reverse phase HPLC chromatography [Gilson 281, Phenomenex Luna C18 column (70*30 mm*3 μm), eluent of Mobile Phase A: H₂O (0.1% TFA) and Mobile Phase B: MeCN @ 220 nm] to provide methyl 5-((1-(3-(benzylthio)-4-((1-(4-fluorophenyl)-1H-pyrazole)-4-sulfonamido)benzyl) azetidin-3-yl)oxy)-2-((1-(4-fluorophenyl)-1H-pyrazole)-4-sulfonamido)benzoate. MS (ESI, m/z): 898.2 [M+H⁺].

### Step 2: methyl 5-((1-(3-(chlorosulfonyl)-4-((1-(4-fluorophenyl)-1H-pyrazole)-4-sulfonamido)benzyl) azetidin-3-yl)oxy)-2-((1-(4-fluorophenyl)-1H-pyrazole)-4-sulfonamido)benzoate (3b)

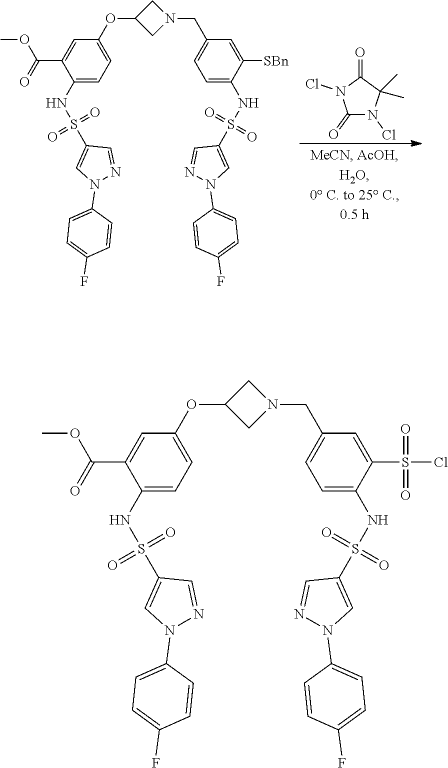

To a stirred mixture of methyl 5-((1-(3-(benzylthio)-4-((1-(4-fluorophenyl)-1H-pyrazole)-4-sulfonamido)benzyl) azetidin-3-yl)oxy)-2-((1-(4-fluorophenyl)-1/-pyrazole)-4-sulfonamido)benzoate (3a; 80 mg, 0.089 mmol) in MeCN (2 mL) and AcOH/H₂O=5:4 (0.1 mL) was added 1,3-dichloro-5,5-dimethylhydantoin (21.06 mg, 0.107 mmol) at 0° C. The reaction was stirred at 25° C. for 30 min, then concentrated in vacuo, and the resulting residue was used immediately in the subsequent step without further purification. MS (ESI, m/z): 856.2 [M+H⁺-18].

### Step 3:2-((1-(4-fluorophenyl)-1H-pyrazole)-4-sulfonamido)-5-((1-(4-((1-(4-fluorophenyl)-1H-pyrazole)-4-sulfonamido)-3-sulfobenzyl) azetidin-3-yl)oxy)benzoic acid (3)

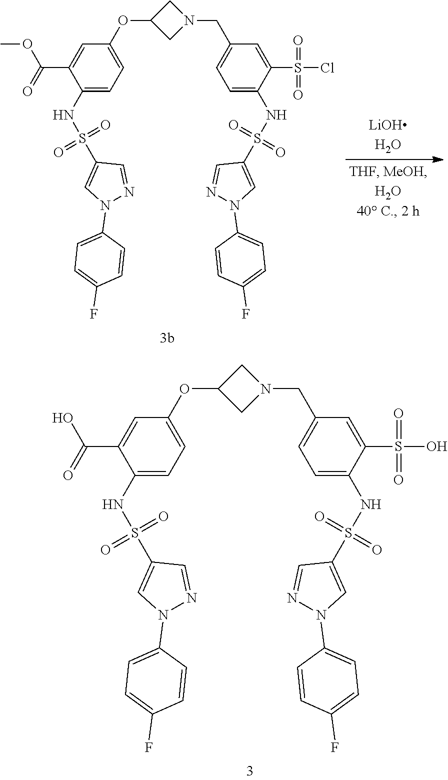 [SMILES: COC(=O)c1cc(OC2CN(Cc3ccc(NS(=O)(=O)c4cnn(-c5ccc(F)cc5)c4)c(S(=O)(=O)Cl)c3)C2)ccc1NS(=O)(=O)c1cnn(-c2ccc(F)cc2)c1 ; O=C(O)c1cc(OC2CN(Cc3ccc(NS(=O)(=O)c4cnn(-c5ccc(F)cc5)c4)c(S(=O)(=O)O)c3)C2)ccc1NS(=O)(=O)c1cnn(-c2ccc(F)cc2)c1]

To a stirred mixture of methyl 5-((1-(3-(chlorosulfonyl)-4-((1-(4-fluorophenyl)-1H-pyrazole)-4-sulfonamido)benzyl) azetidin-3-yl)oxy)-2-((1-(4-fluorophenyl)-1H-pyrazole)-4-sulfonamido)benzoate (3b; 34 mg, 0.039 mmol) in THF (0.2 mL), MeOH (0.2 mL), and H₂O (0.1 mL) was added lithium hydroxide monohydrate (16.32 mg, 0.389 mmol). The reaction mixture was stirred at 40° C. for 2 h, concentrated in vacuo, and acidified to pH=3 with 1 N HCl. The residue was purified by reverse phase HPLC chromatography [Gilson 281, Boston Green ODS column (150*30 mm*5 μm), eluent of Mobile Phase A: H₂O (0.1% TFA) and Mobile Phase B: MeCN at 220 nm] to provide 2-((1-(4-fluorophenyl)-1H-pyrazole)-4-sulfonamido)-5-((3-(4-((1-(4-fluorophenyl)-1H-pyrazole)-4-sulfonamido)-3-(methoxycarbonyl)-phenoxy) azetidin-1-yl)methyl)benzene-sulfonic acid. MS (ESI, m/z): 842.1 [M+H⁺]. ¹H NMR (500 MHz, CD₃OD) δ ppm 8.77 (s, 1H), 8.65 (s, 1H), 7.90 (s, 1H), 7.86 (s, 1H), 7.81 (d, J=8.5 Hz, 1H), 7.78 (s, 1H), 7.72 (d, J=9.0 Hz, 5H), 7.48 (br d, J=7.5 Hz, 1H), 7.39 (br s, 1H), 7.29-7.12 (m, 5H), 5.19-4.98 (m, 1H), 4.56-4.52 (m, 2H), 4.44-4.34 (m, 2H), 4.29-4.09 (m, 2H).

### EXAMPLE 4

### Preparation of Compound 12

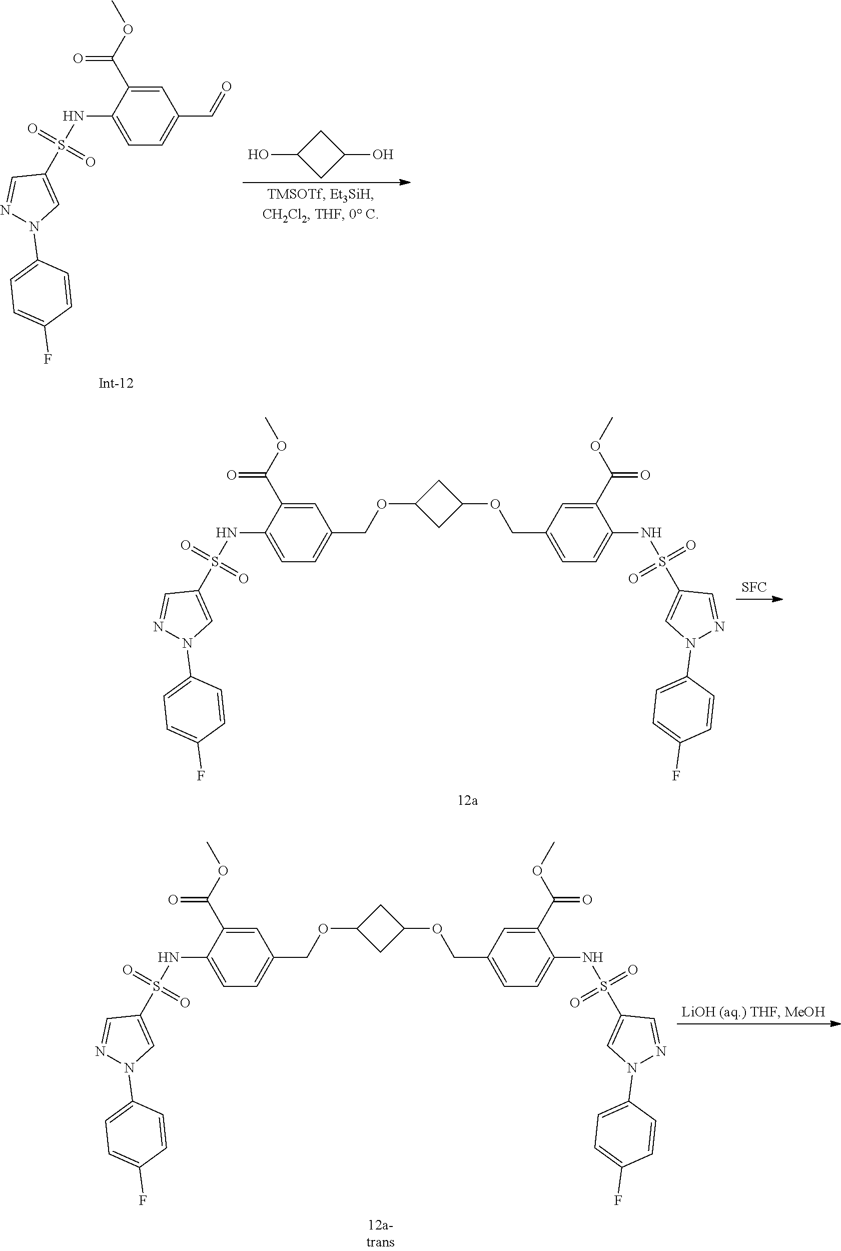

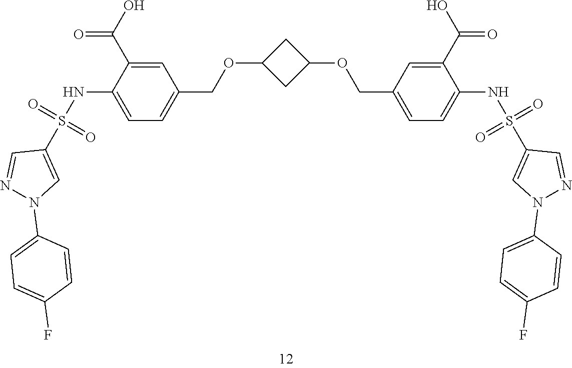 [SMILES: O=C(O)c1cc(COC2CC(OCc3ccc(NS(=O)(=O)c4cnn(-c5ccc(F)cc5)c4)c(C(=O)O)c3)C2)ccc1NS(=O)(=O)c1cnn(-c2ccc(F)cc2)c1]

### Step 1: Methyl 2-(1-(4-fluorophenyl)-1H-pyrazole)-4-sulfonamido)-5-(3-((4-((1-(4-fluorophenyl)-1H-pyrazole)-4-sulfonamido)-3-(methoxycarbonyl)benzyl)oxy)-cyclobutoxy-)methyl)benzoate (12a)

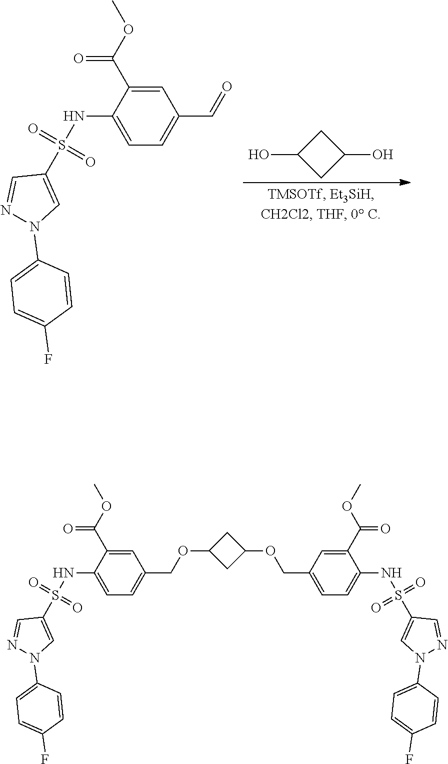 [SMILES: COC(=O)c1cc(C=O)ccc1NS(=O)(=O)c1cnn(-c2ccc(F)cc2)c1 ; OC1CC(O)C1 ; COC(=O)c1cc(COC2CC(OCc3ccc(NS(=O)(=O)c4cnn(-c5ccc(F)cc5)c4)c(C(=O)OC)c3)C2)ccc1NS(=O)(=O)c1cnn(-c2ccc(F)cc2)c1]

A 0° C. solution of methyl 2-((1-(4-fluorophenyl)-1H-pyrazole)-4-sulfonamido)-5-formyl-benzoate (Int-12; 275 mg, 0.681 mmol) in CH₂Cl₂ (4 mL) and THF (1 mL) was treated with cyclobutane-1,3-diol (20 mg, 0.23 mmol) and trimethylsilyl trifluoromethanesulfonate (0.164 mL, 0.908 mmol) and stirred at 0° C. for 10 min. Triethylsilane (0.297 mL, 1.82 mmol) was added and the reaction was stirred at 0° C. for 1 h. H₂O (20 mL) was added and the reaction was extracted with EtOAc (20 mL). The organic fraction was washed with brine, dried over Na₂SO₄, filtered, and concentrated in vacuo. The resulting residue was purified by reverse phase HPLC [Boston Prime C18 column (150 mm*30 mm, 5 μm), eluent of 70 to 100% MeCN/H₂O (+0.05% NH3H₂O+10 mM NH4HCO3)] to provide methyl 2-((1-(4-fluorophenyl)-1H-pyrazole)-4-sulfonamido)-5-((3-((4-((1-(4-fluorophenyl)-1H-pyrazole)-4-sulfonamido)-3-(methoxycarbonyl)benzyl)-oxy)cyclobutoxy)methyl)benzoate. MS (ESI, m/z): 863.1 [M+H⁺].

### Step 2: trans-Methyl 2-(1-(4-fluorophenyl)-1H-pyrazole)-4-sulfonamido)-5-((3-((4-((1-(4-fluorophenyl)-1H-pyrazole)-4-sulfonamido)-3-(methoxycarbonyl)benzyl)oxy)-cyclobutoxy)methyl)benzoate (12a-trans)

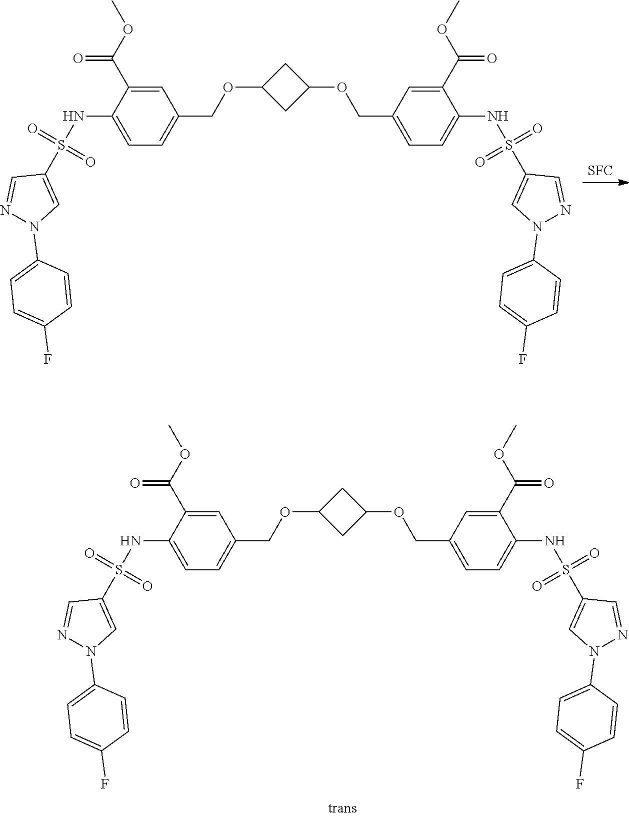

The cis and trans isomers of methyl 2-((1-(4-fluorophenyl)-1H-pyrazole)-4-sulfonamido)-5-((3-((4-((1-(4-fluorophenyl)-1H-pyrazole)-4-sulfonamido)-3-(methoxycarbonyl)-benzyl)oxy)-cyclobutoxy)methyl)benzoate (12a; 130 mg) were separated by chiral Supercritical Fluid Chromatography [DAICEL CHIRALCEL OD-H (250 mm*30 mm, 5 μm), eluent of 0.1% aq. NH₃ in ethanol, 70 mL/min] to provide trans-methyl 2-((1-(4-fluorophenyl)-1H-pyrazole)-4-sulfonamido)-5-((3-((4-((1-(4-fluorophenyl)-1H-pyrazole)-4-sulfonamido)-3-(methoxycarbonyl)benzyl)oxy)-cyclobutoxy)methyl)benzoate (Rt=1.04 min) and cis-methyl 2-((1-(4-fluorophenyl)-1H-pyrazole)-4-sulfonamido)-5-((3-((4-((1-(4-fluorophenyl)-1/-pyrazole)-4-sulfonamido)-3-(methoxycarbonyl)benzyl)oxy)-cyclobutoxy)methyl)benzoate (Rt=1.92 min). trans ¹H NMR (400 MHZ, CDCl₃) δ ppm 10.68 (s, 2H), 8.22 (s, 2H), 7.91 (s, 2H), 7.87 (s, 2H), 7.76 (d, J=8.4 Hz, 2H), 7.61-7.54 (m, 4H), 7.48 (dd, J=2.4, 8.4 Hz, 2H), 7.15 (t, J=8.0 Hz, 4H), 4.31 (s, 4H), 4.20 (quin, J=5.6 Hz, 2H), 3.88 (s, 6H), 2.25 (t, J=5.6 Hz, 4H). cis ¹H NMR (400 MHz, CDCl₃) δ ppm 10.68 (s, 2H), 8.21 (s, 2H), 7.92 (s, 2H), 7.87 (s, 2H), 7.76 (d, J=8.4 Hz, 2H), 7.60-7.54 (m, 4H), 7.47 (dd, J=2.0, 8.4 Hz, 2H), 7.15 (t, J=8.0 Hz, 4H), 4.32 (s, 4H), 3.92-3.86 (m, 6H), 3.73-3.60 (m, 2H), 2.66-2.57 (m, 2H), 2.00-1.92 (m, 2H).

### Step 2: trans-5-(3-((3-Carboxy-4-((1-(4-fluorophenyl)-1H-pyrazole)-4-sulfonamido)benzyl)oxy)cyclobutoxy)methyl)-2-((1-(4-fluorophenyl)-1H-pyrazole)-4-sulfonamido)benzoic acid (12)

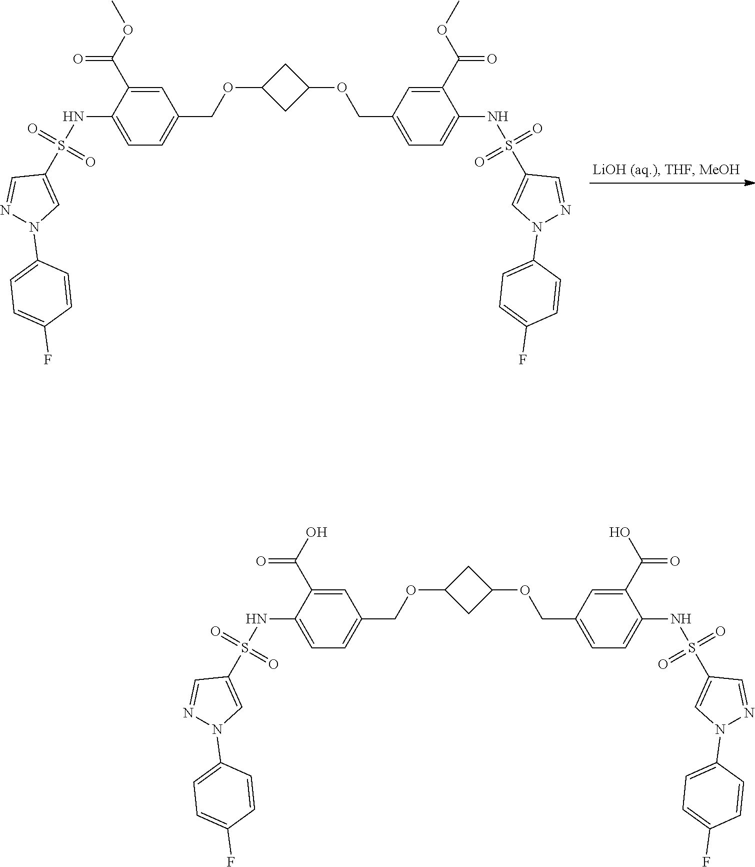 [SMILES: COC(=O)c1cc(COC2CC(OCc3ccc(NS(=O)(=O)c4cnn(-c5ccc(F)cc5)c4)c(C(=O)OC)c3)C2)ccc1NS(=O)(=O)c1cnn(-c2ccc(F)cc2)c1 ; O=C(O)c1cc(COC2CC(OCc3ccc(NS(=O)(=O)c4cnn(-c5ccc(F)cc5)c4)c(C(=O)O)c3)C2)ccc1NS(=O)(=O)c1cnn(-c2ccc(F)cc2)c1]

A mixture of trans-methyl 2-((1-(4-fluorophenyl)-1H-pyrazole)-4-sulfonamido)-5-((3-((4-((1-(4-fluorophenyl)-1H-pyrazole)-4-sulfonamido)-3-(methoxycarbonyl)benzyl)oxy)cyclobutoxy)methyl)benzoate (12a-trans; 80 mg, 0.093 mmol) and lithium hydroxide monohydrate (39 mg, 0.93 mmol) in MeOH (2 mL), THF (2 mL), and H₂O (1 mL) was stirred at 40° C. for 16 h. HCl (2 N) was added until pH˜4. The reaction was concentrated in vacuo and the residue was purified by reverse phase chromatography [Boston Green ODS column, (150 mm*30 mm, 5 μm), eluent of 58 to 88% MeCN/H₂O (+0.1% TFA)] to provide trans-5-((3-((3-carboxy-4-((1-(4-fluorophenyl)-1H-pyrazole)-4-sulfonamido)benzyl)oxy)-cyclobutoxy)methyl)-2-((1-(4-fluorophenyl)-1H-pyrazole)-4-sulfonamido)benzoic acid. MS (ESI) m/z: 833.2 [M−H⁺]. ¹H NMR (500 MHz, CD₃OD) δ ppm 8.71 (s, 2H), 7.95 (d, J=2.0 Hz, 2H), 7.86 (s, 2H), 7.76-7.69 (m, 6H), 7.53 (dd, J=8.5, 2.0 Hz, 2H), 7.22-7.15 (m, 4H), 4.33 (s, 4H), 4.15 (t, J=5.5 Hz, 2H), 2.16 (t, J=5.5 Hz, 4H).

The following compounds (13 through 18) of the present disclosure were made using the methods described in Example 4 above, and substituting the appropriate reactants and/or

| | | Observed |
|---|---|---|
| Compound | Structure | MS |
| 13 | 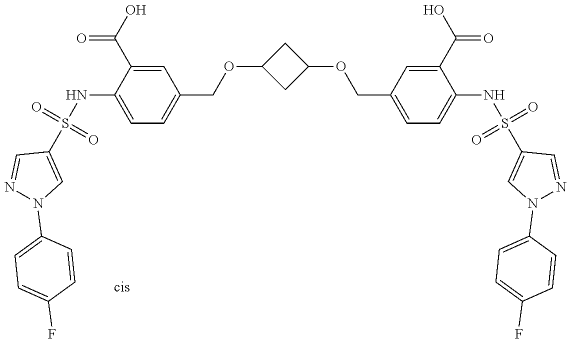 [SMILES: O=C(O)c1cc(COC2CC(OCc3ccc(NS(=O)(=O)c4cnn(-c5ccc(F)cc5)c4)c(C(=O)O)c3)C2)ccc1NS(=O)(=O)c1cnn(-c2ccc(F)cc2)c1] | 835.2 |
| 14 | 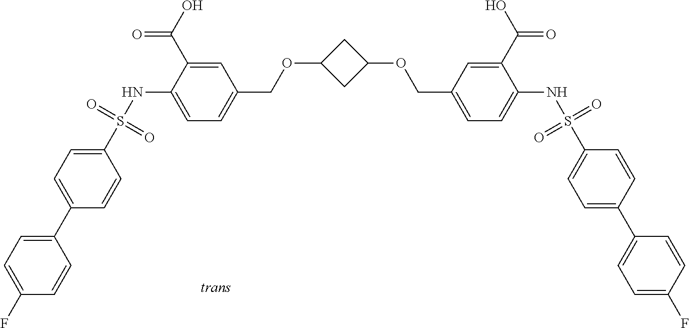 [SMILES: O=C(O)c1cc(COC2CC(OCc3ccc(NS(=O)(=O)c4ccc(-c5ccc(F)cc5)cc4)c(C(=O)O)c3)C2)ccc1NS(=O)(=O)c1ccc(-c2ccc(F)cc2)cc1] | 853.2 |
| 15 | 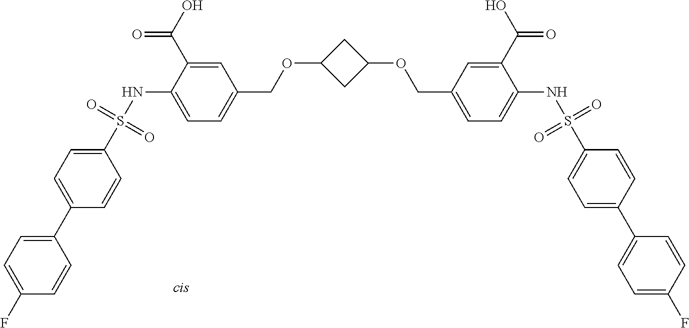 [SMILES: O=C(O)c1cc(COC2CC(OCc3ccc(NS(=O)(=O)c4ccc(-c5ccc(F)cc5)cc4)c(C(=O)O)c3)C2)ccc1NS(=O)(=O)c1ccc(-c2ccc(F)cc2)cc1] | 853.2 |
| 16 | 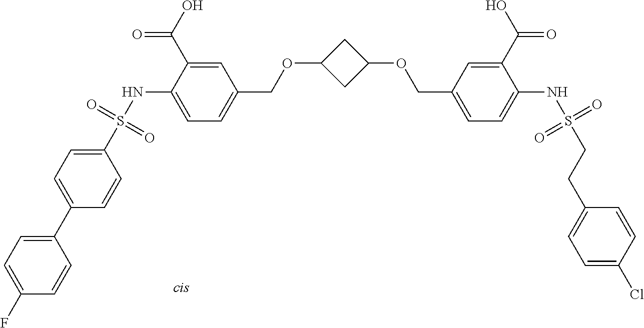 [SMILES: O=C(O)c1cc(COC2CC(OCc3ccc(NS(=O)(=O)c4ccc(-c5ccc(F)cc5)cc4)c(C(=O)O)c3)C2)ccc1NS(=O)(=O)CCc1ccc(Cl)cc1] | 821.2 |
| 17 | 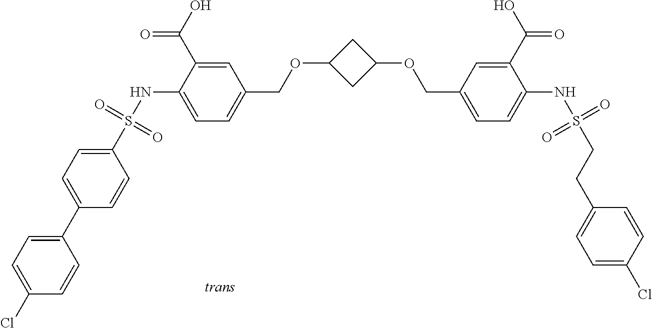 [SMILES: O=C(O)c1cc(COC2CC(OCc3ccc(NS(=O)(=O)c4ccc(-c5ccc(Cl)cc5)cc4)c(C(=O)O)c3)C2)ccc1NS(=O)(=O)CCc1ccc(Cl)cc1] | 837.1 |
| 18 | 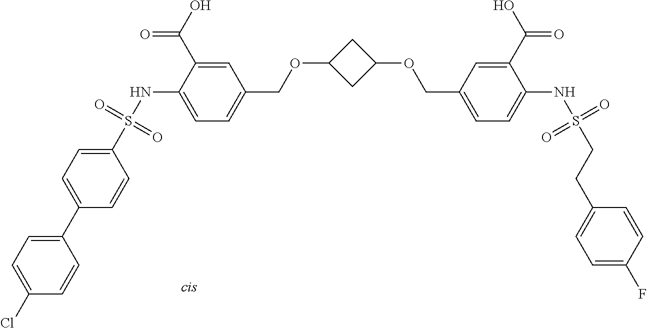 [SMILES: O=C(O)c1cc(COC2CC(OCc3ccc(NS(=O)(=O)c4ccc(-c5ccc(Cl)cc5)cc4)c(C(=O)O)c3)C2)ccc1NS(=O)(=O)CCc1ccc(F)cc1] | 821.2 |

### EXAMPLE 5

### Preparation of Compound 19

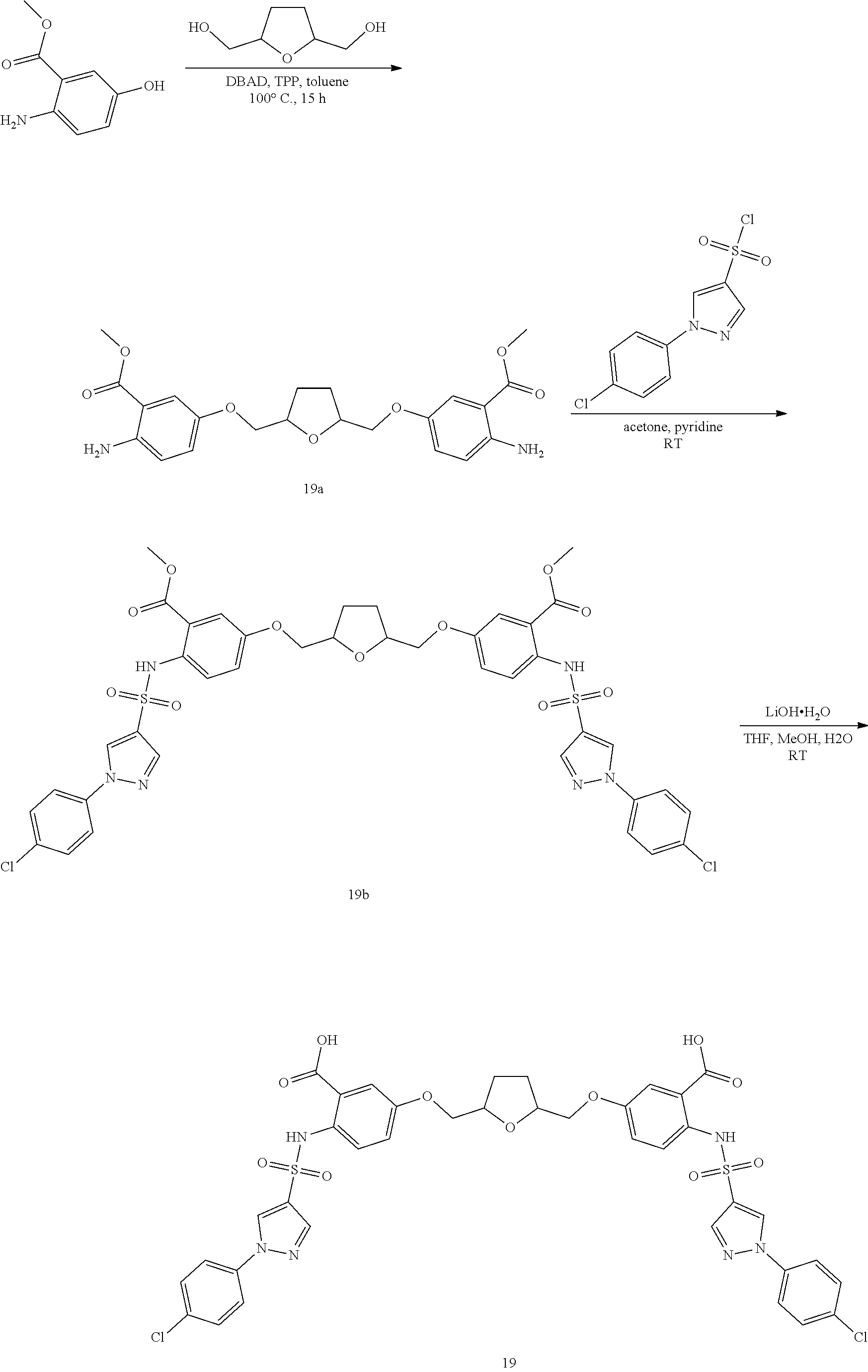

### Step 1: Dimethyl 5,5′-(((tetrahydrofuran-2,5-diyl)bis(methylene)) bis(oxy))bis(2-aminobenzoate) (19a)

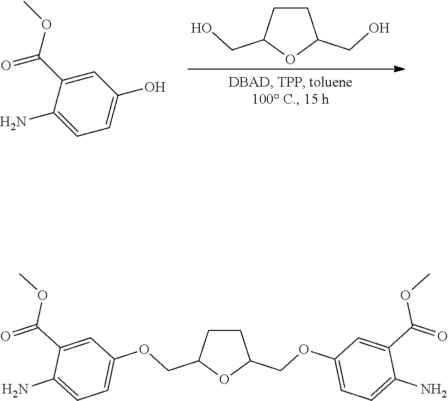 [SMILES: COC(=O)c1cc(O)ccc1N ; OCC1CCC(CO)O1 ; COC(=O)c1cc(OCC2CCC(COc3ccc(N)c(C(=O)OC)c3)O2)ccc1N]

To a solution of (tetrahydrofuran-2,5-diyl)dimethanol (200 mg, 1.51 mmol) in toluene (5 mL) was added methyl 2-amino-5-hydroxybenzoate (506 mg, 3.03 mmol), triphenylphosphine (1032 mg, 3.93 mmol), and DBAD (906 mg, 3.93 mmol) at room temperature. The reaction was stirred at 100° C. for 15 h, concentrated in vacuo, and the residue was purified by flash chromatography on SiO2 [Isco®, SepaFlash® 4 g column, eluent of 50% Pet. ether/hex] to provide dimethyl 5,5′-(((tetrahydrofuran-2,5-diyl)bis(methylene)) bis(oxy))bis(2-aminobenzoate). MS (ESI, m/z): 431.3 [M+H⁺].

### Step 2: Methyl 2-((1-(4-chlorophenyl)-1H-pyrazole)-4-sulfonamido)-5-((5-((4-((1-(4-chlorophenyl)-1H-pyrazole)-4-sulfonamido)-3-(methoxycarbonyl) phenoxy)methyl)-tetrahydrofuran-2-yl) methoxy)benzoate (19b)

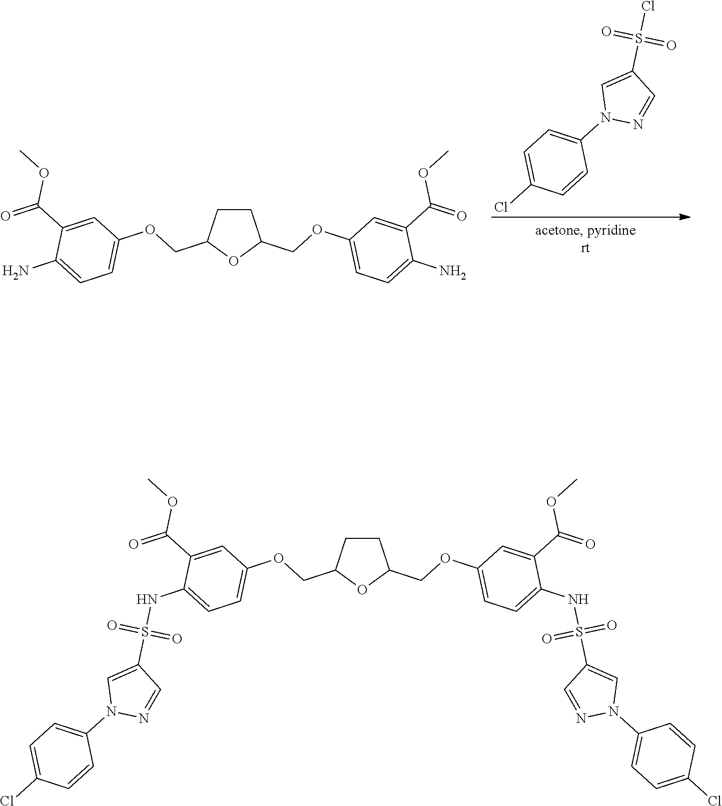

To a solution of dimethyl 5,5′-(((tetrahydrofuran-2,5-diyl)bis(methylene)) bis(oxy))bis(2-aminobenzoate) (19a; 205 mg, 0.476 mmol) in acetone (6 ml) and pyridine (2 ml) was added 1-(4-chlorophenyl)-1H-pyrazole-4-sulfonyl chloride (Int-1; 290 mg, 1.05 mmol) at room temperature. The reaction was stirred for 15 h, quenched with H₂O (10 mL), extracted with EtOAc (20 mL×3), and washed with water (20 mL). The organics were dried over Na₂SO₄, filtered, and concentrated in vacuo. The resulting residue was purified by flash chromatography on SiO2 [Isco®, SepaFlash® 12 g column, eluent of 30% Pet. ether/hexanes] to provide methyl 2-((1-(4-chlorophenyl)-1H-pyrazole)-4-sulfonamido)-5-((5-((4-((1-(4-chlorophenyl)-1H-pyrazole)-4-sulfonamido)-3-(methoxycarbonyl) phenoxy)-methyl)tetrahydrofuran-2-yl) methoxy)benzoate. MS (ESI, m/z): 911.0 [M+H⁺].

### Step 3:5-((5-((3-Carboxy-4-((1-(4-chlorophenyl)-1H-pyrazole)-4-sulfonamido)-phenoxy)methyl)tetrahydrofuran-2-yl) methoxy)-2-((1-(4-chlorophenyl)-1H-pyrazole)-4-sulfonamido)benzoic acid (19)

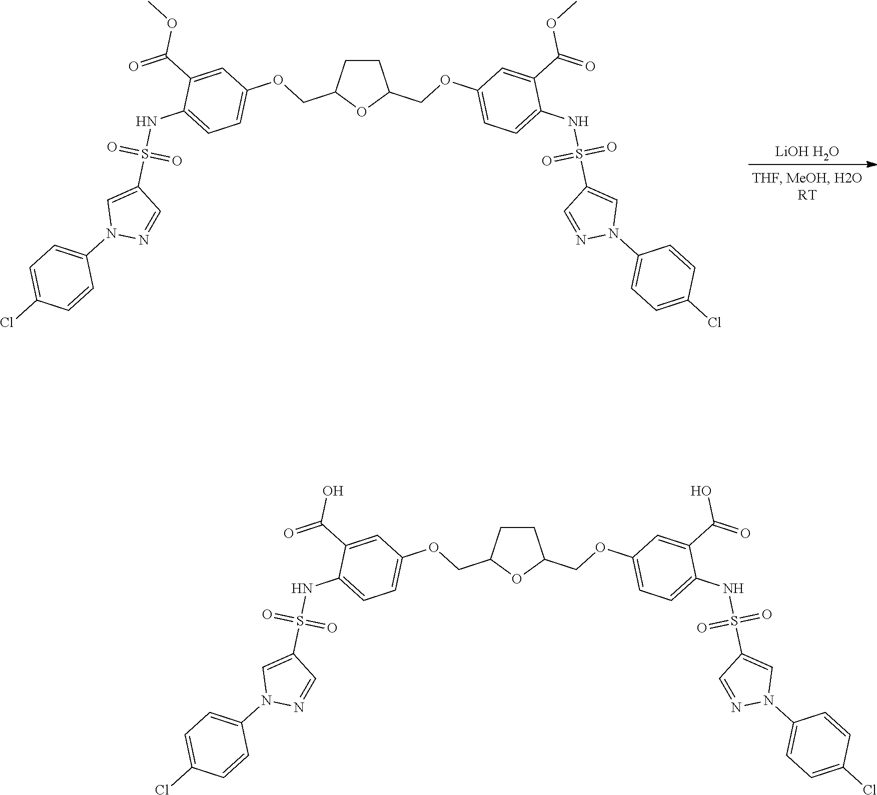 [SMILES: COC(=O)c1cc(OCC2CCC(COc3ccc(NS(=O)(=O)c4cnn(-c5ccc(Cl)cc5)c4)c(C(=O)OC)c3)O2)ccc1NS(=O)(=O)c1cnn(-c2ccc(Cl)cc2)c1 ; "LiOH H2O" ; O=C(O)c1cc(OCC2CCC(COc3ccc(NS(=O)(=O)c4cnn(-c5ccc(Cl)cc5)c4)c(C(=O)O)c3)O2)ccc1NS(=O)(=O)c1cnn(-c2ccc(Cl)cc2)c1]

To a solution of methyl 2-((1-(4-chlorophenyl)-1H-pyrazole)-4-sulfonamido)-5-((5-((4-((1-(4-chlorophenyl)-1H-pyrazole)-4-sulfonamido)-3-(methoxycarbonyl) phenoxy)methyl)tetrahydrofuran-2-yl) methoxy)benzoate (19b; 350 mg, 0.384 mmol) in THF (5 mL), MeOH (5 mL), and H₂O (2 mL) was added lithium hydroxide hydrate (161 mg, 3.84 mmol) at room temperature. The reaction was stirred for 15 h, then acidified with 3 N HCl to pH=2. The reaction was then concentrated in vacuo and the resulting residue was purified by preparative-HPLC [Phenomenex Synergi C18 column (150 mm*30 mm, 4 μm), eluent of MeCN/H₂O (+0.05% HCl)]. Fractions containing the target compound were frozen and dried using lyophilization to provide 5-((5-((3-carboxy-4-((1-(4-chlorophenyl)-1H-pyrazole)-4-sulfonamido) phenoxy)methyl)tetrahydrofuran-2-yl) methoxy)-2-((1-(4-chlorophenyl)-1H-pyrazole)-4-sulfonamido)benzoic acid. MS (ESI) m/z: 883.0 [M+H⁺]. ¹H NMR (400M Hz, CD3OD) δ ppm 8.57 (s, 2H), 7.76-7.71 (m, 2H), 7.69-7.58 (m, 6H), 7.48-7.36 (m, 6H), 7.14 (dd, J=3.2, 9.2 Hz, 2H), 4.29 (br s, 2H), 4.01-3.97 (m, 2H), 3.92-3.87 (m, 2H), 2.06 (br d, J=4.8 Hz, 2H), 1.89 (br s, 2H). NMR indicated for cis isomer only.

The following compounds (20 through 23) of the present disclosure were made using the methods described in Example 5 above, and substituting the appropriate reactants and/or

| Com- | | Observed |
|---|---|---|
| pound | Structure | MS |
| 20 | 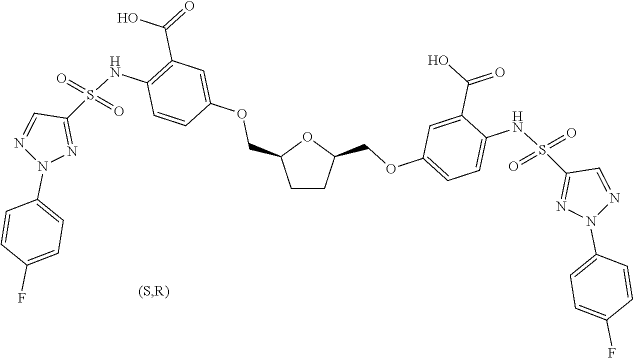 [SMILES: O=C(O)c1cc(OC[C@H]2CC[C@@H](COc3ccc(NS(=O)(=O)c4cnn(-c5ccc(F)cc5)n4)c(C(=O)O)c3)O2)ccc1NS(=O)(=O)c1cnn(-c2ccc(F)cc2)n1] | 853.1 |
| 21 | 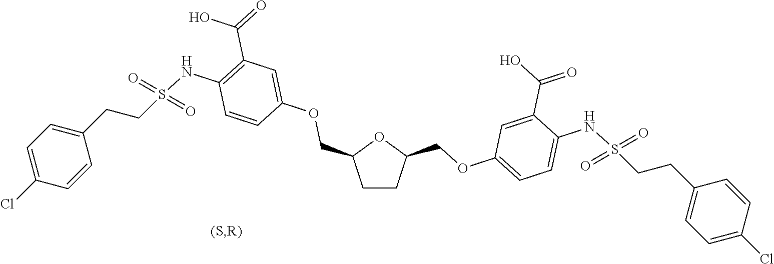 [SMILES: O=C(O)c1cc(OC[C@H]2CC[C@@H](COc3ccc(NS(=O)(=O)CCc4ccc(Cl)cc4)c(C(=O)O)c3)O2)ccc1NS(=O)(=O)CCc1ccc(Cl)cc1] | 807.1 |
| 22 | 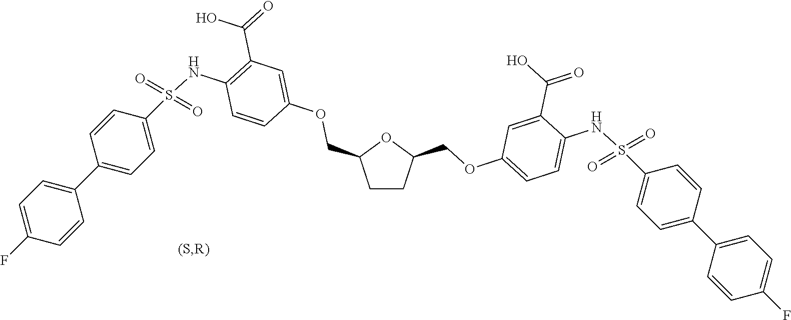 [SMILES: O=C(O)c1cc(OC[C@H]2CC[C@@H](COc3ccc(NS(=O)(=O)c4ccc(-c5ccc(F)cc5)cc4)c(C(=O)O)c3)O2)ccc1NS(=O)(=O)c1ccc(-c2ccc(F)cc2)cc1] | 871.3 |
| 23 | 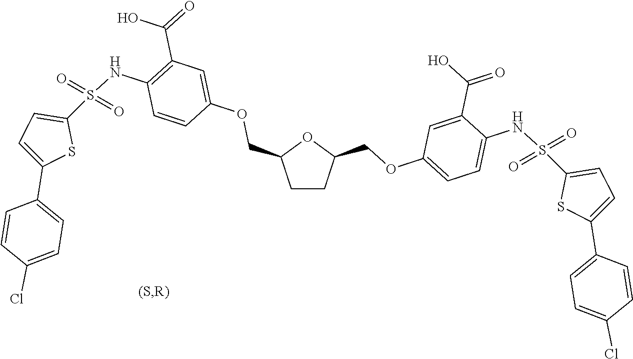 [SMILES: O=C(O)c1cc(OC[C@H]2CC[C@@H](COc3ccc(NS(=O)(=O)c4ccc(-c5ccc(Cl)cc5)s4)c(C(=O)O)c3)O2)ccc1NS(=O)(=O)c1ccc(-c2ccc(Cl)cc2)s1] | 917.2 |

### EXAMPLE 6

### Preparation of Compound 24

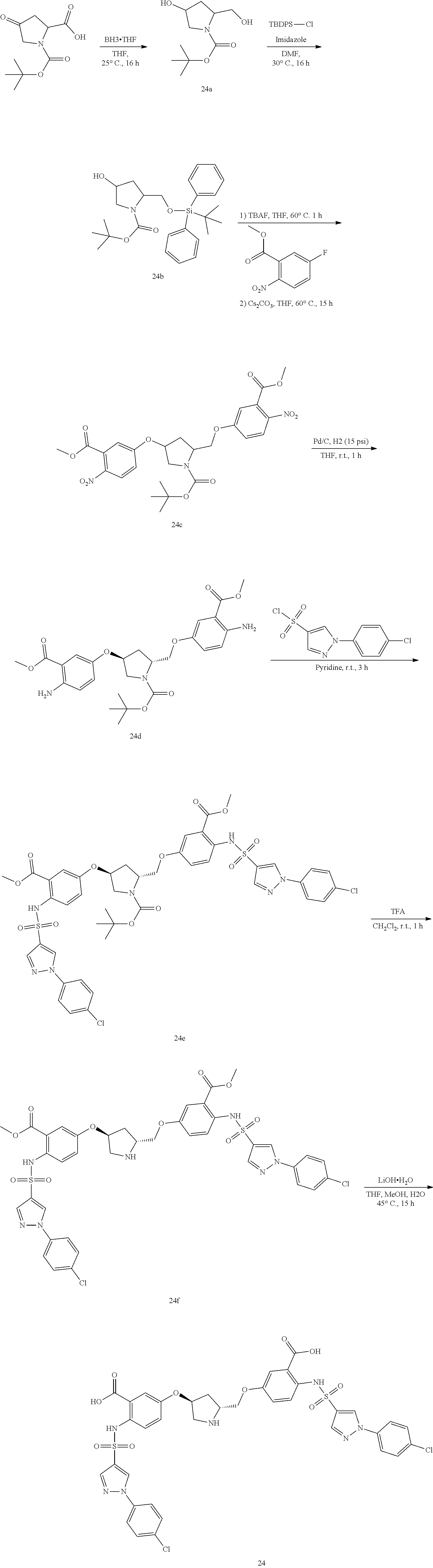

### Step 1: tert-butyl 4-hydroxy-2-(hydroxymethyl) pyrrolidine-1-carboxylate (24a)

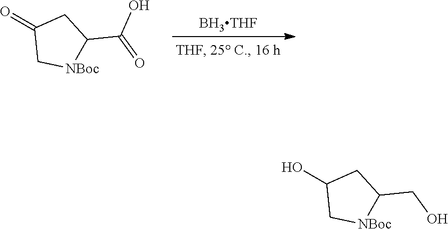 [SMILES: CC(C)(C)OC(=O)N1CC(=O)CC1C(=O)O ; CC(C)(C)OC(=O)N1CC(O)CC1CO ; "BH3·THF"]

To a stirred mixture of 1-(tert-butoxycarbonyl)-4-oxopyrrolidine-2-carboxylic acid (1 g, 4.36 mmol) in THF (10 mL) was added BH3. THF (IM, 26.2 mL, 26.2 mmol) at 0° C., and the reaction was stirred at 25° C. for 16 h. MeOH (10 mL) was added and the mixture was concentrated in vacuo. The resulting residue was purified by flash chromatography on SiO₂ [Isco®, 4 g column, eluent of 0 to 60% EtOAc/Pet. ether gradient at 30 mL/min] to provide tert-butyl 4-hydroxy-2-(hydroxymethyl) pyrrolidine-1-carboxylate.

### Step 2: tert-butyl 2-(((tert-butyldiphenylsilyl)oxy)methyl)-4-hydroxypyrrolidine-1-carboxylate (24b)

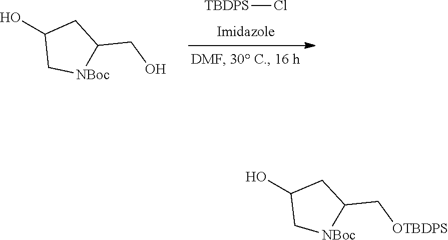

To a solution of tert-butyl 4-hydroxy-2-(hydroxymethyl) pyrrolidine-1-carboxylate (24a) (900 mg, 4.14 mmol) in DMF (20 mL) was added tert-butylchlorodiphenylsilane (1.366 g, 4.97 mmol) and imidazole (846 mg, 12.43 mmol). The mixture was stirred at 30° C. for 16 h, concentrated in vacuo, and the resulting residue was purified by flash chromatography on SiO₂ [Isco8, 20 g column, eluent of 0 to 25% EtOAc/Pet. ether gradient at 35 mL/min] to provide tert-butyl 2-(((tert-butyldiphenylsilyl)-oxy)methyl)-4-hydroxypyrrolidine-1-carboxylate. MS (ESI m/z): 456.3 [M+H⁺].

### Step 3: tert-butyl 4-(3-(methoxycarbonyl)-4-nitrophenoxy)-2-((3-(methoxycarbonyl)-4-nitrophenoxy)methyl) pyrrolidine-1-carboxylate (24c)

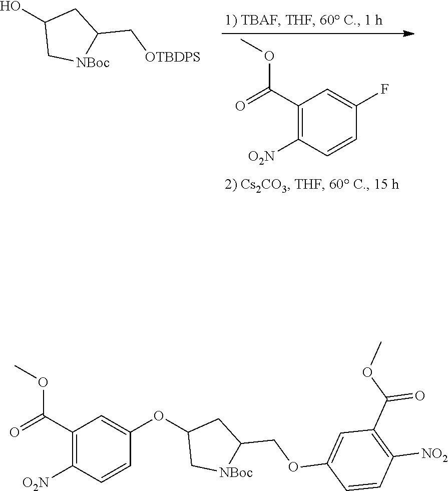

To a stirred solution of tert-butyl 2-(((tert-butyldiphenylsilyl)oxy)methyl)-4-hydroxy-pyrrolidine-1-carboxylate (24b; 160 mg, 0.351 mmol)) in THF (10 mL) was added TBAF (0.702 mL, 0.702 mmol). The reaction was stirred at 60° C. for 1 h, then methyl-5-fluoro-2-nitrobenzoate (210 mg, 1.053 mmol) and Cs₂CO₃ (343 mg, 1.053 mmol) were added. The reaction was stirred at 60° C. for 15 h, then concentrated in vacuo. The resulting residue was purified by flash chromatography on SiO₂ [Isco®, Agela® 4 g column, eluent of 0 to 35% EtOAc/Pet. ether gradient at 30 mL/min| to provide trans-tert-butyl 4-(3-(methoxycarbonyl)-4-nitrophenoxy)-2-((3-(methoxycarbonyl)-4-nitrophenoxy)-methyl) pyrrolidine-1-carboxylate. MS (ESI m/z): 598.1 [M+Na⁺].

### Step 4: Trans-tert-butyl 4-(4-amino-3-(methoxycarbonyl) phenoxy)-2-((4-amino-3-(methoxycarbonyl) phenoxy)methyl) pyrrolidine-1-carboxylate (24d)

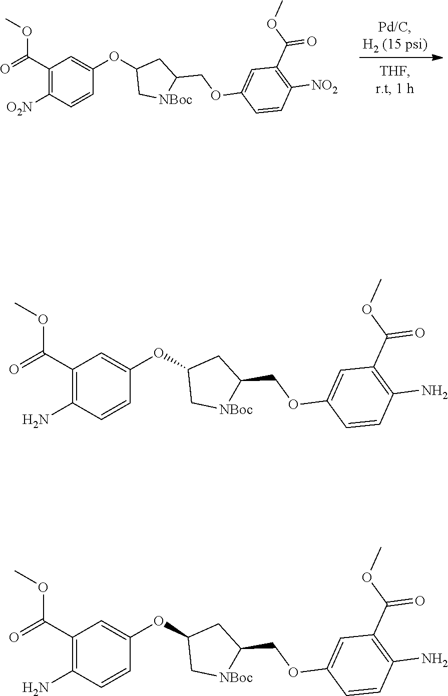 [SMILES: COC(=O)c1cc(OCC2CC(Oc3ccc([N+](=O)[O-])c(C(=O)OC)c3)CN2C(=O)OC(C)(C)C)ccc1[N+](=O)[O-] ; COC(=O)c1cc(OC[C@@H]2C[C@@H](Oc3ccc(N)c(C(=O)OC)c3)CN2C(=O)OC(C)(C)C)ccc1N ; COC(=O)c1cc(OC[C@@H]2C[C@H](Oc3ccc(N)c(C(=O)OC)c3)CN2C(=O)OC(C)(C)C)ccc1N]

To a solution of tert-butyl 4-(3-(methoxycarbonyl)-4-nitrophenoxy)-2-((3-(methoxy-carbonyl)-4-nitrophenoxy)methyl) pyrrolidine-1-carboxylate (24c; (40 mg, 0.070 mmol) in THF (3 mL) was added Pd/C (10%, 7.40 mg, 6.95 μmol). The reaction was stirred at room temperature under H₂ (15 psi) for 1 h, then filtered through Celite™, and the filtrate was concentrated in vacuo. The resulting mixture of cis and trans isomers was separated by Chiral Supercritical Fluid chromatography [Column DAICEL CHIRALPAK IG (250 mm*30 mm, 10 μm), conditions 0.1% NH₃—H₂O in MeOH using a 60% gradient] to provide the first eluting peak (P1, Rt=0.631 min) trans-tert-butyl 4-(4-amino-3-(methoxycarbonyl) phenoxy)-2-((4-amino-3-(methoxycarbonyl) phenoxy)methyl) pyrrolidine-1-carboxylate (20 mg, 0.039 mmol, 25.00% yield) and the second eluting peak (P2, Rt=1.604 min) cis-tert-butyl 4-(4-amino-3-(methoxycarbonyl) phenoxy)-2-((4-amino-3-(methoxycarbonyl)-phenoxy)-methyl) pyrrolidine-1-carboxylate. First eluting isomer: MS (ESI m/z): 516 [M+H⁺]. ¹H NMR (500 MHZ, CD₃OD) δ ppm 7.34 (br s, 1H), 7.29 (br s, 1H), 6.96 (dd, J=3.0, 9.0 Hz, 2H), 6.72 (d, J=9.0 Hz, 2H), 4.23 (br s, 2H), 4.06-3.95 (m, 1H), 3.86 (s, 3H), 3.81 (s, 3H), 3.74 (br s, 1H), 3.56 (br d, J=12.0 Hz, 1H), 2.42 (br d, J=14.5 Hz, 1H), 2.25 (br s, 1H), 1.50 (s, 9H). Second eluting isomer: MS (ESI m/z): 516 [M+H⁺]. ¹H NMR (500 MHz, MeCN-d3) δ ppm 7.03 (br s, 1H), 6.97 (br s, 1H), 6.72-6.56 (m, 2H), 6.38 (br d, J=9.0 Hz, 2H), 5.42 (br d, J=12.5 Hz, 4H), 4.53 (br s, 1H), 3.91 (br dd, J=3.5, 8.5 Hz, 1H), 3.84 (br s, 1H), 3.65 (br t, J=8.5 Hz, 1H), 3.51-3.37 m, 7H), 3.17 (br s, 1H), 1.98 (br s, 1H), 1.70-1.62 (m, 1H), 1.15 (s, 9H).

### Step 5: trans-tert-butyl 4-(4-((1-(4-chlorophenyl)-1H-pyrazole)-4-sulfonamido)-3-(methoxycarbonyl) phenoxy)-2-((4-((1-(4-chlorophenyl)-1H-pyrazole)-4-sulfonamido)-3-(methoxycarbonyl) phenoxy)methyl) pyrrolidine-1-carboxylate (24e)

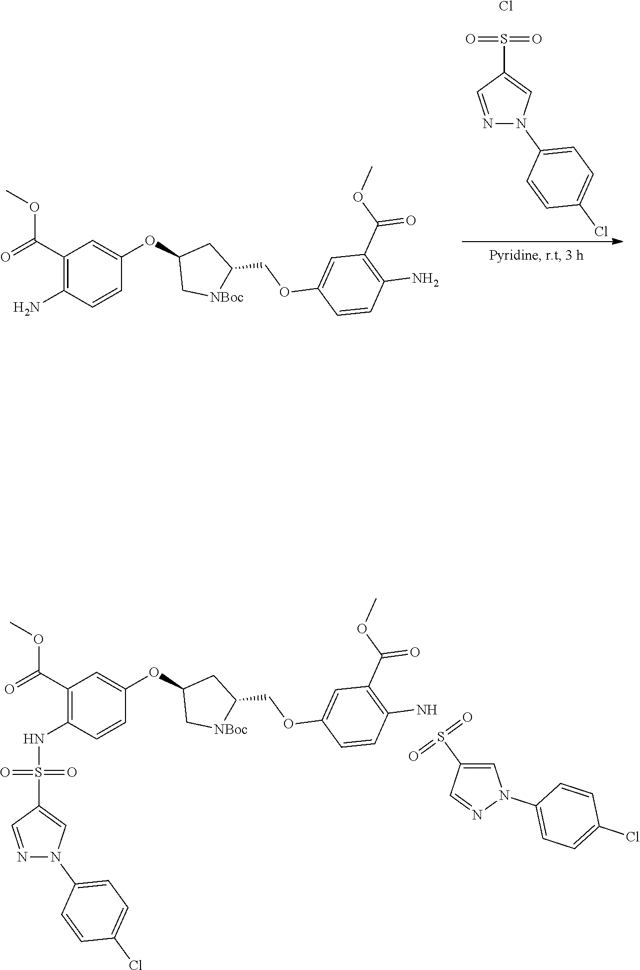

To a solution of trans-tert-butyl 4-(4-amino-3-(methoxycarbonyl) phenoxy)-2-((4-amino-3-(methoxycarbonyl) phenoxy)methyl) pyrrolidine-1-carboxylate (24d; 15 mg, 0.029 mmol) in pyridine (1 mL) was added 1-(4-chlorophenyl)-1H-pyrazole-4-sulfonyl chloride (Int-1; 20.16 mg, 0.073 mmol). The reaction mixture was stirred at room temperature for 3 h, then concentrated in vacuo. The resulting residue was purified by reverse phase chromatography [Phenomenex Synergi C₁₈ (150*21.2 mm, 4 μm), eluent of mobile phase A: H₂O (0.1% TFA) and mobile phase B: MeCN at 220 nm] to provide trans-tert-butyl 4-(4-((1-(4-chlorophenyl)-1H-pyrazole)-4-sulfonamido)-3-(methoxycarbonyl) phenoxy)-2-((4-((1-(4-chlorophenyl)-1H-pyrazole)-4-sulfonamido)-3-(methoxy carbonyl) phenoxy)-methyl) pyrrolidine-1-carboxylate. MS (ESI m/z): 940.0 [M+H⁺-56].

### Step 6: trans-methyl 2-((1-(4-chlorophenyl)-1H-pyrazole)-4-sulfonamido)-5-((5-((4-((1-(4-chlorophenyl)-1H-pyrazole)-4-sulfonamido)-3-(methoxycarbonyl) phenoxy)methyl) pyrrolidin-3-yl)oxy)benzoate (24f)

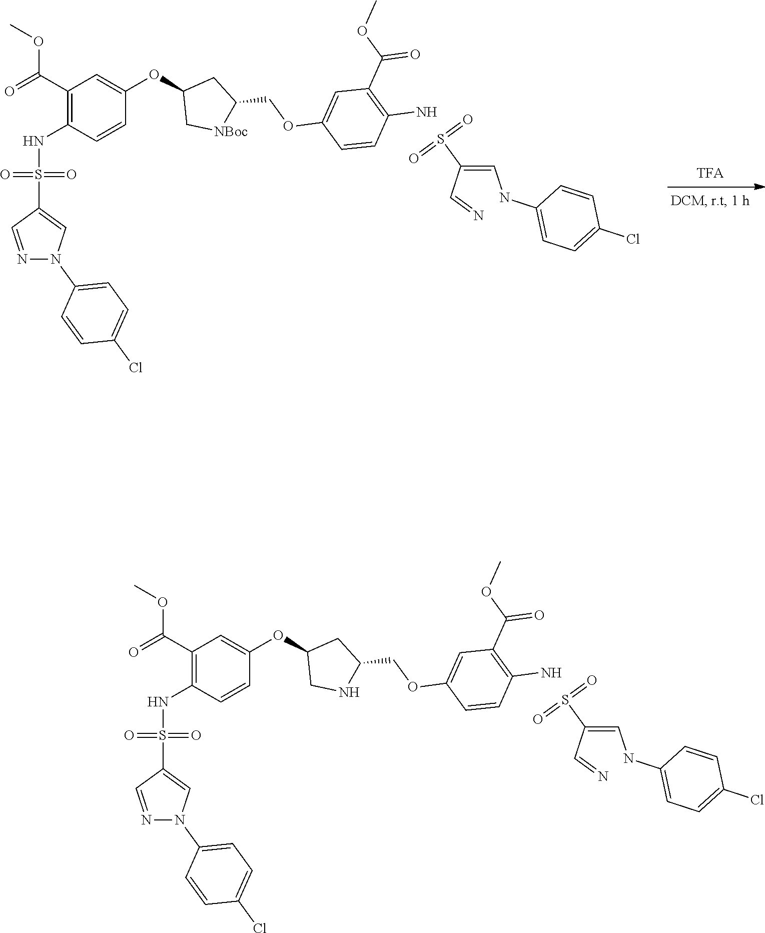

To a solution of trans-tert-butyl 4-(4-((1-(4-chlorophenyl)-1H-pyrazole)-4-sulfonamido)-3-(methoxycarbonyl) phenoxy)-2-((4-((1-(4-chlorophenyl)-1H-pyrazole)-4-sulfonamido)-3-(methoxycarbonyl) phenoxy)methyl) pyrrolidine-1-carboxylate (24e; 20 mg, 0.020 mmol) in CH₂C₁₂ (2 mL) was added TFA (0.2 mL, 2.60 mmol). The reaction was stirred at room temperature for 1 h, then concentrated in vacuo to provide trans-methyl 2-((1-(4-chloro-phenyl)-1H-pyrazole)-4-sulfonamido)-5-((5-((4-((1-(4-chlorophenyl)-1H-pyrazole)-4-sulfonamido)-3-(methoxycarbonyl) phenoxy)methyl) pyrrolidin-3-yl)oxy)benzoate, which was used in the subsequent step without further purification. MS (ESI) m/z: 898.0 [M+H⁺].

### Step 3: trans-5-((5-((3-carboxy-4-((1-(4-chlorophenyl)-1H-pyrazole)-4-sulfonamido)-phenoxy)methyl) pyrrolidin-3-yl)oxy)-2-((1-(4-chlorophenyl)-1H-pyrazole)-4-sulfonamido)benzoic acid (24)

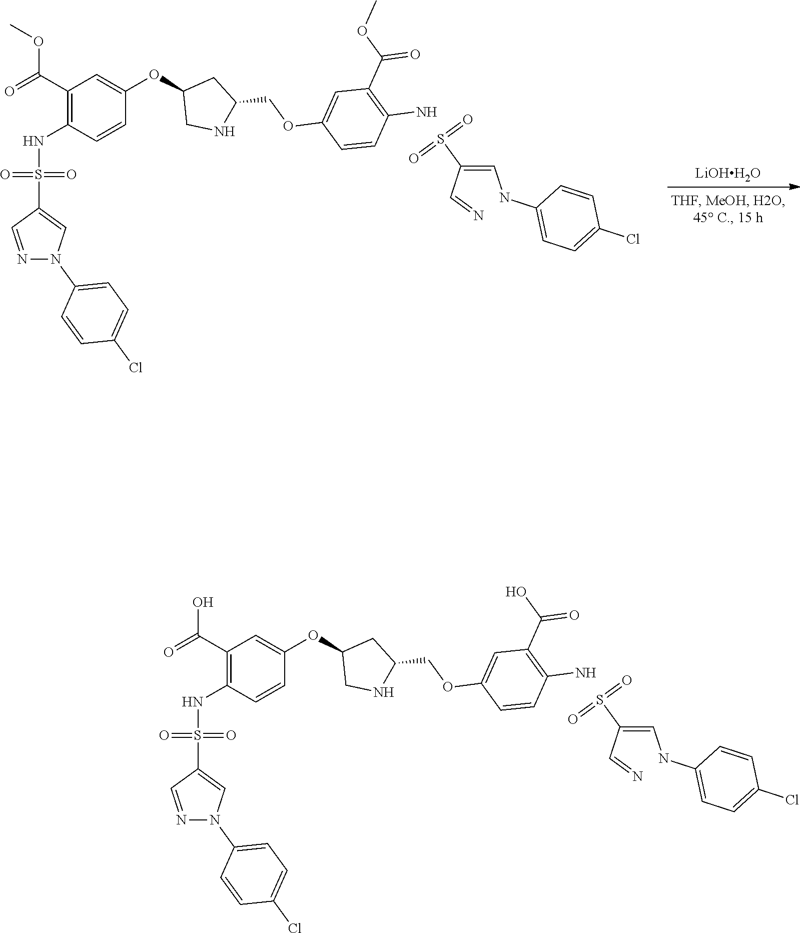

To a solution of trans-methyl 2-((1-(4-chlorophenyl)-1H-pyrazole)-4-sulfonamido)-5-((5-((4-((1-(4-chlorophenyl)-1H-pyrazole)-4-sulfonamido)-3-(methoxycarbonyl)-phenoxy)-methyl) pyrrolidin-3-yl)oxy)benzoate (24f; 17 mg, 0.019 mmol) in THF (1 mL), MeOH (1 mL), and H₂O (0.25 mL) was added lithium hydroxide hydrate (7.95 mg, 0.190 mmol) at room temperature. The reaction was stirred at 45° C. for 15 h, then acidified with 3 N HCl to pH-2. The reaction was concentrated in vacuo and the resulting residue was purified by reverse phase chromatography [Phenomenex Synergi C18 column (150*21.2 mm, 4 μm), eluent of mobile phase A: H₂O (0.1% TFA) and mobile phase B: MeCN at 220 nm] to provide trans-5-((5-((3-carboxy-4-((1-(4-chlorophenyl)-1H-pyrazole)-4-sulfonamido) phenoxy)methyl) pyrrolidin-3-yl)oxy)-2-((1-(4-chlorophenyl)-1H-pyrazole)-4-sulfonamido)benzoic acid. MS (ESI m/z): 866.0 [M−H⁺]. ¹H NMR (400 MHZ, CD₃OD) δ ppm 8.70 (d, J=4.8 Hz, 2H), 7.86-7.66 (m, 8H), 7.57 (t, J=2.8 Hz, 2H), 7.48 (dd, J=1.6, 8.8 Hz, 4H), 7.26 (dd, J=2.8, 9.2 Hz, 2H), 5.24 (br s, 1H), 4.35 (dd, J=2.8, 10.8 Hz, 1H), 4.27 (br d, J=6.8 Hz, 1H), 4.14 (dd, J=7.2, 10.4 Hz, 1H), 3.65-3.56 (m, 2H), 2.45 (br dd, J=7.2, 13.6 Hz, 1H), 2.22 (br t, J=10.4 Hz, 1H).

### EXAMPLE 7

### Preparation of Compound 25

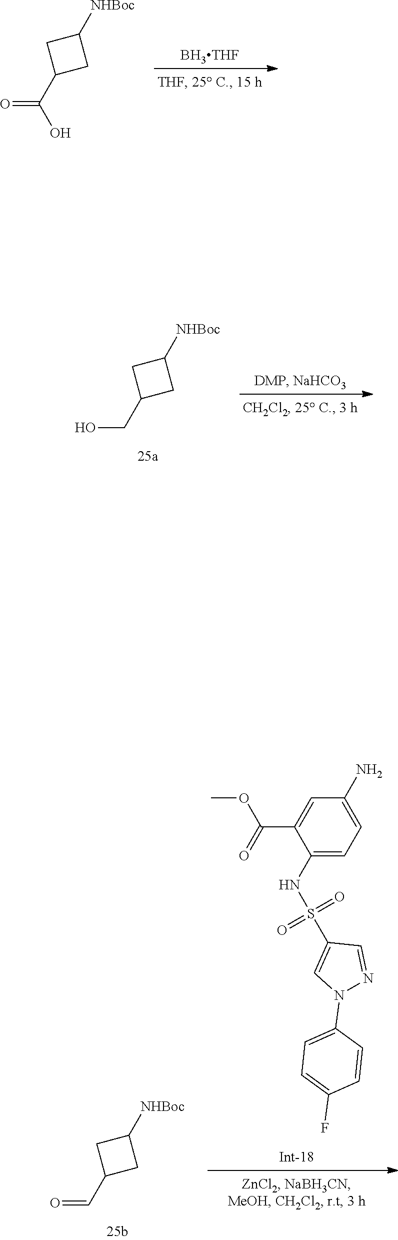

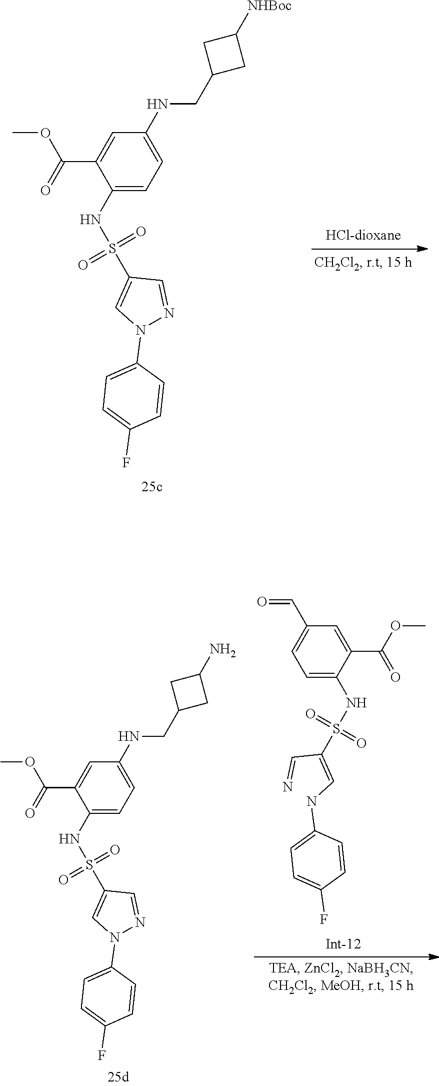

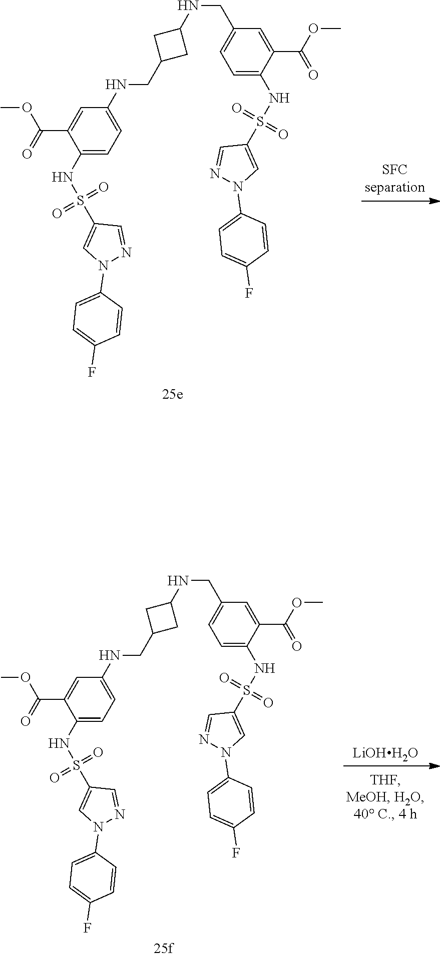

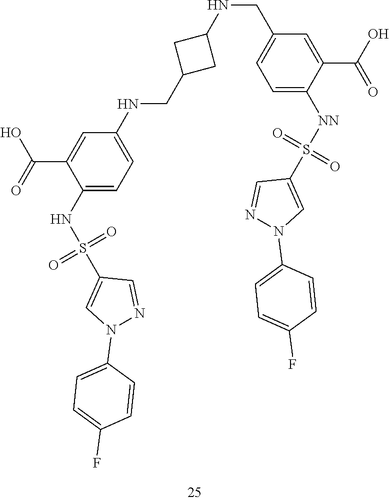

### Step 1: tert-butyl (3-(hydroxymethyl)cyclobutyl) carbamate (25a)

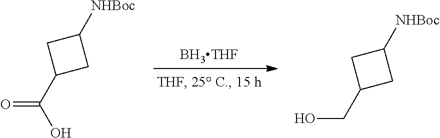 [SMILES: CC(C)(C)OC(=O)NC1CC(C(=O)O)C1 ; CC(C)(C)OC(=O)NC1CC(CO)C1 ; "BH3·THF"]

To a solution of 3-((tert-butoxycarbonyl)amino)cyclobutane-1-carboxylic acid (4 g, 18.58 mmol) in THF (60 mL) was added BH3. THF (1 M, 27.9 mL, 27.9 mmol) at 0° C. The reaction was stirred at 25° C. for 15 h, then quenched with MeOH (30 mL) and concentrated in vacuo to provide tert-butyl (3-(hydroxymethyl)cyclobutyl) carbamate which was used in the subsequent step without further purification.

### Step 2: tert-butyl (3-formylcyclobutyl) carbamate (25b)

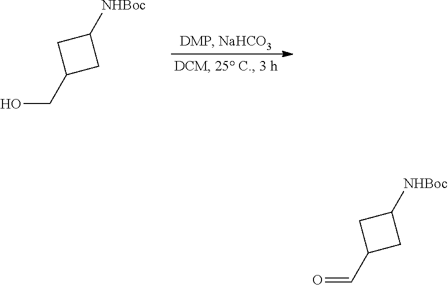 [SMILES: CC(C)(C)OC(=O)NC1CC(CO)C1 ; CC(C)(C)OC(=O)NC1CC(C=O)C1]

To a solution of tert-butyl (3-(hydroxymethyl)cyclobutyl) carbamate (25a; 7.4 g, 36.8 mmol) in CH₂Cl₂ (200 mL) was added DMP (23.39 g, 55.2 mmol) and NaHCO₃ (4.63 g, 55.2 mmol) at 0° C. The reaction mixture was warmed to room temperature and stirred for 3 h, then filtered, and the filtrate was concentrated in vacuo. The resulting residue was purified by flash chromatography on SiO₂ [Isco®, Agela® 120 g column, eluent of 0 to 50% EtOAc/Pet. ether gradient at 35 mL/min| to provide tert-butyl (3-formylcyclobutyl) carbamate.

### Step 3: methyl 5-(((3-((tert-butoxycarbonyl)amino) cyclobutyl)methyl)amino)-2-((1-(4-fluorophenyl)-1H-pyrazole)-4-sulfonamido)benzoate (25c)

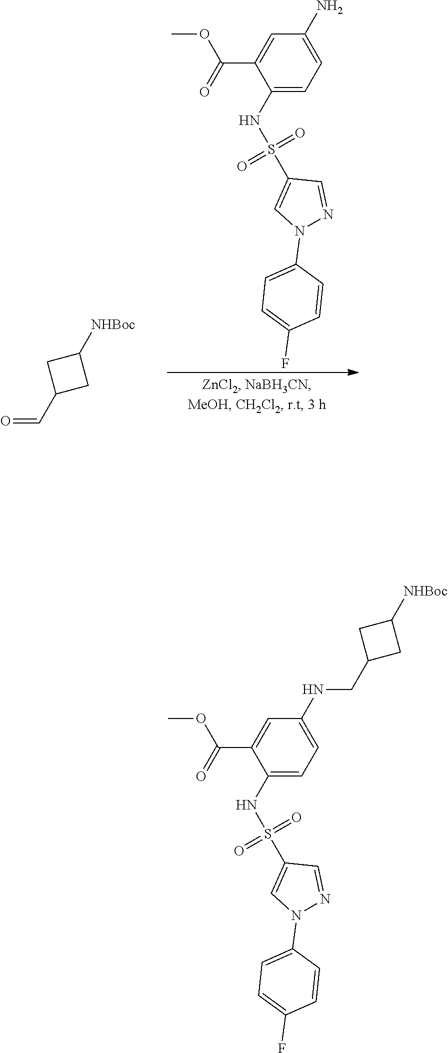 [SMILES: CC(C)(C)OC(=O)NC1CC(C=O)C1 ; COC(=O)c1cc(N)ccc1NS(=O)(=O)c1cnn(-c2ccc(F)cc2)c1 ; COC(=O)c1cc(NCC2CC(NC(=O)OC(C)(C)C)C2)ccc1NS(=O)(=O)c1cnn(-c2ccc(F)cc2)c1]

To a solution of tert-butyl (3-formylcyclobutyl) carbamate (25b; 1 g, 5.02 mmol) and methyl 5-amino-2-((1-(4-fluorophenyl)-1H-pyrazole)-4-sulfonamido)benzoate (Int-18) (1.959 g, 5.02 mmol) in CH₂Cl₂ (36 mL) and MeOH (36 mL) was added zinc (II) chloride (1.368 g, 10.04 mmol) and sodium cyanoborohydride (0.946 g, 15.06 mmol) at room temperature. The reaction mixture stirred for 3 h, then concentrated in vacuo. The resulting residue was diluted with water H₂O (80 mL), and extracted with EtOAc (2×30 mL). The organics was dried over Na₂SO₄, filtered, and concentrated in vacuo. The resulting residue was purified by flash chromatography on SiO₂ [Isco®, Agela® 120 g column, eluent of 0 to 60% MeCN/H₂O gradient at 35 mL/min] to provide methyl 5-(((3-((tert-butoxycarbonyl)amino) cyclobutyl)methyl)amino)-2-((1-(4-fluorophenyl)-1H-pyrazole)-4-sulfonamido)benzoate. MS (ESI, m/z): 574.3 [M+H⁺]Step 4: methyl 5-(((3-aminocyclobutyl)methyl)amino)-2-((1-(4-fluorophenyl)-1H-pyrazole)-4-sulfonamido)benzoate (25d)

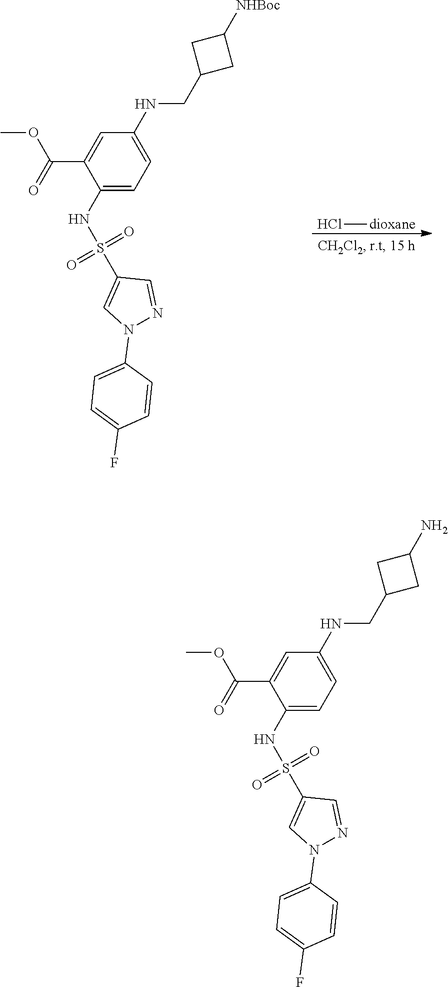

To a solution of methyl 5-(((3-((tert-butoxycarbonyl)amino) cyclobutyl)methyl)amino)-2-((1-(4-fluorophenyl)-1H-pyrazole)-4-sulfonamido)benzoate (25c; 1.5 g, 2.61 mmol) in CH₂Cl₂ (20 mL) was added HCl (4 M in dioxane, 15 mL, 60.0 mmol) at room temperature. The reaction mixture was stirred for 15 h, then concentrated in vacuo to provide the methyl 5-(((3-aminocyclobutyl)methyl)amino)-2-((1-(4-fluorophenyl)-1H-pyrazole)-4-sulfonamido)benzoate, which was used in the subsequent step without further purification. MS (ESI, m/z): 474.4 [M+H⁺]

### Step 5: cis-methyl 2-((1-(4-fluorophenyl)-1H-pyrazole)-4-sulfonamido)-5-(((3-((4-((1-(4-fluorophenyl)-1H-pyrazole)-4-sulfonamido)-3-(methoxycarbonyl)benzyl)amino)cyclobutyl)methyl) amino)benzoate (25e)

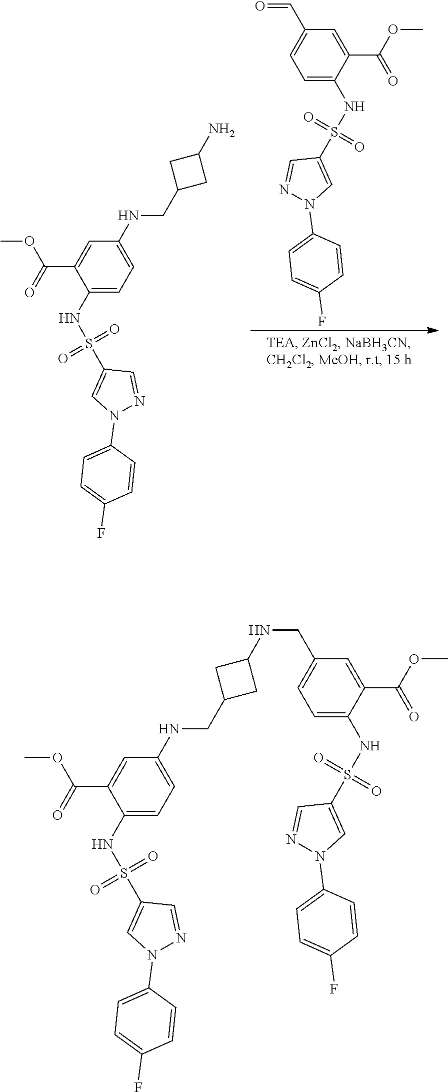

To a stirred mixture of methyl 5-(((3-aminocyclobutyl)methyl)amino)-2-((1-(4-fluorophenyl)-1H-pyrazole)-4-sulfonamido)benzoate (25d; 900 mg, 1.901 mmol) and methyl 2-((1-(4-fluorophenyl)-1H-pyrazole)-4-sulfonamido)-5-formylbenzoate (Int-12; 767 mg, 1.901 mmol) in MeOH (10 mL) and CH₂Cl₂ (10 mL) were added zinc (II) chloride (518 mg, 3.80 mmol) and sodium cyanoborohydride (358 mg, 5.70 mmol) at room temperature and the reaction mixture was stirred for 15 h. The reaction mixture was concentrated in vacuo, and the resulting residue was purified by flash chromatography on SiO₂ (Isco®, Agela® 25 g column, eluent of 0 to 80% EtOAc/Pet. ether gradient at 35 mL/min). The resulting material was a mixture of cis and trans isomers, which were subsequently separated by Chiral Supercritical Fluid chromatography [Column DAICEL CHIRALPAK OG-H (250 mm*30 mm, 5 μm), conditions 0.1% NH₄OH in EtOH using a 45% gradient] to provide cis-methyl 2-((1-(4-fluorophenyl)-1H-pyrazole)-4-sulfonamido)-5-(((3-((4-((1-(4-fluorophenyl)-1H-pyrazole)-4-sulfonamido)-3-(methoxycarbonyl)benzyl)amino)-cyclobutyl)methyl)-amino)benzoate and trans-methyl 2-((1-(4-fluorophenyl)-1/-pyrazole)-4-sulfonamido)-5-(((3-((4-((1-(4-fluorophenyl)-1/-pyrazole)-4-sulfonamido)-3-(methoxycarbonyl)benzyl)amino)cyclobutyl)methyl)-amino)benzoate. Cis isomer: MS (ESI m/z): 861.0 [M+H⁺]. ¹H NMR (400 MHZ, CD₃OD) δ ppm 8.71 (s, 1H), 8.39 (s, 1H), 7.93 (d, J=2.0 Hz, 1H), 7.85 (s, 1H), 7.65-7.73 (m, 6H), 7.54 (dd, J=8.4, 2.0 Hz, 1H), 7.41 (d, J=8.4 Hz, 1H), 7.15-7.22 (m, 4H), 7.01 (d, J=2.8 Hz, 1H), 6.82 (dd, J=8.8, 3.2 Hz, 1H), 3.87 (s, 3H), 3.71 (s, 3H), 3.60 (s, 2H), 3.04 (br t, J=7.6 Hz, 1H), 2.98 (d, J=6.8 Hz, 2H), 2.24 (br d, J=9.2 Hz, 2H), 2.07-2.16 (m, 1H), 1.38-1.48 (m, 2H).

### Step 7: cis-5-(((3-((3-carboxy-4-((1-(4-fluorophenyl)-1H-pyrazole)-4-sulfonamido)benzyl) amino)cyclobutyl)methyl) amino)-2-((1-(4-fluorophenyl)-1H-pyrazole)-4-sulfonamido)benzoic acid (25)

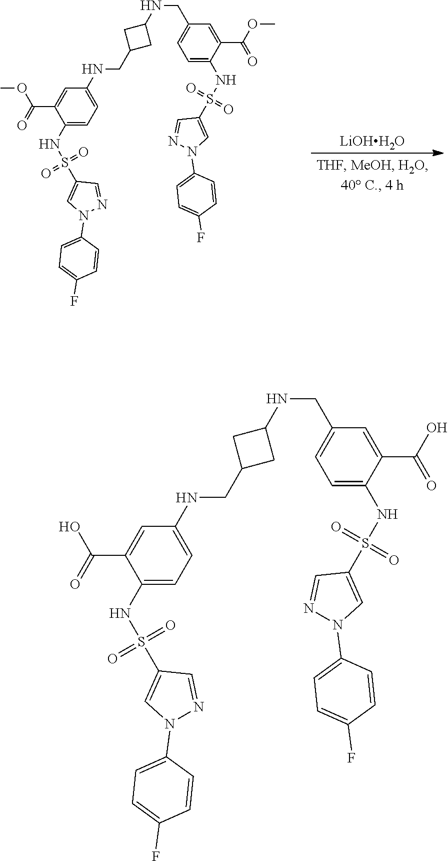

To a solution of cis-methyl 2-((1-(4-fluorophenyl)-1H-pyrazole)-4-sulfonamido)-5-(((3-((4-((1-(4-fluorophenyl)-1H-pyrazole)-4-sulfonamido)-3-(methoxycarbonyl)benzyl)amino)cyclobutyl)methyl) amino)benzoate (25e; 500 mg, 0.581 mmol) in THF (10 mL), MeOH (10 mL) and H₂O (5 mL) was added lithium hydroxide hydrate (244 mg, 5.81 mmol) at room temperature. The reaction mixture was stirred at 40° C. for 4 h, then acidified by the addition of 3N HCl to pH=2. The reaction mixture was concentrated in vacuo and the resulting residue was diluted with water (20 mL), and extracted with EtOAc (3×10 mL). The combined organic layers were dried over anh. Na₂SO₄, filtered, and concentrated in vacuo. The resulting residue was purified by reverse phase chromatography [Gilson 281, Boston Uni C18 column (40 mm*150 mm, 5 μm), eluent of mobile phase A: water (0.05% HCl) and mobile phase B: MeCN at 220 nm] to provide cis-5-(((3-((3-carboxy-4-((1-(4-fluorophenyl)-1H-pyrazole)-4-sulfonamido)benzyl)amino)-cyclobutyl)methyl)amino)-2-((1-(4-fluorophenyl)-1H-pyrazole)-4-sulfonamido)benzoic acid. MS (ESI, m/z): 833.0 [M+H⁺]. ¹H NMR (400 MHZ, CD₃OD) δ ppm 8.83 (s, 1H), 8.64 (s, 1H), 8.16 (d, J=2.0 Hz, 1H), 7.91 (s, 1H), 7.83 (d, J=8.4 Hz, 1H), 7.66-7.79 (m, 7H), 7.58 (d, J=2.0 Hz, 1H), 7.16-7.27 (m, 5H), 4.03 (s, 2H), 3.61-3.71 (m, 1H), 3.27 (br d, J=5.6 Hz, 2H), 2.36-2.49 (m, 3H), 1.89-2.01 (m, 2H).

The following compounds (26 through 36) of the present disclosure were made using the methods described in Example 7 above, and substituting the appropriate reactants and/or

| | | Observed |
|---|---|---|
| Compound | Structure | MS |
| 26 | 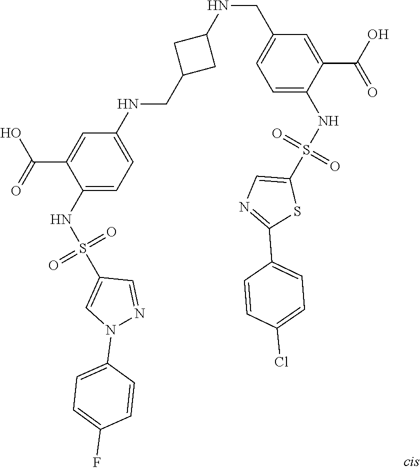 [SMILES: O=C(O)c1cc(NCC2CC(NCc3ccc(NS(=O)(=O)c4cnc(-c5ccc(Cl)cc5)s4)c(C(=O)O)c3)C2)ccc1NS(=O)(=O)c1cnn(-c2ccc(F)cc2)c1] | 866.0 |
| 27 | 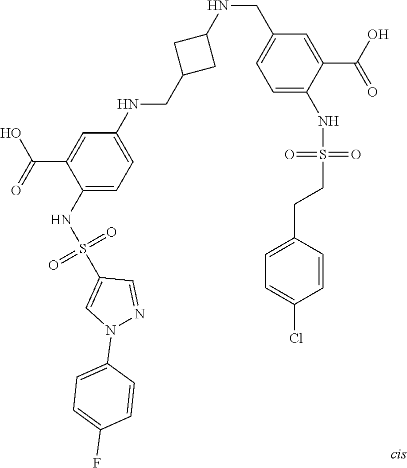 [SMILES: O=C(O)c1cc(CNC2CC(CNc3ccc(NS(=O)(=O)c4cnn(-c5ccc(F)cc5)c4)c(C(=O)O)c3)C2)ccc1NS(=O)(=O)CCc1ccc(Cl)cc1] | 811.0 |
| 28 | 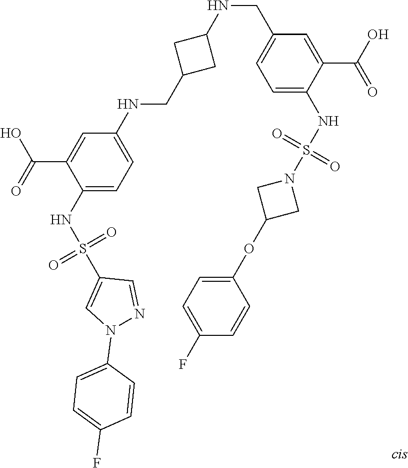 [SMILES: O=C(O)c1cc(NCC2CC(NCc3ccc(NS(=O)(=O)N4CC(Oc5ccc(F)cc5)C4)c(C(=O)O)c3)C2)ccc1NS(=O)(=O)c1cnn(-c2ccc(F)cc2)c1] | 838.1 |
| 29 | 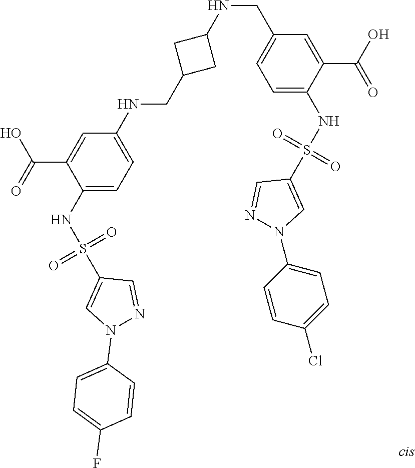 [SMILES: O=C(O)c1cc(CNC2CC(CNc3ccc(NS(=O)(=O)c4cnn(-c5ccc(F)cc5)c4)c(C(=O)O)c3)C2)ccc1NS(=O)(=O)c1cnn(-c2ccc(Cl)cc2)c1] | 849.0 |
| 30 | 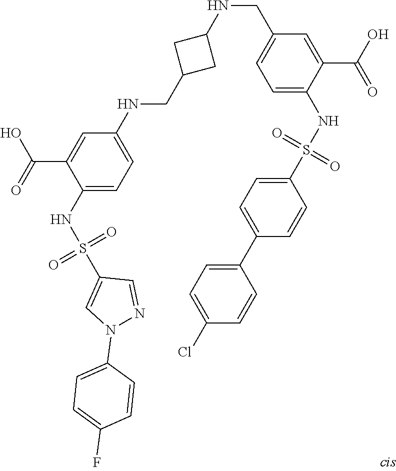 [SMILES: O=C(O)c1cc(CNC2CC(CNc3ccc(NS(=O)(=O)c4cnn(-c5ccc(F)cc5)c4)c(C(=O)O)c3)C2)ccc1NS(=O)(=O)c1ccc(-c2ccc(Cl)cc2)cc1] | 859.2 |
| 31 | 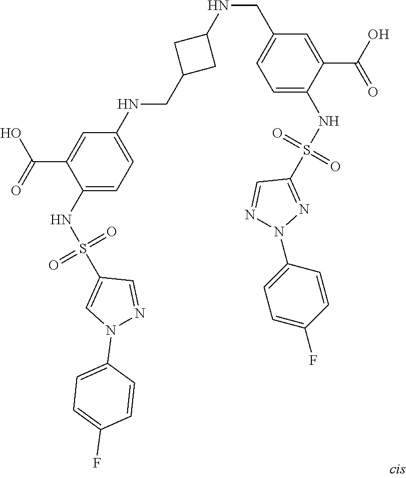 [SMILES: O=C(O)c1cc(NCC2CC(NCc3ccc(NS(=O)(=O)c4cnn(-c5ccc(F)cc5)n4)c(C(=O)O)c3)C2)ccc1NS(=O)(=O)c1cnn(-c2ccc(F)cc2)c1] | 834.2 |
| 32 | 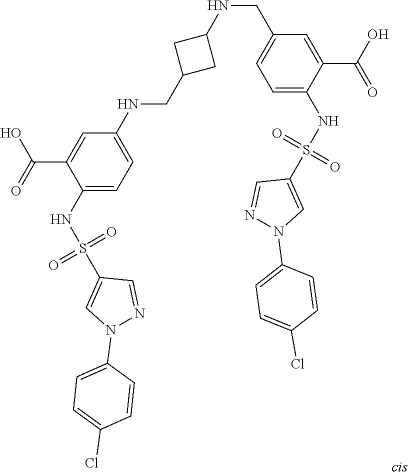 [SMILES: O=C(O)c1cc(CNC2CC(CNc3ccc(NS(=O)(=O)c4cnn(-c5ccc(Cl)cc5)c4)c(C(=O)O)c3)C2)ccc1NS(=O)(=O)c1cnn(-c2ccc(Cl)cc2)c1] | 865.1 |
| 33 | 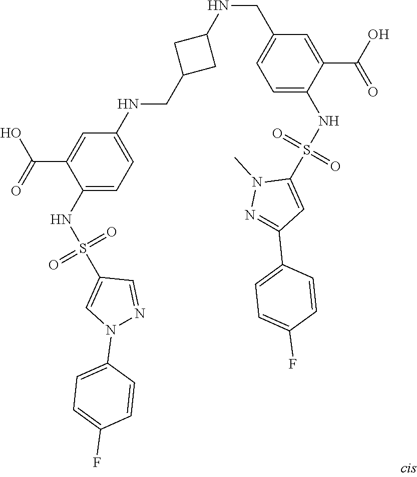 [SMILES: Cn1nc(-c2ccc(F)cc2)cc1S(=O)(=O)Nc1ccc(CNC2CC(CNc3ccc(NS(=O)(=O)c4cnn(-c5ccc(F)cc5)c4)c(C(=O)O)c3)C2)cc1C(=O)O] | 847.2 |
| 34 | 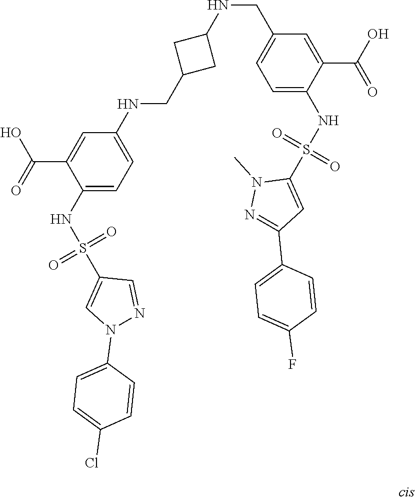 [SMILES: Cn1nc(-c2ccc(F)cc2)cc1S(=O)(=O)Nc1ccc(CNC2CC(CNc3ccc(NS(=O)(=O)c4cnn(-c5ccc(Cl)cc5)c4)c(C(=O)O)c3)C2)cc1C(=O)O] | 863.1 |
| 35 | 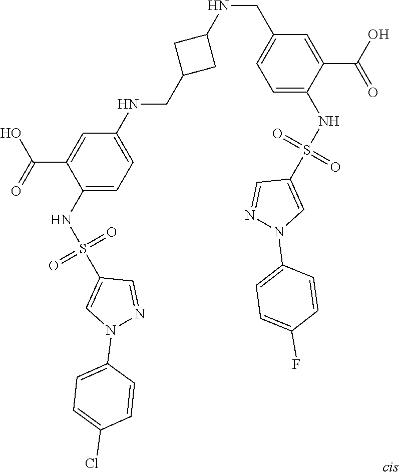 [SMILES: O=C(O)c1cc(CNC2CC(CNc3ccc(NS(=O)(=O)c4cnn(-c5ccc(Cl)cc5)c4)c(C(=O)O)c3)C2)ccc1NS(=O)(=O)c1cnn(-c2ccc(F)cc2)c1] | 849.2 |
| 36 | 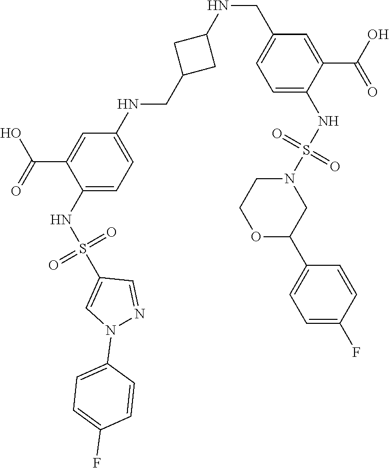 [SMILES: O=C(O)c1cc(NCC2CC(NCc3ccc(NS(=O)(=O)N4CCOC(c5ccc(F)cc5)C4)c(C(=O)O)c3)C2)ccc1NS(=O)(=O)c1cnn(-c2ccc(F)cc2)c1] | 852.2 |

### EXAMPLE 8

### Preparation of Compound 37

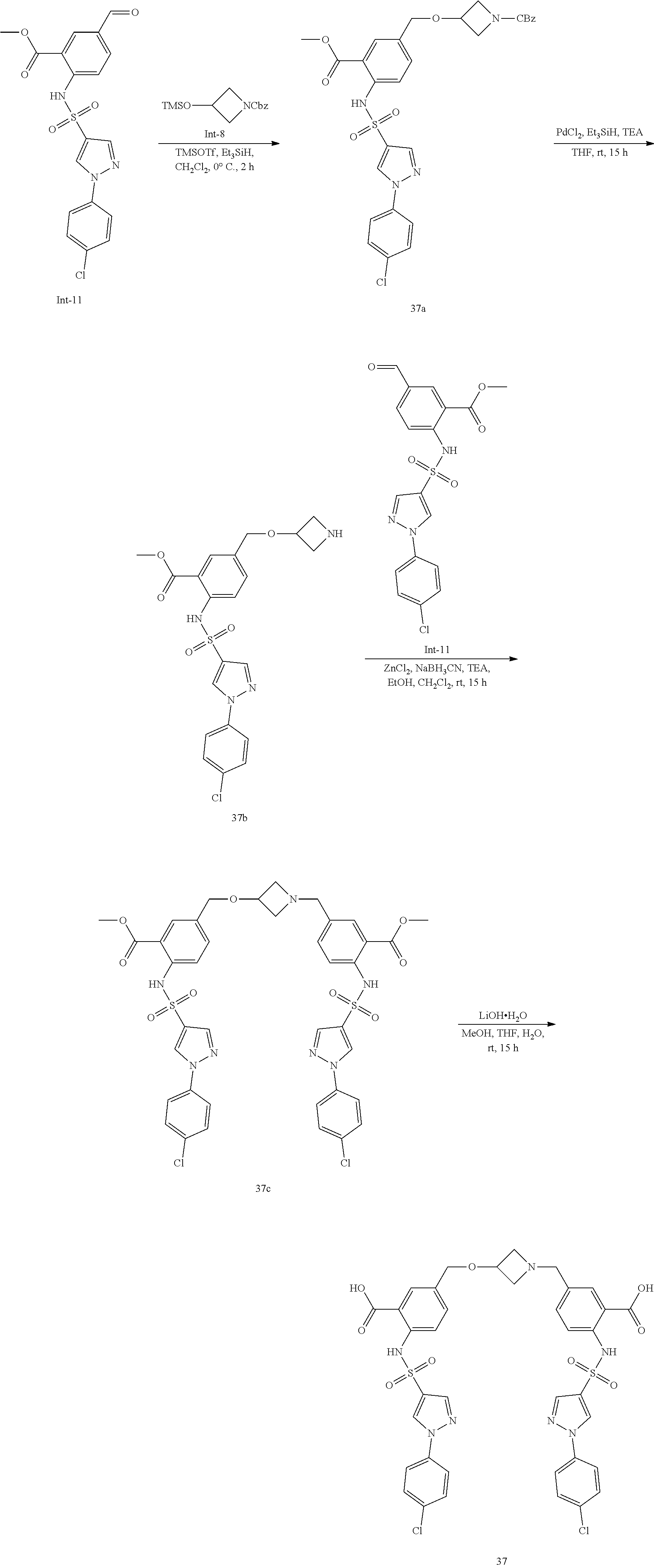

### Step 1: benzyl 3-(4-(1-(4-chlorophenyl)-1H-pyrazole-4-sulfonamido)-3-(methoxy-carbonyl)benzyl)oxy) azetidine-1-carboxylate (37a)

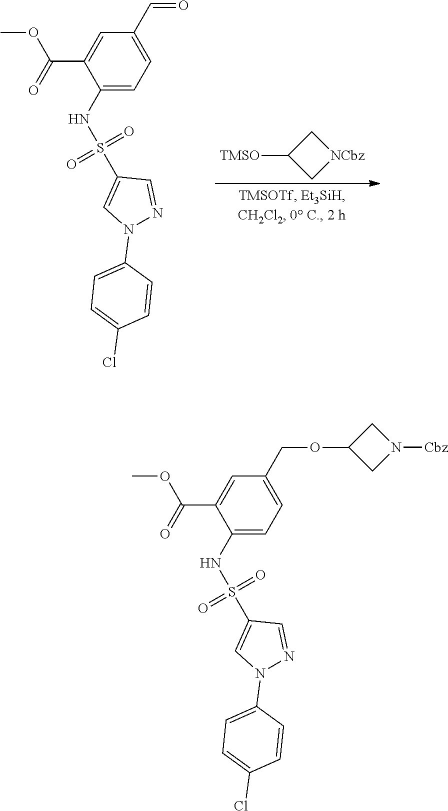

To a 0° C. solution of methyl 2-(1-(4-chlorophenyl)-1H-pyrazole-4-sulfonamido)-5-formylbenzoate (Int-11; 300 mg, 0.715 mmol) in CH₂Cl₂ (10 mL) were added benzyl 3-((trimethylsilyl)oxy) azetidine-1-carboxylate (Int-8; 399 mg, 1.429 mmol) and trimethylsilyl trifluoromethanesulfonate (0.258 mL, 1.429 mmol). The reaction mixture was stirred at 0° C. for 0.5 h. Next, triethylsilane (0.467 mL, 2.86 mmol) was added and the reaction mixture was stirred at 0° C. for 1 h. H₂O (20 mL) was added and the mixture was extracted with CH₂Cl₂ (20 mL). The organics were washed with brine (20 mL), dried over anh. Na₂SO₄, filtered, and concentrated in vacuo. The resulting residue was purified by flash chromatography on SiO₂ [Isco®, Agela® 4 g column, eluent of 0) to 30% EtOAc/Pet. ether gradient at 30 mL/min] to provide benzyl 3-((4-(1-(4-chlorophenyl)-1H-pyrazole-4-sulfonamido)-3-(methoxycarbonyl)benzyl)oxy) azetidine-1-carboxylate. MS (ESI, m/z): 611.1 [M+H⁺].

### Step 2: methyl 5-((azetidin-3-yloxy)methyl)-2-(1-(4-chlorophenyl)-1H-pyrazole-4-sulfonamido)benzoate (37b)

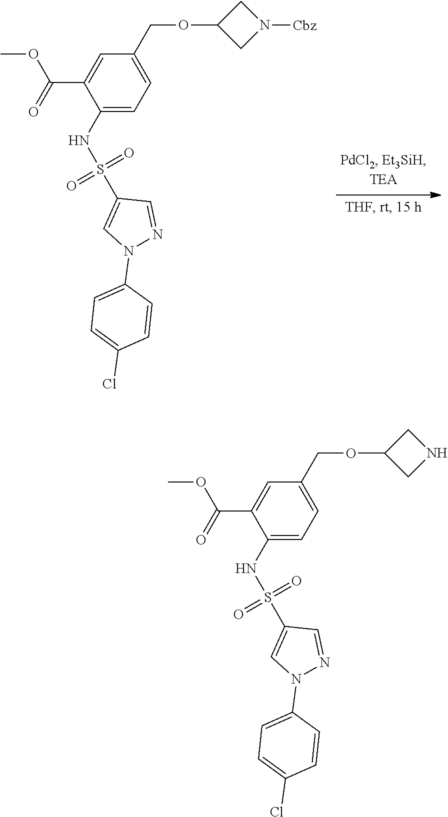 [SMILES: COC(=O)c1cc(COC2CN(C(=O)OCc3ccccc3)C2)ccc1NS(=O)(=O)c1cnn(-c2ccc(Cl)cc2)c1 ; COC(=O)c1cc(COC2CNC2)ccc1NS(=O)(=O)c1cnn(-c2ccc(Cl)cc2)c1]

To a solution of benzyl 3-((4-(1-(4-chlorophenyl)-1 H-pyrazole-4-sulfonamido)-3-(methoxycarbonyl)benzyl)oxy) azetidine-1-carboxylate (37a; 300 mg, 0.491 mmol) in THF (10 mL) was added triethylsilane (0.198 mL, 1.227 mmol), PdCl2 (8.71 mg, 0.049 mmol), and TEA (0.068 mL, 0.491 mmol). The reaction mixture was stirred at room temperature for 15 h, concentrated in vacuo and the resulting residue was purified by reverse phase chromatography [YMC-Actus, Triart C18 column (150*30 mm, 5 μm), eluant of mobile phase A: H₂ O (0.1% TFA) and mobile phase B: MeCN (0.1% TFA) to provide methyl 5-((azetidin-3-yloxy)methyl)-2-(1-(4-chlorophenyl)-1H-pyrazole-4-sulfonamido)benzoate. MS (ESI, m/z): 477.1 [M+H⁺]. 1H NMR (400 MHZ, CDCl₃) δ ppm 8.31-8.24 (m, 1H), 7.95-7.88 (m, 2H), 7.77 (d, J=8.0 Hz, 1H), 7.56 (d, J=8.4 Hz, 2H), 7.51-7.41 (m, 3H), 4.44-4.31 (m, 3H), 3.94-3.89 (m, 3H), 3.79-3.73 (m, 2H), 3.72-3.64 (m, 2H).

### Step 3: methyl 2-(1-(4-chlorophenyl)-1H-pyrazole-4-sulfonamido)-5-(((1-(4-(1-(4-chlorophenyl)-1H-pyrazole-4-sulfonamido)-3-(methoxycarbonyl)benzyl) azetidin-3-yl)oxy)methyl)benzoate (37c)

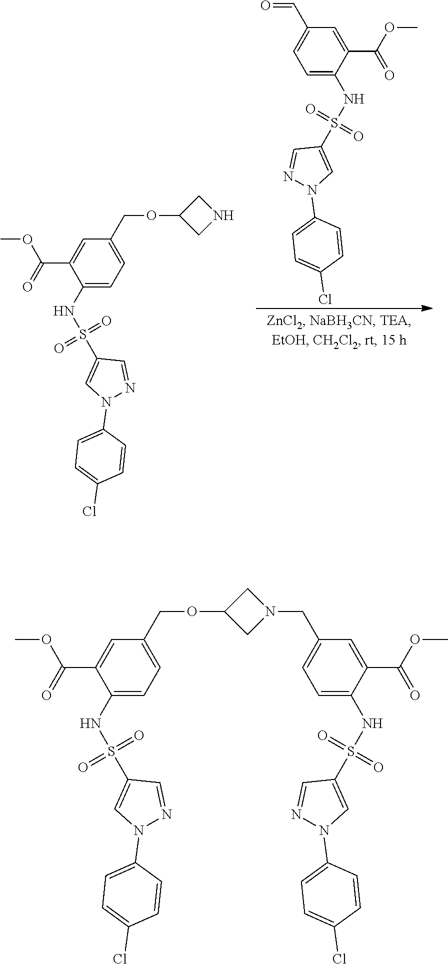

To a solution of methyl 5-((azetidin-3-yloxy)methyl)-2-(1-(4-chlorophenyl)-1H-pyrazole-4-sulfonamido)benzoate (37b; 56.8 mg, 0.119 mmol) in EtOH (0.75 mL) and CH₂Cl₂ (0.75 mL) was added methyl 2-(1-(4-chlorophenyl)-1H-pyrazole-4-sulfonamido)-5-formylbenzoate (Int-11; 50 mg, 0.119 mmol), TEA (0.083 mL, 0.595 mmol), zinc (II) chloride (32.5 mg, 0.238 mmol), and NaBH₃CN (29.9 mg, 0.476 mmol) at room temperature. The reaction mixture was stirred for 15 h, concentrated in vacuo and the resulting residue was purified directly by preparative thin-layer chromatography on SiO₂ (eluting with CH₂Cl₂/MeOH=20:1) to provide methyl 2-(1-(4-chlorophenyl)-1H-pyrazole-4-sulfonamido)-5-(((1-(4-(1-(4-chlorophenyl)-1H-pyrazole-4-sulfonamido)-3-(methoxycarbonyl)benzyl) azetidin-3-yl)oxy)methyl)benzoate. MS (ESI, m/z): 880.0 [M+H⁺].

### Step 4:5-(((1-(3-carboxy-4-(1-(4-chlorophenyl)-1H-pyrazole-4-sulfonamido)benzyl) azetidin-3-yl)oxy)methyl)-2-(1-(4-chlorophenyl)-1H-pyrazole-4-sulfon amido)benzoic acid (37)

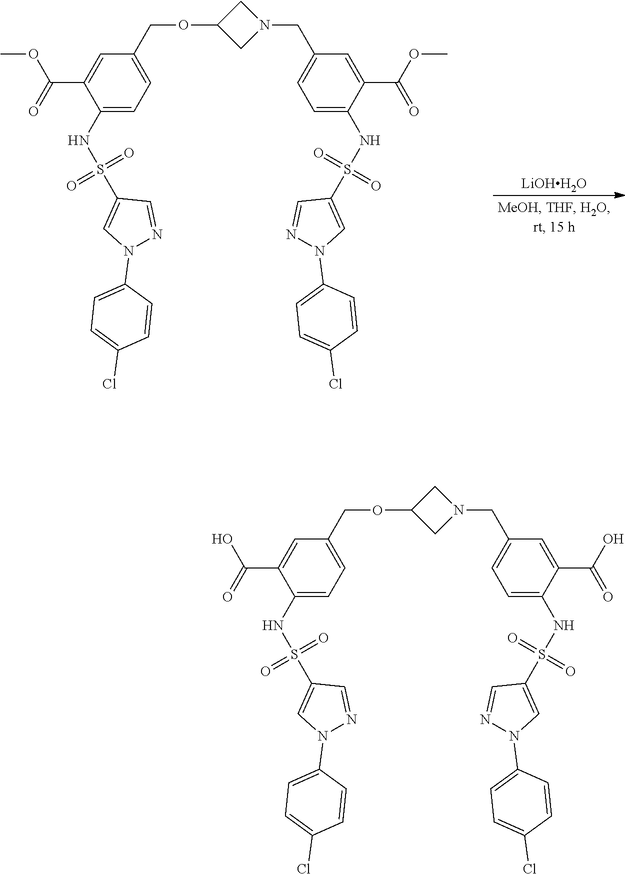

To a solution of methyl 2-(1-(4-chlorophenyl)-1H-pyrazole-4-sulfonamido)-5-(((1-(4-(1-(4-chlorophenyl)-1H-pyrazole-4-sulfonamido)-3-(methoxycarbonyl)benzyl) azetidin-3-yl)oxy)methyl)benzoate (37c; 60 mg, 0.068 mmol) in MeOH (0.5 mL), H₂O (0.5 mL), and THF (0.5 mL) was added lithium hydroxide monohydrate (28.6 mg, 0.681 mmol). The reaction mixture was stirred at room temperature for 15 h. HCl (6 M) was added dropwise to the reaction mixture to pH=5, and the reaction mixture was concentrated in vacuo. The resulting residue was purified by reverse phase chromatography [Phenomenex, Synergi C18 column (150*30 mm, 4 μm), eluent of mobile phase A: water (0.05% HCl) and mobile phase B: MeCN at 220 nM] to provide 5-(((1-(3-carboxy-4-(1-(4-chlorophenyl)-1H-pyrazole-4-sulfonamido)benzyl) azetidin-3-yl)oxy)methyl)-2-(1-(4-chlorophenyl)-1H-pyrazole-4-sulfonamido)benzoic acid. MS (ESI, m/z): 850.0 [M−H⁺]. ¹H NMR (400 MHZ, DMSO-d₆) δ ppm 9.36 (d, J=14.4 Hz, 2H), 8.25 (s, 1H), 8.18 (s, 1H), 8.11 (s, 1H), 7.96-7.85 (m, 5H), 7.75-7.53 (m, 8H), 4.40-3.90 (m, 9H).

The following compound (38) of the present disclosure were made using the methods described in Example 8 above, and substituting the appropriate reactants and/or reagents:

| | | Observed |
|---|---|---|
| Compound | Structure | MS |
| 38 | 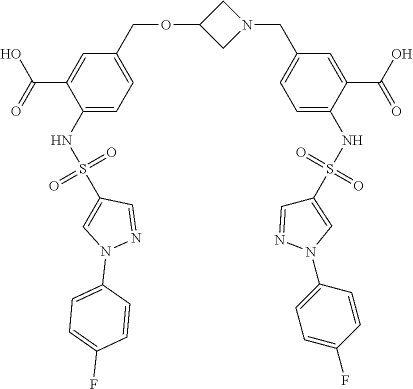 [SMILES: O=C(O)c1cc(COC2CN(Cc3ccc(NS(=O)(=O)c4cnn(-c5ccc(F)cc5)c4)c(C(=O)O)c3)C2)ccc1NS(=O)(=O)c1cnn(-c2ccc(F)cc2)c1] | 818.1 |

### EXAMPLE 9

### Preparation of Compound 39

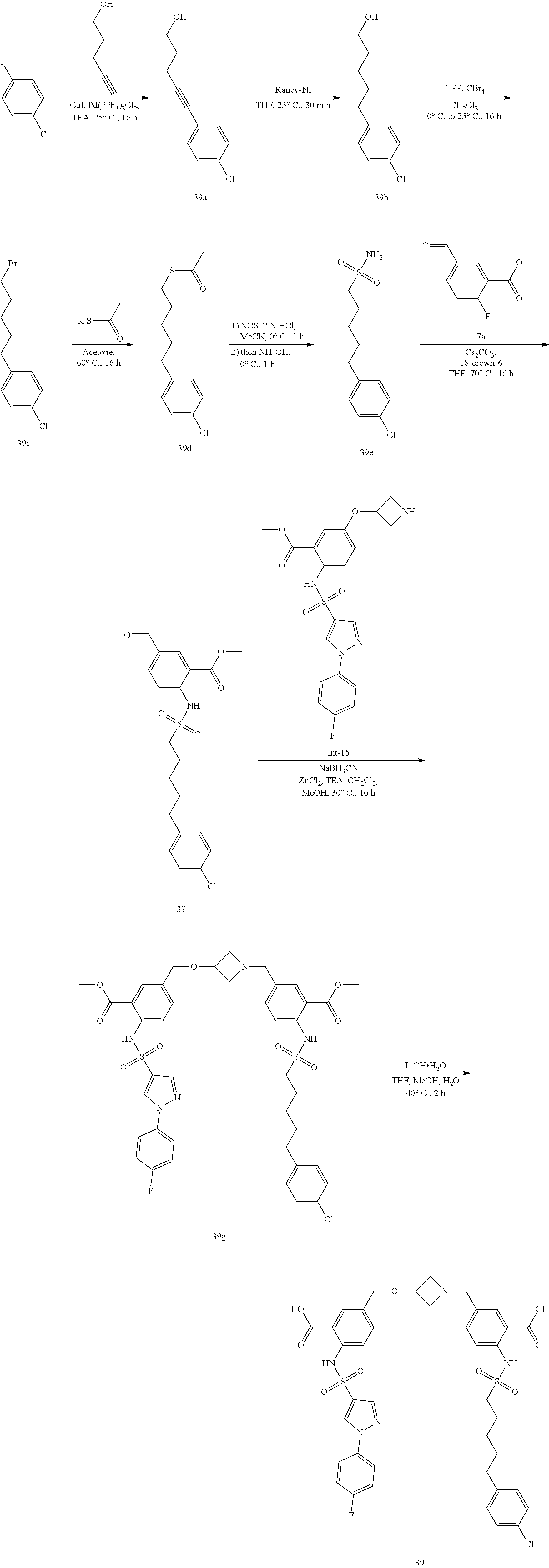

### Step 1:5-(4-chlorophenyl) pent-4-yn-1-ol (39a)

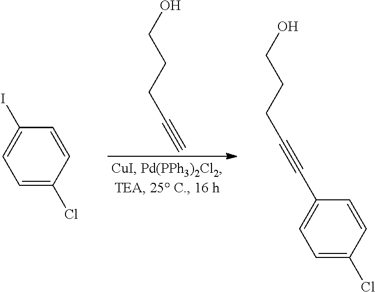

To a stirred mixture of 1-chloro-4-iodobenzene (2 g, 8.39 mmol) and pent-4-yn-1-ol (0.776 g, 9.23 mmol) in TEA (10 mL) were added bis(triphenylphosphine)palladium (II) dichloride (0.589 g, 0.839 mmol) and Cul (0.319 g, 1.677 mmol). The reaction mixture was stirred at 25° C. for 16 h under N₂. Then sat. aq. NH₄Cl (50 mL) was added and the mixture was extracted with EtOAc (2×100 mL). The combined organics were washed with brine (50 mL), dried over anh. Na₂SO₄, filtered, and concentrated in vacuo. The resulting residue was purified by flash chromatography on SiO₂ (Isco®, 20 g column, eluent of 0 to 3% EtOAc/Pet. ether gradient at 30 mL/min) to provide 5-(4-chloro-phenyl) pent-4-yn-1-ol. MS (ESI, m/z): 195.2 [M+H⁺]. ¹H NMR (500 MHZ, CDCl₃) δ ppm 7.33-7.29 (m, 2H), 7.27-7.23 (m, 2H), 3.82 (t, J=6.0 Hz, 2H), 2.53 (t, J=7.0 Hz, 2H), 1.86 (quin, J=6.5 Hz, 2H).

### Step 2:5-(4-chlorophenyl) pentan-1-ol (39b)

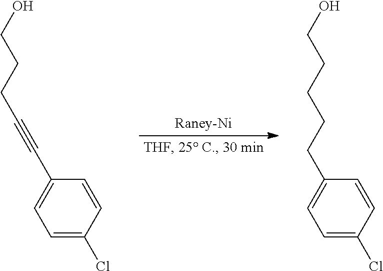 [SMILES: OCCCC#Cc1ccc(Cl)cc1 ; OCCCCCc1ccc(Cl)cc1]

To a stirred mixture of 5-(4-chlorophenyl) pent-4-yn-1-ol (39a; 200 mg, 1.027 mmol) in THF (5 mL) was added Raney nickel (60.3 mg, 1.027 mmol). The reaction mixture was stirred at 25° C. for 30 min under H₂ (15 psi), then filtered, washing the filter cake was washed with EtOAc (50 mL). The filtrate was concentrated in vacuo to provide 5-(4-chlorophenyl) pentan-1-ol, which was used in the subsequent step without further purification.

### Step 3:1-(5-bromopentyl)-4-chlorobenzene (39c)

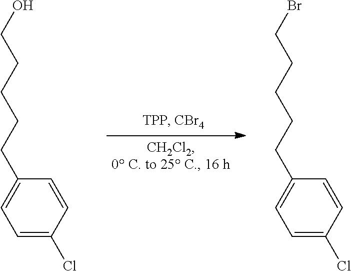 [SMILES: OCCCCCc1ccc(Cl)cc1 ; Clc1ccc(CCCCCBr)cc1]

To a stirred mixture of 5-(4-chlorophenyl) pentan-1-ol (39b; 180 mg, 0.906 mmol) in CH₂Cl₂ (4 mL) was added PPh3 (356 mg, 1.359 mmol) and CBr4 (451 mg, 1.359 mmol) at 0° C. The reaction mixture was stirred at 25° C. for 16 h under N₂. The reaction mixture was concentrated in vacuo, and the resulting residue was purified directly by flash chromatography on SiO₂ [Isco®, 4 g column, eluent of 0 to 100% EtOAc/Pet. ether gradient at 30 mL/min] to provide 1-(5-bromopentyl)-4-chlorobenzene. ¹H NMR (400 MHZ, CDCl₃) δ ppm 7.23-7.27 (m, 2H) 7.11 (d, J=8.07 Hz, 2H) 3.41 (t, J=6.72 Hz, 2H) 2.60 (t, J=7.58 Hz, 2H) 1.85-1.92 (m, 2H) 1.63 (br t, J=7.58 Hz, 2H) 1.45-1.52 (m, 2H).

### Step 4: S-(5-(4-chlorophenyl) pentyl) ethanethioate (39d)

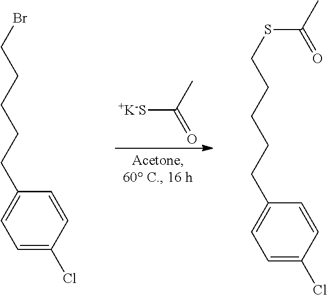

To a solution of 1-(5-bromopentyl)-4-chlorobenzene (39c; 200 mg, 0.765 mmol)) in acetone (5 mL) was added potassium thioacetate (87 mg, 0.765 mmol). The reaction was stirred at 60° C. for 16 h, concentrated in vacuo, and the resulting residue was purified by flash chromatography on SiO₂ [Isco®, 4 g column, eluent of 0 to 3% EtOAc/Pet. ether gradient at 20 mL/min] to give S-(5-(4-chlorophenyl) pentyl) ethanethioate. MS (ESI, m/z): 280.4 [M+Na⁺].

### Step 5:5-(4-chlorophenyl) pentane-1-sulfonamide (39e)

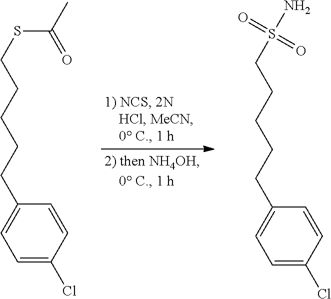 [SMILES: CC(=O)SCCCCCc1ccc(Cl)cc1 ; NS(=O)(=O)CCCCCc1ccc(Cl)cc1]

To a solution of S-(5-(4-chlorophenyl) pentyl) ethanethioate (39d; 90 mg, 0.350 mmol) in MeCN (4 mL) was added 2 N HCl (0.018 mL, 0.035 mmol) and NCS (187 mg, 1.402 mmol) at 0° C. The reaction mixture was stirred at 0° C. for 1 h, and NH₄OH (2 mL) was added. The reaction was stirred at 0° C. for 1 h, and H₂O (30 mL) was added. The reaction mixture was extracted with EtOAc (2×30 mL). The combined organics were washed with brine (30 mL), dried over anh. Na₂SO₄, filtered and concentrated in vacuo. The resulting residue was purified by preparative reverse phase chromatography [Phenomenex Synergi C18 column (150*21.2 mm, 4 μm), eluent of mobile phase A: water (0.1% TFA) and mobile phase B: MeCN at 220 nm] to provide [5-(4-chlorophenyl) pentane-1-sulfonamide. ¹H NMR (500 MHz, CDCl₃) δ ppm 7.27-7.24 (m, 2H), 7.10 (d, J=8.0 Hz, 2H), 4.57 (br s, 2H), 3.17-3.05 (m, 2H), 2.61 (t, J=7.5 Hz, 2H), 1.94-1.85 (m, 2H), 1.69-1.63 (m, 2H), 1.47 (quin, J=7.5 Hz, 2H).

### Step 6: methyl 2-((5-(4-chlorophenyl) pentyl) sulfonamido)-5-formylbenzoate (39f)

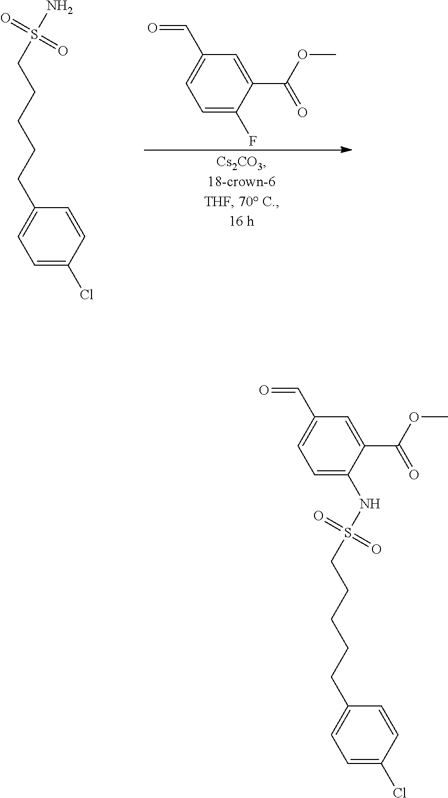 [SMILES: NS(=O)(=O)CCCCCc1ccc(Cl)cc1 ; COC(=O)c1cc(C=O)ccc1F ; COC(=O)c1cc(C=O)ccc1NS(=O)(=O)CCCCCc1ccc(Cl)cc1]

To a stirred mixture of 5-(4-chlorophenyl) pentane-1-sulfonamide (39e; 60 mg, 0.229 mmol) in THF (2 mL) were added methyl 2-fluoro-5-formylbenzoate (45.9 mg, 0.252 mmol), cesium carbonate (224 mg, 0.688 mmol), and 18-crown-6 (60.6 mg, 0.229 mmol). The reaction mixture was stirred at 70° C. for 16 h, then filtered. The filtrate was concentrated in vacuo, and the resulting residue was purified by flash chromatography on SiO₂ [Isco2, 4 g column, eluent of 0 to 20% EtOAc/Pet. ether gradient at 20 mL/min] to provide methyl 2-((5-(4-chlorophenyl) pentyl) sulfonamido)-5-formylbenzoate. MS (ESI, m/z): 424.2 [M+H⁺].

### Step 7: methyl 2-(5-(4-chlorophenyl) pentyl) sulfonamido)-5-(3-(4-((1-(4-fluorophenyl)-1H-pyrazole)-4-sulfonamido)-3-(methoxycarbonyl) phenoxy) azetidin-1-yl)methyl)benzoate (39g)

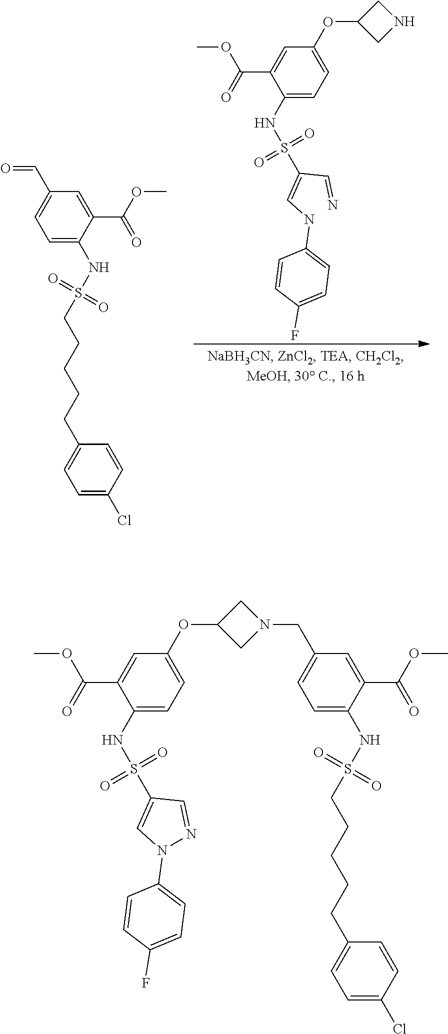

To a stirred mixture of methyl 5-(azetidin-3-yloxy)-2-((1-(4-fluorophenyl)-1H-pyrazole)-4-sulfonamido)benzoate (39f; 30 mg, 0.067 mmol), methyl 2-((5-(4-chlorophenyl) pentyl)-sulfonamido)-5-formylbenzoate (Int-15; 28.5 mg, 0.067 mmol), and zinc (II) chloride (18.32 mg, 0.134 mmol) in MeOH (0.5 mL) and CH₂Cl₂ (2 mL) was added TEA (0.028 ml, 0.202 mmol). The mixture was stirred at 30° C. for 0.5 h. Sodium cyanoborohydride (12.67 mg, 0.202 mmol) was added and the reaction mixture was stirred at 30° C. for 16 h. The reaction mixture was concentrated in vacuo and the resulting residue was purified by preparative reverse phase chromatography [Phenomenex Synergi C18 column (150*21.2 mm, 4 μm), eluent of mobile phase A: water (0.1% TFA) and mobile phase B: MeCN at 220 nm] to provide methyl 2-((5-(4-chlorophenyl) pentyl) sulfonamido)-5-((3-(4-((1-(4-fluorophenyl)-1H-pyrazole)-4-sulfonamido)-3-(methoxycarbonyl) phenoxy) azetidin-1-yl)methyl)-benzoate. MS (ESI, m/z): 854.2 [M+H⁺]. 1H NMR (400 MHZ, CD₃OD) δ ppm 8.66 (s, 1H), 8.21 (d, J=2.0 Hz, 1H), 7.83-7.77 (m, 2H), 7.77-7.66 (m, 4H), 7.37 (d, J=3.0 Hz, 1H), 7.27-7.18 (m, 4H), 7.16 (dd, J=3.0, 9.0 Hz, 1H), 7.09 (d, J=8.0 Hz, 2H), 5.13 (br s, 1H), 4.61 (br dd, J=6.0, 12.0 Hz, 2H), 4.46 (s, 2H), 4.24 (br d, J=8.0 Hz, 2H), 3.96 (s, 3H), 3.84 (s, 3H), 3.29-3.23 (m, 2H), 2.53 (t, J=7.5 Hz, 2H), 1.81-1.74 (m, 2H), 1.60-1.51 (m, 2H), 1.43-1.36 (m, 2H).

### Step 8:5-((3-(3-carboxy-4-((1-(4-fluorophenyl)-1H-pyrazole)-4-sulfonamido) phenoxy) azetidin-1-yl)methyl)-2-((5-(4-chlorophenyl) pentyl) sulfonamido)benzoic acid (39)

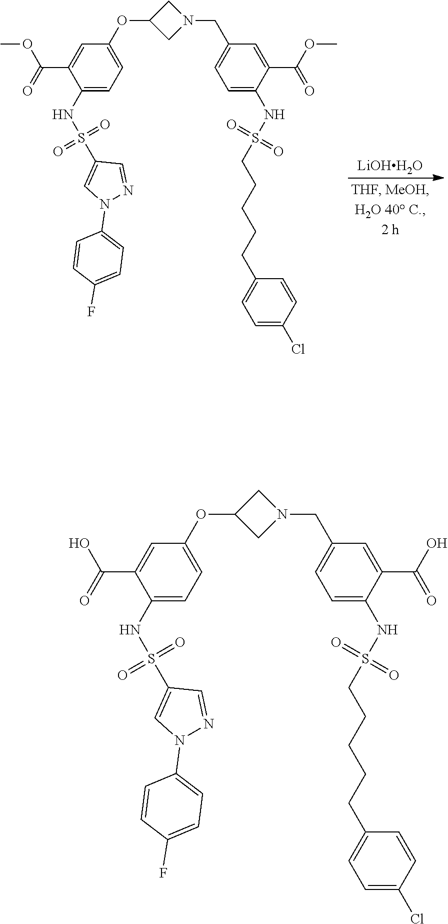

To a stirred mixture of methyl 2-((5-(4-chlorophenyl) pentyl) sulfonamido)-5-((3-(4-((1-(4-fluorophenyl)-1H-pyrazole)-4-sulfonamido)-3-(methoxycarbonyl) phenoxy) azetidin-1-yl)methyl)benzoate (39g; 30 mg, 0.035 mmol) in THF (0.2 mL), MeOH (0.2 mL), and water (0.1 mL) was added lithium hydroxide hydrate (14.73 mg, 0.351 mmol). The mixture was stirred at 40° C. for 2 h, then concentrated in vacuo, and the resulting mixture was acidified to pH=3 with 1 N HCl. The mixture was purified by preparative reverse phase chromatography [Welch Xtimate C18 column (150*25 mm, 5 μm), eluent of mobile phase A: water (0.1% TFA) and mobile phase B: MeCN at 220 nm] to provide 5-((3-(3-carboxy-4-((1-(4-fluorophenyl)-1H-pyrazole)-4-sulfonamido)-phenoxy) azetidin-1-yl)methyl)-2-((5-(4-chlorophenyl) pentyl) sulfonamido)benzoic acid. MS (ESI, m/z): 824.2 [M−H⁺]. ¹H NMR (500 MHZ, CD₃OD) δ ppm 8.65 (s, 1H), 8.25 (d, J=2.0 Hz, 1H), 7.81-7.76 (m, 2H), 7.75-7.70 (m, 3H), 7.66 (dd, J=2.0, 8.5 Hz, 1H), 7.41 (d, J=3.0 Hz, 1H), 7.25-7.17 (m, 4H), 7.14 (dd, J=3.0, 9.0 Hz, 1H), 7.08 (d, J=8.0 Hz, 2H), 5.17-5.08 (m, 1H), 4.59 (br dd, J=6.5, 12.0 Hz, 2H), 4.45 (s, 2H), 4.23 (br dd, J=3.5, 12.0 Hz, 2H), 3.26-3.20 (m, 2H), 2.51 (t, J=7.5 Hz, 2H), 1.81-1.74 (m, 2H), 1.58-1.50 (m, 2H), 1.37 (quin, J=7.5 Hz, 2H).

The following compounds (40 through 43) of the present disclosure were made using the methods described in Example 9 above, and substituting the appropriate reactants and/or reagents:

| | | Observed |
|---|---|---|
| Compound | Structure | MS |
| 40 | 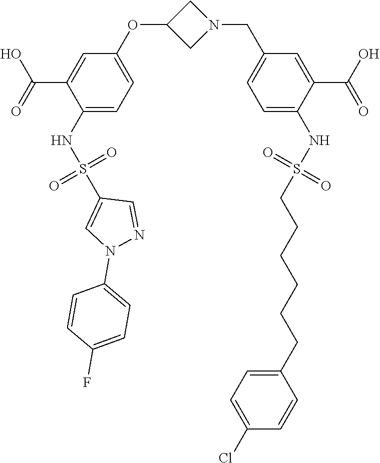 [SMILES: O=C(O)c1cc(CN2CC(Oc3ccc(NS(=O)(=O)c4cnn(-c5ccc(F)cc5)c4)c(C(=O)O)c3)C2)ccc1NS(=O)(=O)CCCCCCc1ccc(Cl)cc1] | 838.1 [M − H⁺] |
| 41 | 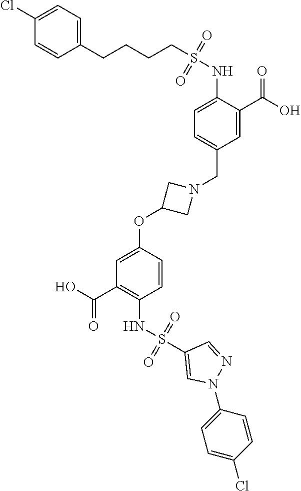 [SMILES: O=C(O)c1cc(CN2CC(Oc3ccc(NS(=O)(=O)c4cnn(-c5ccc(Cl)cc5)c4)c(C(=O)O)c3)C2)ccc1NS(=O)(=O)CCCCc1ccc(Cl)cc1] | 828.2 |
| 42 | 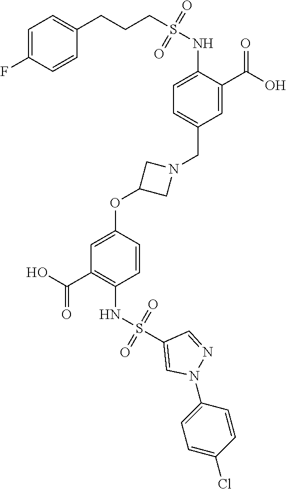 [SMILES: O=C(O)c1cc(CN2CC(Oc3ccc(NS(=O)(=O)c4cnn(-c5ccc(Cl)cc5)c4)c(C(=O)O)c3)C2)ccc1NS(=O)(=O)CCCc1ccc(F)cc1] | 798.3 |
| 43 | 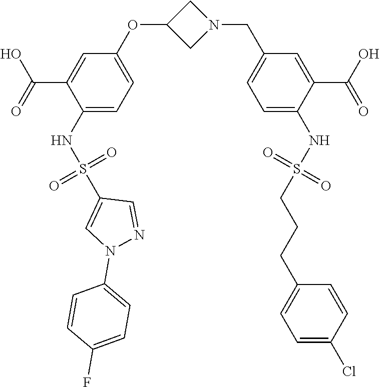 [SMILES: O=C(O)c1cc(CN2CC(Oc3ccc(NS(=O)(=O)c4cnn(-c5ccc(F)cc5)c4)c(C(=O)O)c3)C2)ccc1NS(=O)(=O)CCCc1ccc(Cl)cc1] | 796.1 [M − H⁺] |

### EXAMPLE 10

### Preparation of Compound 44

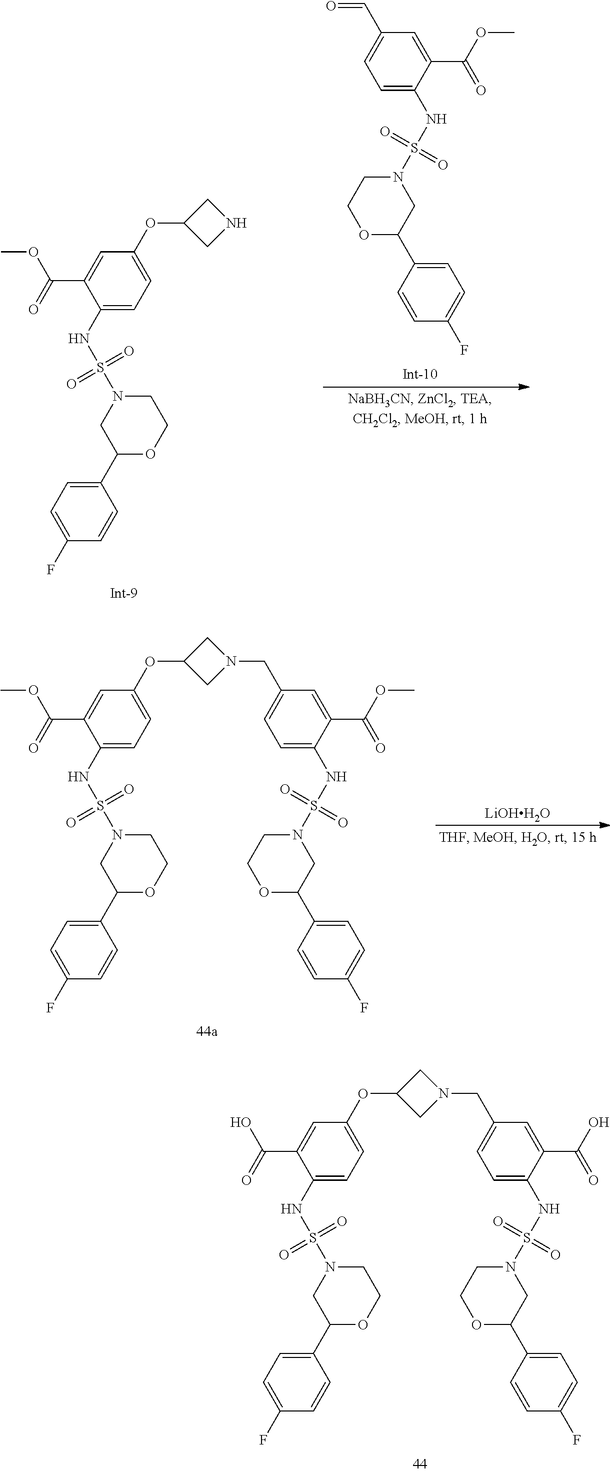

### Step 1: methyl 2-((2-(4-fluorophenyl) morpholine)-4-sulfonamido)-5-((1-(4-((2-(4-fluorophenyl) morpholine)-4-sulfonamido)-3-(methoxycarbonyl)benzyl) azetidin-3-yl)oxy)benzoate (44a)

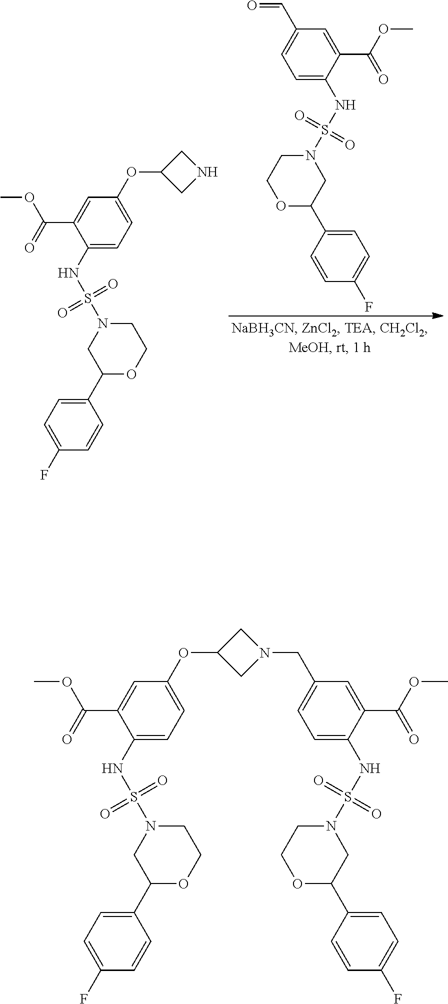

To a solution of methyl 5-(azetidin-3-yloxy)-2-((2-(4-fluorophenyl) morpholine)-4-sulfonamido)benzoate (Int-9; 40 mg, 0.086 mmol) and methyl 2-((2-(4-fluorophenyl)-morpholine)-4-sulfonamido)-5-formylbenzoate (Int-10; 36.3 mg, 0.086 mmol) in CH₂Cl₂ (3 mL) and MeOH (1 mL) was added TEA (0.060 mL, 0.430 mmol), zinc (II) chloride (23.42 mg, 0.172 mmol) and sodium cyanoborohydride (21.60 mg, 0.344 mmol). The reaction mixture was stirred at room temperature for 1 h. H₂O (30 mL) was added and the mixture was extracted with EtOAc (30 mL). The organics were washed with brine (30 mL), dried over anh. Na₂SO₄, filtered, and concentrated in vacuo. The resulting residue was purified by preparative reverse phase chromatography [Boston Prime C18 column (150*30 mm, 5 μm), eluent of mobile phase A: water (0.05% NH₄OH+10 mM NH₄HCO3) and mobile phase B: MeCN at 220 nm] to provide methyl 2-((2-(4-fluoro-phenyl) morpholine)-4-sulfonamido)-5-((1-(4-((2-(4-fluorophenyl) morpholine)-4-sulfonamido)-3-(methoxycarbonyl)benzyl) azetidin-3-yl)oxy)benzoate. MS (ESI, m/z): 872.2 [M+H⁺].

### Step 2:5-((1-(3-carboxy-4-((2-(4-fluorophenyl) morpholine)-4-sulfonamido)benzyl) azetidin-3-yl)oxy)-2-((2-(4-fluorophenyl) morpholine)-4-sulfonamido)benzoic acid (44)

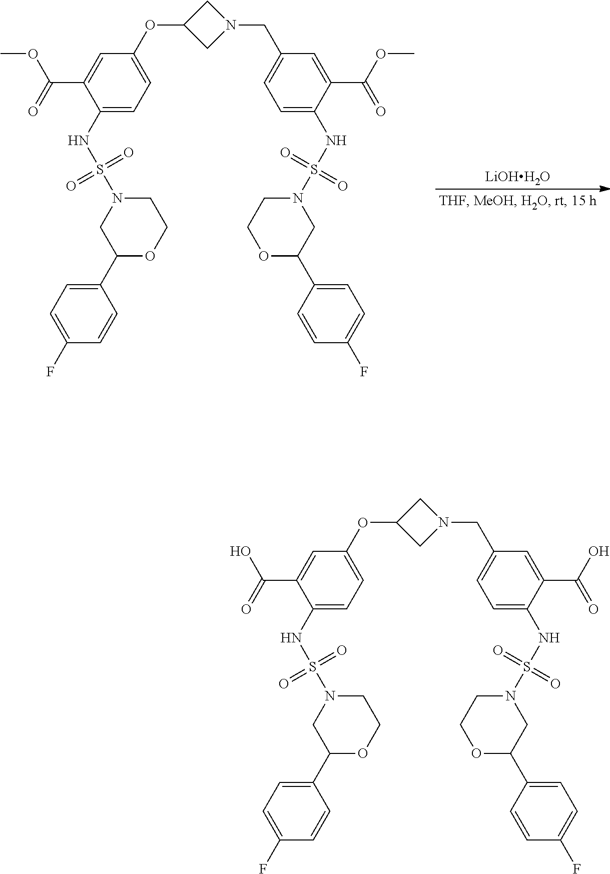

To a solution of methyl 2-((2-(4-fluorophenyl) morpholine)-4-sulfonamido)-5-((1-(4-((2-(4-fluorophenyl) morpholine)-4-sulfonamido)-3-(methoxycarbonyl)benzyl) azetidin-3-yl)oxy)benzoate (44a; 30 mg, 0.034 mmol) in MeOH (0.5 mL), THF (0.5 mL), and H₂O (0.5 mL) was added lithium hydroxide monohydrate (14.44 mg, 0.344 mmol). The reaction mixture was stirred at room temperature for 15 h. HCl (6 M) was added to pH=5, then water (10 mL) was added, and the mixture was extracted with EtOAc (10 mL). The organics were washed with brine (10 mL), dried over anh. Na₂SO₄, filtered, and concentrated in vacuo. The resulting residue was purified by preparative reverse phase chromatography [Phenomenex Synergi C18 column (150*30 mm, 4 μm), eluent of mobile phase A: water (0.05% HCl) and mobile phase B: MeCN at 220 nm] to provide 5-((1-(3-carboxy-4-((2-(4-fluorophenyl) morpholine)-4-sulfonamido)benzyl) azetidin-3-yl)oxy)-2-((2-(4-fluorophenyl) morpholine)-4-sulfonamido)benzoic acid. MS (ESI, m/z): 842.2 [M−H⁺]. ¹H NMR (400 MHZ, CD₃OD) δ ppm 8.27 (d, J=2.0 Hz, 1H), 7.82-7.78 (m, 1H), 7.69 (br d, J=9.2 Hz, 2H), 7.52 (d, J=2.8 Hz, 1H), 7.32 (dt, J=5.6, 8.4 Hz, 4H), 7.15 (dd, J=3.2, 9.2 Hz, 1H), 7.05 (t, J=8.8 Hz, 4H), 5.20-5.15 (m, 1H), 4.63 (dd, J=6.4, 12.4 Hz, 2H), 4.49 (s, 2H), 4.42-4.38 (m, 1H), 4.35-4.24 (m, 3H), 4.05-3.95 (m, 2H), 3.71-3.52 (m, 6H), 3.04-2.90 (m, 2H), 2.71 (ddd, J=10.4, 12.0, 19.6 Hz, 2H).

The following compounds (45 through 47) of the present disclosure were made using the methods described in Example 10 above, and substituting the appropriate reactants and/or

| | | Observed |
|---|---|---|
| Compound | Structure | MS |
| 45 | 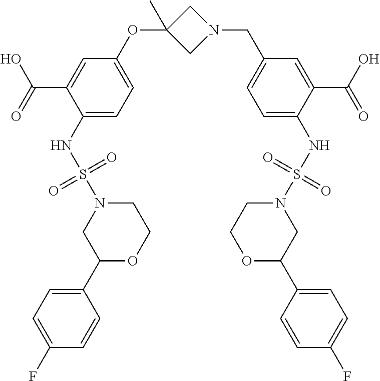 [SMILES: CC1(Oc2ccc(NS(=O)(=O)N3CCOC(c4ccc(F)cc4)C3)c(C(=O)O)c2)CN(Cc2ccc(NS(=O)(=O)N3CCOC(c4ccc(F)cc4)C3)c(C(=O)O)c2)C1] | 856.2 [M − H⁺] |
| 46 | 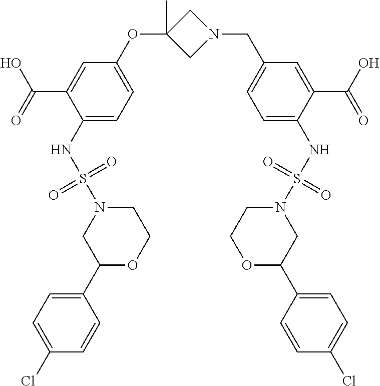 [SMILES: CC1(Oc2ccc(NS(=O)(=O)N3CCOC(c4ccc(Cl)cc4)C3)c(C(=O)O)c2)CN(Cc2ccc(NS(=O)(=O)N3CCOC(c4ccc(Cl)cc4)C3)c(C(=O)O)c2)C1] | 888.2 [M − H⁺]. |
| 47 | 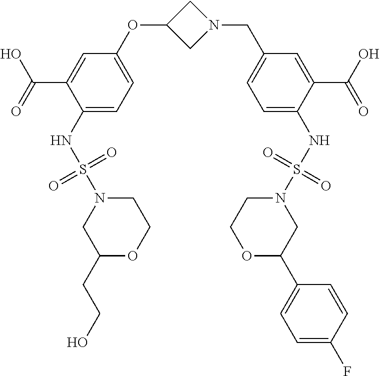 [SMILES: O=C(O)c1cc(OC2CN(Cc3ccc(NS(=O)(=O)N4CCOC(c5ccc(F)cc5)C4)c(C(=O)O)c3)C2)ccc1NS(=O)(=O)N1CCOC(CCO)C1] | 792.2 [M − H⁺] |

### EXAMPLE 11

### Preparation of Compound 48

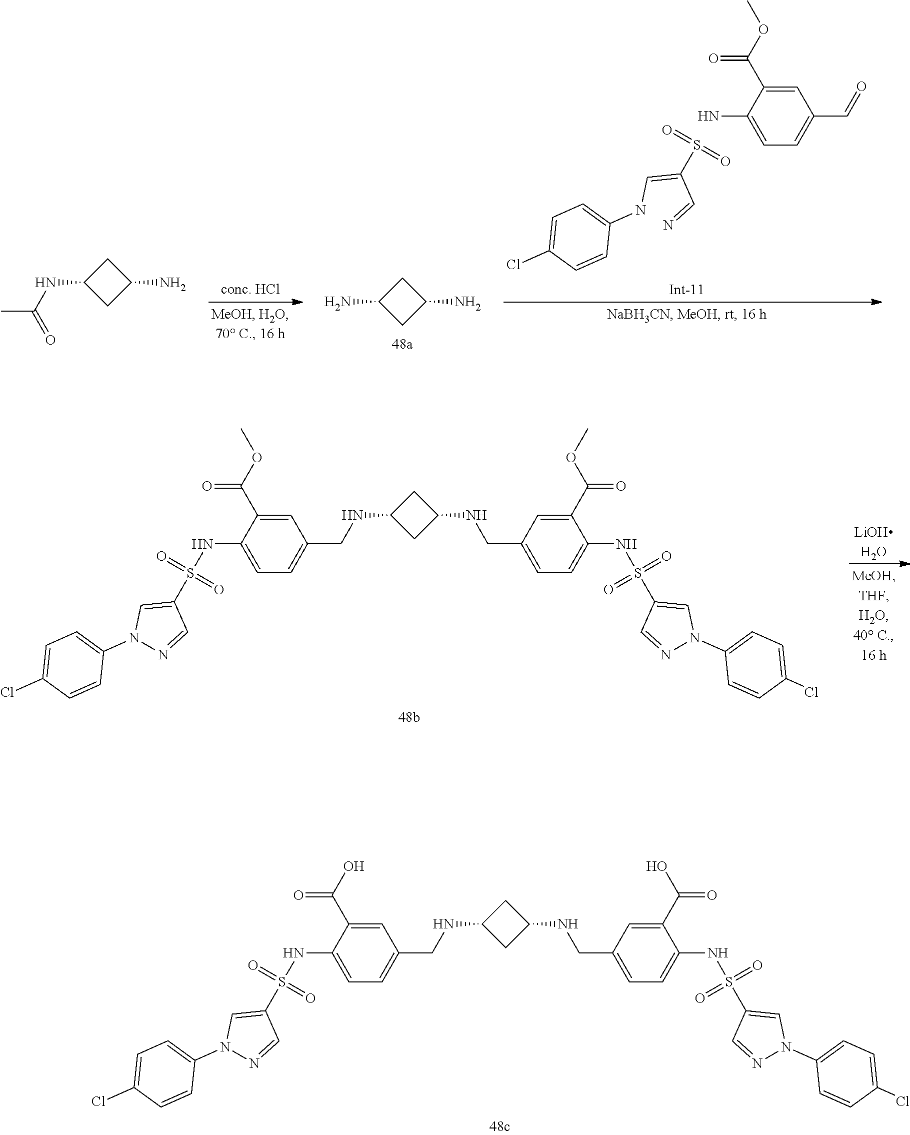

### Step 1: (1s,3s)-cyclobutane-1,3-diamine (48a)

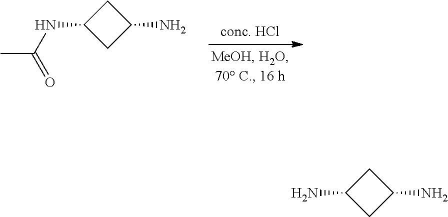

A mixture of N-((1S,3S)-3-aminocyclobutyl) acetamide (150 mg, 1.17 mmol) and 12N aq. HCl (1 mL) in MeOH (2 mL), and H₂O (1 mL) was stirred at 70° C. for 16 h. Then the reaction mixture was concentrated in vacuo to provide (1S,3S)-cyclobutane-1,3-diamine, which was used in the subsequent step without further purification. ¹H NMR (400 MHZ, DMSO) δ ppm 8.63 (br s, 4H), 3.49 (br d, J=5.6 Hz, 2H), 2.65-2.54 (m, 2H), 2.46-2.34 (m, 2H).

### Step 2: methyl 2-((1-(4-chlorophenyl)-1H-pyrazole)-4-sulfonamido)-5-((((1s, 3s)-3-((4-((1-(4-chlorophenyl)-1H-pyrazole)-4-sulfonamido)-3-(methoxycarbonyl)benzyl)-amino)cyclobutyl) amino)methyl)benzoate (48b)

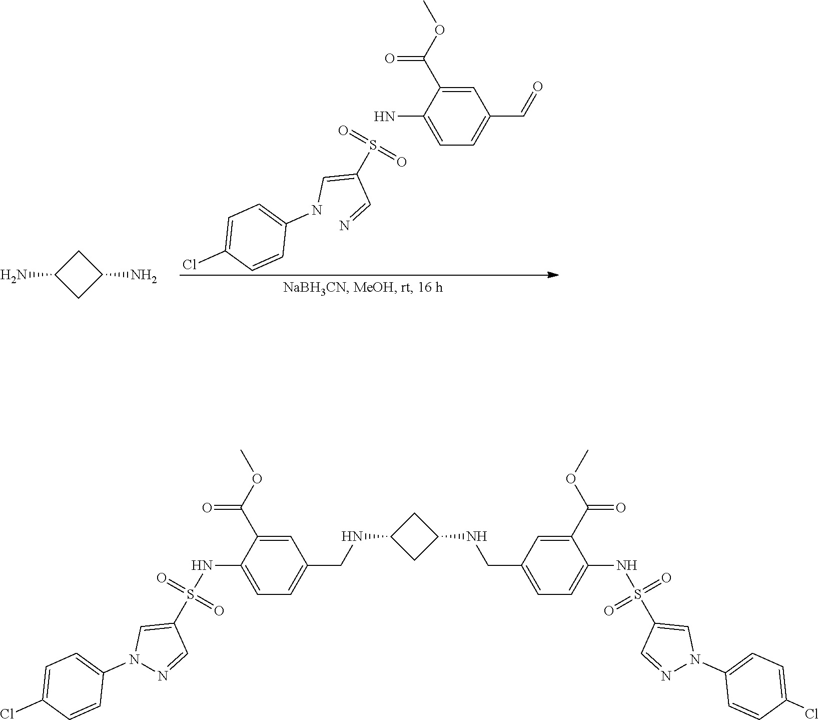

To a solution of (1S,3S)-cyclobutane-1,3-diamine (48a; 20 (30 mg, 0.348 mmol) in MeOH (2 mL) was added methyl 2-((1-(4-chlorophenyl)-1H-pyrazole)-4-sulfonamido)-5-formylbenzoate (Int-11; 292 mg, 0.697 mmol) at room temperature. The reaction mixture was stirred at room temperature for 16 h. Next, NaBH₃CN (66 mg, 1.050 mmol) was added to the mixture. The resulting mixture was stirred at room temperature for an additional 4 h. Tthe solvent was removed in vacuo and the resulting residue was purified by preparative reverse phase chromatography [Boston Green ODS column (150*30 mm, 5 μm), eluent of mobile phase A: water (0.1% TFA) and mobile phase B: MeCN at 220 nm] to provide methyl 2-((1-(4-chlorophenyl)-1H-pyrazole)-4-sulfonamido)-5-((((1s,3s)-3-((4-((1-(4-chlorophenyl)-1H-pyrazole)-4-sulfonamido)-3-(methoxy-carbonyl)benzyl)amino)cyclobutyl) amino)methyl)benzoate. MS (ESI) m/z: 893.1 [M+H⁺]. ¹H NMR (500 MHZ, CD₃OD) δ ppm 8.90 (s, 2H), 8.15 (d, J=2.0 Hz, 2H), 7.96 (s, 2H), 7.84 (d, J=8.5 Hz, 2H), 7.76-7.71 (m, 4H), 7.68 (dd, J=8.5, 2.0 Hz, 2H), 7.50-7.43 (m, 4H), 4.09 (s, 4H), 3.92 (s, 6H), 3.67 (t, J=8.0 Hz, 2H), 2.75-2.60 (m, 2H), 2.56-2.47 (m, 2H).

### Step 3:5-((((1s, 3s)-3-((3-carboxy-4-((1-(4-chlorophenyl)-1H-pyrazole)-4-sulfonamido)benzyl) amino)cyclobutyl) amino)methyl)-2-((1-(4-chlorophenyl)-1H-pyrazole)-4-sulfonamido)benzoic acid (48)

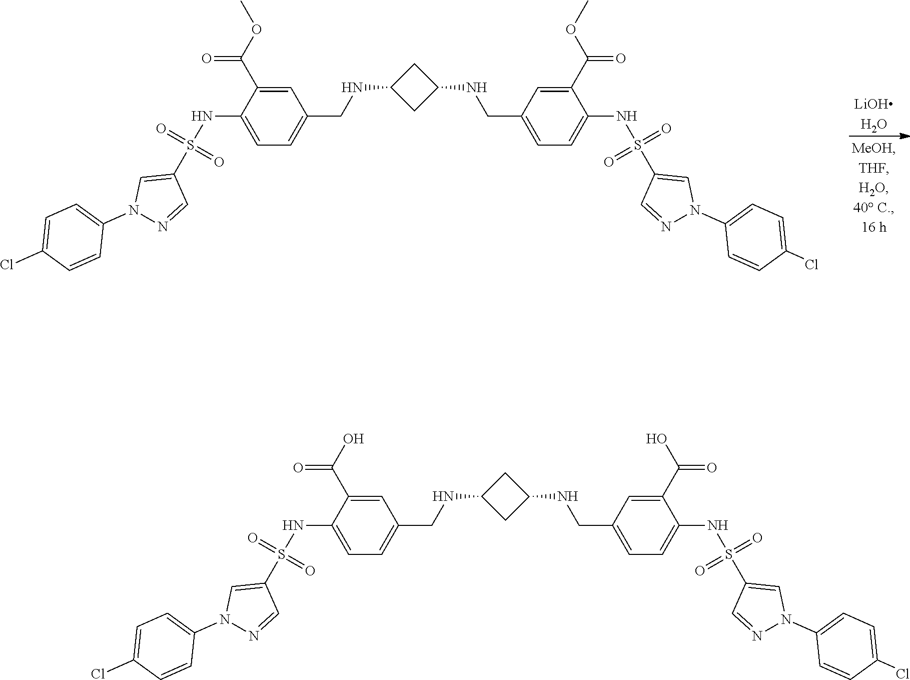 [SMILES: COC(=O)c1cc(CN[C@H]2C[C@@H](NCc3ccc(NS(=O)(=O)c4cnn(-c5ccc(Cl)cc5)c4)c(C(=O)OC)c3)C2)ccc1NS(=O)(=O)c1cnn(-c2ccc(Cl)cc2)c1 ; "LiOH·H2O" ; O=C(O)c1cc(CN[C@H]2C[C@@H](NCc3ccc(NS(=O)(=O)c4cnn(-c5ccc(Cl)cc5)c4)c(C(=O)O)c3)C2)ccc1NS(=O)(=O)c1cnn(-c2ccc(Cl)cc2)c1]

A mixture of methyl 2-((1-(4-chlorophenyl)-1H-pyrazole)-4-sulfonamido)-5-((((1s,3s)-3-((4-((1-(4-chlorophenyl)-1H-pyrazole)-4-sulfonamido)-3-(methoxycarbonyl)benzyl)amino)cyclobutyl) amino)methyl)benzoate (48b; 50 mg, 0.056 mmol) and lithium hydroxide monohydrate (24 mg, 0.572 mmol) in MeOH (4 mL), THF (2 mL), DMF (2 mL), and water (2 mL) was stirred at 40° C. for 16 h. HCl (2 M) was added to pH=4. The reaction mixture was concentrated in vacuo, and the resulting residue was purified by preparative reverse phase chromatography [Phenomenex Synergi C18 column (150*30 mm, 4 μm), eluent of mobile phase A: water (0.05% HCl) and mobile phase B: MeCN at 220 nm] to provide 5-((((1s,3s)-3-((3-carboxy-4-((1-(4-chlorophenyl)-1H-pyrazole)-4-sulfonamido)benzyl)amino)cyclobutyl) amino)-methyl)-2-((1-(4-chlorophenyl)-1H-pyrazole)-4-sulfonamido)benzoic acid. MS (ESI) m/z: 863.1 [M−H⁺]. ¹H NMR (400 MHZ, CD₃OD) δ ppm 8.89 (s, 2H), 8.20 (d, J=2.0 Hz, 2H), 7.95 (s, 2H), 7.89-7.82 (m, 2H), 7.78-7.71 (m, 6H), 7.46 (d, J=8.8 Hz, 4H), 4.11 (s, 4H), 3.69 (br t, J=7.6 Hz, 2H), 2.75-2.64 (m, 4H).

The following compounds (49 through 51) of the present disclosure were made using the methods described in Example 11 above, and substituting the appropriate reactants and/or

| Com- | | Observed |
|---|---|---|
| pound | Structure | MS |
| 49 | 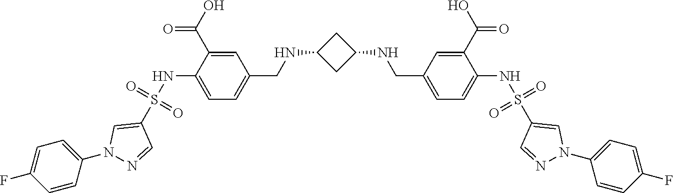 [SMILES: O=C(O)c1cc(CN[C@H]2C[C@@H](NCc3ccc(NS(=O)(=O)c4cnn(-c5ccc(F)cc5)c4)c(C(=O)O)c3)C2)ccc1NS(=O)(=O)c1cnn(-c2ccc(F)cc2)c1] | 833.0 |
| 50 | 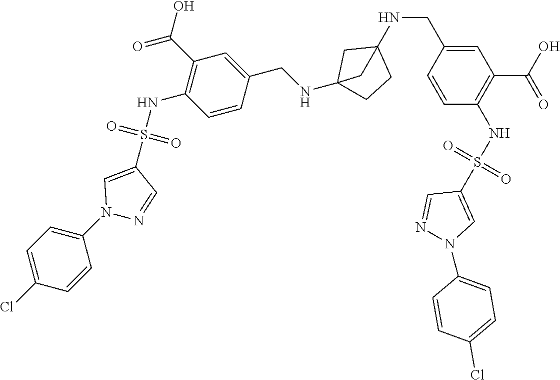 [SMILES: O=C(O)c1cc(CNC23CCC(NCc4ccc(NS(=O)(=O)c5cnn(-c6ccc(Cl)cc6)c5)c(C(=O)O)c4)(C2)C3)ccc1NS(=O)(=O)c1cnn(-c2ccc(Cl)cc2)c1] | 889.2 [M − H⁺] |
| 51 | 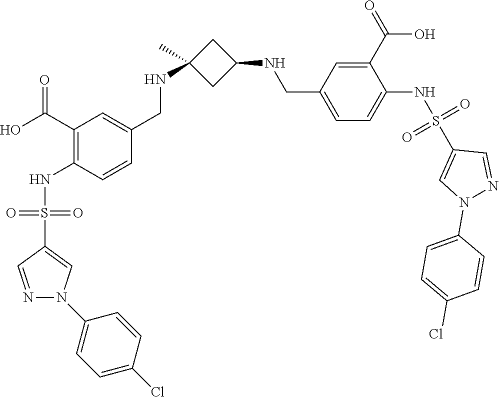 [SMILES: C[C@]1(NCc2ccc(NS(=O)(=O)c3cnn(-c4ccc(Cl)cc4)c3)c(C(=O)O)c2)C[C@@H](NCc2ccc(NS(=O)(=O)c3cnn(-c4ccc(Cl)cc4)c3)c(C(=O)O)c2)C1] | 879.6 |

### EXAMPLE 12

### Preparation of Compound 52

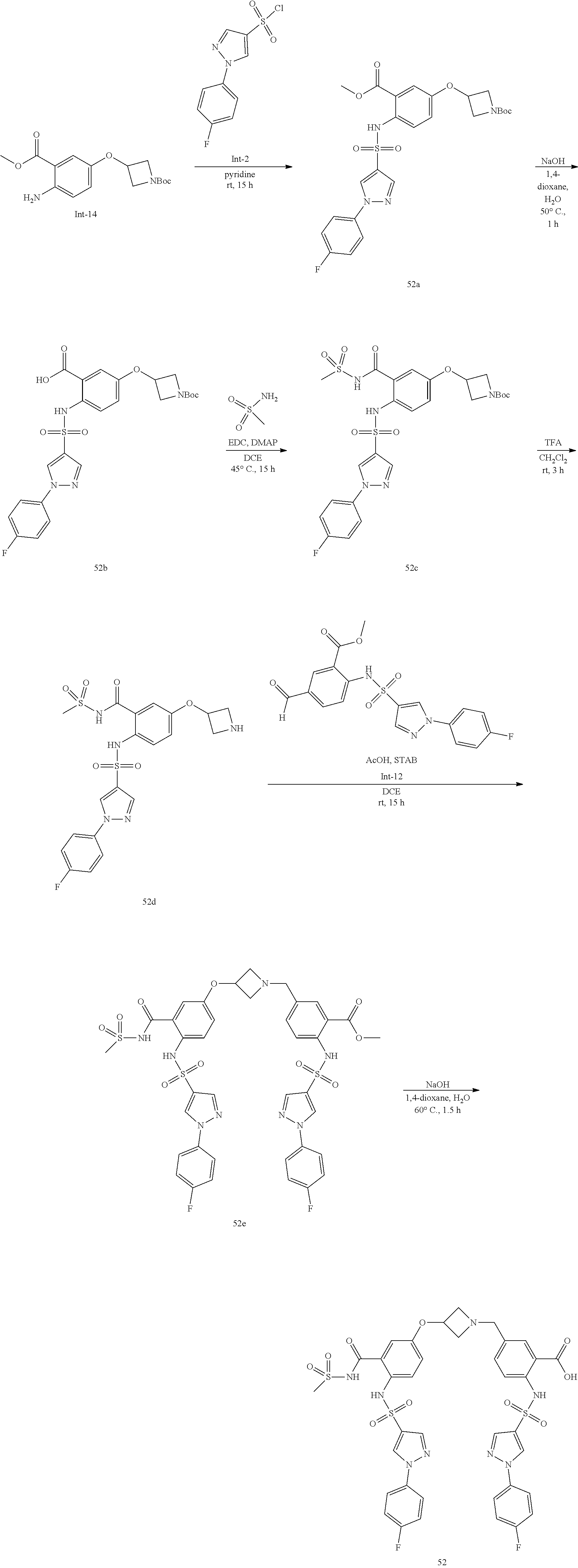

### Step 1: tert-butyl 3-(4-((1-(4-fluorophenyl)-1H-pyrazole)-4-sulfonamido)-3-(methoxy-carbonyl) phenoxy) azetidine-1-carboxylate (52a)

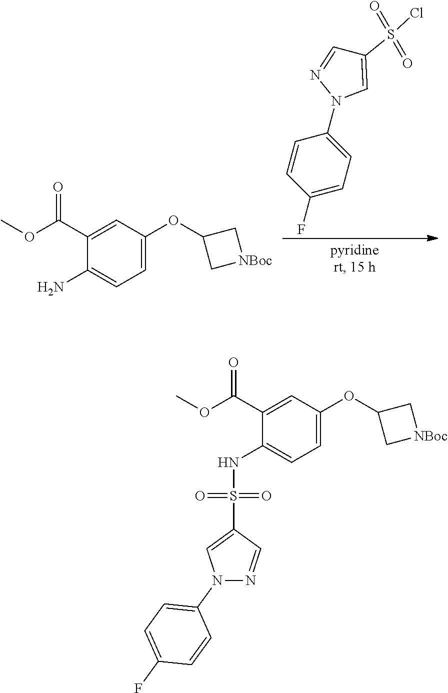 [SMILES: COC(=O)c1cc(OC2CN(C(=O)OC(C)(C)C)C2)ccc1N ; O=S(=O)(Cl)c1cnn(-c2ccc(F)cc2)c1 ; COC(=O)c1cc(OC2CN(C(=O)OC(C)(C)C)C2)ccc1NS(=O)(=O)c1cnn(-c2ccc(F)cc2)c1]

A solution of tert-butyl 3-(4-amino-3-(methoxycarbonyl) phenoxy) azetidine-1-carboxylate (Int-Z; 2 g, 6.20 mmol) and 1-(4-fluorophenyl)-1H-pyrazole-4-sulfonyl chloride (Int-2; 1.698 g, 6.51 mmol) in pyridine (21.39 mL) was stirred at room temperature for 15 h. Additional 1-(4-fluorophenyl)-1H-pyrazole-4-sulfonyl chloride (Int-2; 0.849 g, 3.26 mmol) was added and stirring was continued at room temperature for 1 h. The reaction mixture was concentrated in vacuo, and the resulting residue was purified by preparative reverse phase chromatography [Luna 5 μm C18 column (100*30 mm, 5 μm), eluent of mobile phase A: water (0.1% TFA) and mobile phase B: MeCN at 220 nm] to provide tert-butyl 3-(4-((1-(4-fluorophenyl)-1H-pyrazole)-4-sulfonamido)-3-(methoxycarbonyl) phenoxy) azetidine-1-carboxylate. MS (ESI, m/z): 547.1 [M+H¹].

### Step 2:5-((1-(tert-butoxycarbonyl) azetidin-3-yl)oxy)-2-((1-(4-fluorophenyl)-1H-pyrazole)-4-sulfonamido)benzoic acid (52b)

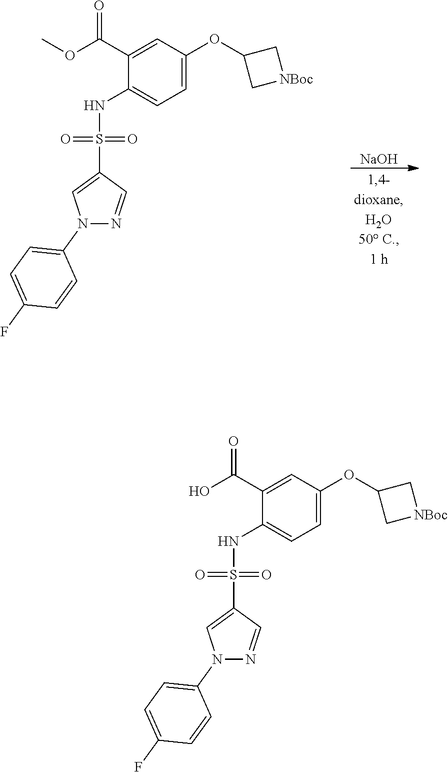 [SMILES: COC(=O)c1cc(OC2CN(C(=O)OC(C)(C)C)C2)ccc1NS(=O)(=O)c1cnn(-c2ccc(F)cc2)c1 ; "NaOH" ; CC(C)(C)OC(=O)N1CC(Oc2ccc(NS(=O)(=O)c3cnn(-c4ccc(F)cc4)c3)c(C(=O)O)c2)C1]

To a solution of tert-butyl 3-(4-((1-(4-fluorophenyl)-1H-pyrazole)-4-sulfonamido)-3-(methoxycarbonyl) phenoxy) azetidine-1-carboxylate (52a; 2 g, 3.66 mmol) in 1,4-dioxane (36.6 mL) was added 2 M aq. NaOH (18.30 mL, 36.6 mmol). The reaction mixture was heated to 50° C. and stirred for 1 h, then concentrated in vacuo. The resulting residue was diluted with H₂O (10 mL) and 1N HCl was added portion-wise to pH ˜2-3 at which point a precipitate formed. The mixture was filtered and the precipitate was washed with water (2×15 mL). The solid was further dried in vacuo to provide 5-((1-(tert-butoxycarbonyl) azetidin-3-yl)oxy)-2-((1-(4-fluorophenyl)-1H-pyrazole)-4-sulfonamido)-benzoic acid, which was used in the subsequent step without further purification. MS (ESI, m/z): 477.2 [M+H⁺-56].

### Step 3: tert-butyl 3-(4-((1-(4-fluorophenyl)-1H-pyrazole)-4-sulfonamido)-3-((methyl-sulfonyl) carbamoyl) phenoxy) azetidine-1-carboxylate (52c)

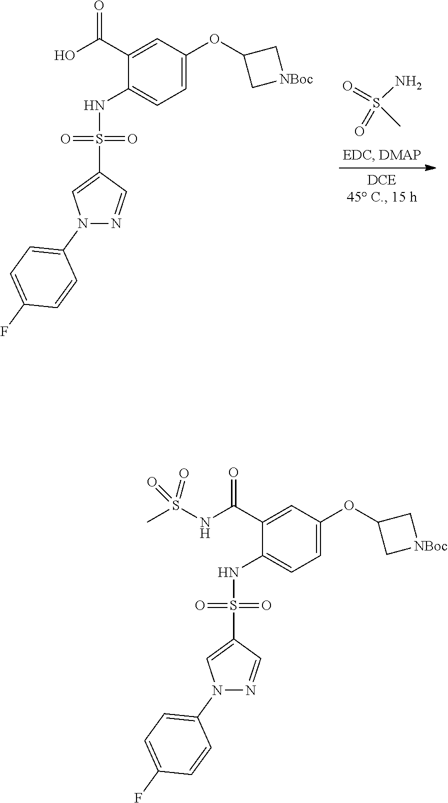

A solution of 5-((1-(tert-butoxycarbonyl) azetidin-3-yl)oxy)-2-((1-(4-fluorophenyl)-1H-pyrazole)-4-sulfonamido)benzoic acid (52b; 100 mg, 0.188 mmol), methanesulfonamide (17.86 mg, 0.188 mmol), EDC (43.2 mg, 0.225 mmol) and DMAP (27.5 mg, 0.225 mmol) in DCE (1878 μl) were stirred at 45° C. for 15 h. The reaction mixture was diluted with H₂O (10 mL) and extracted with CH₂Cl₂ (3×15 mL). The combined organics were dried over anh. Mg2SO₄, filtered, and concentrated in vacuo. The resulting residue was purified by preparative reverse phase chromatography [Luna 5u C18 column (100*30 mm, 5 μm), eluent of mobile phase A: water (0.1% TFA) and mobile phase B: MeCN at 220 nm] to provide tert-butyl 3-(4-((1-(4-fluorophenyl)-1H-pyrazole)-4-sulfonamido)-3-((methylsulfonyl) carbamoyl)-phenoxy) azetidine-1-carboxylate. MS (ESI, m/z): 554.1 [M+H⁺-56].

### Step 4:5-(azetidin-3-yloxy)-2-((1-(4-fluorophenyl)-1H-pyrazole)-4-sulfonamido)-N-(methylsulfonyl)benzamide (52d)

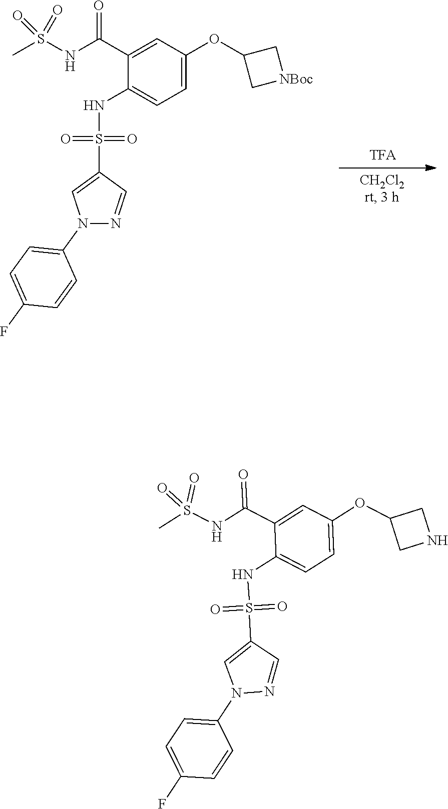 [SMILES: CC(C)(C)OC(=O)N1CC(Oc2ccc(NS(=O)(=O)c3cnn(-c4ccc(F)cc4)c3)c(C(=O)NS(C)(=O)=O)c2)C1 ; CS(=O)(=O)NC(=O)c1cc(OC2CNC2)ccc1NS(=O)(=O)c1cnn(-c2ccc(F)cc2)c1]

To a solution of tert-butyl 3-(4-((1-(4-fluorophenyl)-1H-pyrazole)-4-sulfonamido)-3-((methylsulfonyl) carbamoyl) phenoxy) azetidine-1-carboxylate (52c; 27.1 mg, 0.044 mmol) in CH₂C₁₂ (445 μL) was added TFA (51.4 μl, 0.667 mmol). The reaction mixture was stirred at room temperature for 3 h, then concentrated in vacuo to provide crude 5-(azetidin-3-yloxy)-2-((1-(4-fluorophenyl)-1H-pyrazole)-4-sulfonamido)-N-(methylsulfonyl)benzamide, which was used in the subsequent step without further purification. MS (ESI, m/z): 510.1 [M+H⁺].

### Step 5: methyl 2-((1-(4-fluorophenyl)-1H-pyrazole)-4-sulfonamido)-5-((3-(4-((1-(4-fluorophenyl)-1H-pyrazole)-4-sulfonamido)-3-((methylsulfonyl) carbamoyl) phenoxy)-azetidin-1-yl)methyl)benzoate (52e)

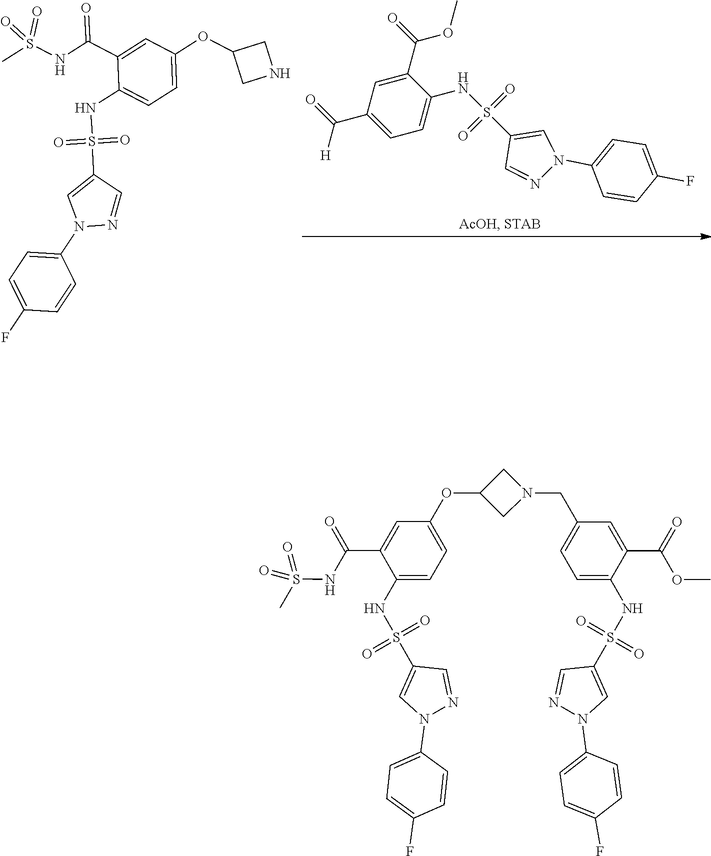

To a solution of 5-(azetidin-3-yloxy)-2-((1-(4-fluorophenyl)-1H-pyrazole)-4-sulfonamido)-N-(methylsulfonyl)benzamide (52d; 27 mg, 0.044 mmol), and methyl 2-((1-(4-fluorophenyl)-1H-pyrazole)-4-sulfonamido)-5-formylbenzoate (Int-12; 19.71 mg, 0.049 mmol) in DCE (444 μl) was added AcOH (10.17 μl, 0.178 mmol). The reaction mixture was stirred at room temperature for 1 h. Sodium triacetoxy borohydride (28.2 mg, 0.133 mmol) was added and the reaction mixture was stirred at room temperature for 15 h. The reaction mixture was diluted with H₂O (10 mL) and aq. sodium bicarbonate was added slowly and dropwise until the solution was basic. The reaction mixture was extracted with CH₂Cl₂ (3×15 mL) and the organics were dried over anh. Mg₂SO₄, filtered, and concentrated in vacuo. The resulting residue was purified by preparative reverse phase chromatography [Luna 5u C18 column (100*30 mm, 5 μm), eluent of mobile phase A: water (0.1% TFA) and mobile phase B: MeCN at 220 nm] to provide methyl 2-((1-(4-fluorophenyl)-1H-pyrazole)-4-sulfonamido)-5-((3-(4-((1-(4-fluorophenyl)-1H-pyrazole)-4-sulfonamido)-3-((methylsulfonyl) carbamoyl) phenoxy) azetidin-1-yl)methyl)benzoate. MS (ESI, m/z): 897.1 [M+H⁺].

### Step 6:2-((1-(4-fluorophenyl)-1H-pyrazole)-4-sulfonamido)-5-((3-(4-((1-(4-fluorophenyl)-1H-pyrazole)-4-sulfonamido)-3-((methylsulfonyl) carbamoyl) phenoxy) azetidin-1-yl)methyl)benzoic acid (52)

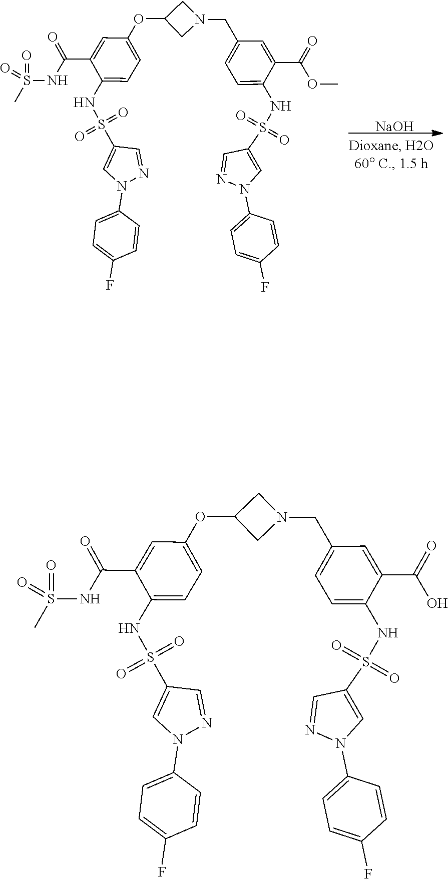 [SMILES: COC(=O)c1cc(CN2CC(Oc3ccc(NS(=O)(=O)c4cnn(-c5ccc(F)cc5)c4)c(C(=O)NS(C)(=O)=O)c3)C2)ccc1NS(=O)(=O)c1cnn(-c2ccc(F)cc2)c1 ; "NaOH" ; CS(=O)(=O)NC(=O)c1cc(OC2CN(Cc3ccc(NS(=O)(=O)c4cnn(-c5ccc(F)cc5)c4)c(C(=O)O)c3)C2)ccc1NS(=O)(=O)c1cnn(-c2ccc(F)cc2)c1]

To a solution of methyl 2-((1-(4-fluorophenyl)-1H-pyrazole)-4-sulfonamido)-5-((3-(4-((1-(4-fluorophenyl)-1H-pyrazole)-4-sulfonamido)-3-((methylsulfonyl) carbamoyl) phenoxy)-azetidin-1-yl)methyl)benzoate (52e; 12.5 mg, 0.014 mmol) in 1,4-dioxane (119 μl) was added 1 M aq. NaOH (139 μl, 0.139 mmol). The reaction mixture was heated to 60° C. for 1.5 h. After cooling to room temperature, TFA (10.74 μl, 0.139 mmol) was added and the reaction was concentrated in vacuo. The resulting residue was purified by preparative reverse phase chromatography [C₁₈ column, eluent of mobile phase A: water (0.1% TFA) and mobile phase B: MeCN at 220 nm] to provide 2-((1-(4-fluorophenyl)-1H-pyrazole)-4-sulfonamido)-5-((3-(4-((1-(4-fluorophenyl)-1H-pyrazole)-4-sulfonamido)-3-((methylsulfonyl) carbamoyl) phenoxy) azetidin-1-yl)methyl)benzoic acid. MS (ESI, m/z): 883.0 [M+H⁺]. ¹H NMR (500 MHZ, CD₃OD) δ (ppm) 8.84 (s, 1H), 8.56 (s, 1H), 8.18 (s, 1H), 7.94 (s, 1H), 7.84 (d, J=8.6 Hz, 1H), 7.74 (td, J=13.2, 11.2, 6.6 Hz, 5H), 7.67 (d, J=8.6 Hz, 1H), 7.41 (d, J=8.9 Hz, 1H), 7.22 (t, J=8.5 Hz, 4H), 7.12 (d, J=2.2 Hz, 1H), 7.06 (dd, J=8.9, 2.6 Hz, 1H), 5.11 (s, 1H), 4.59 (dd, J=11.8, 6.4 Hz, 2H), 4.43 (s, 2H), 4.22 (d, J=9.4 Hz, 2H), 3.28 (s, 3H).

The following compound (53) of the present disclosure were made using the methods described in Example 12 above, and substituting the appropriate reactants and/or reagents:

| | | Observed |
|---|---|---|
| Compound | Structure | MS |
| 53 | 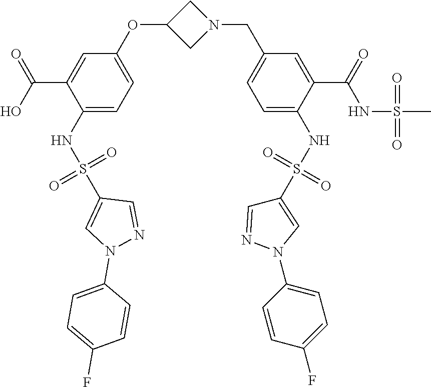 [SMILES: CS(=O)(=O)NC(=O)c1cc(CN2CC(Oc3ccc(NS(=O)(=O)c4cnn(-c5ccc(F)cc5)c4)c(C(=O)O)c3)C2)ccc1NS(=O)(=O)c1cnn(-c2ccc(F)cc2)c1] | 833.0 |

### EXAMPLE 13

### Preparation of Compound 54

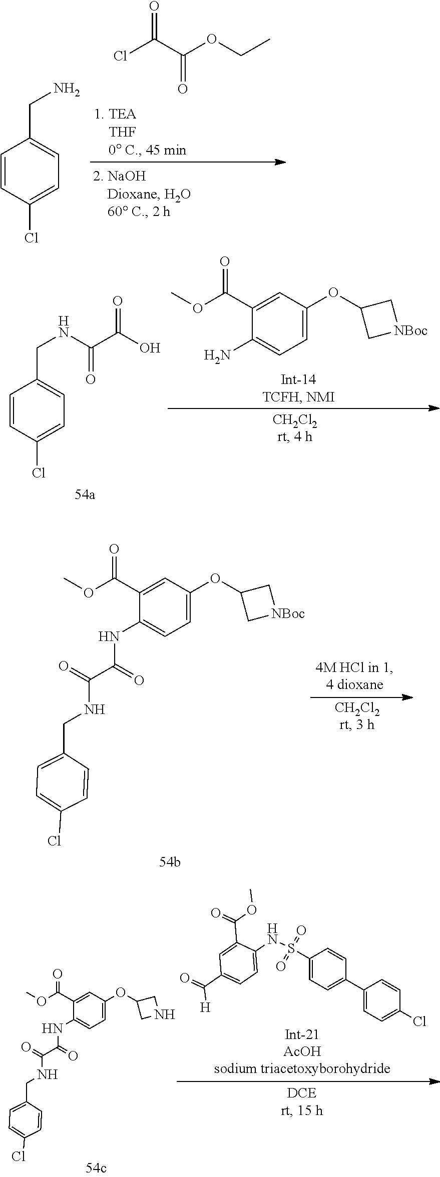

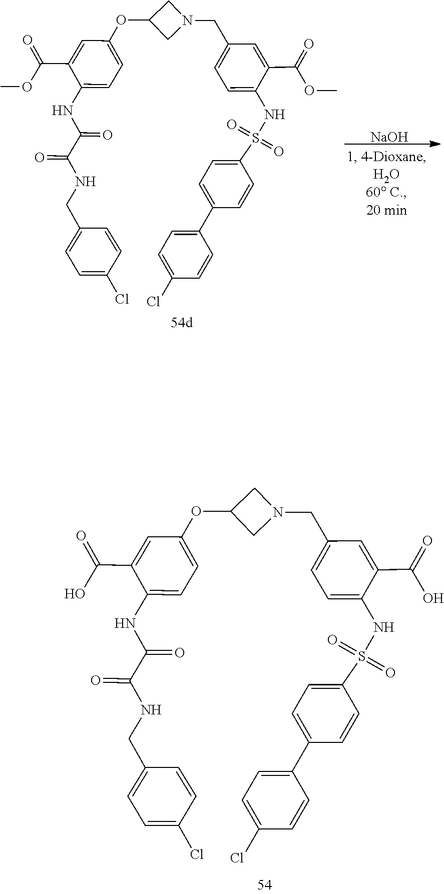

### Step 1:2-((4-chlorobenzyl)amino)-2-oxoacetic acid (54a)

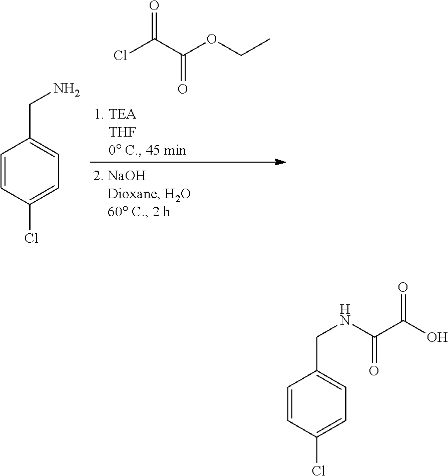

A solution of (4-chlorophenyl) methanamine (0.430 mL, 3.53 mmol) and TEA (1.477 mL, 10.59 mmol) was stirred in THF (35.3 mL) and cooled to 0° C. Ethyl oxalyl chloride (1.130 ml, 10.59 mmol) was added dropwise and a precipitate formed. The reaction was stirred at 0° C. for 45 min, then filtered and concentrated in vacuo. The resulting residue was re-dissolved in 1,4-dioxane (30 mL) and 1M aqueous NaOH (8.83 mL, 17.66 mmol) was added. The reaction mixture was heated at 60° C. for 2 h. The reaction mixture was concentrated in vacuo and IN HCl (20 mL) was added. The mixture was stirred for 15 min, then the resulting precipitate was filtered and dried in vacuo to provide crude 2-((4-chlorobenzyl)-amino)-2-oxoacetic acid, which was used in the subsequent step without further purification. MS (ESI, m/z): 214.0 [M+H⁺].

### Step 2: tert-butyl 3-(4-(2-((4-chlorobenzyl)amino)-2-oxoacetamido)-3-(methoxycarbonyl)-phenoxy) azetidine-1-carboxylate (54b)

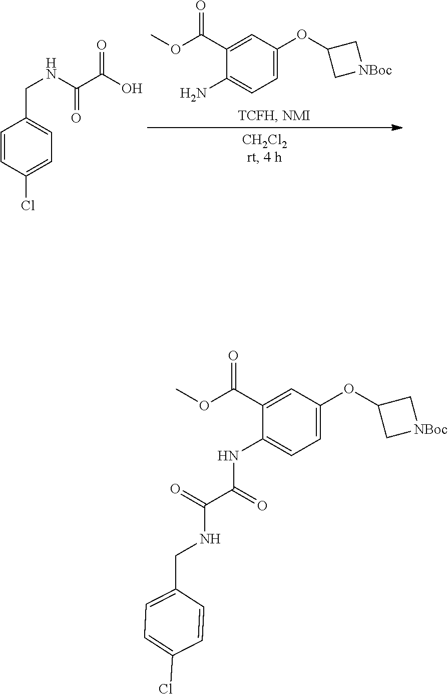 [SMILES: O=C(O)C(=O)NCc1ccc(Cl)cc1 ; COC(=O)c1cc(OC2CN(C(=O)OC(C)(C)C)C2)ccc1N ; COC(=O)c1cc(OC2CN(C(=O)OC(C)(C)C)C2)ccc1NC(=O)C(=O)NCc1ccc(Cl)cc1]

A solution of tert-butyl 3-(4-amino-3-(methoxycarbonyl) phenoxy) azetidine-1-carboxylate (Int-14; 125 mg, 0.388 mmol), 2-((4-chlorobenzyl)amino)-2-oxoacetic acid (52a; 166 mg, 0.776 mmol), TCFH (381 mg, 1.36 mmol) and 1-methylimidazole (37.1 μL, 0.465 mmol) in CH₂C₁₂ (3.88 mL μl) was stirred at room temperature for 4 h. The reaction mixture was diluted with H₂O (10 mL) and extracted with CH₂Cl₂ (3×15 mL). The orgnaics were dried over anh. Mg₂SO₄, filtered, and concentrated in vacuo. The resulting residue was purified by flash chromatography on SiO₂ [Isco® 12 g Gold Redisep column, eluent of 0 to 100% EtOAc/EtOH (3:1) in hexanes] to provide tert-butyl 3-(4-(2-((4-chlorobenzyl)amino)-2-oxoacetamido)-3-(methoxycarbonyl) phenoxy) azetidine-1-carboxylate. MS (ESI, m/z): 517.2 [M+H⁺].

### Step 3: methyl 5-(azetidin-3-yloxy)-2-(2-((4-chlorobenzyl)amino)-2-oxoacetamido)-benzoate (54c)

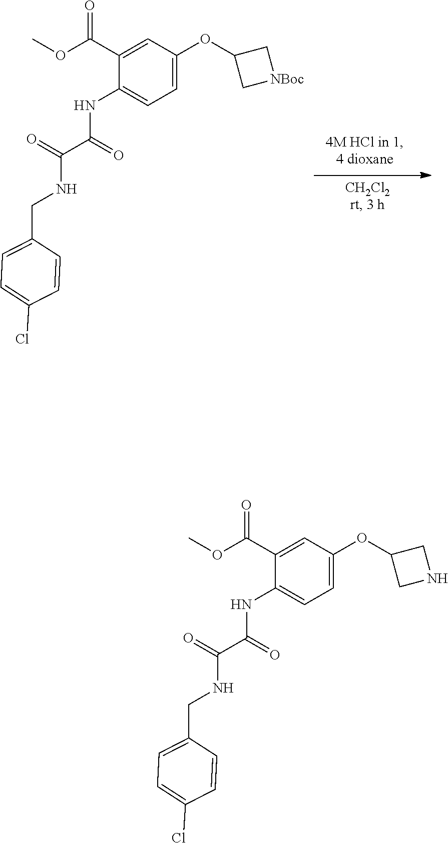 [SMILES: COC(=O)c1cc(OC2CN(C(=O)OC(C)(C)C)C2)ccc1NC(=O)C(=O)NCc1ccc(Cl)cc1 ; COC(=O)c1cc(OC2CNC2)ccc1NC(=O)C(=O)NCc1ccc(Cl)cc1]

To a solution of tert-butyl 3-(4-(2-((4-chlorobenzyl)amino)-2-oxoacetamido)-3-(methoxy-carbonyl) phenoxy) azetidine-1-carboxylate (54b; 120 mg, 0.232 mmol) in CH₂Cl₂ (927 μL) was added 4M HCl in 1,4-dioxane (463 μl, 1.853 mmol). The reaction mixture was stirred at room temperature for 3 h, then concentrated in vacuo to provide crude methyl 5-(azetidin-3-yloxy)-2-(2-((4-chlorobenzyl)amino)-2-oxoacetamido)benzoate, which was used in the subsequent step without further purification. MS (ESI, m/z): 418.2 [M+H⁺].

### Step 4: methyl 2-((4′-chloro-[1, l′-biphenyl])-4-sulfonamido)-5-((3-(4-(2-((4-chlorobenzyl)amino)-2-oxoacetamido)-3-(methoxycarbonyl) phenoxy) azetidin-1-yl)methyl)benzoate (54d)

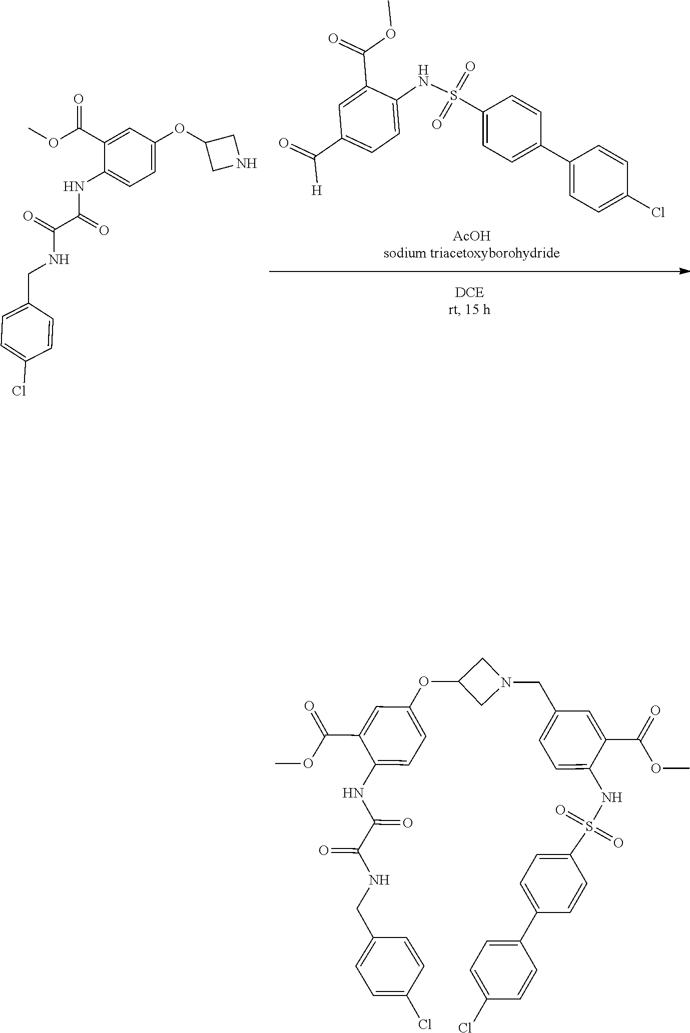

To a solution of methyl 2-((4′-chloro-[1,1′-biphenyl])-4-sulfonamido)-5-formylbenzoate (Int-21; 16.65 mg, 0.039 mmol) and methyl 5-(azetidin-3-yloxy)-2-(2-((4-chlorobenzyl)-amino)-2-oxoacetamido)benzoate (54c; 16 mg, 0.035 mmol) in DCE (352 μl) was added AcOH (8.06 μl, 0.141 mmol). The reacton mixture was stirred at room temperature for 1 h. Sodium triacetoxy borohydride (22.39 mg, 0.106 mmol) was added and the resulting solution was stirred at room temperature for 15 h. Then the reaction mixture was diluted with H₂O (10 mL) and aq. sodium bicarbonate was added slowly and dropwise until the solution was basic. The aqueous layer was extracted with CH₂Cl₂ (3×15 mL) and the combined organics were dried over anh. Mg₂SO₄, filtered, and concentrated in vacuo to provide crude methyl 2-((4′-chloro-[1,1′-biphenyl])-4-sulfonamido)-5-((3-(4-(2-((4-chlorobenzyl)amino)-2-oxoacetamido)-3-(methoxycarbonyl) phenoxy) azetidin-1-yl)methyl)benzoate, which was used in the subsequent step without further purification. MS (ESI, m/z): 831.4 [M+H⁺].

### Step 5:5-((1-(3-carboxy-4-((4′-chloro-[1,1′-biphenyl])-4-sulfonamido)benzyl) azetidin-3-yl)oxy)-2-(2-((4-chlorobenzyl)amino)-2-oxoacetamido)benzoic acid (54)

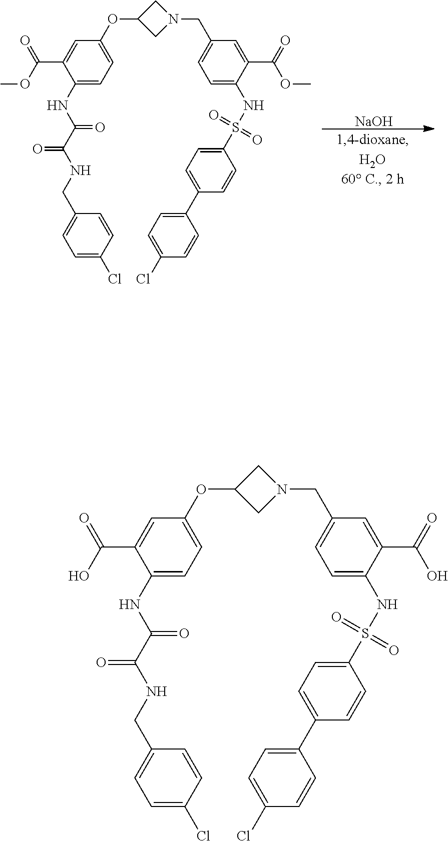

To a solution of methyl 2-((4′-chloro-[1,1′-biphenyl])-4-sulfonamido)-5-((3-(4-(2-((4-chlorobenzyl)amino)-2-oxoacetamido)-3-(methoxycarbonyl) phenoxy) azetidin-1-yl)methyl)benzoate (54d; 29.3 mg, 0.035 mmol) in 1,4-dioxane (301 μL) was added 1M aq. NaOH (352 μl, 0.352 mmol). The reaction was heated to 60° C. for 20 min. After cooling to room temperature, TFA (27.1 μl, 0.352 mmol) was added and the reaction mixture was concentrated in vacuo. The resulting reside was purified by preparative reverse phase chromatography [C₁₈ column, eluent of mobile phase A: water (0.1% TFA) and mobile phase B: MeCN at 220 nm] to provide 5-((1-(3-carboxy-4-((4′-chloro-[1,1′-biphenyl])-4-sulfonamido)benzyl) azetidin-3-y1)oxy)-2-(2-((4-chlorobenzyl)amino)-2-oxoacetamido)benzoic acid. MS (ESI, m/z): 803.1 [M+H⁺]. ¹H NMR (500 MHZ, DMSO) δ (ppm) 9.67 (t, J=6.3 Hz, 1H), 8.59 (d, J=9.2 Hz, 1H), 8.07 (s, 1H), 7.92 (d, J=7.9 Hz, 1H), 7.85 (d, J=7.6 Hz, 2H), 7.73 (d, J=8.5 Hz, 2H), 7.54 (d, J=8.2 Hz, 2H), 7.44-7.36 (m, 4H), 7.32 (d, J=8.3 Hz, 4H), 7.22 (d, J=6.1 Hz, 1H), 4.98-5.05 (m, 1H), 4.43-4.53 (m, 2H), 4.36 (d, J=6.3 Hz, 2H), 4.16-4.28 (m, 2H), 4.05-4.14 (m, 2H).

The following compounds (56, and 58 through 63) of the present disclosure were made using the methods described in Example 13 above, and substituting the appropriate reactants and/or

| | | Observed |
|---|---|---|
| Compound | Structure | MS |
| 56 | 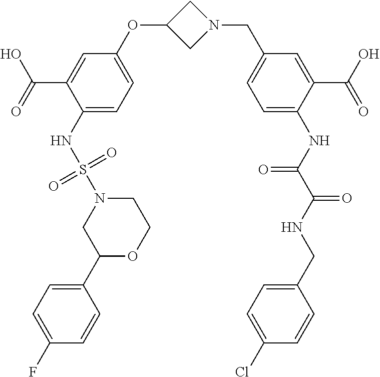 [SMILES: O=C(NCc1ccc(Cl)cc1)C(=O)Nc1ccc(CN2CC(Oc3ccc(NS(=O)(=O)N4CCOC(c5ccc(F)cc5)C4)c(C(=O)O)c3)C2)cc1C(=O)O] | 796.2 |
| 58 | 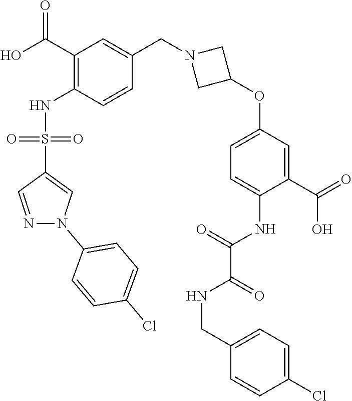 [SMILES: O=C(NCc1ccc(Cl)cc1)C(=O)Nc1ccc(OC2CN(Cc3ccc(NS(=O)(=O)c4cnn(-c5ccc(Cl)cc5)c4)c(C(=O)O)c3)C2)cc1C(=O)O] | 793.3 |
| 59 | 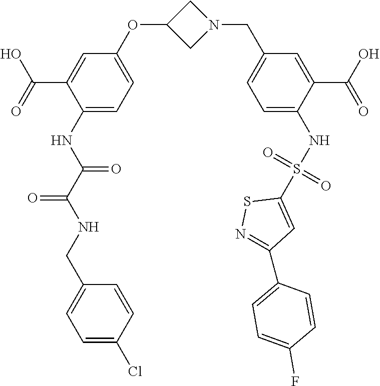 [SMILES: O=C(NCc1ccc(Cl)cc1)C(=O)Nc1ccc(OC2CN(Cc3ccc(NS(=O)(=O)c4cc(-c5ccc(F)cc5)ns4)c(C(=O)O)c3)C2)cc1C(=O)O] | 793.9 |
| 60 | 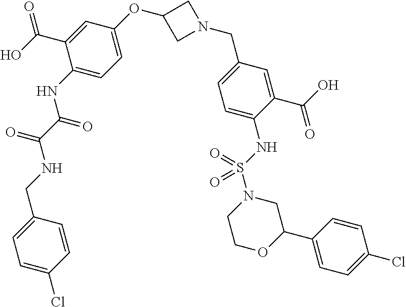 [SMILES: O=C(NCc1ccc(Cl)cc1)C(=O)Nc1ccc(OC2CN(Cc3ccc(NS(=O)(=O)N4CCOC(c5ccc(Cl)cc5)C4)c(C(=O)O)c3)C2)cc1C(=O)O] | 812.0 |
| 61 | 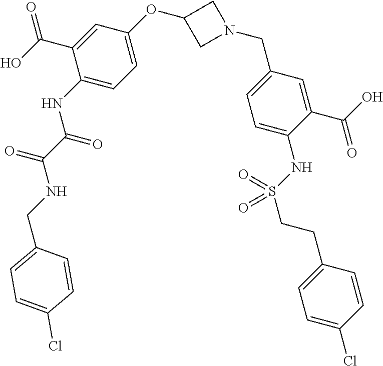 [SMILES: O=C(NCc1ccc(Cl)cc1)C(=O)Nc1ccc(OC2CN(Cc3ccc(NS(=O)(=O)CCc4ccc(Cl)cc4)c(C(=O)O)c3)C2)cc1C(=O)O] | 755.1 |
| 62 | 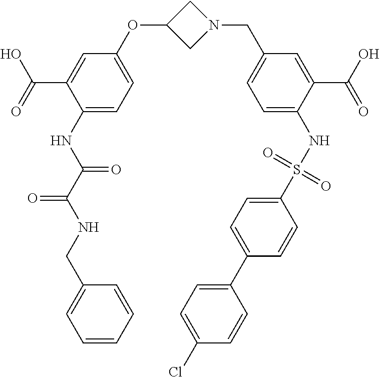 [SMILES: O=C(NCc1ccccc1)C(=O)Nc1ccc(OC2CN(Cc3ccc(NS(=O)(=O)c4ccc(-c5ccc(Cl)cc5)cc4)c(C(=O)O)c3)C2)cc1C(=O)O] | 769.2 |
| 63 | 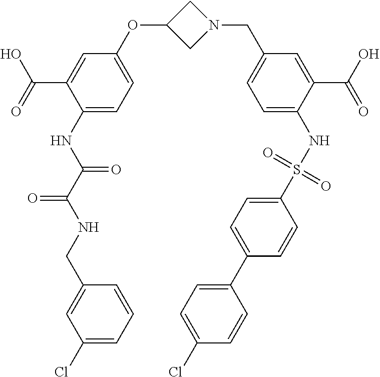 [SMILES: O=C(NCc1cccc(Cl)c1)C(=O)Nc1ccc(OC2CN(Cc3ccc(NS(=O)(=O)c4ccc(-c5ccc(Cl)cc5)cc4)c(C(=O)O)c3)C2)cc1C(=O)O] | 803.1 |

### EXAMPLE 14

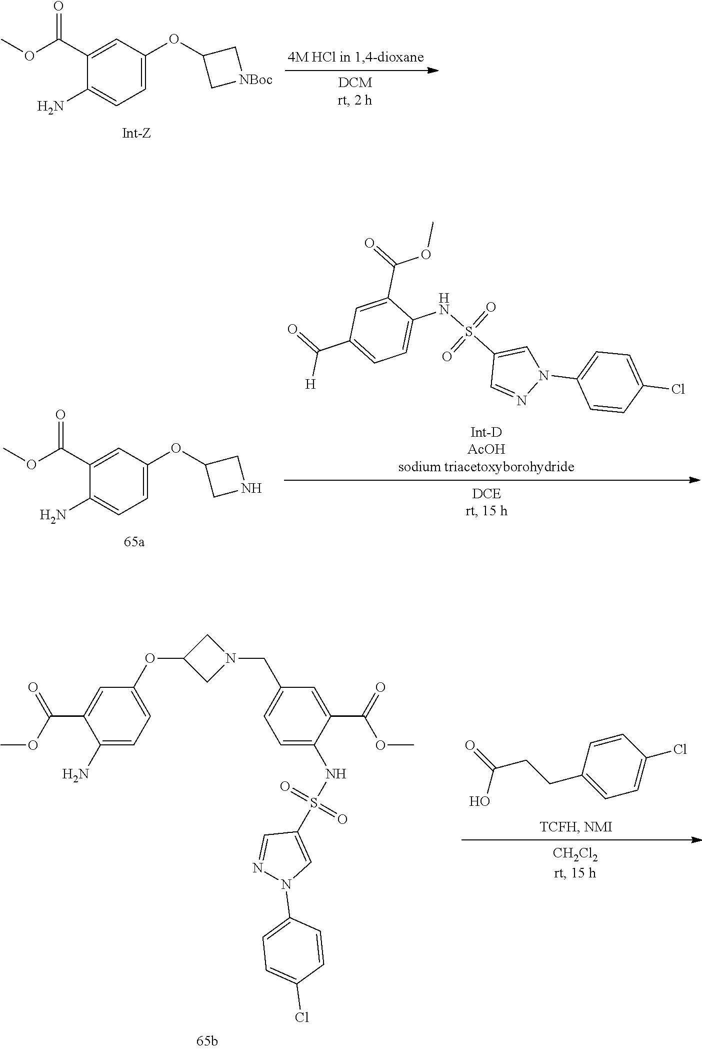

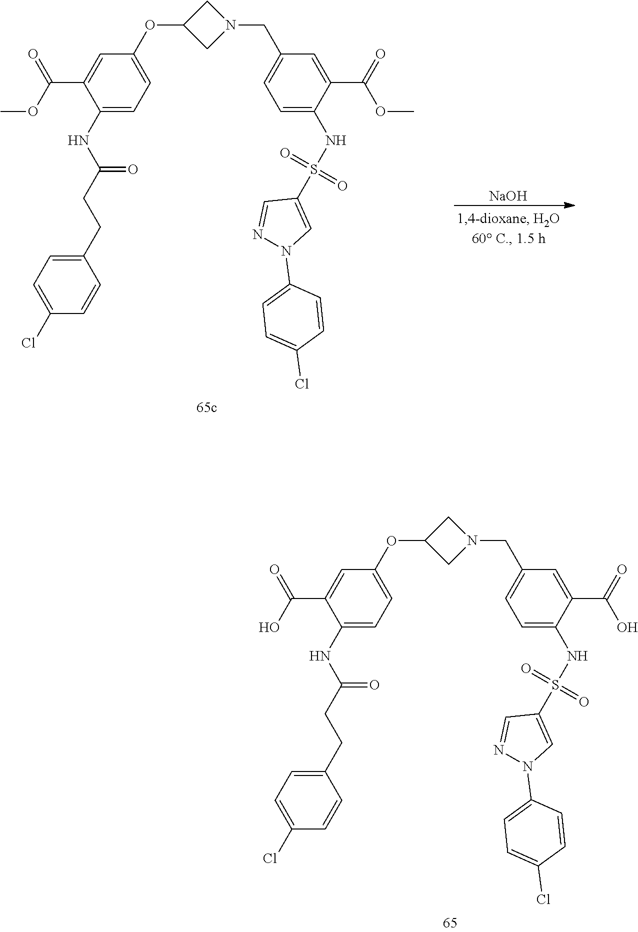

### Step 1: methyl 2-amino-5-(azetidin-3-yloxy)benzoate (65a)

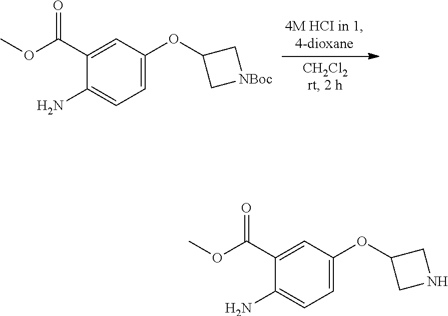 [SMILES: COC(=O)c1cc(OC2CN(C(=O)OC(C)(C)C)C2)ccc1N ; COC(=O)c1cc(OC2CNC2)ccc1N]

To a solution of tert-butyl 3-(4-amino-3-(methoxycarbonyl) phenoxy) azetidine-1-carboxylate (3 g, 9.31 mmol) in 1,4-dioxane (30 mL) was added HCl (4 M in 1,4-dioxane, 23.27 mL, 93 mmol). The reaction was stirred at room temperature for 2 h then concentrated in vacuo to provide crude methyl 2-amino-5-(azetidin-3-yloxy)benzoate, which was used in the subsequent step without further purification. MS (ESI, m/z): 233.2 [M+H⁺]. 10 Step 2: methyl 2-amino-5-((1-(4-((1-(4-chlorophenyl)-1H-pyrazole)-4-sulfonamido)-3-(methoxycarbonyl)benzyl) azetidin-3-yl)oxy)benzoate (65b)

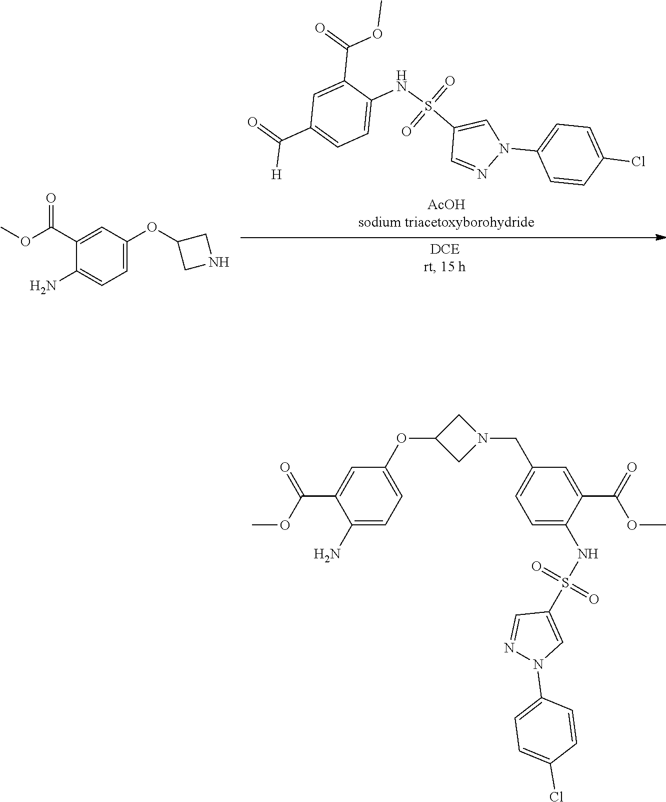

To a solution of methyl 2-amino-5-(azetidin-3-yloxy)benzoate (65a; 2.07 g, 9.31 mmol) in CH₂Cl₂ (46.5 mL) and MeOH (46.5 mL) were added methyl 2-((1-(4-chlorophenyl)-1H-pyrazole)-4-sulfonamido)-5-formylbenzoate (Int-11; 3.91 g, 9.31 mmol), TEA (3.89 ml, 27.9 mmol), and a solution of zinc (II) chloride (1.9 M in 2MeTHF, 9.80 mL, 18.61 mmol). The reaction was stirred at room temperature for 1 h. odium cyanoborohydride (1.170 g, 18.61 mmol) was added portion-wise and the reaction mixture was stirred at room temperature for 15 h. The reaction mixture was concentrated in vacuo, and the resulting residue was diluted with H₂O (100 mL) and extracted with CH₂Cl₂ (3×50 mL). The combined organics were washed with brine (100 mL) dried over anh. Na₂SO₄, filtered, and concentrated in vacuo. The resulting residue was purified by preparative reverse phase chromatography [C18 column, eluent of mobile phase A: water (0.1% TFA) and mobile phase B: MeCN at 220 nm] to provide methyl 2-amino-5-((1-(4-((1-(4-chlorophenyl)-1H-pyrazole)-4-sulfonamido)-3-(methoxycarbonyl)benzyl) azetidin-3-yl)oxy)benzoate. MS (ESI, m/z): 626.4 [M+H⁺].

### Step 3: methyl 2-((1-(4-chlorophenyl)-1H-pyrazole)-4-sulfonamido)-5-((3-(4-(3-(4-chlorophenyl) propanamido)-3-(methoxycarbonyl) phenoxy) azetidin-1-yl)methyl)benzoate (65c)

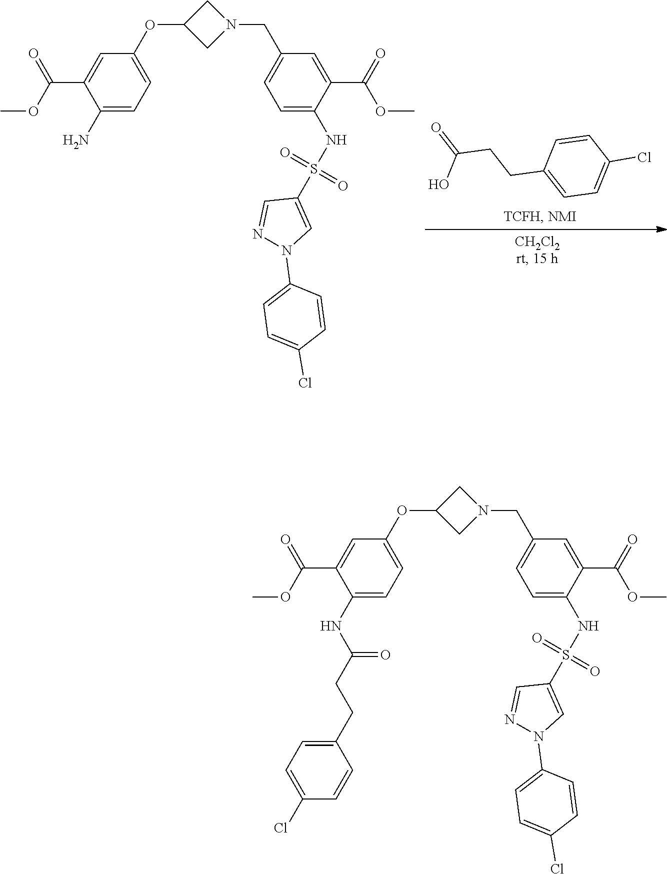

A solution of methyl 2-amino-5-((1-(4-((1-(4-chlorophenyl)-1H-pyrazole)-4-sulfonamido)-3-(methoxycarbonyl)benzyl) azetidin-3-yl)oxy)benzoate (65b; 30 mg, 0.048 mmol), 3-(4-chlorophenyl) propanoic acid (22.12 mg, 0.120 mmol), TCFH (16.13 mg, 0.058 mmol) and 1-methylimidazole (13.37 μL, 0.168 mmol) was stirred in CH₂Cl₂ (479 μL) at room temperature for 15 h. The reaction mixture was concentrated in vacuo to provide crude methyl 2-((1-(4-chlorophenyl)-1H-pyrazole)-4-sulfonamido)-5-((3-(4-(3-(4-chlorophenyl)-propanamido)-3-(methoxycarbonyl) phenoxy) azetidin-1-yl)methyl)benzoate, which was used in the subsequent step without further purification. MS (ESI, m/z): 792.1 [M+H⁺].

### Step 4:5-((1-(3-carboxy-4-((1-(4-chlorophenyl)-1H-pyrazole)-4-sulfonamido)-benzyl) azetidin-3-yl)oxy)-2-(3-(4-chlorophenyl) propanamido)benzoic acid (65)

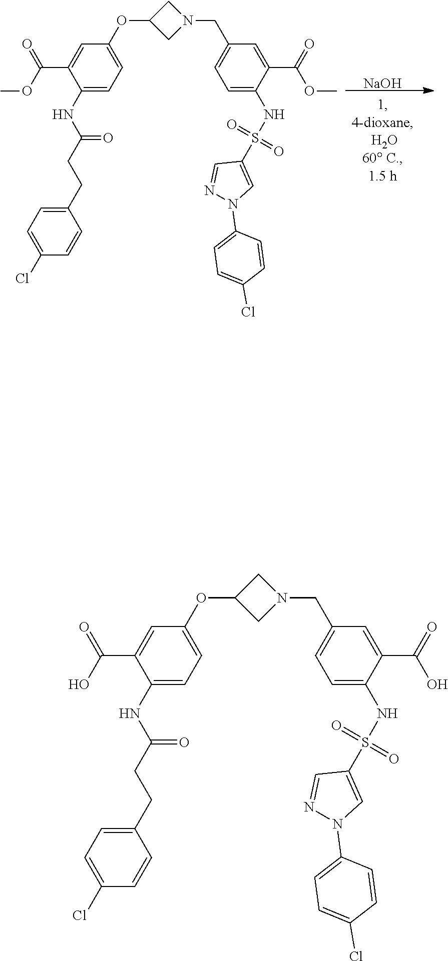

To a solution of methyl 2-((1-(4-chlorophenyl)-1H-pyrazole)-4-sulfonamido)-5-((3-(4-(3-(4-chlorophenyl) propanamido)-3-(methoxycarbonyl) phenoxy) azetidin-1-yl)methyl)-benzoate (65c; 38 mg, 0.048 mmol) in 1,4-dioxane (410 μL) was added 1 M aq. NaOH (479 μL, 0.479 mmol). The reaction was heated to 60° C. for 30 min, then cooled followed by addition of TFA (36.9 μL, 0.479 mmol). The reaction was concentrated in vacuo. The resulting residue was purified by preparative reverse phase chromatography [C18 column, eluent of mobile phase A: water (0.1% TFA) and mobile phase B: MeCN at 220 nm] to provide 5-((1-(3-carboxy-4-((1-(4-chlorophenyl)-1H-pyrazole)-4-sulfonamido)-benzyl) azetidin-3-yl)oxy)-2-(3-(4-chlorophenyl)-propanamido)benzoic acid. MS (ESI, m/z): 764.0 [M+H⁺]. ¹H NMR (500 MHZ, DMSO) & (ppm) 10.76 (s, 1H), 9.21 (s, 1H), 8.29 (d, J=9.0 Hz, 1H), 8.03-8.13 (m, 2H), 7.91 (d, J=8.9 Hz, 2H), 7.57 (d, J=8.7 Hz, 3H), 7.35-7.25 (m, 3H), 7.12 (d, J=9.3 Hz, 1H), 4.92-5.05 (m, 1H), 4.26-4.56 (m, 4H), 3.98-4.14 (m, 2H), 2.89-2.92 (t, J=7.6 Hz, 2H), 2.66-2.69 (dd, J=16.3, 8.7 Hz, 2H).

The following compounds (66, 67, 69, 71 through 75) of the present disclosure were made using the methods described in Example 14 above, and substituting the appropriate reactants and/or

| | | Observed |
|---|---|---|
| Compound | Structure | MS |
| 66 | 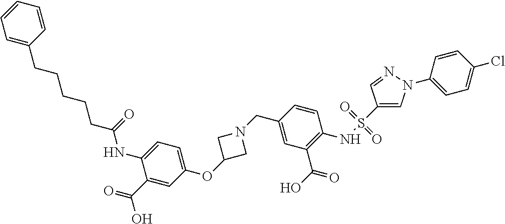 [SMILES: O=C(CCCCCc1ccccc1)Nc1ccc(OC2CN(Cc3ccc(NS(=O)(=O)c4cnn(-c5ccc(Cl)cc5)c4)c(C(=O)O)c3)C2)cc1C(=O)O] | 772.1 |
| 67 | 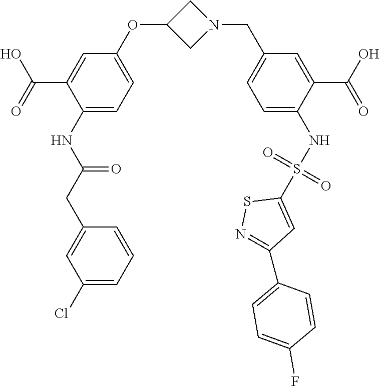 [SMILES: O=C(Cc1cccc(Cl)c1)Nc1ccc(OC2CN(Cc3ccc(NS(=O)(=O)c4cc(-c5ccc(F)cc5)ns4)c(C(=O)O)c3)C2)cc1C(=O)O] | 751.0 |
| 69 | 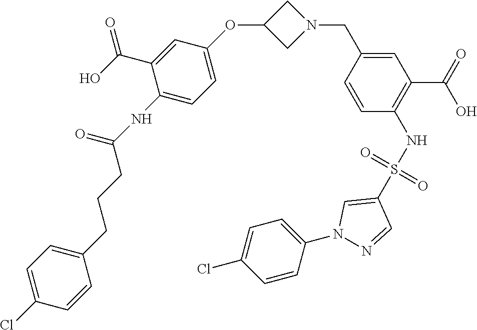 [SMILES: O=C(CCCc1ccc(Cl)cc1)Nc1ccc(OC2CN(Cc3ccc(NS(=O)(=O)c4cnn(-c5ccc(Cl)cc5)c4)c(C(=O)O)c3)C2)cc1C(=O)O] | 778.1 |
| 71 | 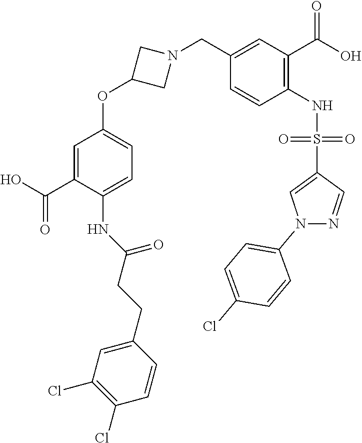 [SMILES: O=C(CCc1ccc(Cl)c(Cl)c1)Nc1ccc(OC2CN(Cc3ccc(NS(=O)(=O)c4cnn(-c5ccc(Cl)cc5)c4)c(C(=O)O)c3)C2)cc1C(=O)O] | 800.0 |
| 72 | 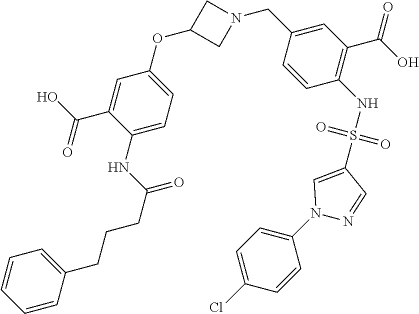 [SMILES: O=C(CCCc1ccccc1)Nc1ccc(OC2CN(Cc3ccc(NS(=O)(=O)c4cnn(-c5ccc(Cl)cc5)c4)c(C(=O)O)c3)C2)cc1C(=O)O] | 744.2 |
| 73 | 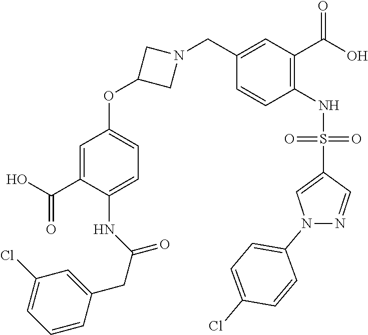 [SMILES: O=C(Cc1cccc(Cl)c1)Nc1ccc(OC2CN(Cc3ccc(NS(=O)(=O)c4cnn(-c5ccc(Cl)cc5)c4)c(C(=O)O)c3)C2)cc1C(=O)O] | 750.1 |
| 74 | 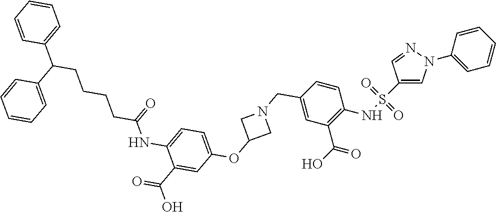 [SMILES: O=C(CCCCC(c1ccccc1)c1ccccc1)Nc1ccc(OC2CN(Cc3ccc(NS(=O)(=O)c4cnn(-c5ccccc5)c4)c(C(=O)O)c3)C2)cc1C(=O)O] | 848.1 |
| 75 | 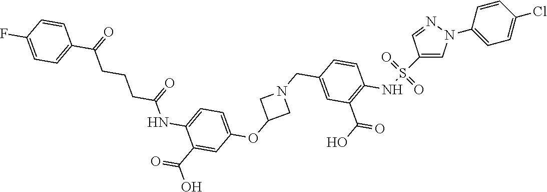 [SMILES: O=C(CCCC(=O)c1ccc(F)cc1)Nc1ccc(OC2CN(Cc3ccc(NS(=O)(=O)c4cnn(-c5ccc(Cl)cc5)c4)c(C(=O)O)c3)C2)cc1C(=O)O] | 790.1 |

### EXAMPLE 15

### Preparation of Compound 78

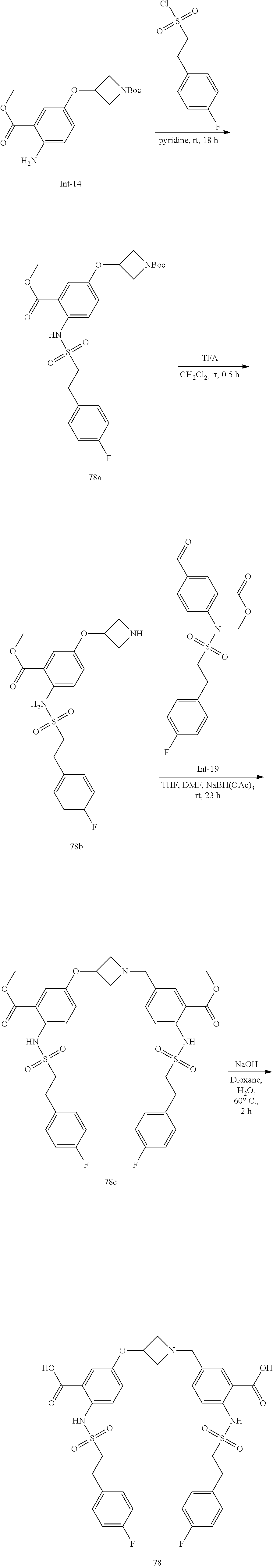

### Step 1: tert-butyl 3-(4-((2-(4-fluorophenyl)ethyl) sulfonamido)-3-(methoxycarbonyl)-phenoxy) azetidine-1-carboxylate (78a)

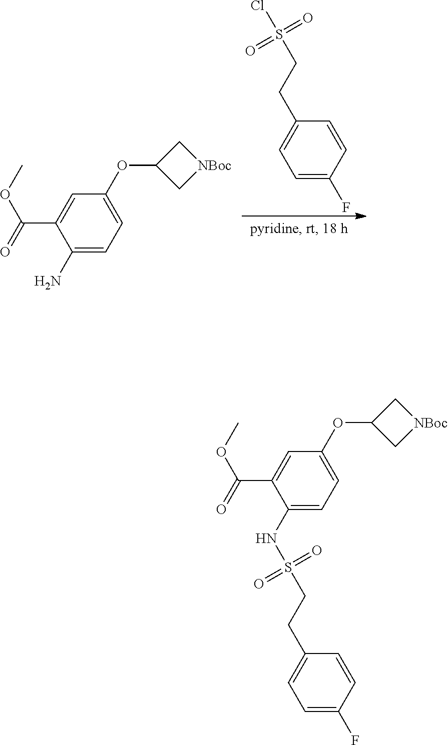 [SMILES: COC(=O)c1cc(OC2CN(C(=O)OC(C)(C)C)C2)ccc1N ; O=S(=O)(Cl)CCc1ccc(F)cc1 ; COC(=O)c1cc(OC2CN(C(=O)OC(C)(C)C)C2)ccc1NS(=O)(=O)CCc1ccc(F)cc1]

To a solution of tert-butyl 3-(4-amino-3-(methoxycarbonyl) phenoxy) azetidine-1-carboxylate (Int-14; 750 mg, 2.09 mmol) in pyridine (12 mL) was added 2-(4-fluoro-phenyl) ethane-1-sulfonyl chloride (512 mg, 2.30 mmol) at room temperature. The reaction mixture was stirred for 18 h. Additional 2-(4-fluorophenyl) ethane-1-sulfonyl chloride (205 mg, 0.92 mmol) was added and the reaction mixture was stirred for 24 h. Then reaction mixture was concentrated in vacuo and the resulting residue was purified by flash chromatography on SiO₂ [Isco®, SepaFlash® Silica Flash Column (40 g), eluent of 0 to 20% EtOAc/(1:1 Hexanes/CH₂Cl₂) gradient at 40 mL/min] to provide tert-butyl 3-(4-((2-(4-fluorophenyl)ethyl) sulfonamido)-3-(methoxycarbonyl) phenoxy) azetidine-1-carboxylate. MS (ESI, m/z): 453.2 [M+H⁺].

### Step 2: methyl 5-(azetidin-3-yloxy)-2-((2-(4-fluorophenyl)ethyl) sulfonamido)benzoate (78b)

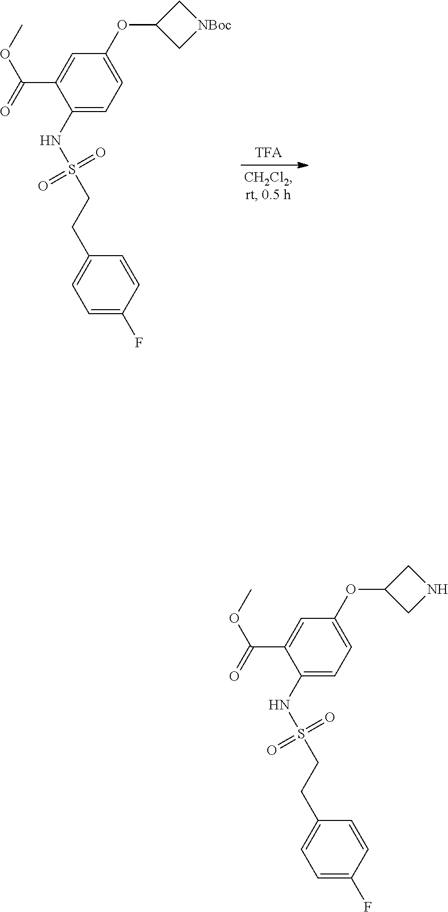 [SMILES: COC(=O)c1cc(OC2CN(C(=O)OC(C)(C)C)C2)ccc1NS(=O)(=O)CCc1ccc(F)cc1 ; COC(=O)c1cc(OC2CNC2)ccc1NS(=O)(=O)CCc1ccc(F)cc1]

To a solution of tert-butyl 3-(4-((2-(4-fluorophenyl)ethyl) sulfonamido)-3-(methoxy-carbonyl) phenoxy) azetidine-1-carboxylate (78a; 833 mg, 1.64 mmol) in CH₂Cl₂ (10 mL) was added TFA (6.3 mL, 82 mmol) at room temperature. The reaction was stirred for 0.5 h, then concentrated in vacuo. The resulting residue was dissolved in 1,4-dioxane and lyophilized to providea crude methyl 5-(azetidin-3-yloxy)-2-((2-(4-fluorophenyl)ethyl) sulfonamido)benzoate, which was used in the subsequent step without further purification. MS (ESI, m/z): 409.2 [M+H⁺].

### Step 3: methyl 2-((2-(4-fluorophenyl)ethyl) sulfonamido)-5-((1-(4-((2-(4-fluorophenyl)-ethyl) sulfonamido)-3-(methoxycarbonyl)benzyl) azetidin-3-yl)oxy)benzoate (78c)

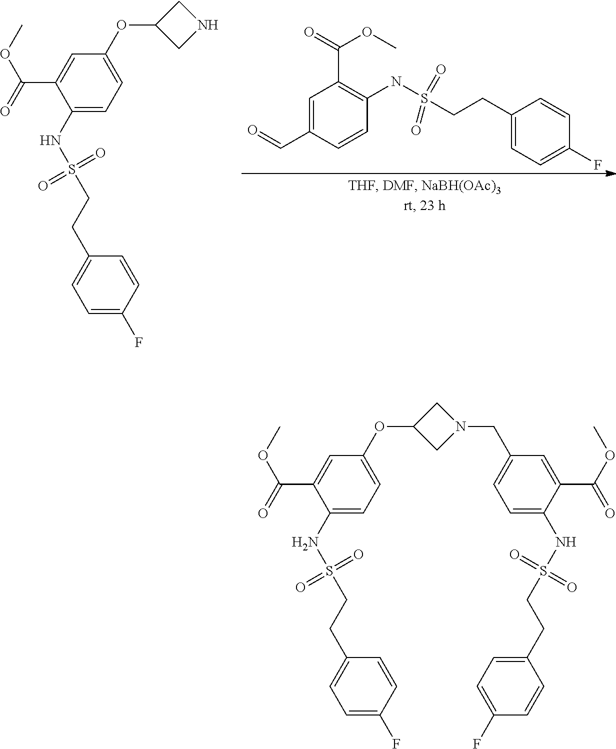

To a solution of methyl 5-(azetidin-3-yloxy)-2-((2-(4-fluorophenyl)ethyl)-sulfonamido)-benzoate (78b; 8.6 mg, 0.021 mmol) in THF (250 μL) and DMF (50 μL) were added methyl 2-((2-(4-fluorophenyl)ethyl) sulfonamido)-5-formylbenzoate (Int-19; 7.7 mg, 0.021 mmol) and solid-supported MP-(OAc) 3BH (180 mg, 0.417 mmol, loading @ 2.32 mmol/g) at room temperature. The resulting mixture was stirred at room temperature for 20 h. Then the mixture was filtered through an OptiChem SPE cartridge and the solvent was removed in vacuo to provide the crude methyl 2-((2-(4-fluorophenyl)ethyl) sulfonamido)-5-((1-(4-((2-(4-fluorophenyl)ethyl) sulfonamido)-3-(methoxycarbonyl)benzyl) azetidin-3-yl)oxy)benzoate, which was used in the next step without further purification.

### Step 4:5-((1-(3-carboxy-4-((2-(4-fluorophenyl)ethyl) sulfonamido)benzyl) azetidin-3-yl)oxy)-2-((2-(4-fluorophenyl)ethyl) sulfonamido)benzoic acid (78)

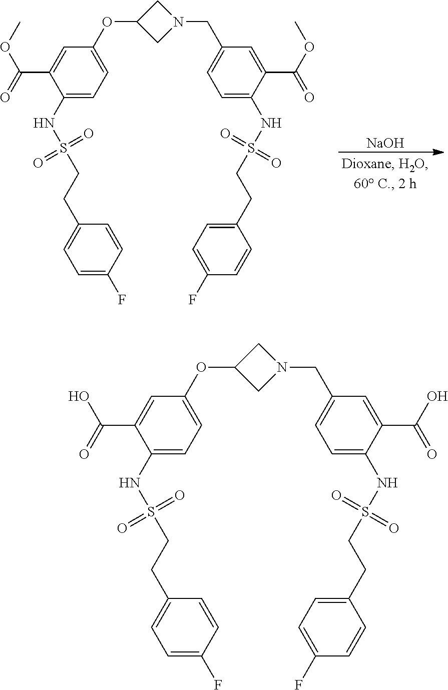 [SMILES: COC(=O)c1cc(CN2CC(Oc3ccc(NS(=O)(=O)CCc4ccc(F)cc4)c(C(=O)OC)c3)C2)ccc1NS(=O)(=O)CCc1ccc(F)cc1 ; "NaOH" ; O=C(O)c1cc(CN2CC(Oc3ccc(NS(=O)(=O)CCc4ccc(F)cc4)c(C(=O)O)c3)C2)ccc1NS(=O)(=O)CCc1ccc(F)cc1]

To a solution of methyl 2-((2-(4-fluorophenyl)ethyl) sulfonamido)-5-((1-(4-((2-(4-fluoro-phenyl)ethyl) sulfonamido)-3-(methoxycarbonyl)benzyl) azetidin-3-yl)oxy)benzoate (78c; 16 mg, 0.021 mmol) in 1,4-dioxane (0.25 mL) was added a 1.25 M aq. solution of NaOH (0.34 mL, 0.42 mmol) at room temperature. The reaction mixture was heated to 60° C. and stirred for 2 h, then cooled to room temperature and acidified with TFA (50 μL). The reaction mixture was concentrated in vacuo, and the resulting residue was purified by preparative reverse phase chromatography [Waters, Sunfire C18 column (100*19 mm, 5 μm), eluent of Mobile Phase A: H₂O (0.1% TFA) and Mobile Phase B: MeCN at 215 nm] to provide 5-((1-(3-carboxy-4-((2-(4-fluorophenyl)ethyl) sulfonamido)-benzyl) azetidin-3-yl)oxy)-2-((2-(4-fluorophenyl)ethyl) sulfonamido)benzoic acid. MS (ESI, m/z): 730.3 [M−H⁺]. ¹H NMR (500 MHz, DMSO-d₆) δ 8.13 (d, J=1.8 Hz, 1H), 7.66 (d, J=8.7 Hz, 1H), 7.58 (d, J=8.5 Hz, 1H), 7.51 (d, J=9.0 Hz, 1H), 7.35 (d, J=3.0 Hz, 1H), 7.23-7.12 (m, 5H), 7.02 (q, J=8.8 Hz, 4H), 5.09 (s, 1H), 4.47 (s, 2H), 4.40 (s, 2H), 4.13 (s, 1H), 3.49-3.45 (m, 4H), 2.97 (dt, J=18.6, 7.8 Hz, 4H).

The following compound (79) of the present disclosure were made using the methods described in Example 15 above, and substituting the appropriate reactants and/or reagents:

| | | Observed |
|---|---|---|
| Compound | Structure | MS |
| 79 | 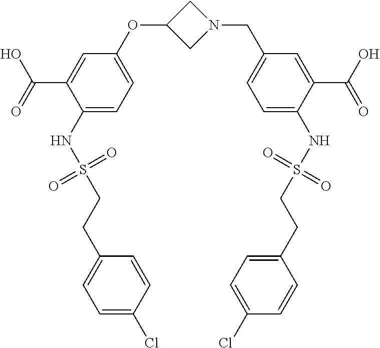 [SMILES: O=C(O)c1cc(CN2CC(Oc3ccc(NS(=O)(=O)CCc4ccc(Cl)cc4)c(C(=O)O)c3)C2)ccc1NS(=O)(=O)CCc1ccc(Cl)cc1] | 762.4 |

### EXAMPLE 16

### Preparation of Compound 80

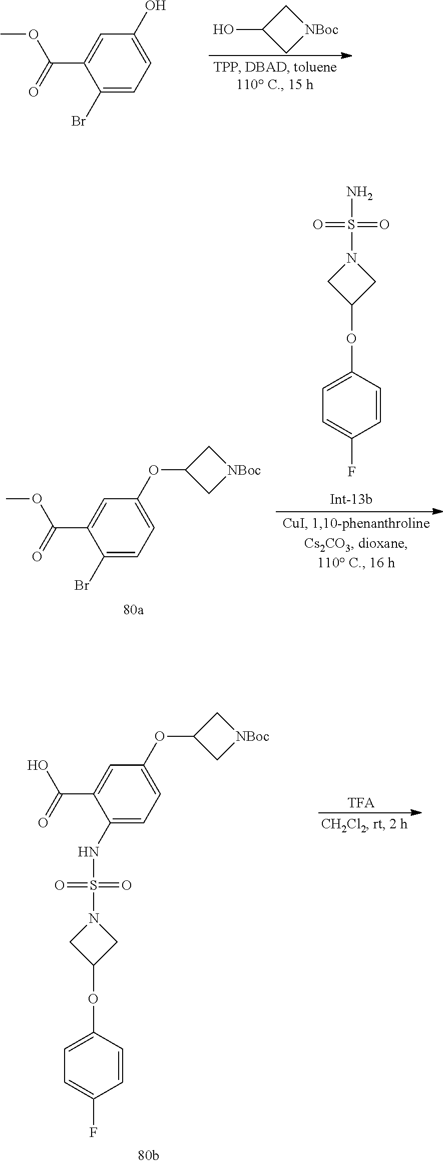 [SMILES: COC(=O)c1cc(O)ccc1Br ; CC(C)(C)OC(=O)N1CC(O)C1 ; COC(=O)c1cc(OC2CN(C(=O)OC(C)(C)C)C2)ccc1Br ; NS(=O)(=O)N1CC(Oc2ccc(F)cc2)C1 ; CC(C)(C)OC(=O)N1CC(Oc2ccc(NS(=O)(=O)N3CC(Oc4ccc(F)cc4)C3)c(C(=O)O)c2)C1]

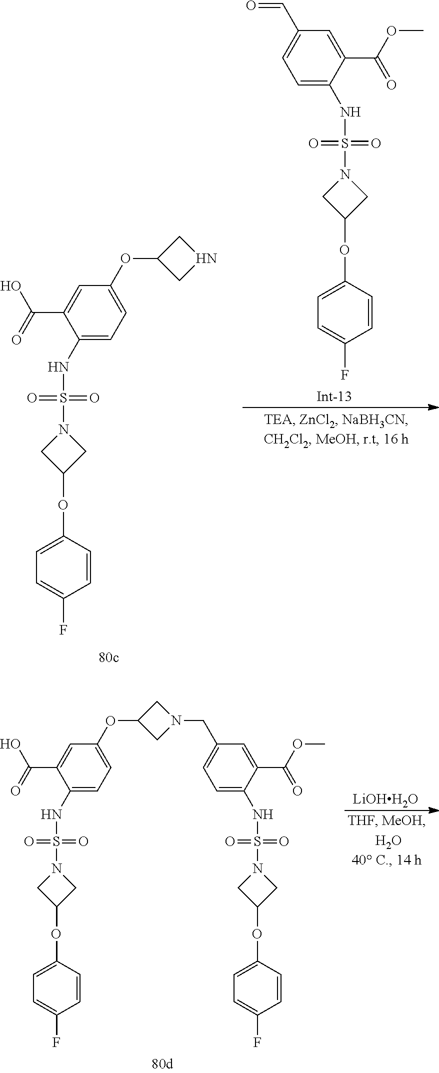 [SMILES: O=C(O)c1cc(OC2CNC2)ccc1NS(=O)(=O)N1CC(Oc2ccc(F)cc2)C1 ; COC(=O)c1cc(C=O)ccc1NS(=O)(=O)N1CC(Oc2ccc(F)cc2)C1 ; COC(=O)c1cc(CN2CC(Oc3ccc(NS(=O)(=O)N4CC(Oc5ccc(F)cc5)C4)c(C(=O)O)c3)C2)ccc1NS(=O)(=O)N1CC(Oc2ccc(F)cc2)C1 ; "LiOH·H2O"]

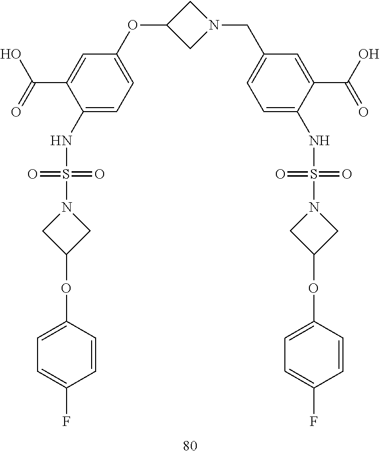 [SMILES: O=C(O)c1cc(CN2CC(Oc3ccc(NS(=O)(=O)N4CC(Oc5ccc(F)cc5)C4)c(C(=O)O)c3)C2)ccc1NS(=O)(=O)N1CC(Oc2ccc(F)cc2)C1]

### Step 1: tert-butyl 3-(4-bromo-3-(methoxycarbonyl) phenoxy) azetidine-1-carboxylate (80a)

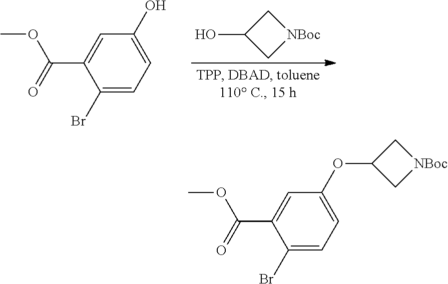 [SMILES: COC(=O)c1cc(O)ccc1Br ; CC(C)(C)OC(=O)N1CC(O)C1 ; COC(=O)c1cc(OC2CN(C(=O)OC(C)(C)C)C2)ccc1Br]

To a solution of methyl 2-bromo-5-hydroxybenzoate (2 g, 8.66 mmol) in toluene (15 mL) was added tert-butyl 3-hydroxyazetidine-1-carboxylate (1.799 g, 10.39 mmol), DBAD (2.59 g, 11.25 mmol) and triphenylphosphine (2.95 g, 11.25 mmol) at room temperature. The reaction mixture was stirred at 110° C. for 15 h. After cooling to room temperature, the reaction was diluted with H₂O (15 mL) and extracted with EtOAc (2×20 mL). The combined organics were dried over anh. Na₂SO₄, filtered and concentrated in vacuo. The resulting residue was purified by flash chromatography on SiO₂ [Isco®, Agela® 12 g Flash Column, eluent of 0 to 25% EtOAc/Pet. ether gradient at 35 mL/min) to provide tert-butyl 3-(4-bromo-3-(methoxycarbonyl) phenoxy) azetidine-1-carboxylate. MS (ESI) m/z: 332.1 [M+H⁺-56].

### Step 2:5-((1-(tert-butoxycarbonyl) azetidin-3-yl)oxy)-2-((3-(4-fluorophenoxy) azetidine)-1-sulfonamido)benzoic acid (80b)

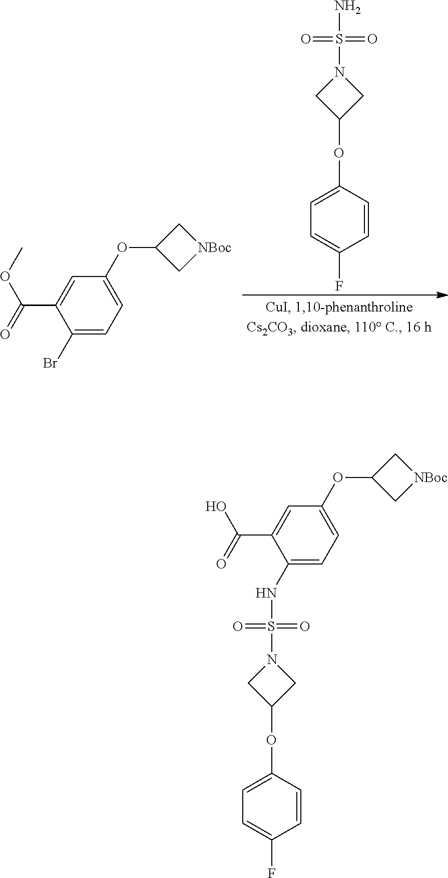 [SMILES: COC(=O)c1cc(OC2CN(C(=O)OC(C)(C)C)C2)ccc1Br ; NS(=O)(=O)N1CC(Oc2ccc(F)cc2)C1 ; CC(C)(C)OC(=O)N1CC(Oc2ccc(NS(=O)(=O)N3CC(Oc4ccc(F)cc4)C3)c(C(=O)O)c2)C1]

A mixture of 1,10-phenanthroline (9.33 mg, 0.052 mmol), Cs₂CO₃ (211 mg, 0.647 mmol), copper (I) iodide (4.93 mg, 0.026 mmol), 3-(4-fluorophenoxy) azetidine-1-sulfonamide (Int-13b; 41.4 mg, 0.168 mmol) and tert-butyl 3-(4-bromo-3-(methoxycarbonyl) phenoxy)-azetidine-1-carboxylate (80a; 50 mg, 0.129 mmol) in 1,4-dioxane (1 mL) was purged with N₂ for 1 min at room temperature, then stirred at 110° C. for 16 h. The reaction mixture was concentrated in vacuo and the resulting residue was purified by preparative reverse phase chromatography [Phenomenex Synergi C₁₈ (150*21.2 mm, 4 μm), eluent of Mobile Phase A: H₂O (0.1% TFA) and Mobile Phase B: MeCN @ 215 nm] to provide 5-((1-(tert-butoxycarbonyl) azetidin-3-yl)oxy)-2-((3-(4-fluorophenoxy)-azetidine)-1-sulfonamido)benzoic acid. MS (ESI) m/z: 482.1 [M+H⁺-56].

### Step 3:5-(azetidin-3-yloxy)-2-((3-(4-fluorophenoxy) azetidine)-1-sulfonamido)benzoic acid (80c)

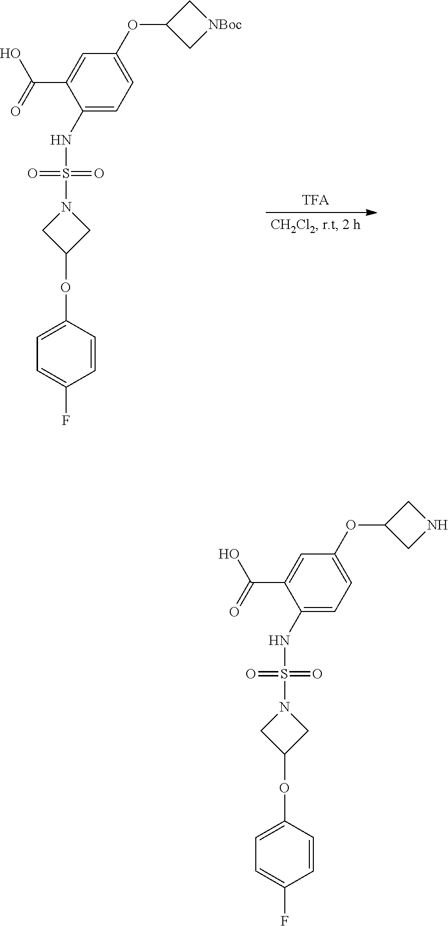 [SMILES: CC(C)(C)OC(=O)N1CC(Oc2ccc(NS(=O)(=O)N3CC(Oc4ccc(F)cc4)C3)c(C(=O)O)c2)C1 ; O=C(O)c1cc(OC2CNC2)ccc1NS(=O)(=O)N1CC(Oc2ccc(F)cc2)C1]

To a solution of 5-((1-(tert-butoxycarbonyl) azetidin-3-yl)oxy)-2-((3-(4-fluorophenoxy) azetidine)-1-sulfonamido)benzoic acid (80b; 20 mg, 0.037 mmol) in CH₂Cl₂ (2 mL), was added TFA (1 mL, 12.98 mmol) at room temperature, and the reaction mixture was stirred for 2 h, then concentrated in vacuo to provide the crude 5-(azetidin-3-yloxy)-2-((3-(4-fluorophenoxy) azetidine)-1-sulfonamido)benzoic acid, which was used in the subsequent step without further purification. MS (ESI) m/z: 438.1 [M+H⁺].

### Step 4:2-((3-(4-fluorophenoxy) azetidine)-1-sulfonamido)-5-((1-(4-((3-(4-fluorophenoxy) azetidine)-1-sulfonamido)-3-(methoxycarbonyl)benzyl) azetidin-3-yl)oxy)benzoic acid (80d)

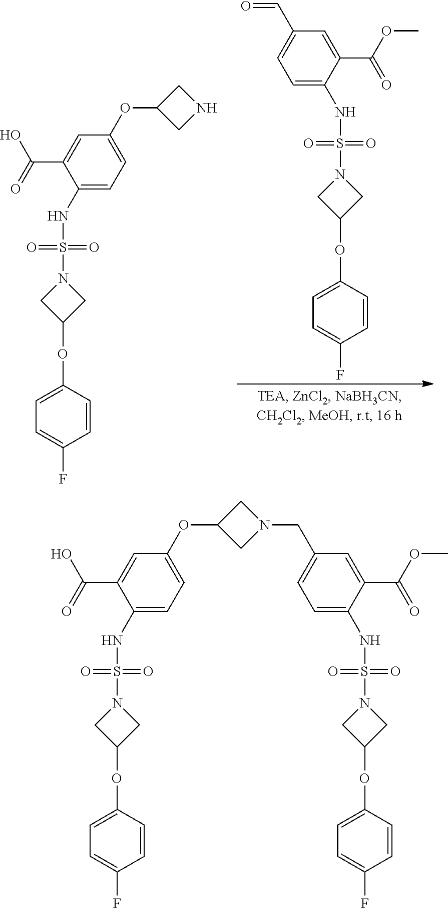 [SMILES: O=C(O)c1cc(OC2CNC2)ccc1NS(=O)(=O)N1CC(Oc2ccc(F)cc2)C1 ; COC(=O)c1cc(C=O)ccc1NS(=O)(=O)N1CC(Oc2ccc(F)cc2)C1 ; COC(=O)c1cc(CN2CC(Oc3ccc(NS(=O)(=O)N4CC(Oc5ccc(F)cc5)C4)c(C(=O)O)c3)C2)ccc1NS(=O)(=O)N1CC(Oc2ccc(F)cc2)C1]

To a stirred mixture of zinc (II) chloride (9.35 mg, 0.069 mmol) and 5-(azetidin-3-yloxy)-2-((3-(4-fluorophenoxy) azetidine)-1-sulfonamido)benzoic acid (80c; 15 mg, 0.034 mmol) in CH₂C₁₂ (1 mL) and MeOH (1 mL) was added methyl 2-((3-(4-fluorophenoxy) azetidine)-1-sulfonamido)-5-formylbenzoate (14.00 mg, 0.034 mmol) and TEA (0.014 mL, 0.103 mmol), followed by sodium cyanoborohydride (4.31 mg, 0.069 mmol) at room temperature. The reaction mixture was stirred at room temperature for 16 h. The reaction mixture was concentrated in vacuo to provide the crude 2-((3-(4-fluorophenoxy) azetidine)-1-sulfonamido)-5-((1-(4-((3-(4-fluorophenoxy) azetidine)-1-sulfonamido)-3-(methoxycarbonyl)benzyl) azetidin-3-yl)oxy)benzoic acid, which was used in the subsequent step without further purification. MS (ESI) m/z: 830.1 [M+H⁺].

### Step 5:5-((1-(3-carboxy-4-((3-(4-fluorophenoxy) azetidine)-1-sulfonamido)benzyl) azetidin-3-yl)oxy)-2-((3-(4-fluorophenoxy) azetidine)-1-sulfonamido)benzoic acid (80)

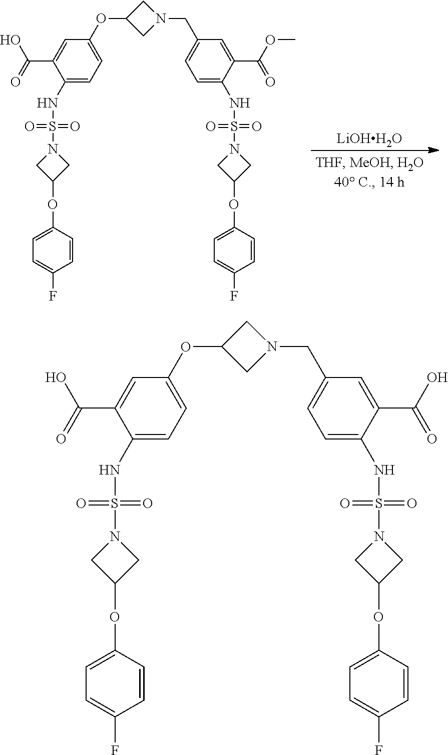

To a solution of 2-((3-(4-fluorophenoxy) azetidine)-1-sulfonamido)-5-((1-(4-((3-(4-fluorophenoxy) azetidine)-1-sulfonamido)-3-(methoxycarbonyl)benzyl) azetidin-3-yl)oxy)benzoic acid (80d; 20 mg, 0.024 mmol) in THF (2 mL), MeOH (2 mL) and H₂O (1 mL) was added lithium hydroxide monohydrate (5.06 mg, 0.121 mmol) at room temperature. The reaction mixture was stirred at 40° C. for 14 h, then adjusted to pH=7 with aq. HCl. The reacton mixture was concentrated in vacuo and the resulting residue was purified by preparative reverse phase chromatography [Phenomenex Synergi C₁₈ (150*21.2 mm, 4 μm), eluent of Mobile Phase A: H₂O (0.1% TFA) and Mobile Phase B: MeCN at 215 nm] to provide 5-((1-(3-carboxy-4-((3-(4-fluorophenoxy) azetidine)-1-sulfonamido)benzyl) azetidin-3-yl)oxy)-2-((3-(4-5 fluorophenoxy) azetidine)-1-sulfon-amido)benzoic acid. MS (ESI) m/z: 814.3 [M−H⁺]. JH NMR (500 MHZ, CD₃OD) δ ppm 8.20 (d, J=2.5 Hz, 1H), 7.79 (d, J=8.5 Hz, 1H), 7.69 (d, J=9.0 Hz, 1H), 7.60 (br d, J=8.5 Hz, 1H), 7.48 (d, J=3.0 Hz, 1H), 7.09 (dd, J=3.0, 9.0 Hz, 1H), 6.98 (t, J=8.5 Hz, 4H), 6.78-6.72 (m, 4H), 5.12 (br s, 1H), 4.90 (br s, 2H), 4.60 (dd, J=6.5, 12.0 Hz, 2H), 4.45 (s, 2H), 4.29-4.19 (m, 6H), 3.90 (ddd, J=5.0, 9.5, 19.5 Hz, 4H).

### EXAMPLE 17

### Preparation of Compounds 80

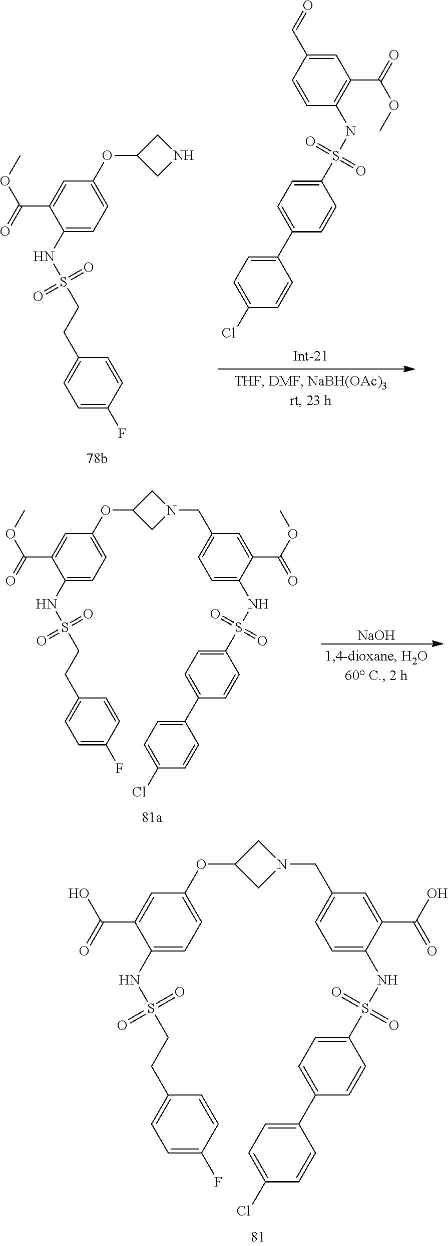

### Step 1: methyl 2-((4′-chloro-[1,1′-biphenyl])-4-sulfonamido)-5-((3-(4-((2-(4-fluorophenyl)-ethyl) sulfonamido)-3-(methoxycarbonyl) phenoxy) azetidin-1-yl)methyl)benzoate (81a)

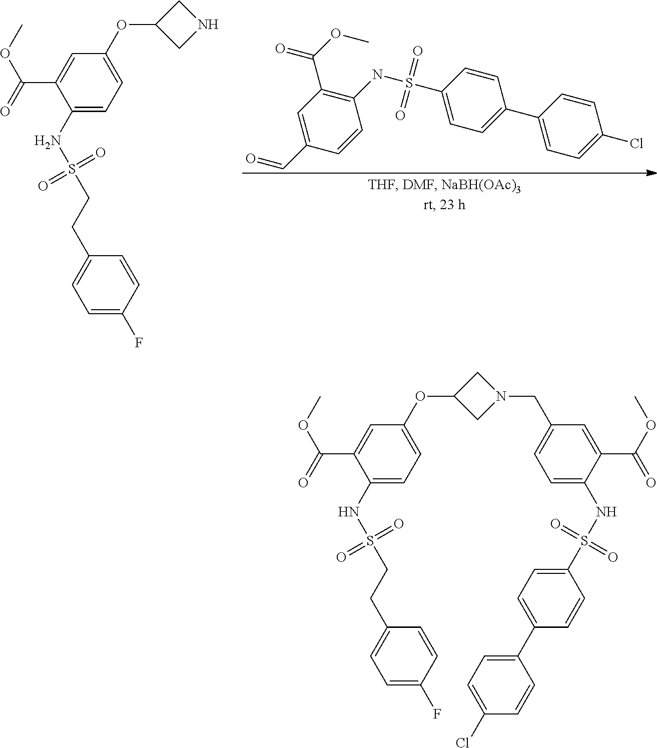

To a solution of methyl 5-(azetidin-3-yloxy)-2-((2-(4-fluorophenyl)ethyl)-sulfonamido)-benzoate (78b; 9.0 mg, 0.021 mmol) and methyl 2-((4′-chloro-[1,1′-biphenyl])-4-sulfonamido)-5-formylbenzoate (Int-21; 7.7 mg, 0.021 mmol) in THF (250 μL) and DMF (50 μL) was added solid supported MP-(OAc) 3BH (180 mg, 0.417 mmol, loading @ 2.32 mmol/g) at room temperature. The reaction mixture was stirred at room temperature for 20 h, then filtered through an OptiChem SPE cartridge. The filtrate was concentrated in vacuo to provide crude methyl 2-((2-(4-fluorophenyl)ethyl) sulfonamido)-5-((1-(4-((2-(4-fluorophenyl)ethyl)-sulfonamido)-3-(methoxycarbonyl)benzyl) azetidin-3-yl)oxy)benzoate, which was used in the subsequent step without further purification.

### Step 2:5-((3-(3-carboxy-4-((2-(4-fluorophenyl)ethyl) sulfonamido) phenoxy) azetidin-1-yl)methyl)-2-((4′-chloro-[1, l′-biphenyl])-4-sulfonamido)benzoic acid (81)

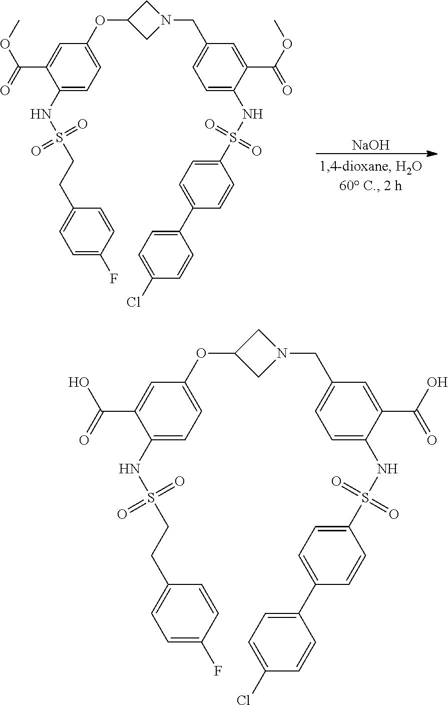 [SMILES: COC(=O)c1cc(OC2CN(Cc3ccc(NS(=O)(=O)c4ccc(-c5ccc(Cl)cc5)cc4)c(C(=O)OC)c3)C2)ccc1NS(=O)(=O)CCc1ccc(F)cc1 ; "NaOH" ; O=C(O)c1cc(OC2CN(Cc3ccc(NS(=O)(=O)c4ccc(-c5ccc(Cl)cc5)cc4)c(C(=O)O)c3)C2)ccc1NS(=O)(=O)CCc1ccc(F)cc1]

To a solution of methyl 2-((4′-chloro-[1,l′-biphenyl])-4-sulfonamido)-5-((3-(4-((2-(4-fluorophenyl)ethyl) sulfonamido)-3-(methoxycarbonyl) phenoxy) azetidin-1-yl)-methyl)-benzoate (17 mg, 0.021 mmol) in 1,4-dioxane (0.25 mL) was added 1.25 M sodium hydroxide (0.34 mL, 0.42 mmol) in H₂O at room temperature. The reaction mixture was stirred at 60° C. for 2 h, cooled to room temperature, and acidified with TFA (50 μL). The reaction mixture was concentrated in vacuo and the resulting residue was purified by preparative reverse phase chromatography [Waters, Sunfire C18 column (100*19 mm, 5 μm), eluent of Mobile Phase A: H₂O (0.1% TFA) and Mobile Phase B: MeCN @ 215 nm] to provide 5-((3-(3-carboxy-4-((2-(4-fluorophenyl)ethyl) sulfonamido) phenoxy) azetidin-1-yl)methyl)-2-((4′-chloro-[1,l′-biphenyl])-4-sulfonamido)benzoic acid. MS (ESI, m/z): 794.3 [M−H⁺]. ¹H NMR (500 MHZ, DMSO-d₆) δ 8.04 (s, 1H), 7.88 (d, J=8.5 Hz, 2H), 7.81 (d, J=8.5 Hz, 2H), 7.71 (d, J=8.6 Hz, 2H), 7.56-7.43 (m, 5H), 7.33 (d, J=3.0 Hz, 1H), 7.18 (dd, J=8.5, 5.6 Hz, 2H), 7.10 (dd, J=9.0, 3.1 Hz, 1H), 7.03 (t, J=8.8 Hz, 2H), 5.04 (s, 1H), 4.41 (s, 2H), 4.29 (s, 2H), 4.07 (s, 1H), 3.45-3.37 (m, 2H), 2.97-2.91 (m, 2H).

The following compounds (82 through 86) of the present disclosure were made using the methods described in Example 17 above, and substituting the appropriate reactants and/or

| | | Observed |
|---|---|---|
| Compound | Structure | MS |
| 82 | 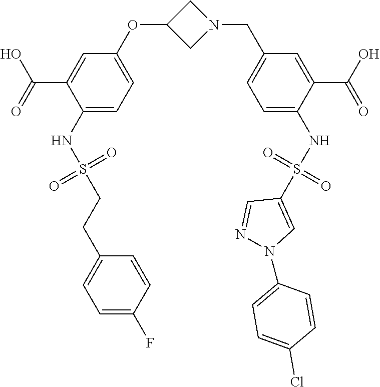 [SMILES: O=C(O)c1cc(OC2CN(Cc3ccc(NS(=O)(=O)c4cnn(-c5ccc(Cl)cc5)c4)c(C(=O)O)c3)C2)ccc1NS(=O)(=O)CCc1ccc(F)cc1] | 784.2 |
| 83 | 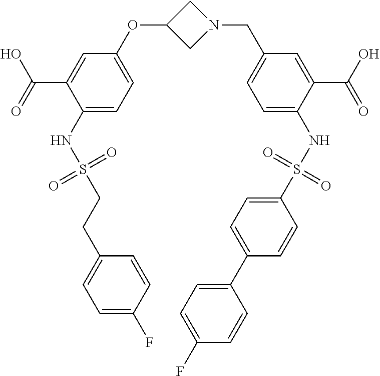 [SMILES: O=C(O)c1cc(OC2CN(Cc3ccc(NS(=O)(=O)c4ccc(-c5ccc(F)cc5)cc4)c(C(=O)O)c3)C2)ccc1NS(=O)(=O)CCc1ccc(F)cc1] | 778.3 |
| 84 | 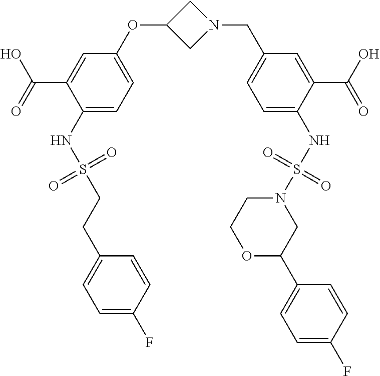 [SMILES: O=C(O)c1cc(OC2CN(Cc3ccc(NS(=O)(=O)N4CCOC(c5ccc(F)cc5)C4)c(C(=O)O)c3)C2)ccc1NS(=O)(=O)CCc1ccc(F)cc1] | 785.2 (M − H⁺) |
| 85 | 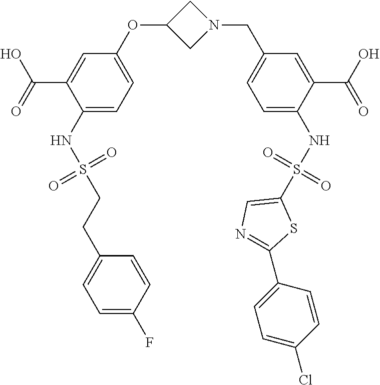 [SMILES: O=C(O)c1cc(OC2CN(Cc3ccc(NS(=O)(=O)c4cnc(-c5ccc(Cl)cc5)s4)c(C(=O)O)c3)C2)ccc1NS(=O)(=O)CCc1ccc(F)cc1] | 801.2 |
| 86 | 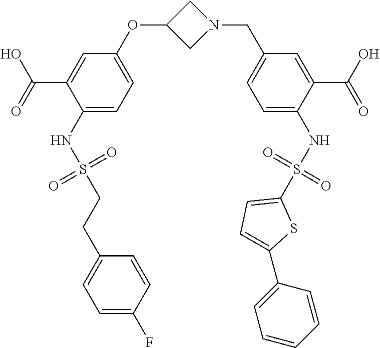 [SMILES: O=C(O)c1cc(OC2CN(Cc3ccc(NS(=O)(=O)c4ccc(-c5ccccc5)s4)c(C(=O)O)c3)C2)ccc1NS(=O)(=O)CCc1ccc(F)cc1] | 766.3 |

### EXAMPLE 18

### Preparation of Compound 87

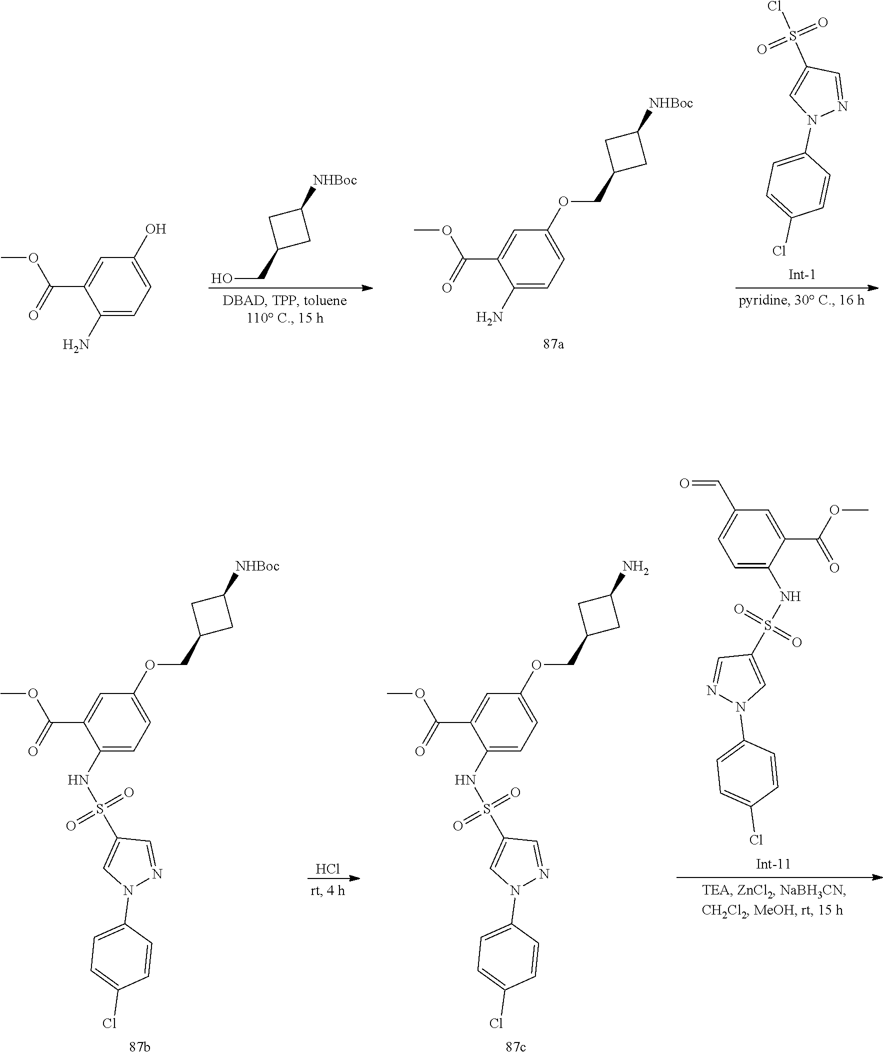

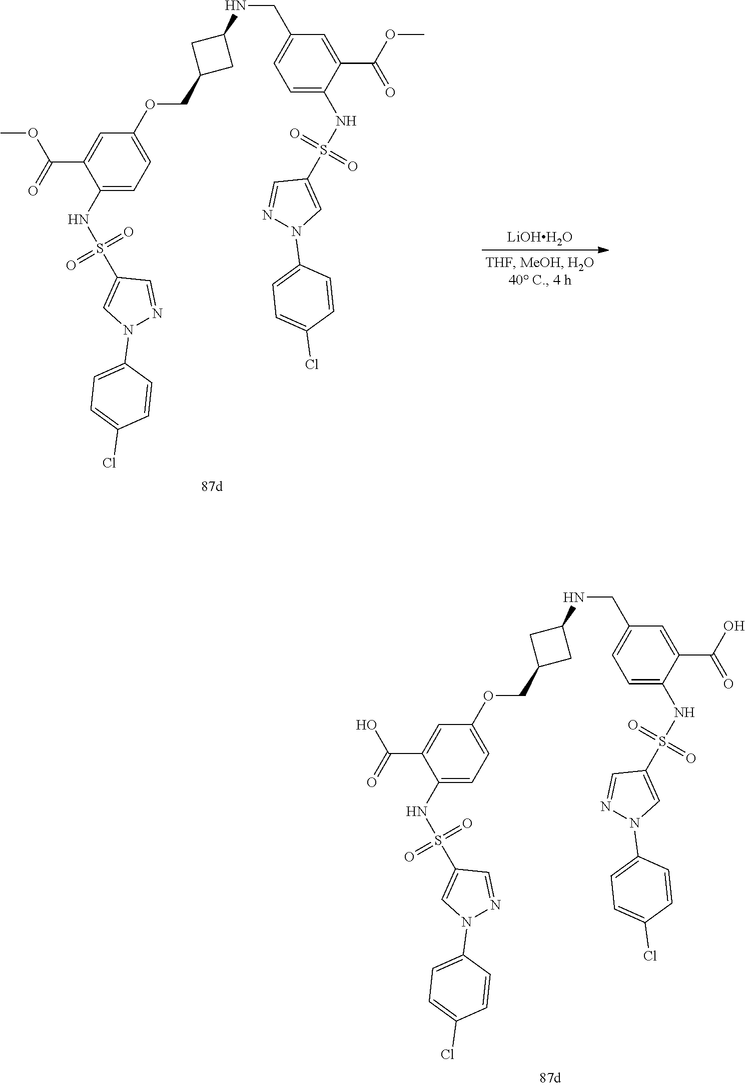

### Step 1: methyl 2-amino-5-(((1s, 3s)-3-((tert-butoxycarbonyl)amino) cyclobutyl) methoxy)benzoate (87a)

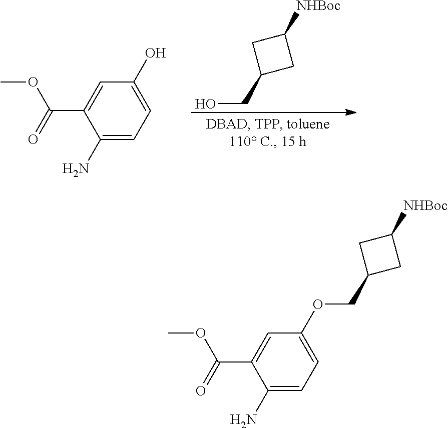 [SMILES: COC(=O)c1cc(O)ccc1N ; CC(C)(C)OC(=O)N[C@H]1C[C@@H](CO)C1 ; COC(=O)c1cc(OC[C@H]2C[C@@H](NC(=O)OC(C)(C)C)C2)ccc1N]

A mixture of methyl 2-amino-5-hydroxybenzoate (1.827 g, 10.93 mmol), tert-butyl ((1s,3s)-3-(hydroxymethyl)cyclobutyl) carbamate (2 g, 9.94 mmol), PPh3 (3.91 g, 14.91 mmol), DBAD (3.43 g, 14.91 mmol) in toluene (80 mL) was stirred at 110° C. for 15 h under N₂. After cooling to room temperature, the reaction mixture was diluted with EtOAc (200 mL), washed with HCl (0.5 N, 2×200 mL), dried over anh. Na₂SO₄, filtered and the filtrate was concentrated in vacuo.

The resulting residue was purified by flash chromatography on SiO₂ [Isco®, 40 g Silica Flash Column, eluent of 0 to 27% EtOAc/Pet. ether gradient at 35 mL/min] to provide methyl 2-amino-5-(((1s,3s)-3-((tert-butoxycarbonyl)amino) cyclobutyl)-methoxy)benzoate. MS (ESI, m/z): 351.3 [M+H⁺]Step 2: methyl 5-(((1s, 3s)-3-((tert-butoxycarbonyl)amino) cyclobutyl) methoxy)-2-((1-(4-chlorophenyl)-1H-pyrazole)-4-sulfonamido)benzoate (87b)

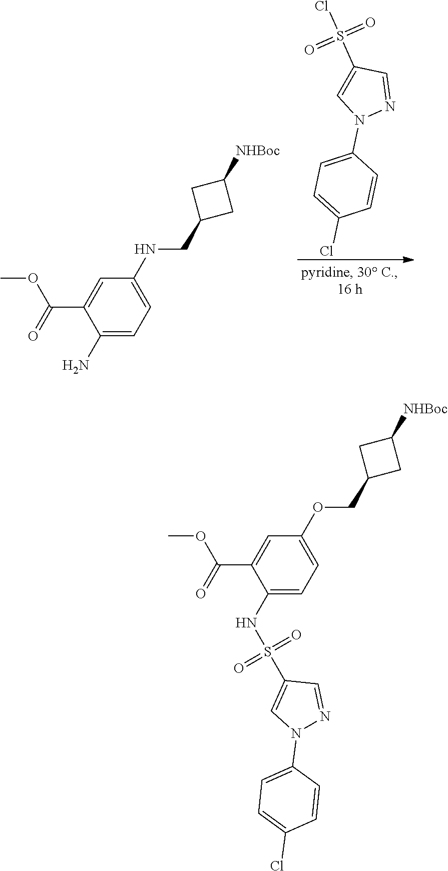 [SMILES: COC(=O)c1cc(NC[C@H]2C[C@@H](NC(=O)OC(C)(C)C)C2)ccc1N ; O=S(=O)(Cl)c1cnn(-c2ccc(Cl)cc2)c1 ; COC(=O)c1cc(OC[C@H]2C[C@@H](NC(=O)OC(C)(C)C)C2)ccc1NS(=O)(=O)c1cnn(-c2ccc(Cl)cc2)c1]

To a solution of methyl 2-amino-5-(((1s,3s)-3-((tert-butoxycarbonyl)amino) cyclobutyl) methoxy)benzoate (87a; 500 mg, 1.427 mmol) in pyridine (5 mL) was added 1-(4-chlorophenyl)-1H-pyrazole-4-sulfonyl chloride (Int-1; 395 mg, 1.427 mmol) at room temperature, then stirred for 16 h. Then reaction mixture was concentrated in vacuo, and the resulting residue was purified by flash chromatography on SiO₂ [Isco®, Agela® Flash 4 g Column, eluent of 15% EtOac/Pet. ether isocratic gradient at 30 mL/min) to provide methyl 5-(((1s,3s)-3-((tert-butoxycarbonyl)amino) cyclobutyl) methoxy)-2-((1-(4-chlorophenyl)-1H-pyrazole)-4-sulfonamido)benzoate. MS (ESI, m/z): 591.2 [M+H⁺]

### Step 3: methyl 5-(((1s, 3s)-3-aminocyclobutyl) methoxy)-2-((1-(4-chlorophenyl)-1H-pyrazole)-4-sulfonamido)benzoate (87c)

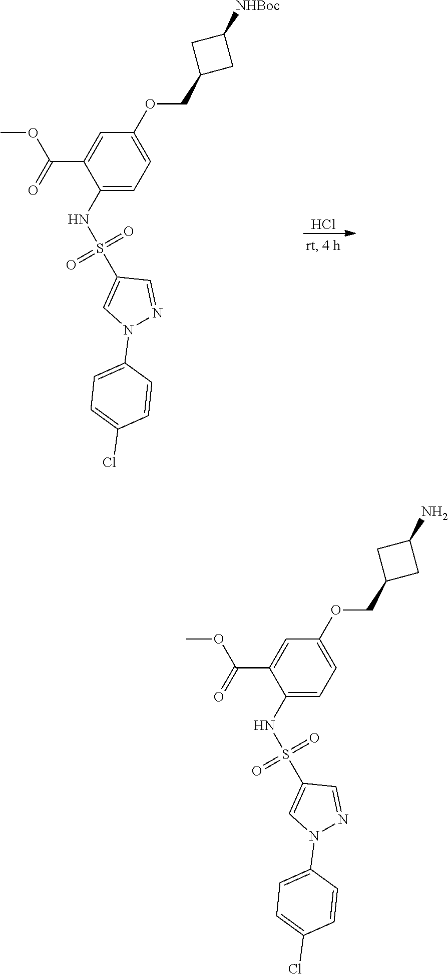 [SMILES: COC(=O)c1cc(OC[C@H]2C[C@@H](NC(=O)OC(C)(C)C)C2)ccc1NS(=O)(=O)c1cnn(-c2ccc(Cl)cc2)c1 ; Cl ; COC(=O)c1cc(OC[C@H]2C[C@@H](N)C2)ccc1NS(=O)(=O)c1cnn(-c2ccc(Cl)cc2)c1]

A mixture of methyl 5-(((1s,3s)-3-((tert-butoxycarbonyl)amino) cyclobutyl) methoxy)-2-((1-(4-chlorophenyl)-1H-pyrazole)-4-sulfonamido)benzoate (87b; 165 mg, 0.279 mmol) and HCl (4 N in 1,4-dioxane, 3 mL) was stirred at room temperature for 4 h. The reaction mixture was concentrated in vacuo to provide the crude methyl 5-(((1s,3s)-3-aminocyclobutyl) methoxy)-2-((1-(4-chlorophenyl)-1/-pyrazole)-4-sulfonamido)benzoate which was used in the subsequent step without further purification. MS ((ESI, m/z): 491.1 [M+H⁺].

### Step 4: methyl 2-((1-(4-chlorophenyl)-1H-pyrazole)-4-sulfonamido)-5-(((1s, 3s)-3-((4-((1-(4-chlorophenyl)-1H-pyrazole)-4-sulfonamido)-3-(methoxycarbonyl)benzyl)amino)cyclobutyl) methoxy)benzoate (87d)

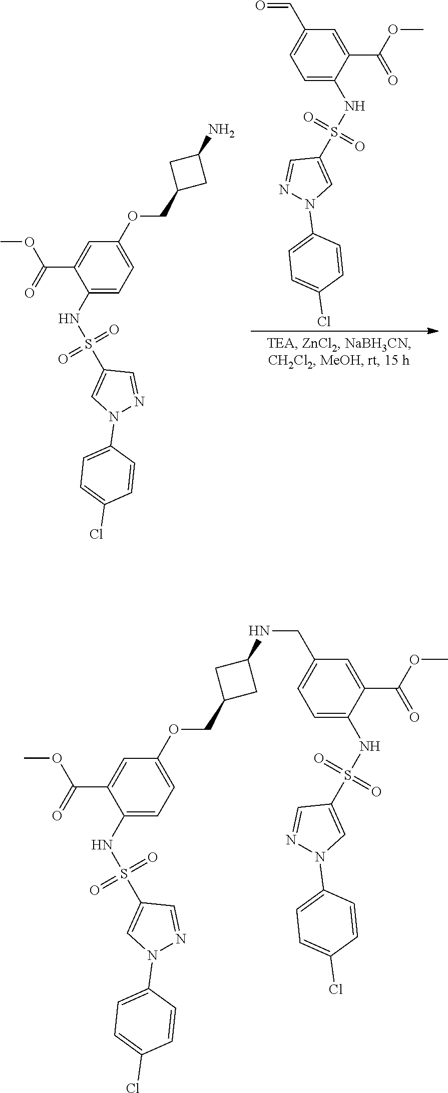

To a solution of methyl 5-(((1S,3S)-3-aminocyclobutyl) methoxy)-2-((1-(4-chlorophenyl)-1H-pyrazole)-4-sulfonamido)benzoate (87c; 65 mg, 0.132 mmol) in MeOH (5 mL) and CH₂Cl₂ (5.00 mL) were added TEA (9.23 μl, 0.066 mmol), methyl 2-((1-(4-chlorophenyl)-1H-pyrazole)-4-sulfonamido)-5-formylbenzoate (Int-11; 55.6 mg, 0.132 mmol), NaBH₃CN (24.96 mg, 0.397 mmol), and zinc (II) chloride (36.1 mg, 0.265 mmol) at room temperature. The reaction mixture was stirred at room temperature for 15 h, then concentrated in vacuo, and the reaction mixture was diluted with H₂O (10 mL), and extracted with EtOAc (3×8 mL). The combined organics were dried over anh. Na₂SO₄, filtered, and concentrated in vacuo to get provide 2-((1-(4-chlorophenyl)-1H-pyrazole)-4-sulfonamido)-5-(((1s,3s)-3-((4-((1-(4-chlorophenyl)-1H-pyrazole)-4-sulfonamido)-3-(methoxycarbonyl)benzyl)amino)cyclobutyl) methoxy), which was used for the subsequent step without further purification. MS (ESI, m/z): 896.1 [M+H⁺]Step 5:5-(((1s, 3s)-3-((3-carboxy-4-((1-(4-chlorophenyl)-1H-pyrazole)-4-sulfonamid o)benzyl)amino)cyclobutyl) methoxy)-2-((1-(4-chlorophenyl)-1H-pyrazole)-4-sulfonamido)benzoic acid (87)

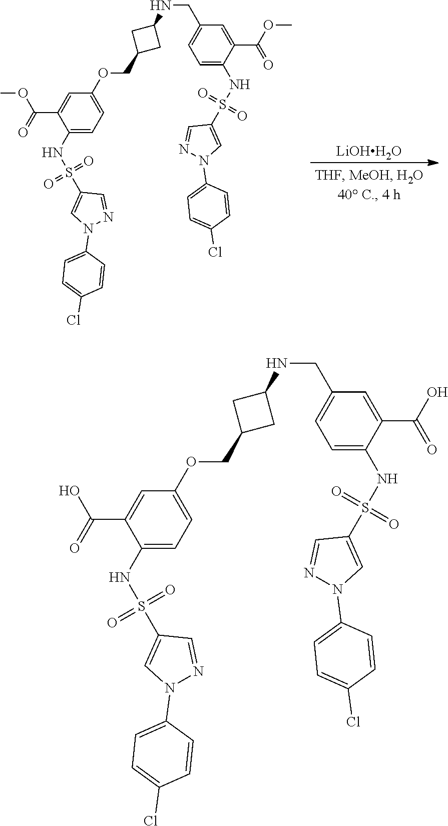

To a solution of methyl 2-((1-(4-chlorophenyl)-1H-pyrazole)-4-sulfonamido)-5-(((1s,3s)-3-((4-((1-(4-chlorophenyl)-1H-pyrazole)-4-sulfonamido)-3-(methoxycarbonyl)benzyl)amino)cyclobutyl) methoxy)benzoate (87d; 110 mg, 0.123 mmol) in THF (2 mL), MeOH (1 mL), and H₂O (1 mL) was added lithium hydroxide hydrate (31.0 mg, 0.738 mmol) at room temperature. The reaction mixture was stirred at 40° C. for 4 h, then diluted with aq. HCl to pH=˜ 7 (weakly acidic). After concentrating in vacuo, the resulting residue was purified by preparative reverse phase chromatography [Gilson 281, Phenomenex Gemini-NX (150*30 mm, 5 μm), eluent of Mobile Phase A: H₂O (0.1% HCl) and Mobile Phase B: MeCN at 220 nm] to provide 5-(((1s,3s)-3-((3-carboxy-4-((1-(4-chlorophenyl)-1H-pyrazole)-4-sulfonamido)-benzyl)-amino)cyclobutyl) methoxy)-2-((1-(4-chlorophenyl)-1H-pyrazole)-4-sulfonamido)-benzoic acid. MS (ESI, m/z): 866.1 [M+H⁺]. ¹H NMR (500 MHZ, CD₃OD) δ ppm 8.87 (s, 1H), 8.65 (s, 1H), 8.18 (d, J=2.0 Hz, 1H), 7.92 (s, 1H), 7.82-7.88 (m, 1H), 7.64-7.77 (m, 7H), 7.41-7.54 (m, 5H), 7.17 (dd, J=9.5, 2.98 Hz, 1H), 4.08 (s, 2H), 3.89 (d, J=5.5 Hz, 2H), 3.69 (br t, J=8.5 Hz, 1H), 2.54 (br d, J=7.5 Hz, 1H), 2.33-2.45 (m, 2H), 1.91-2.09 (m, 2H).

The following compounds (88 through 96) of the present disclosure were made using the methods described in Examples 18 above, and substituting the appropriate reactants and/or

| | | Observed |
|---|---|---|
| Compound | Structure | MS |
| 88 | 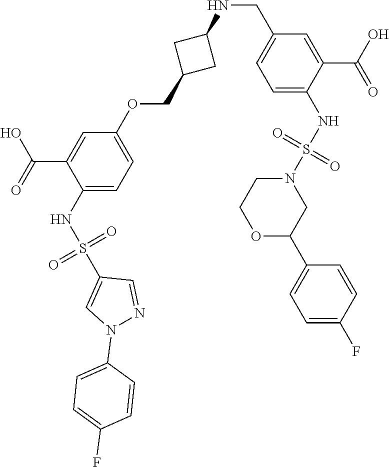 [SMILES: O=C(O)c1cc(OC[C@H]2C[C@@H](NCc3ccc(NS(=O)(=O)N4CCOC(c5ccc(F)cc5)C4)c(C(=O)O)c3)C2)ccc1NS(=O)(=O)c1cnn(-c2ccc(F)cc2)c1] | 853.2 |
| 89 | 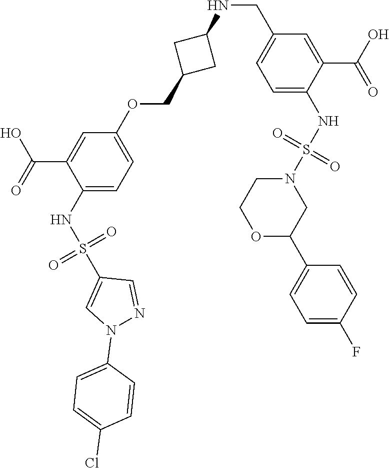 [SMILES: O=C(O)c1cc(OC[C@H]2C[C@@H](NCc3ccc(NS(=O)(=O)N4CCOC(c5ccc(F)cc5)C4)c(C(=O)O)c3)C2)ccc1NS(=O)(=O)c1cnn(-c2ccc(Cl)cc2)c1] | 869.2 |
| 90 | 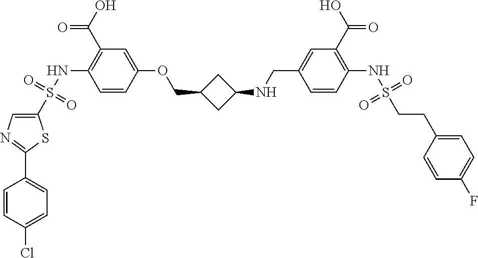 [SMILES: O=C(O)c1cc(CN[C@H]2C[C@@H](COc3ccc(NS(=O)(=O)c4cnc(-c5ccc(Cl)cc5)s4)c(C(=O)O)c3)C2)ccc1NS(=O)(=O)CCc1ccc(F)cc1] | 829.4 |
| 91 | 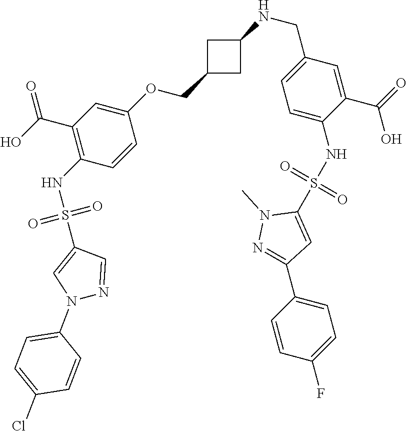 [SMILES: Cn1nc(-c2ccc(F)cc2)cc1S(=O)(=O)Nc1ccc(CN[C@H]2C[C@@H](COc3ccc(NS(=O)(=O)c4cnn(-c5ccc(Cl)cc5)c4)c(C(=O)O)c3)C2)cc1C(=O)O] | 864.4 |
| 92 | 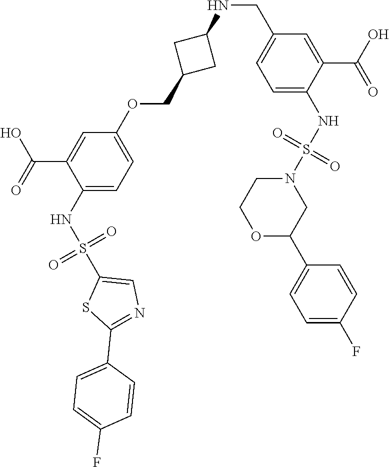 [SMILES: O=C(O)c1cc(OC[C@H]2C[C@@H](NCc3ccc(NS(=O)(=O)N4CCOC(c5ccc(F)cc5)C4)c(C(=O)O)c3)C2)ccc1NS(=O)(=O)c1cnc(-c2ccc(F)cc2)s1] | 870.1 |
| 93 | 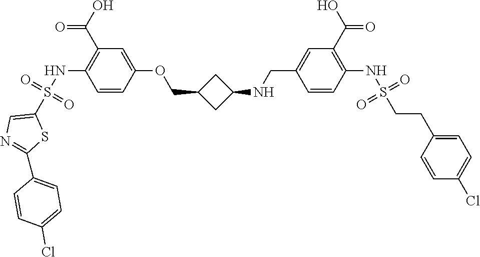 [SMILES: O=C(O)c1cc(CN[C@H]2C[C@@H](COc3ccc(NS(=O)(=O)c4cnc(-c5ccc(Cl)cc5)s4)c(C(=O)O)c3)C2)ccc1NS(=O)(=O)CCc1ccc(Cl)cc1] | 845.3 |
| 94 | 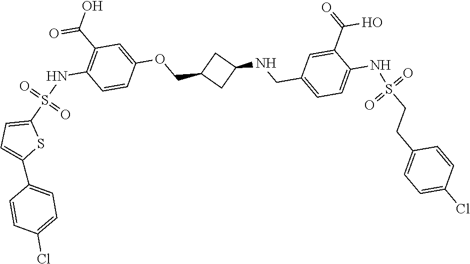 [SMILES: O=C(O)c1cc(CN[C@H]2C[C@@H](COc3ccc(NS(=O)(=O)c4ccc(-c5ccc(Cl)cc5)s4)c(C(=O)O)c3)C2)ccc1NS(=O)(=O)CCc1ccc(Cl)cc1] | 844.1 |
| 95 | 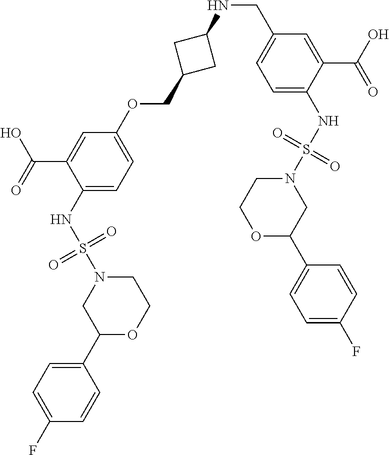 [SMILES: O=C(O)c1cc(CN[C@H]2C[C@@H](COc3ccc(NS(=O)(=O)N4CCOC(c5ccc(F)cc5)C4)c(C(=O)O)c3)C2)ccc1NS(=O)(=O)N1CCOC(c2ccc(F)cc2)C1] | 872.3 |
| 96 | 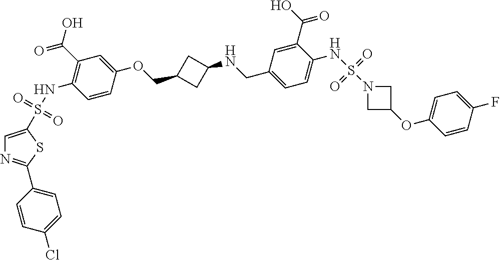 [SMILES: O=C(O)c1cc(OC[C@H]2C[C@@H](NCc3ccc(NS(=O)(=O)N4CC(Oc5ccc(F)cc5)C4)c(C(=O)O)c3)C2)ccc1NS(=O)(=O)c1cnc(-c2ccc(Cl)cc2)s1] | 872.4 |

### EXAMPLE 19

### Preparation of Compound 97

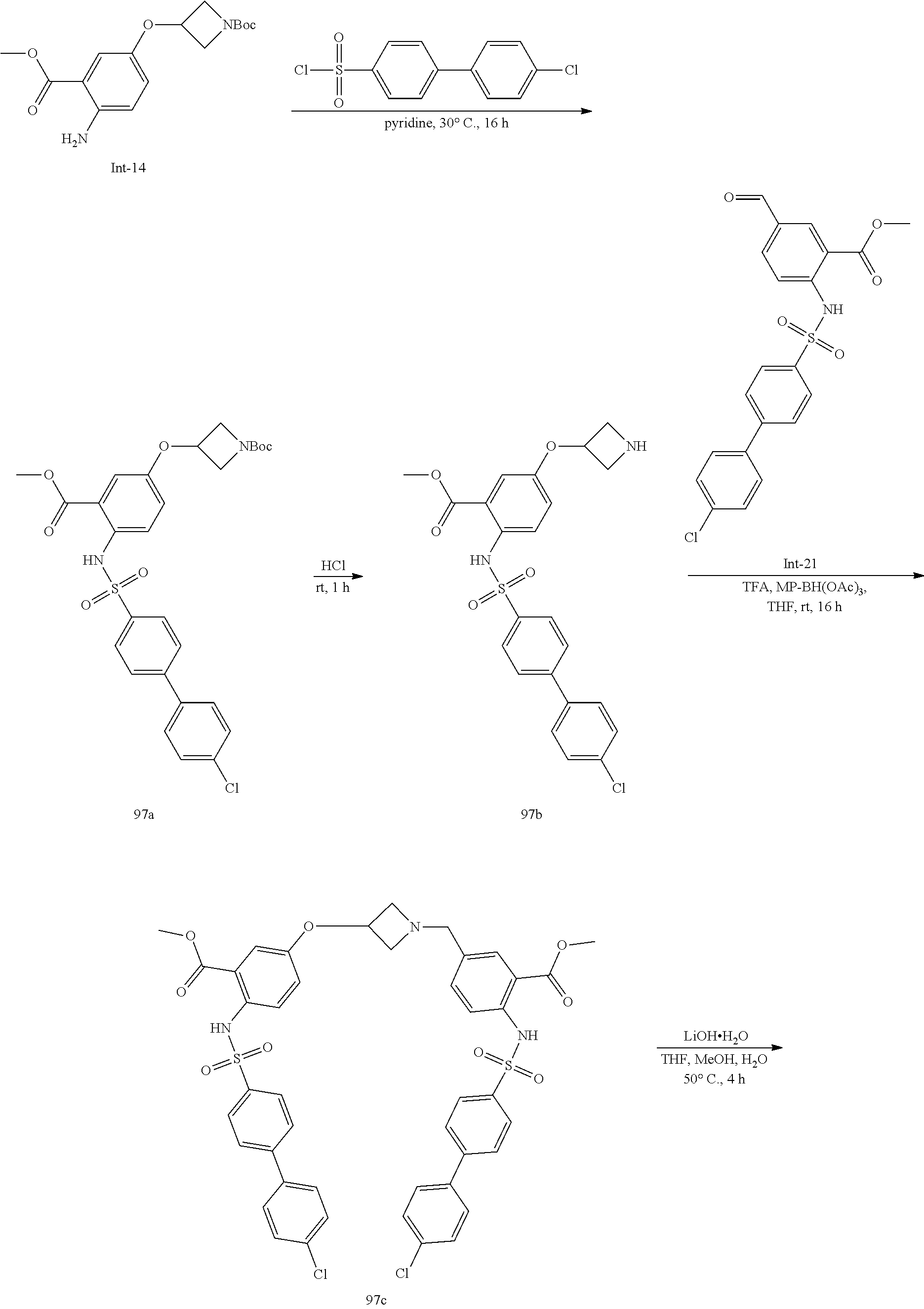

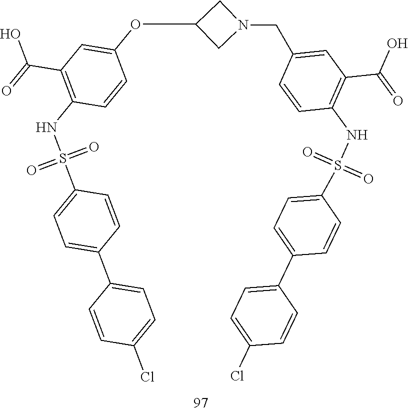 [SMILES: O=C(O)c1cc(CN2CC(Oc3ccc(NS(=O)(=O)c4ccc(-c5ccc(Cl)cc5)cc4)c(C(=O)O)c3)C2)ccc1NS(=O)(=O)c1ccc(-c2ccc(Cl)cc2)cc1]

### Step 1: tert-butyl 3-(4-((4′-chloro-(1,1′-biphenyl))-4-sulfonamido)-3-(methoxycarbonyl) phenoxy) azetidine-1-carboxylate (97a)

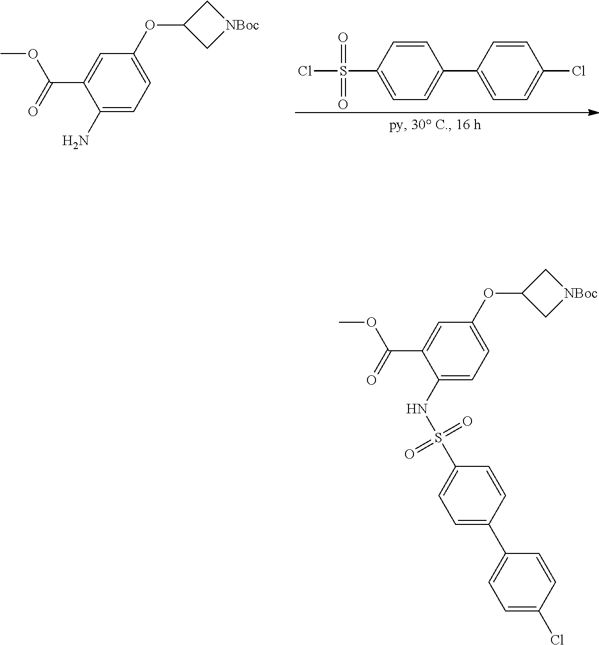 [SMILES: COC(=O)c1cc(OC2CN(C(=O)OC(C)(C)C)C2)ccc1N ; O=S(=O)(Cl)c1ccc(-c2ccc(Cl)cc2)cc1 ; COC(=O)c1cc(OC2CN(C(=O)OC(C)(C)C)C2)ccc1NS(=O)(=O)c1ccc(-c2ccc(Cl)cc2)cc1]

To a solution of tert-butyl 3-(4-amino-3-(methoxycarbonyl) phenoxy) azetidine-1-carboxylate (Int-14; 220 mg, 0.682 mmol) in pyridine (5 mL) was added 4′-chloro-[1,1′-biphenyl]-4-sulfonyl chloride (206 mg, 0.716 mmol) at room temperature. The reaction mixture was stirred at room temperature for 16 h, concentrated in vacuo, and the resulting residue was acidified with IN HCl, diluted with H₂O (30 mL), and extracted with EtOAc (2×30 mL). The combined organics were washed with brine (30 mL), dried over anh. MgSO4, filtered, and concentrated in vacuo. The resulting residue was purified by flash chromatography on SiO₂ [Isco®, Silica Flash 24 g Column, eluent of 0 to 60% EtOAc/Pet. ether gradient at 35 mL/min] to provide tert-butyl 3-(4-((4′-chloro-[1,l′-biphenyl])-4-sulfonamido)-3-(methoxycarbonyl) phenoxy) azetidine-1-carboxylate. MS (ESI, m/z): 573.5 [M+H⁺]. ¹H NMR (400 MHZ, CDCl₃) δ ppm10.14 (s, 1H), 7.82 (d, J=8.8 Hz, 2H), 7.71 (d, J=9.2 Hz, 1H), 7.58 (d, J=8.8 Hz, 2H), 7.52-7.36 (m, 4H), 7.20 (d, J=2.8 Hz, 1H), 6.94 (dd, J=2.8, 9.2 Hz, 1H), 4.82 (tt, J=4.0, 6.4 Hz, 1H), 4.27 (dd, J=6.0, 9.6 Hz, 2H), 3.94 (dd, J=4.0, 10.4 Hz, 2H), 3.81 (s, 3H), 1.43 (s, 9H).

### Step 2: methyl 5-(azetidin-3-yloxy)-2-((4′-chloro-(1,l′-biphenyl))-4-sulfonamido)benzoate (97b)

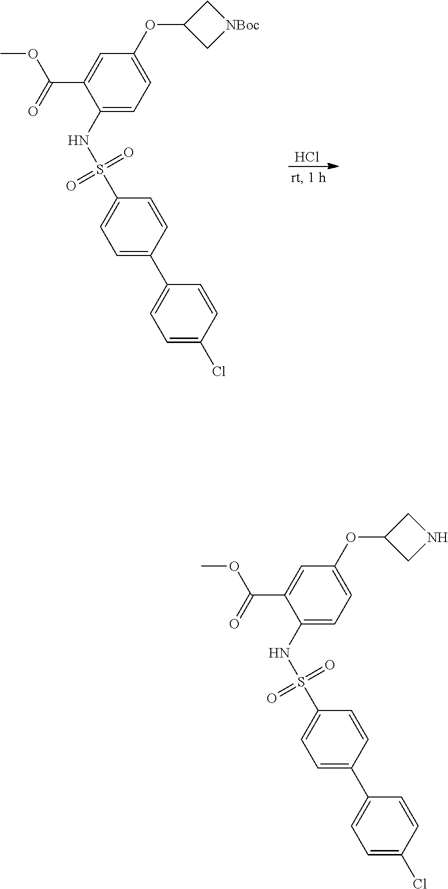 [SMILES: COC(=O)c1cc(OC2CN(C(=O)OC(C)(C)C)C2)ccc1NS(=O)(=O)c1ccc(-c2ccc(Cl)cc2)cc1 ; Cl ; COC(=O)c1cc(OC2CNC2)ccc1NS(=O)(=O)c1ccc(-c2ccc(Cl)cc2)cc1]

tert-Butyl 3-(4-((4′-chloro-[1,l′-biphenyl])-4-sulfonamido)-3-(methoxycarbonyl) phenoxy) azetidine-1-carboxylate (97a; 1.1 g, 1.920 mmol) was dissolved in HCl (4 N in 1,4-dioxane, 20 mL) and stirred at room temperature for 1 h, then concentrated in vacuo to provide crude methyl 5-(azetidin-3-yloxy)-2-((4′-chloro-[1,l′-biphenyl])-4-sulfonamido)benzoate, which was used in the subsequent step without further purification. MS (ESI, m/z): 473.1 [M+H⁺].

### Step 3: methyl 2-(4′-chloro-(1, l′-biphenyl)-4-sulfonamido)-5-(1-(4-(4′-chloro- (1, l′-biphenyl))-4-sulfonamido)-3-(methoxycarbonyl)benzyl) azetidin-3-yl)oxy)benzoate (97c)

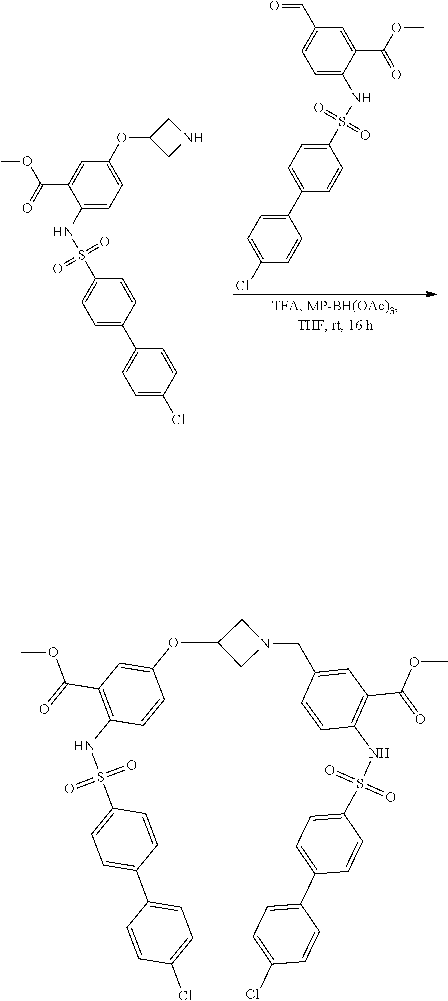

To a solution of methyl 5-(azetidin-3-yloxy)-2-((4′-chloro-(1,1′-biphenyl|)-4-sulfonamido)benzoate (97b; 100 mg, 0.196 mmol) and methyl 2-((4′-chloro-(1,1′-biphenyl])-4-sulfonamido)-5-formylbenzoate (Int-21; 88 mg, 0.201 mmol) in THF (5 mL) was added TFA (75 μl, 0.98 mmol), at room temperature. The reaction mixture was stirred for 10 min, and MP-Triacetoxyborohydride (207 mg, 0.49 mmol, loading: 2.36 mmol/g) was added. The resulting mixture was stirred at room temperature for 16 h and then filtered. The filtrate was basified with sat. Na₂CO₃, diluted with H₂O (20 mL) and extracted with EtOAc (2×20 mL). The combined organic layers were washed with brine (20 mL), dried over anh. MgSO4, filtered, and concentrated in vacuo to provide methyl 2-((4′-chloro-(1,1′-biphenyl)-4-sulfonamido)-5-((1-(4-((4′-chloro-(1,1′-biphenyl)-4-sulfonamido)-3-(methoxycarbonyl)benzyl) azetidin-3-yl)oxy)benzoate, which was used in the subsequent step without further purification. MS (ESI, m/z): 886.1 [M+H⁺]

### Step 4:5-((1-(3-carboxy-4-((4′-chloro-(1,l′-biphenyl))-4-sulfonamido)benzyl) azetidin-3-yl)oxy)-2-((4′-chloro-(1,l′-biphenyl))-4-sulfonamido)benzoic acid (97)

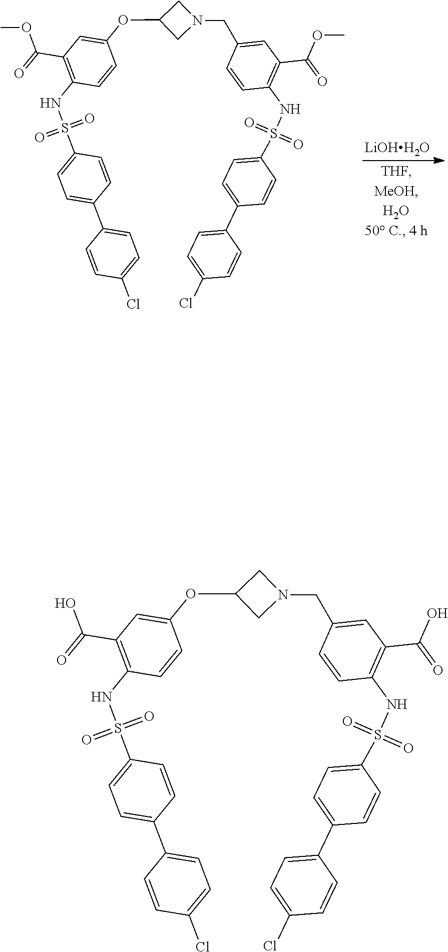

To a solution of methyl 2-((4′-chloro-(1,1′-biphenyl)-4-sulfonamido)-5-((1-(4-((4′-chloro-(1,1′-biphenyl)-4-sulfonamido)-3-(methoxycarbonyl)benzyl) azetidin-3-yl)oxy)benzoate (97c; 187 mg, 0.211 mmol) in THF (6 mL), MeOH (3 mL) and H₂O (3 mL) was added lithium hydroxide hydrate (88.6 mg, 2.11 mmol) at room temperature. The reaction was stirred the stirred at 50° C. for 4 h. Dilute aq. hydrochloric acid was added to pH˜7 (weakly acidic). The reaction mixture was concentrated in vacuo, and the resulting residue was purified by preparative reverse phase chromatography [Waters 2767, YMC column (150*20 mm, 5 μm), eluent of Mobile Phase A:

H₂O (0.1% TFA) and Mobile Phase B: MeCN (0.1% TFA) at 220 nm] to provide 5-((1-(3-carboxy-4-((4′-chloro-(1,1′-biphenyl))-4-sulfonamido)benzyl) azetidin-3-yl)oxy)-2-((4′-chloro-(1,1′-biphenyl))-4-sulfonamido)benzoic acid. MS (ESI, m/z): 858.3 [M+H⁺]. ¹H NMR (600 MHz, DMSO) δ ppm 7.98 (d, J=2.2 Hz, 1H), 7.87 (m, 2H), 7.78 (m, 6H), 7.67 (m, 4H), 7.51 (m, 4H), 7.47 (d, J=8.5 Hz, 1H), 7.45 (d, J=9.0 Hz, 1H), 7.42 (dd, J=8.6, 2.3 Hz, 1H), 7.28 (d, J=3.1 Hz, 1H), 7.01 (dd, J=9.0, 3.1 Hz, 1H), 4.95 (br m, 1H), 4.26 (br m, 2H), 4.16 (br m, 2H), 3.86 (br m, 2H).

The following compound (98) of the present disclosure were made using the methods described in Example 19 above, and substituting the appropriate reactants and/or reagents:

| | | Observed |
|---|---|---|
| Compound | Structure | MS |
| 98 | 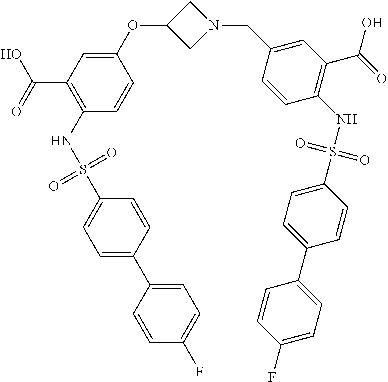 [SMILES: O=C(O)c1cc(CN2CC(Oc3ccc(NS(=O)(=O)c4ccc(-c5ccc(F)cc5)cc4)c(C(=O)O)c3)C2)ccc1NS(=O)(=O)c1ccc(-c2ccc(F)cc2)cc1] | 826.2 |

### EXAMPLE 20

### Preparation of Compound 99

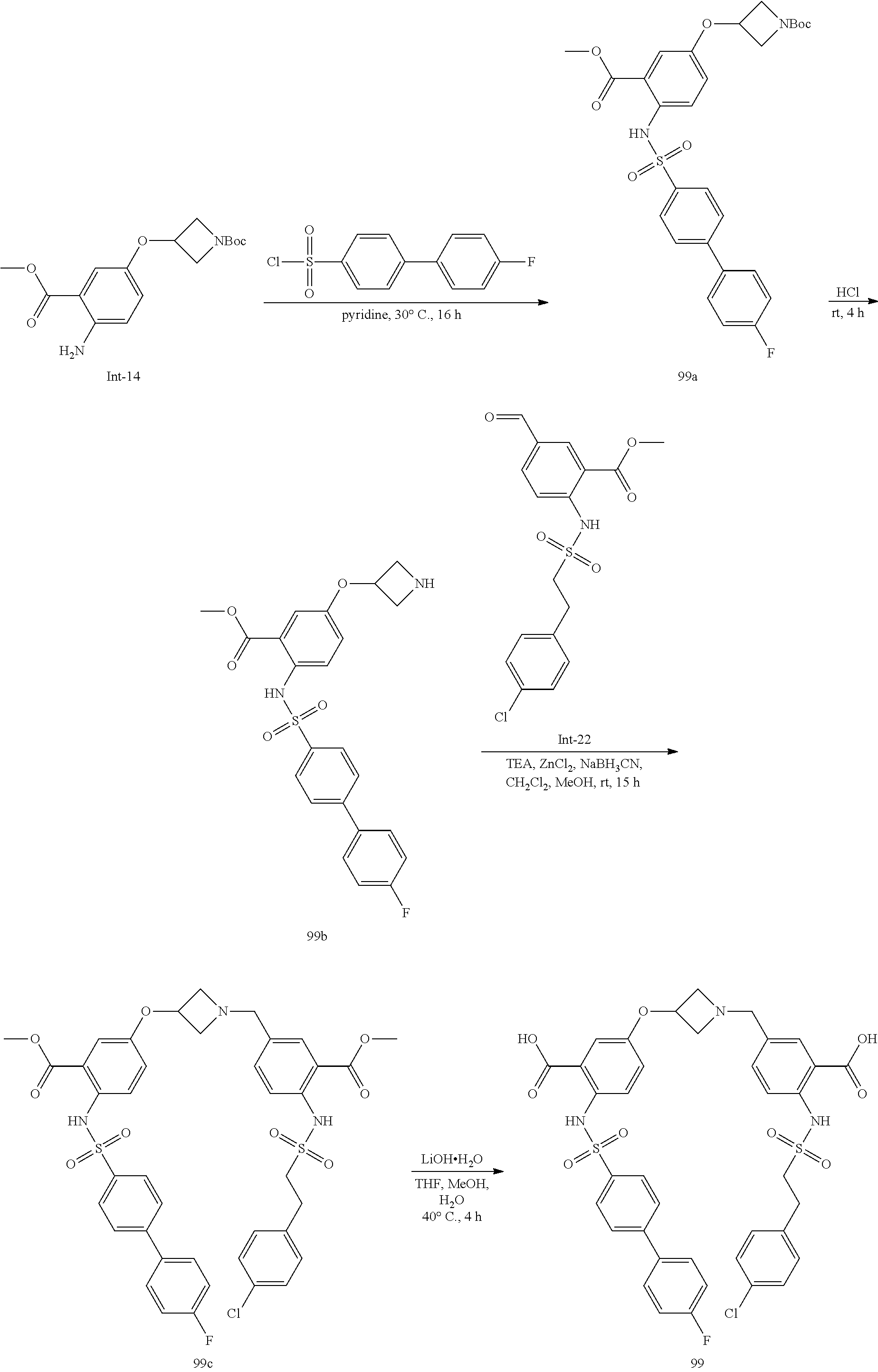

### Step 1: tert-butyl 3-(4-((4′-fluoro-(1,1′-biphenyl))-4-sulfonamido)-3-(methoxycarbonyl) phenoxy) azetidine-1-carboxylate (99a)

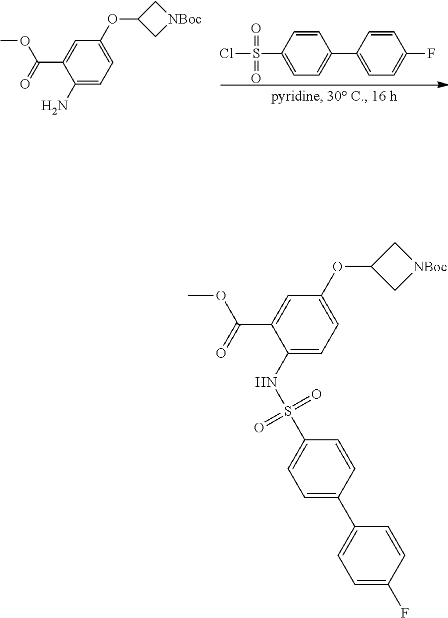 [SMILES: COC(=O)c1cc(OC2CN(C(=O)OC(C)(C)C)C2)ccc1N ; O=S(=O)(Cl)c1ccc(-c2ccc(F)cc2)cc1 ; COC(=O)c1cc(OC2CN(C(=O)OC(C)(C)C)C2)ccc1NS(=O)(=O)c1ccc(-c2ccc(F)cc2)cc1]

To a solution of tert-butyl 3-(4-amino-3-(methoxycarbonyl) phenoxy) azetidine-1-carboxylate (Int-14; 216 mg, 0.669 mmol) in pyridine (5 mL) was added 4′-fluoro-[1,1′-biphenyl]-4-sulfonyl chloride (190 mg, 0.702 mmol) at room temperature, which was then stirred for 16 h. The reaction mixture was concentrated in vacuo and the resulting residue was acidified with 1 N HCl, diluted with H₂O (30 mL), and extracted with EtOAc (2×30 mL). The organics were washed with brine (30 mL), dried over anh. MgSO4, filtered, and concentrated in vacuo. The resulting residue was purified by flash chromatography on SiO₂ [Isco®, Silica Flash 24 g Column, eluent of 0 to 60% EtOAc/Pet.ether gradient at 30 mL/min] to provide tert-butyl 3-(4-((4′-fluoro-[1,1′-biphenyl])-4-sulfonamido)-3-(methoxycarbonyl) phenoxy) azetidine-1-carboxylate. MS (ESI, m/z): 557.2 [M+H⁺].

### Step 2: methyl 5-(azetidin-3-yloxy)-2-((4′-fluoro-[1,l′-biphenyl])-4-sulfonamido)benzoate (99b)

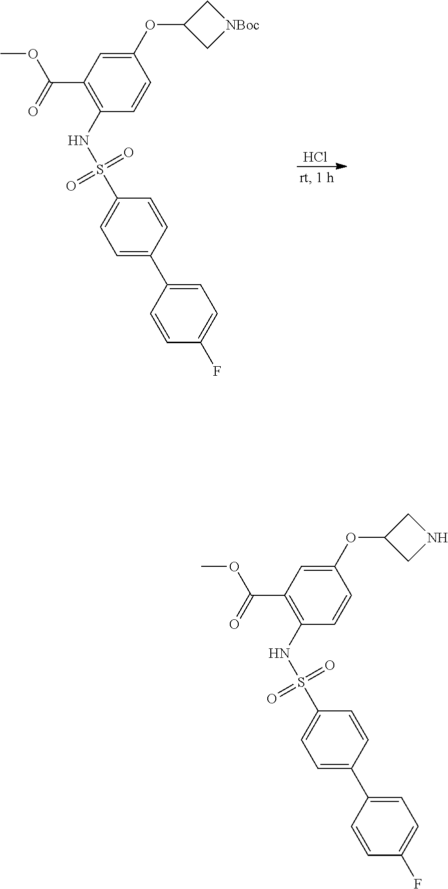 [SMILES: COC(=O)c1cc(OC2CN(C(=O)OC(C)(C)C)C2)ccc1NS(=O)(=O)c1ccc(-c2ccc(F)cc2)cc1 ; Cl ; COC(=O)c1cc(OC2CNC2)ccc1NS(=O)(=O)c1ccc(-c2ccc(F)cc2)cc1]

A mixture of tert-butyl 3-(4-((4′-fluoro-[1,1′-biphenyl])-4-sulfonamido)-3-(methoxy-carbonyl) phenoxy) azetidine-1-carboxylate (99a; 400 mg, 0.719 mmol) in HCl (4 N in EtOAc, 10 mL) was stirred at room temperature for 1 h. The reaction mixture was concentrated in vacuo to provide methyl 5-(azetidin-3-yloxy)-2-((4′-fluoro-[1,1′-biphenyl])-4-sulfonamido)benzoate, which was used in next subsequent step without further purification. MS (ESI, m/z): 457.1 [M+H⁺].

### Step 3: methyl 2-(2-(4-chlorophenyl)ethyl) sulfonamido)-5-(3-(4-((4′-fluoro-[1, l′-biphenyl])-4-sulfonamido)-3-(methoxycarbonyl) phenoxy) azetidin-1-yl)methyl)benzoate (99c)

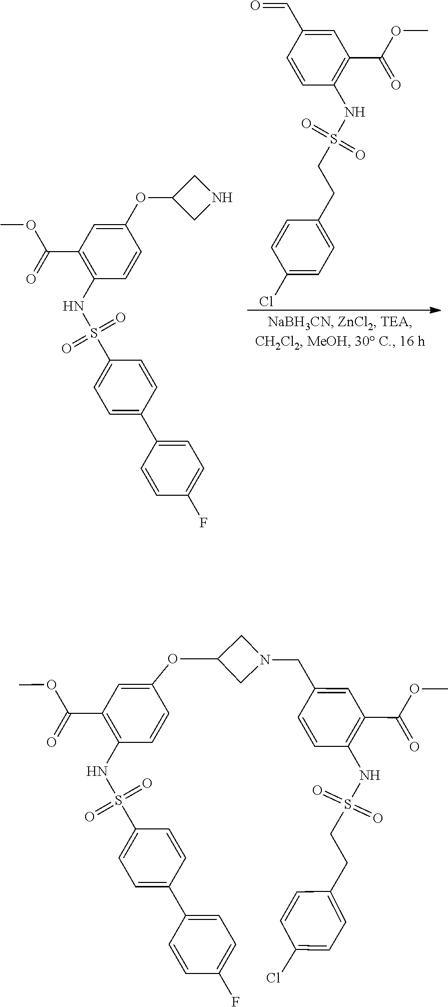

To a stirred mixture of methyl 5-(azetidin-3-yloxy)-2-((4′-fluoro-[1,1′-biphenyl])-4-sulfonamido)benzoate (99b; 320 mg, 0.701 mmol), methyl 2-((2-(4-chlorophenyl)ethyl) sulfonamido)-5-formylbenzoate (Int-22; 268 mg, 0.701 mmol), and zinc (II) chloride (191 mg, 1.402 mmol) in MeOH (2 mL) and CH₂Cl₂ (8 mL) was added TEA (0.293 mL, 2.103 mmol). The mixture was stirred at 30° C. for 0.5 h. Sodium cyanoborohydride (132 mg, 2.103 mmol) was added and the reaction mixture was stirred at 30° C. for 16 h. After concentrating in vacuo, the reaction mixture was diluted with EtOAc (100 mL), washed with aq. NH₄Cl (saturated, 50 mL), dried over anh. Na₂SO₄, filtered and concentrated in vacuo. The resulting residue was purified by preparative reverse phase chromatography [Boston Uni C18 column (150*40 mm, 5 μm), eluent of Mobile Phase A: H₂O (0.1% TFA) and Mobile Phase B: MeCN (0.1% TFA) at 220 nm] to provide methyl 2-((2-(4-chlorophenyl)ethyl) sulfonamido)-5-((3-(4-((4′-fluoro-[1,1′-biphenyl])-4-sulfonamido)-3-(methoxycarbonyl) phenoxy) azetidin-1-yl)methyl)benzoate. MS (ESI) m/z: 822.2 [M+H′].

### Step 4:5-((1-(3-carboxy-4-((2-(4-chlorophenyl)ethyl) sulfonamido)benzyl) azetidin-3-yl)oxy)-2-((4′-fluoro-[1, l′-biphenyl])-4-sulfonamido)benzoic acid (99)

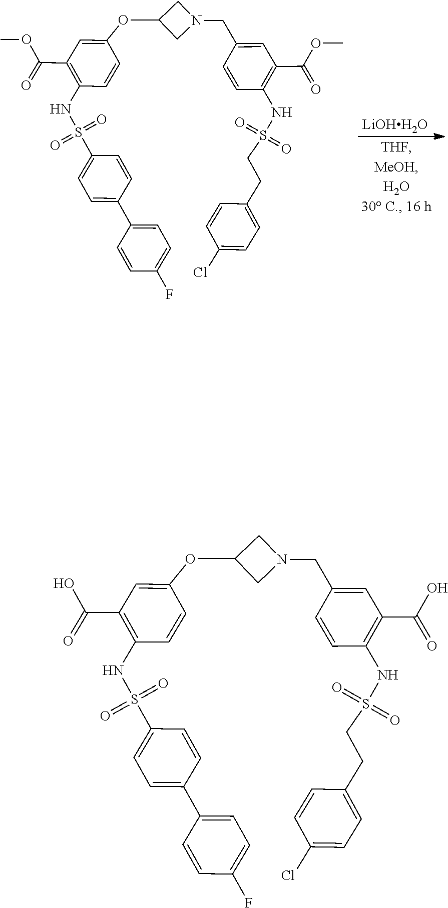

To a stirred mixture of methyl 2-((2-(4-chlorophenyl)ethyl) sulfonamido)-5-((3-(4-((4′-fluoro-[1, l′-biphenyl])-4-sulfonamido)-3-(methoxycarbonyl) phenoxy) azetidin-1-yl)methyl)benzoate (99c; 450 mg, 0.547 mmol) in THF (2 mL), MeOH (2 mL) and H₂O (1 mL) was added lithium hydroxide hydrate (230 mg, 5.47 mmol). The mixture was stirred at 30° C. for 16 h. Then the solvent was removed in vacuo, and the residue was acidified to pH=3 with IN aq. HCl. Then the mixture was purified by preparative reverse phase chromatography [Boston Green ODS column (150*30 mm, 5 μm), eluent of Mobile Phase A: H₂O (0.05% TFA) and Mobile Phase B: MeCN at 220 nm] to provide 5-((1-(3-carboxy-4-((2-(4-chlorophenyl)ethyl) sulfonamido)benzyl) azetidin-3-yl)oxy)-2-((4′-fluoro-[1,1′-biphenyl])-4-sulfonamido)benzoic acid. MS (ESI) m/z: 792.1 [M−H⁺]. ¹H NMR (500 MHZ, CD₃OD) δ ppm 8.17 (d, J=2.0 Hz, 1H), 7.79 (d, J=8.5 Hz, 2H), 7.73-7.69 (m, 2H), 7.69-7.59 (m, 5H), 7.38 (d, J=3.0 Hz, 1H), 7.21-7.15 (m, 2H), 7.14-7.06 (m, 5H), 5.11 (br s, 1H), 4.59 (dd, J=6.5, 12.0 Hz, 2H), 4.44 (s, 2H), 4.22 (br d, J=9.0 Hz, 2H), 3.59 (t, J=7.5 Hz, 2H), 3.06 (t, J=7.5 Hz, 2H)

The following compounds (100 through 108) of the present disclosure were made using the methods described in Example 20 above, and substituting the appropriate reactants and/or

| | | Observed |
|---|---|---|
| Compound | Structure | MS |
| 100 | 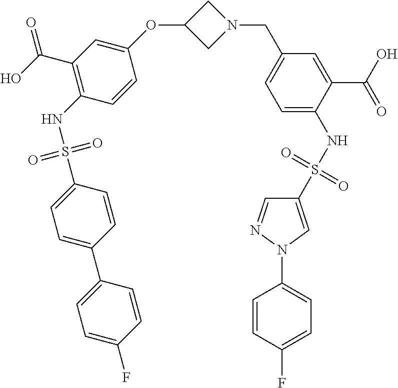 [SMILES: O=C(O)c1cc(CN2CC(Oc3ccc(NS(=O)(=O)c4ccc(-c5ccc(F)cc5)cc4)c(C(=O)O)c3)C2)ccc1NS(=O)(=O)c1cnn(-c2ccc(F)cc2)c1] | 816.4 |
| 101 | 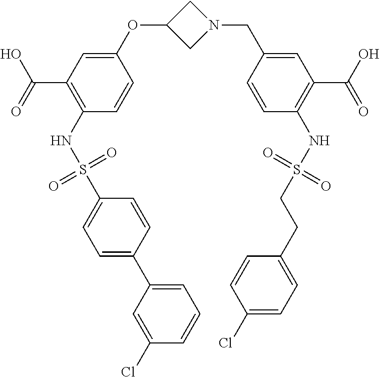 [SMILES: O=C(O)c1cc(CN2CC(Oc3ccc(NS(=O)(=O)c4ccc(-c5cccc(Cl)c5)cc4)c(C(=O)O)c3)C2)ccc1NS(=O)(=O)CCc1ccc(Cl)cc1] | 809.9 |
| 102 | 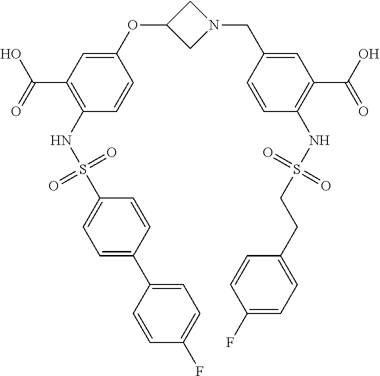 [SMILES: O=C(O)c1cc(CN2CC(Oc3ccc(NS(=O)(=O)c4ccc(-c5ccc(F)cc5)cc4)c(C(=O)O)c3)C2)ccc1NS(=O)(=O)CCc1ccc(F)cc1] | 778.5 |
| 103 | 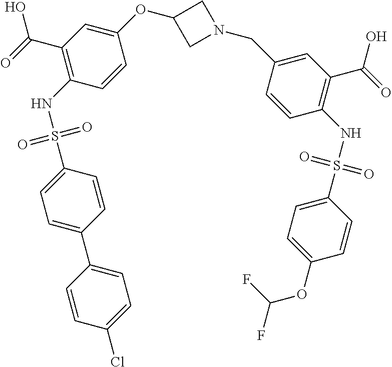 [SMILES: O=C(O)c1cc(CN2CC(Oc3ccc(NS(=O)(=O)c4ccc(-c5ccc(Cl)cc5)cc4)c(C(=O)O)c3)C2)ccc1NS(=O)(=O)c1ccc(OC(F)F)cc1] | 814.1 |
| 104 | 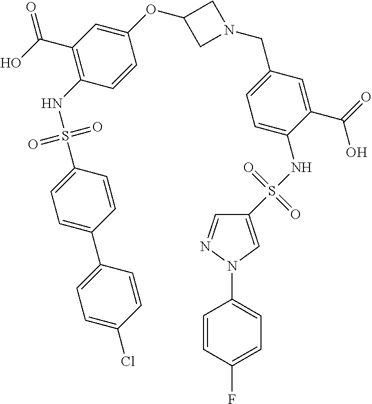 [SMILES: O=C(O)c1cc(CN2CC(Oc3ccc(NS(=O)(=O)c4ccc(-c5ccc(Cl)cc5)cc4)c(C(=O)O)c3)C2)ccc1NS(=O)(=O)c1cnn(-c2ccc(F)cc2)c1] | 832.5 |
| 105 | 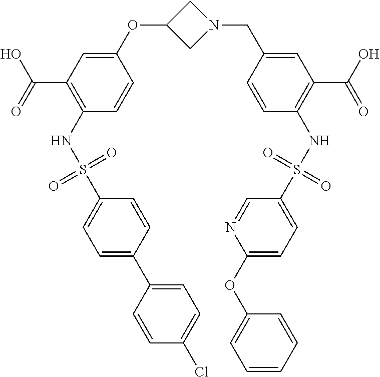 [SMILES: O=C(O)c1cc(CN2CC(Oc3ccc(NS(=O)(=O)c4ccc(-c5ccc(Cl)cc5)cc4)c(C(=O)O)c3)C2)ccc1NS(=O)(=O)c1ccc(Oc2ccccc2)nc1] | 841.2 |
| 106 | 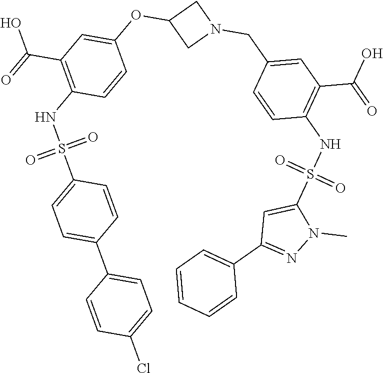 [SMILES: Cn1nc(-c2ccccc2)cc1S(=O)(=O)Nc1ccc(CN2CC(Oc3ccc(NS(=O)(=O)c4ccc(-c5ccc(Cl)cc5)cc4)c(C(=O)O)c3)C2)cc1C(=O)O] | 828.0 |
| 107 | 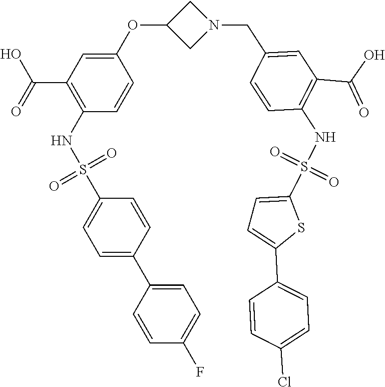 [SMILES: O=C(O)c1cc(OC2CN(Cc3ccc(NS(=O)(=O)c4ccc(-c5ccc(Cl)cc5)s4)c(C(=O)O)c3)C2)ccc1NS(=O)(=O)c1ccc(-c2ccc(F)cc2)cc1] | 847.9 |
| 108 | 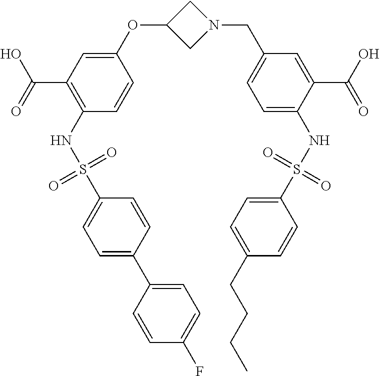 [SMILES: CCCCc1ccc(S(=O)(=O)Nc2ccc(CN3CC(Oc4ccc(NS(=O)(=O)c5ccc(-c6ccc(F)cc6)cc5)c(C(=O)O)c4)C3)cc2C(=O)O)cc1] | 788.3 |

### EXAMPLE 21

### Preparation of Compound 114

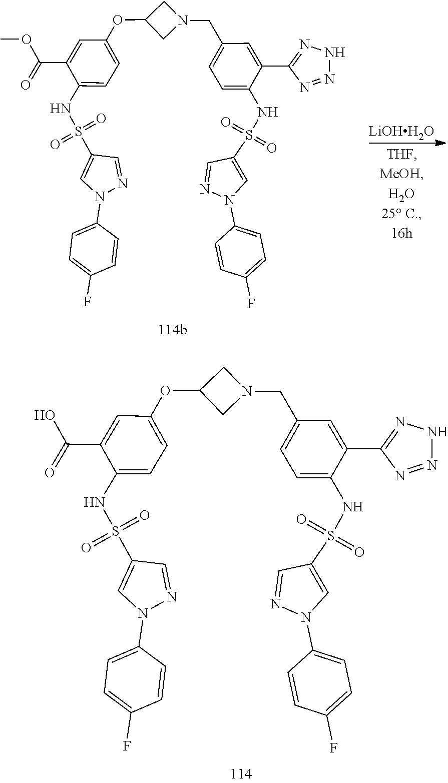 [SMILES: COC(=O)c1cc(OC2CN(Cc3ccc(NS(=O)(=O)c4cnn(-c5ccc(F)cc5)c4)c(-c4nn[nH]n4)c3)C2)ccc1NS(=O)(=O)c1cnn(-c2ccc(F)cc2)c1 ; "LiOH·H2O" ; O=C(O)c1cc(OC2CN(Cc3ccc(NS(=O)(=O)c4cnn(-c5ccc(F)cc5)c4)c(-c4nn[nH]n4)c3)C2)ccc1NS(=O)(=O)c1cnn(-c2ccc(F)cc2)c1]

### Step 1: methyl 5-((1-(3-cyano-4-((1-(4-fluorophenyl)-1H-pyrazole)-4-sulfonamido)benzyl) azetidin-3-yl)oxy)-2-((1-(4-fluorophenyl)-1H-pyrazole)-4-sulfonamido)benzoate (114a)

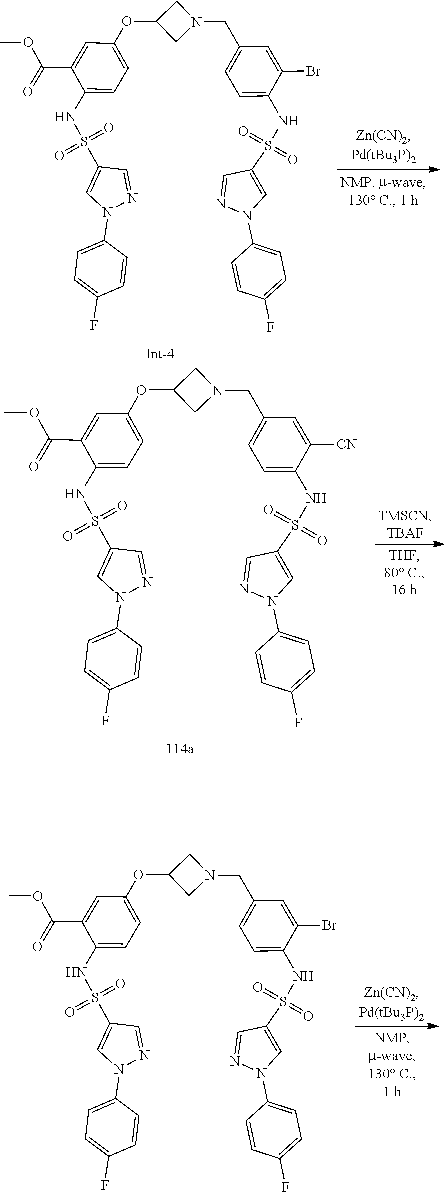 [SMILES: COC(=O)c1cc(OC2CN(Cc3ccc(NS(=O)(=O)c4cnn(-c5ccc(F)cc5)c4)c(Br)c3)C2)ccc1NS(=O)(=O)c1cnn(-c2ccc(F)cc2)c1 ; COC(=O)c1cc(OC2CN(Cc3ccc(NS(=O)(=O)c4cnn(-c5ccc(F)cc5)c4)c(C#N)c3)C2)ccc1NS(=O)(=O)c1cnn(-c2ccc(F)cc2)c1 ; COC(=O)c1cc(OC2CN(Cc3ccc(NS(=O)(=O)c4cnn(-c5ccc(F)cc5)c4)c(Br)c3)C2)ccc1NS(=O)(=O)c1cnn(-c2ccc(F)cc2)c1]

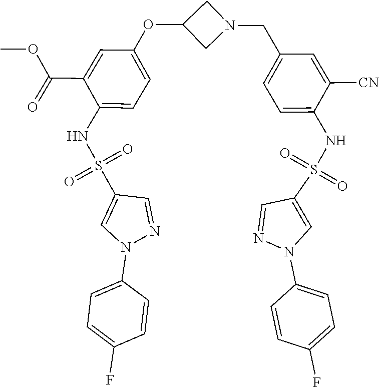 [SMILES: COC(=O)c1cc(OC2CN(Cc3ccc(NS(=O)(=O)c4cnn(-c5ccc(F)cc5)c4)c(C#N)c3)C2)ccc1NS(=O)(=O)c1cnn(-c2ccc(F)cc2)c1]

To a stirred mixture of methyl 5-((1-(3-bromo-4-((1-(4-fluorophenyl)-1H-pyrazole)-4-sulfonamido)benzyl) azetidin-3-yl)oxy)-2-((1-(4-fluorophenyl)-1H-pyrazole)-4-sulfonamido)benzoate (Int-4; 370 mg, 0.433 mmol) and zinc cyanide (508 mg, 4.33 mmol) in NMP (4 mL) was added bis(tri-ter(-butylphosphine)palladium (0) (44.2 mg, 0.087 mmol) in a glove box. The resulting mixture was irradiated at 130° C. for 1 h under N₂. Sat. aq. sodium hydrogen carbonate (20 mL) was added and the resulting mixture was extracted with EtOAc (2×20 mL). The combined organics were washed with brine (3×20 mL), dried over anh. Na₂SO₄, filtered. The filtrate was concentrated in vacuo and the resulting residue was purified by flash chromatography on SiO₂ [Isco®, Silica Flash 12 g column, eluent of 0 to 70% EtOAc/Pet. Ether gradient at 30 mL/min| to provide methyl 5-((1-(3-cyano-4-((1-(4-fluorophenyl)-1H-pyrazole)-4-sulfonamido)-benzyl) azetidin-3-yl)oxy)-2-((1-(4-fluorophenyl)-1H-pyrazole)-4-sulfonamido)benzoate. MS (ESI) m/z: 801.2 [M+H⁺].

### Step 2: methyl 2-((1-(4-fluorophenyl)-1H-pyrazole)-4-sulfonamido)-5-((1-(4-((1-(4-fluorophenyl)-1H-pyrazole)-4-sulfonamido)-3-(2H-tetrazol-5-yl)benzyl) azetidin-3-yl)oxy)benzoate (114b)

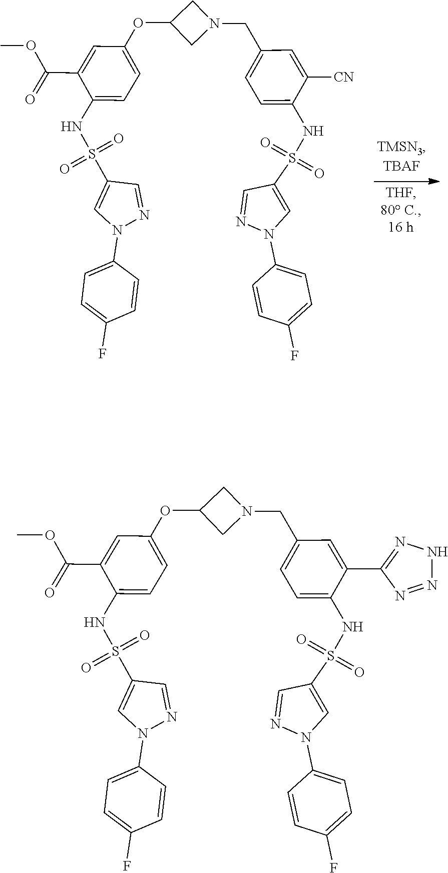 [SMILES: COC(=O)c1cc(OC2CN(Cc3ccc(NS(=O)(=O)c4cnn(-c5ccc(F)cc5)c4)c(C#N)c3)C2)ccc1NS(=O)(=O)c1cnn(-c2ccc(F)cc2)c1 ; COC(=O)c1cc(OC2CN(Cc3ccc(NS(=O)(=O)c4cnn(-c5ccc(F)cc5)c4)c(-c4nn[nH]n4)c3)C2)ccc1NS(=O)(=O)c1cnn(-c2ccc(F)cc2)c1]

To a stirred mixture of methyl 5-((1-(3-cyano-4-((1-(4-fluorophenyl)-1H-pyrazole)-4-sulfonamido)benzyl) azetidin-3-yl)oxy)-2-((1-(4-fluorophenyl)-1H-pyrazole)-4-sulfonamido)benzoate (114a; 180 mg, 0.225 mmol) and TMSN3 (0.045 ml, 0.337 mmol) in THF (2 mL) was added TBAF (0.112 mL, 0.112 mmol). The reaction mixture was stirred at 80° C. for 16 h, concentrated in vacuo, and the resulting residue was purified by flash chromatography on SiO₂ [Isco®, Silica Flash 12 g column, eluent of 0 to 100% EtOAc/Pet. ether gradient at 20 mL/min] to provide product, which was further purified by reverse phase chromatography [Gilson 281, Phenomenex Synergi C18 column (150 mm*30 mm, 4 μm), eluent of mobile phase A: water (0.1% TFA) and mobile phase B: MeCN at 220 nm] to provide methyl 2-((1-(4-fluorophenyl)-1H-pyrazole)-4-sulfonamido)-5-((1-(4-((1-(4-fluorophenyl)-1H-pyrazole)-4-sulfonamido)-3-(2H-tetrazol-5-yl)benzyl) azetidin-3-yl)oxy)benzoate. MS (ESI) m/z: 844.2 [M+H⁺].

### Step 3:2-((1-(4-fluorophenyl)-1H-pyrazole)-4-sulfonamido)-5-((1-(4-((1-(4-fluoro phenyl)-1H-pyrazole)-4-sulfonamido)-3-(2H-tetrazol-5-yl)benzyl) azetidin-3-yl)oxy)benzoic acid (114)

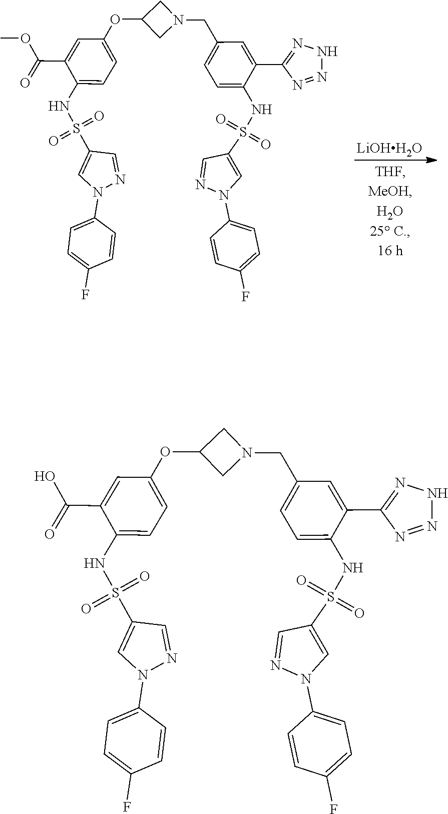 [SMILES: COC(=O)c1cc(OC2CN(Cc3ccc(NS(=O)(=O)c4cnn(-c5ccc(F)cc5)c4)c(-c4nn[nH]n4)c3)C2)ccc1NS(=O)(=O)c1cnn(-c2ccc(F)cc2)c1 ; "LiOH·H2O" ; O=C(O)c1cc(OC2CN(Cc3ccc(NS(=O)(=O)c4cnn(-c5ccc(F)cc5)c4)c(-c4nn[nH]n4)c3)C2)ccc1NS(=O)(=O)c1cnn(-c2ccc(F)cc2)c1]

To a stirred mixture of methyl 2-((1-(4-fluorophenyl)-1H-pyrazole)-4-sulfonamido)-5-((1-(4-((1-(4-fluorophenyl)-1H-pyrazole)-4-sulfonamido)-3-(2H-tetrazol-5-yl)benzyl) azetidin-3-yl)oxy)benzoate (114b; 20 mg, 0.024 mmol) in THF (0.2 mL), MeOH (0.200 mL), and H₂O (0.1 mL) was added lithium hydroxide monohydrate (9.95 mg, 0.237 mmol). The mixture was stirred at 25° C. for 16 h, then concentrated in vacuo. The resulting residue was acidified to pH=3 with IN aq. HCl, then purified by reverse phase chromatography [Boston Green ODS column (150 mm*30 mm, 5 μm), eluent of mobile phase A: water (0.1% TFA) and mobile phase B: MeCN at 220 nm] to provide 2-((1-(4-fluorophenyl)-1H-pyrazole)-4-sulfonamido)-5-((1-(4-((1-(4-fluorophenyl)-1H-pyrazole)-4-sulfonamido)-3-(2H-tetrazol-5-yl)benzyl) azetidin-3-yl)oxy)benzoic acid. MS (ESI) m/z: 830.2 [M+H⁺]. ¹H NMR (500 MHZ, CD₃OD) δ ppm 8.64 (d, J=9.0 Hz, 2H), 8.11 (d, J=2.0 Hz, 1H), 7.88 (d, J=8.5 Hz, 1H), 7.75 (d, J=9.0 Hz, 2H), 7.71-7.70 (m, 3H), 7.67-7.63 (m, 2H), 7.61 (dd, J=2.0, 8.5 Hz, 1H), 7.40 (d, J=3.0 Hz, 1H), 7.23-7.16 (m, 4H), 7.12 (dd, J=3.0, 9.0 Hz, 1H), 5.14-5.09 (m, 1H), 4.64-4.60 (m, 2H), 4.48 (s, 2H), 4.27-4.24 (m, 2H).

### EXAMPLE 22

### Preparation of Compound 115

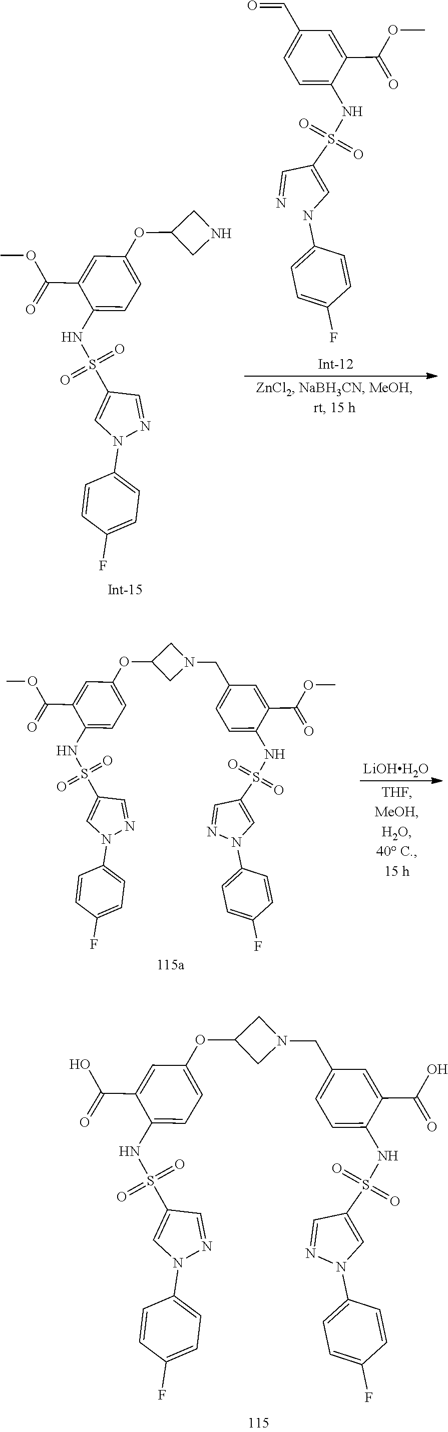

### Step 1: methyl 2-(1-(4-fluorophenyl)-1H-pyrazole-4-sulfonamido)-5-((1-(4-(1-(4-fluorophenyl)-1H-pyrazole-4-sulfonamido)-3-(methoxycarbonyl)benzyl) azetidin-3-yl)oxy)benzoate (115a)

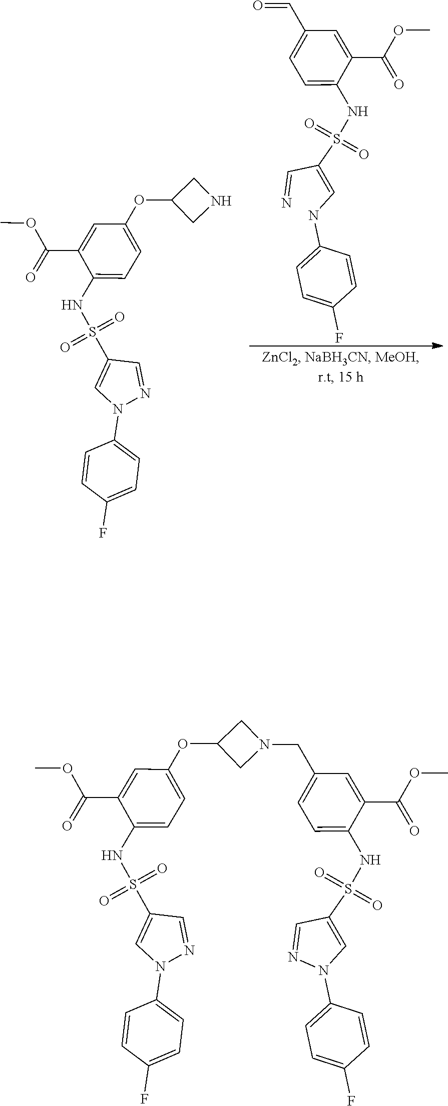

To a solution of methyl 5-(azetidin-3-yloxy)-2-(1-(4-fluorophenyl)-1H-pyrazole-4-sulfonamido)benzoate (Int-15; 2.7 g, 6.05 mmol) in MeOH (10 mL) and DMF (10 mL) was added methyl 2-(1-(4-fluorophenyl)-1H-pyrazole-4-sulfonamido)-5-formylbenzoate (Int-12; 2.49 g, 6.17 mmol), zinc (II) chloride (1.648 g, 12.10 mmol), and NaBH₃CN (1.140 g, 18.14 mmol) at room temperature. The reaction mixture was stirred at room temperature for 15 h, concentrated in vacuo, and the resulting residue was diluted with H₂O (50 mL) and extracted with EtOAc (3×20 mL). The combined organic layers were dried over anh. Na₂SO₄, filtered, and concentrated in vacuo. The resulting residue was purified by flash chromatography on SiO₂ [Isco®, SepaFlash® Silica Flash 40 g column, eluent of 0 to 100% EtOAc/Pet.ether gradient at 60 mL/min] to provide methyl 2-(1-(4-fluorophenyl)-1H-pyrazole-4-sulfonamido)-5-((1-(4-(1-(4-fluorophenyl)-1H-pyrazole-4-sulfonamido)-3-(methoxy carbonyl)benzyl) azetidin-3-yl)oxy)benzoat. MS (ESI) m/z: 834.1 [M+H⁺]. ¹H NMR (400 MHZ, CD₃OD) δ ppm 8.80 (s, 1H), 8.62 (s, 1H), 8.06 (s, 1H), 7.92 (s, 1H), 7.81 (d, J=8.8 Hz, 1H), 7.77 (s, 1H), 7.69-7.76 (m, 5H), 7.61-7.65 (m, 1H), 7.31 (d, J=2.8 Hz, 1H), 7.21 (dt, J=8.8, 4.4 Hz, 4H), 7.10 (dd, J=9.2, 3.2 Hz, 1H), 4.97 (br s, 1H), 4.23 (br s, 2H), 4.12 (br s, 2H), 3.90 (s, 3H), 3.80-3.84 (m, 5H).

### Step 2:5-((1-(3-carboxy-4-(1-(4-fluorophenyl)-1H-pyrazole-4-sulfonamido)benzyl) azetidin-3-yl)oxy)-2-(1-(4-fluorophenyl)-1H-pyrazole-4-sulfonamido)benzoic acid (115)

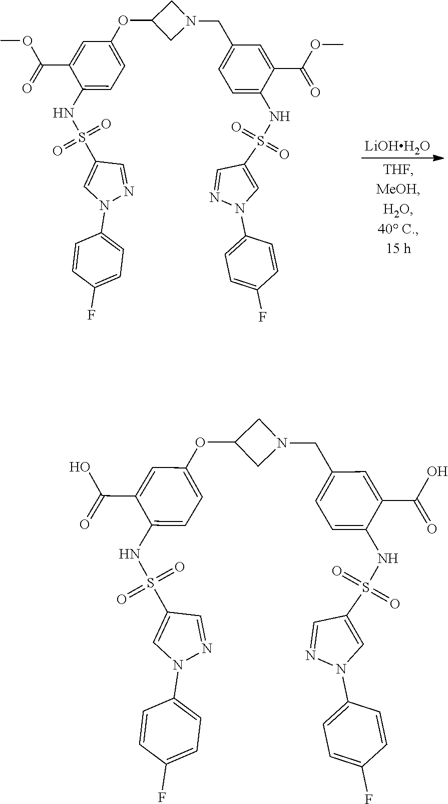

To a solution of methyl 2-(1-(4-fluorophenyl)-1H-pyrazole-4-sulfonamido)-5-((1-(4-(1-(4-fluorophenyl)-1H-pyrazole-4-sulfonamido)-3-(methoxycarbonyl)benzyl) azetidin-3-yl)oxy)benzoate (115a; 3.78 g, 4.53 mmol) in THF (20 mL), MeOH (20 mL), and H₂O (10 mL) was added lithium hydroxide hydrate (1.902 g, 45.3 mmol) at room temperature. The reaction mixture was stirred at 40° for 15 h, then acidified to pH=2 with 3 N aq. HCl, concentrated in vacuo, and the resulting residue was diluted with H₂O (50 mL) and extracted with EtOAc (3×20 mL). The combined organics were dried over anh. Na₂SO₄, filtered, and concentrated in vacuo. The resulting residue was purified by reverse phase chromatography [Gilson 281, Phenomenex Synergi C18 column (150 mm*30 mm, 4 μm), eluent of mobile phase A: water (0.05% HCl) and mobile phase B: MeCN at 220 nm] to provide 5-((1-(3-carboxy-4-(1-(4-fluorophenyl)-1H-pyrazole-4-sulfonamido)-benzyl)-azetidin-3-yl)oxy)-2-(1-(4-fluorophenyl)-1H-pyrazole-4-sulfonamido)benzoic acid.

MS (ESI) m/z: 804.2 [M−H⁺]. ¹H NMR (500 MHZ, CD₃OD) δ ppm 8.82 (s, 1H), 8.65 (s, 1H), 8.19 (d, J=2.0 Hz, 1H), 7.93 (s, 1H), 7.85 (d, J=8.5 Hz, 1H), 7.70-7.78 (m, 6H), 7.67-7.70 (m, 1H), 7.40 (d, J=3.0 Hz, 1H), 7.16-7.24 (m, 4H), 7.11 (dd, J=9.0, 3.0 Hz, 1H), 5.06-5.15 (m, 1H), 4.58 (br dd, J=12.0, 6.5 Hz, 2H), 4.44 (s, 2H), 4.21 (br dd, J=12.0, 4.0 Hz, 2H).

The following compounds (116-118 and 120-121) of the present disclosure were made using the methods described in Example 22 above, and substituting the appropriate reactants and/or reagents:

| | | Observed |
|---|---|---|
| Compound | Structure | MS |
| 116 | 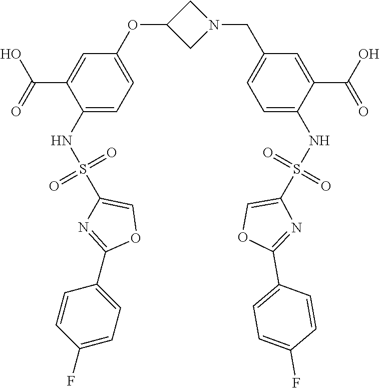 [SMILES: O=C(O)c1cc(CN2CC(Oc3ccc(NS(=O)(=O)c4coc(-c5ccc(F)cc5)n4)c(C(=O)O)c3)C2)ccc1NS(=O)(=O)c1coc(-c2ccc(F)cc2)n1] | 805.9 |
| 117 | 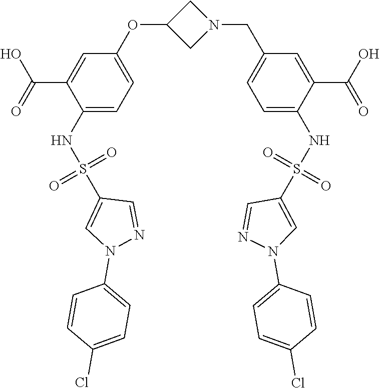 [SMILES: O=C(O)c1cc(CN2CC(Oc3ccc(NS(=O)(=O)c4cnn(-c5ccc(Cl)cc5)c4)c(C(=O)O)c3)C2)ccc1NS(=O)(=O)c1cnn(-c2ccc(Cl)cc2)c1] | 836.2 |
| 118 | 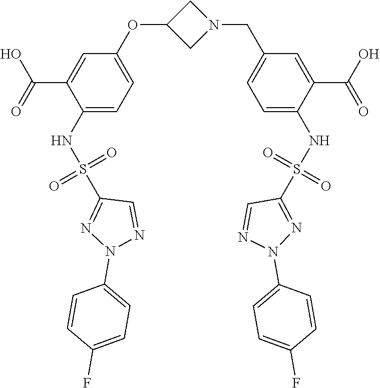 [SMILES: O=C(O)c1cc(CN2CC(Oc3ccc(NS(=O)(=O)c4cnn(-c5ccc(F)cc5)n4)c(C(=O)O)c3)C2)ccc1NS(=O)(=O)c1cnn(-c2ccc(F)cc2)n1] | 806.2 |
| 120 | 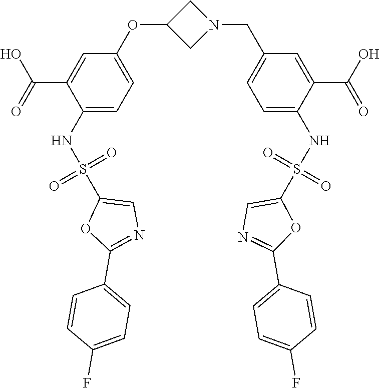 [SMILES: O=C(O)c1cc(CN2CC(Oc3ccc(NS(=O)(=O)c4cnc(-c5ccc(F)cc5)o4)c(C(=O)O)c3)C2)ccc1NS(=O)(=O)c1cnc(-c2ccc(F)cc2)o1] | 806.1 |
| 121 | 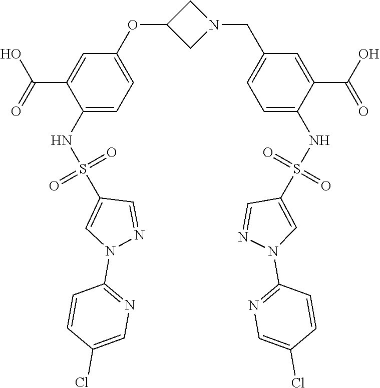 [SMILES: O=C(O)c1cc(CN2CC(Oc3ccc(NS(=O)(=O)c4cnn(-c5ccc(Cl)cn5)c4)c(C(=O)O)c3)C2)ccc1NS(=O)(=O)c1cnn(-c2ccc(Cl)cn2)c1] | 838.0 |

Illustrative compounds of the present disclosure were tested in one or more of the above assays and results are provided in the table below:

### EXAMPLE 123

### Target Identification by Isolation of Compound-Resistant Mutants

To identify the potential target of the claimed molecules mutants in A. baumannii were isolated as described here in Example 123 that are resistant to elevated concentrations of the test article. Briefly, rare, spontaneous resistant mutants were identified following growth of bacteria on solid growth media containing a test article. Colonies that grew in the presence of elevated concentrations of the test article were picked with a sterile loop and purified by restreaking on solid growth media containing the test article. Genomic DNA was purified from resistant isolates and subjected to whole genome sequencing. Following comparison of the genome sequences of resistant mutants to that of wild-type, parental strain using established computational sequence alignment, comparison and annotation methods, substitutions in the DNA sequence were observed that made nonsynonymous amino acid changes in the translated sequence of the MsbA open reading frame. An example of target identification by isolation and characterization of resistant mutants can be found in Howe, et al., Nature, 526 (7575): 672-7, 2015.

Target identification was done essentially as described previously with minor modifications (Howe, J. A., et al. Selective small-molecule inhibition of an RNA structural element. Nature (2015). 526:672-7.). A. baumannii strain ATCC19606 (obtained from the American Type Culture Collection, www.atcc.org) was incubated overnight in CAMHB at 37° C., 200 RPM to late-exponential phase (approximately 2×10⁹ colony forming units (cfu)/ml). One hundred μl of the above culture was spread on each of CAMH agar plates containing 2-fold escalating liquid minimum inhibitory concentration (MIC) levels of compound. The plates were incubated at 37° C. for 48 hours. Resistant isolates that arose were counted and re-streaked on plates containing four-fold MIC concentration of respective compound. The frequency of resistance (FOR) was determined, dividing the number of resistant isolates by the viable cfu in the late-exponential inoculum. Genomic DNA from purified resistant isolates was prepared and subjected to whole genome sequencing. Polymorphism were identified in the resistant isolates by comparison to the wild-type parental strain. Analysis indicated single amino acid substitutions in MsbA were associated with resistance to examples of the claimed molecules.

Following identification of A. baumannii isolates expressing specific mutations in MsbA that confer resistance to initial hit molecules, the strains can be further used to assess on-target activity of modified molecules. To assess on-target activity of additional test molecules using these resistant mutants, the antimicrobial potency (as described in Example 124) of test molecules was compared with that of the wild-type, parental strain. If the test molecule was less potent, generally having, for example, an MITC95 of at least 2 to 4-fold higher against the resistant mutant than the wild-type parental strain then it was considered to be acting through MsbA inhibition.

### EXAMPLE 124

Antimicrobial potency

The concentrations of compounds required to inhibit the growth of various strains of bacteria were determined in an assay that assessed bacterial growth by measuring optical density at 600 nm (OD600). Test articles were dissolved in 100% DMSO and serially diluted two-fold from their maximal concentration in 100% DMSO. Forty-nine μl of bacterial inoculum (at approx. 2×105 cfu/mL in CAMHB) were put into wells of an assay plate. One μl of compound or DMSO was transferred from the source plate to the assay plate. The completed assay plates were then incubated at 35+2degC for 18-22 h. Bacterial growth was measured by reading OD600 on a spectrophotometer. Data analysis calculates the lowest concentration of compound which results in >95% growth inhibition (minimum inhibitory threshold concentration 95%, MITC95). Antimicrobial potency of test compounds was determined, and the results of that testing are shown in Table 1.

TABLE 1
Illustrative compounds of the present disclosure
were tested in one or more of the above assays
and results are provided in the table below:

| | Antibacterial | |
|---|---|---|
| | assay 1^{a} | |
| Compound | (MITC95) | |
| 1 | 277 | nM |
| 2 | 69.2 | nM |
| 3 | 277 | nM |
| 12 | 138 | nM |
| 13 | 196 | nM |
| 14 | 24.5 | nM |
| 15 | 24.5 | nM |
| 16 | <48.8 | nM |
| 17 | <48.8 | nM |
| 18 | 12.2 | nM |
| 19 | 24.5 | nM |
| 20 | 24.5 | nM |
| 21 | 48.9 | nM |
| 22 | 48.9 | nM |
| 23 | 34.6 | nM |
| 24 | 277 | nM |
| 25 | 155 | nM |
| 26 | 48.9 | nM |
| 27 | 391 | nM |
| 28 | 196 | nM |
| 29 | 48.9 | nM |
| 30 | 277 | nM |
| 31 | 196 | nM |
| 32 | 24.5 | nM |
| 33 | 48.9 | nM |
| 34 | 48.9 | nM |
| 35 | 48.9 | nM |
| 36 | 48.9 | nM |
| 37 | 69.2 | nM |
| 38 | 391 | nM |
| 39 | N/A | |
| 40 | 48.9 | nM |
| 41 | 12.2 | nM |
| 42 | 48.9 | nM |
| 43 | 48.9 | nM |
| 44 | 391 | nM |
| 45 | 97.8 | nM |
| 46 | 391 | nM |
| 47 | 1570 | nM |
| 48 | 321 | nM |
| 49 | 1560 | nM |
| 50 | 391 | nM |
| 51 | 3130 | nM |
| 52 | 553 | nM |
| 53 | N/A | |
| 54 | 34.6 | nM |
| 56 | 97.8 | nM |
| 58 | 24.5 | nM |
| 59 | 24.5 | nM |
| 60 | 12.2 | nM |
| 61 | 196 | nM |
| 62 | <48.8 | nM |
| 63 | 196 | nM |
| 65 | 48.9 | nM |
| 66 | 48.9 | nM |
| 67 | 24.5 | nM |
| 69 | 24.5 | nM |
| 71 | 97.8 | nM |
| 72 | 97.8 | nM |
| 73 | 196 | nM |
| 74 | 310 | nM |
| 75 | 277 | nM |
| 78 | 782 | nM |
| 79 | 97.8 | nM |
| 80 | 12.2 | nM |
| 81 | 12.2 | nM |
| 82 | 34.6 | nM |
| 83 | 34.6 | nM |
| 84 | 69.1 | nM |
| 85 | 17.3 | nM |
| 86 | 24.5 | nM |
| 87 | 24.5 | nM |
| 88 | 12.2 | nM |
| 89 | <3.06 | nM |
| 90 | 8.65 | nM |
| 91 | 17.3 | nM |
| 92 | 6.11 | nM |
| 93 | 6.11 | nM |
| 94 | 8.6 | nM |
| 95 | 12.2 | nM |
| 96 | 12.2 | nM |
| 97 | 97.8 | nM |
| 98 | 69.2 | nM |
| 99 | 24.5 | nM |
| 100 | 48.9 | nM |
| 101 | 24.5 | nM |
| 102 | 48.9 | nM |
| 103 | 24.5 | nM |
| 104 | 34.6 | nM |
| 105 | 24.5 | nM |
| 106 | 48.9 | nM |
| 107 | 48.9 | nM |
| 108 | 48.9 | nM |
| 114 | 246 | nM |
| 115 | 277 | nM |
| 116 | 97.8 | nM |
| 117 | 48.9 | nM |
| 118 | 24.5 | nM |
| 120 | 48.9 | nM |
| 121 | 277 | nM |

N/A = data not available
^{a}= data generated using the assay described in Example 124

While the disclosure has been described and illustrated with reference to certain particular embodiments thereof, those skilled in the art will appreciate that various adaptations, changes, modifications, substitutions, deletions, or additions of procedures and protocols may be made without departing from the spirit and scope of the disclosure. For example, effective dosages other than the particular dosages as set forth herein above may be applicable as a consequence of variations in the responsiveness of the mammal being treated for any of the indications with the compounds of the disclosure indicated above. Likewise, the specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present disclosure. It is intended, therefore, that the disclosure be defined by the scope of the claims which follow and that such claims be interpreted as broadly as is reasonable.

1. A compound represented by structural Formula I: 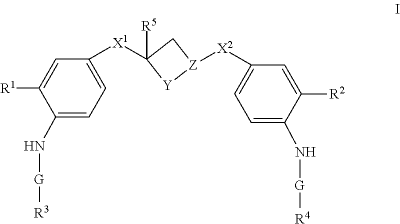
X¹ and X² are independently selected from —O—, —CH₂—, —CH₂O—, —OCH₂—, —NHCH₂—, and —CH₂NH—;
Y is selected from —CHRⁱ—, —O—, and —NH—;
Z is selected from —N—, —CH—, —CH—CH₂, and —C—(CH₂—) (CH₂—);
R¹ and R² are independently selected from —(CH₂)ₙC(O)OR, —C(O) NHSO₂C₁₋₆alkyl, —SO₂OH, —SO₂OCl, and tetrazolyl;
R is selected from H and C₁₋₆alkyl;
R^{j} is selected from H, C₁₋₆alkyl, aryl, heterocycloalkyl, and heteroaryl, said alkyl, aryl, heterocycloalkyl and heteroaryl optionally substituted with 1 to 3 groups of R^{a};
each G is independently selected from —S(O₂)—, —C(O)C(O)NH—, and —C(O)—;
R³ and R⁴ are independently selected from —C₁₋₆alkyl, —(CH₂)₁₋₄C(O), C₆₋₁₀aryl, heterocycloalkyl, and heteroaryl, said aryl, heterocycloalkyl and heteroaryl optionally substituted with 1 to 3 groups of R^{a},
R⁵ is selected from hydrogen and C₁₋₆alkyl;
R^{a} is selected from C₁₋₆alkyl, —OC₁₋₃haloalkyl, phenyl, —Ophenyl, and pyridyl, said alkyl, phenyl and pyridyl optionally substituted with 1 to 3 groups of R^{b};
R^{b} is selected from OH, phenyl, and halogen; and
n is 0, 1, 2, or 3.

2. The compound according to claim 1, or a pharmaceutically acceptable salt thereof, wherein X¹ and X² respectively, are selected from 1) —O—and CH₂; 2) —OCH₂— and —CH₂O—; 3) both are —NHCH₂—; 4) —O—and —CH₂O—; 5) both are —CH₂O—.

3. The compound according to claim 1, or a pharmaceutically acceptable salt thereof, wherein Y is —CH₂—.

4. The compound according to claim 1, or a pharmaceutically acceptable salt thereof, wherein Z is —CH—.

5. The compound according to claim 1, or a pharmaceutically acceptable salt thereof, wherein Z is —N—.

6. The compound of claim 1, or a pharmaceutically acceptable salt thereof, wherein R¹ and R² are both —(CH₂)ₙC(O)OR.

7. The compound of claim 1 or a pharmaceutically acceptable salt thereof, wherein R¹ and R² are both —(CH₂)ₙC(O)OH.

8. The compound according to claim 1, or a pharmaceutically acceptable salt thereof, wherein G is —S(O₂)—, or —C(O)C(O)NH—.

9. The compound according to claim 1, or a pharmaceutically acceptable salt thereof, wherein R³ and R⁴ are both optionally substituted C₆₋₁₀aryl, heteroaryl or heterocycloalkyl.

10. The compound according to claim 1, or a pharmaceutically acceptable salt thereof, wherein one of R³ and R⁴ is pyrazolyl and the other is selected from phenyl, morpholinyl, azetidinyl, oxazolidinyl, pyrazolyl, triazolyl, thienyl, thiazolyl, and oxazolyl, each R³ and R⁴ optionally substituted with 1 to 3 groups of R^{a}.

11. The compound according to claim 1, or a pharmaceutically acceptable salt thereof, wherein R³ and R⁴ are both pyrazolyl optionally substituted with 1 to 3 groups of R^{a}.

12. The compound according to claim 1, or a pharmaceutically acceptable salt thereof, wherein one of R³ and R⁴ is C₁-6alkyl and the other is selected from C₁₋₆alkyl, phenyl, morpholinyl, azetidinyl, oxazolidinyl, pyrazolyl, triazolyl, thienyl, thiazolyl, and oxazolyl, each R³ and R⁴ optionally substituted with 1 to 3 groups of R^{a}.

13. The compound according to claim 1, or a pharmaceutically acceptable salt thereof, wherein R^{a} is phenyl, or —Ophenyl, said phenyl moiety optionally substituted with 1 to 3 groups of R^{b}.

14. The compound according to claim 13, or a pharmaceutically acceptable salt thereof, wherein said phenyl moiety of phenyl or Ophenyl is substituted with 1 to 3 groups of chlorine or fluorine.

15. The compound according to claim 1 represented by structural Formula II, or a pharmaceutically acceptable salt thereof: 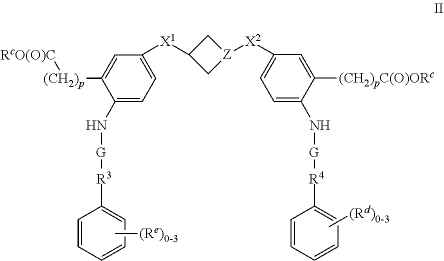
wherein X¹, X², Z, and G, are as defined in claims 1, R³ and R⁴ are independently selected from —C₁₋₆alkyl, —(CH₂)₁₋₄C(O), aryl, heterocycloalkyl, and heteroaryl, said aryl, heterocycloalkyl and heteroaryl optionally substituted with 1 to 2 groups of R^{a}; each R^{c} is independently selected from hydrogen, CH₃, and CH₂CH₃, p is 0 or 1, and R^{c} and R^{d}, when present, are independently selected from chlorine and fluorine.

16. The compound of Formula II, or a pharmaceutically acceptable salt thereof according to claim 15 wherein X¹ and X², respectively are selected from 1) —O—and CH₂, 2) —CH₂O—and —CH₂—, 3) —OCH₂— and —NHCH₂—, 4) —NHCH₂—, 5) CH₂NH and —NHCH₂—; R^{c} and R^{d}, when present, are independently selected from chlorine and fluorine; Z is N or CH.

17. The compound of Formula II or a pharmaceutically acceptable salt thereof according to claim 15, wherein R³ and R⁴ are both optionally substituted aryl, heteroaryl or heterocycloalkyl.

18. The compound of Formula II, or a pharmaceutically acceptable salt thereof according to claim 15, wherein one of R³ and R⁴ is pyrazolyl and the other is selected from phenyl, morpholinyl, azetidinyl, oxazolidinyl, pyrazolyl, triazolyl, thienyl, thiazolyl, and oxazolyl, said R³ and R⁴ optionally substituted with 1 to 3 groups of R^{a}.

19. The compound of Formula II, or a pharmaceutically acceptable salt thereof according to claim 15, wherein R³ and R⁴ are both optionally substituted pyrazolyl.

20. The compound according to claim 1 represented by structural Formula III, or a pharmaceutically acceptable salt thereof: 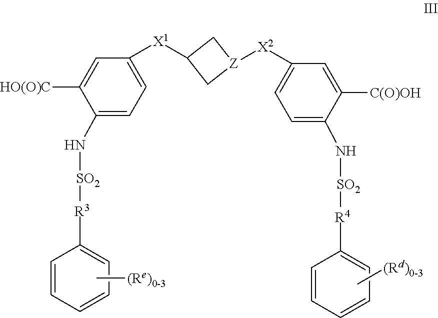
wherein X¹, X², and Z, are as defined in claims 1, R³ and R⁴ are independently selected from —C₁₋₆alkyl, —(CH₂)₁₋₄C(O), C₆₋₁₀aryl, C₃₋₁₀heterocycloalkyl, and C₃₋₁₀heteroaryl, said aryl, heterocycloalkyl and heteroaryl optionally substituted with 1 to 2 groups of R^{a}, and R^{c} and R^{d}, when present, are independently selected from chlorine and fluorine.

21. A compound, or a pharmaceutically acceptable salt thereof selected from:

| Compound | Structure |
|---|---|
| 1 | 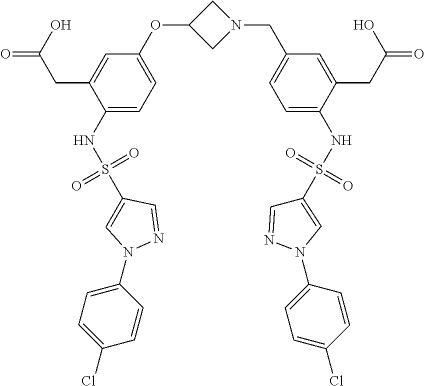 [SMILES: O=C(O)Cc1cc(CN2CC(Oc3ccc(NS(=O)(=O)c4cnn(-c5ccc(Cl)cc5)c4)c(CC(=O)O)c3)C2)ccc1NS(=O)(=O)c1cnn(-c2ccc(Cl)cc2)c1] |
| 2 | 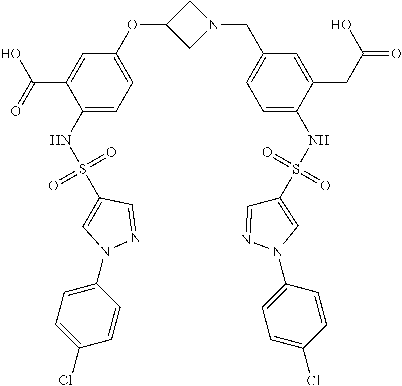 [SMILES: O=C(O)Cc1cc(CN2CC(Oc3ccc(NS(=O)(=O)c4cnn(-c5ccc(Cl)cc5)c4)c(C(=O)O)c3)C2)ccc1NS(=O)(=O)c1cnn(-c2ccc(Cl)cc2)c1] |
| 3 | 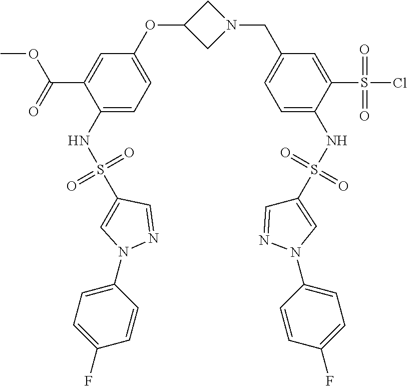 [SMILES: COC(=O)c1cc(OC2CN(Cc3ccc(NS(=O)(=O)c4cnn(-c5ccc(F)cc5)c4)c(S(=O)(=O)Cl)c3)C2)ccc1NS(=O)(=O)c1cnn(-c2ccc(F)cc2)c1] |
| 12 | 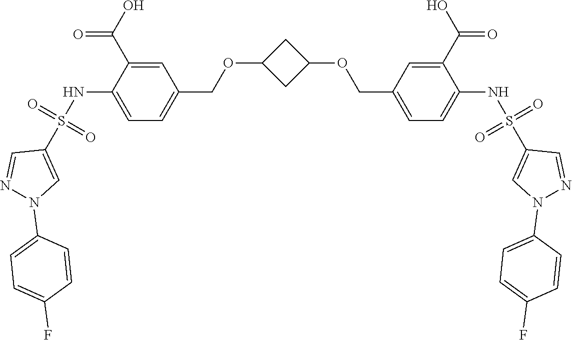 [SMILES: O=C(O)c1cc(COC2CC(OCc3ccc(NS(=O)(=O)c4cnn(-c5ccc(F)cc5)c4)c(C(=O)O)c3)C2)ccc1NS(=O)(=O)c1cnn(-c2ccc(F)cc2)c1] |
| 13 | 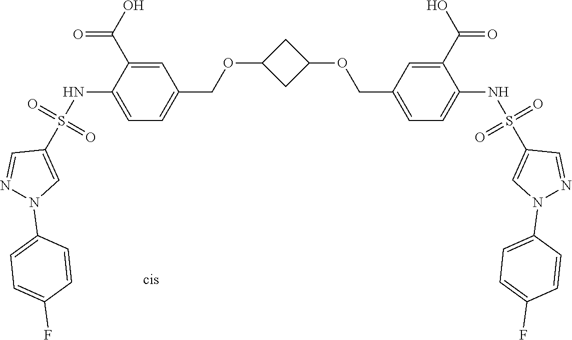 [SMILES: O=C(O)c1cc(COC2CC(OCc3ccc(NS(=O)(=O)c4cnn(-c5ccc(F)cc5)c4)c(C(=O)O)c3)C2)ccc1NS(=O)(=O)c1cnn(-c2ccc(F)cc2)c1] |
| 14 | 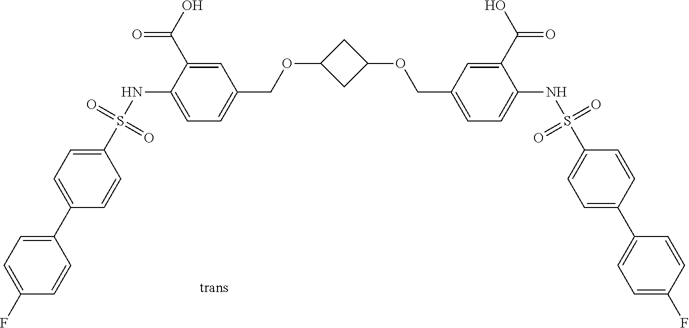 [SMILES: O=C(O)c1cc(COC2CC(OCc3ccc(NS(=O)(=O)c4ccc(-c5ccc(F)cc5)cc4)c(C(=O)O)c3)C2)ccc1NS(=O)(=O)c1ccc(-c2ccc(F)cc2)cc1] |
| 15 | 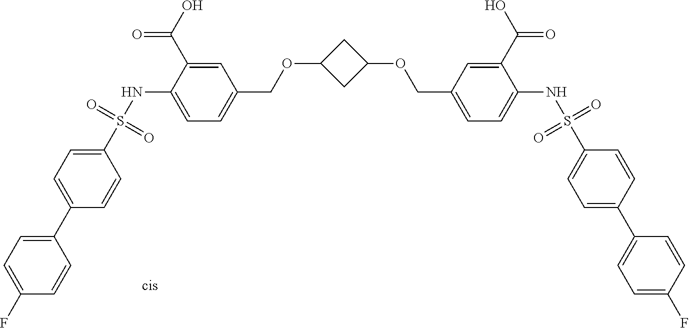 [SMILES: O=C(O)c1cc(COC2CC(OCc3ccc(NS(=O)(=O)c4ccc(-c5ccc(F)cc5)cc4)c(C(=O)O)c3)C2)ccc1NS(=O)(=O)c1ccc(-c2ccc(F)cc2)cc1] |
| 16 | 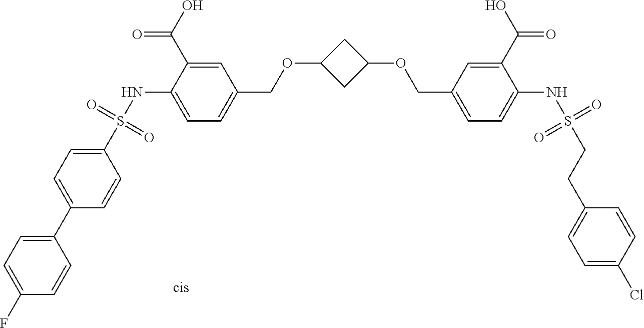 [SMILES: O=C(O)c1cc(COC2CC(OCc3ccc(NS(=O)(=O)c4ccc(-c5ccc(F)cc5)cc4)c(C(=O)O)c3)C2)ccc1NS(=O)(=O)CCc1ccc(Cl)cc1] |
| 17 | 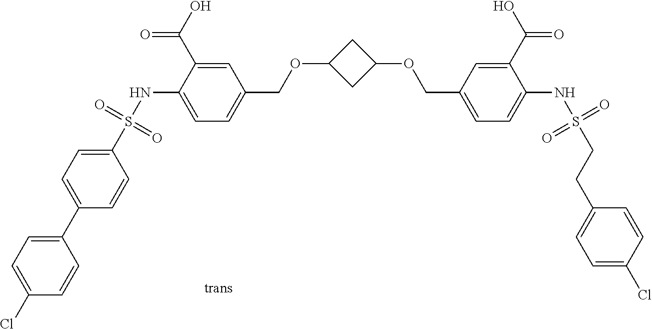 [SMILES: O=C(O)c1cc(COC2CC(OCc3ccc(NS(=O)(=O)c4ccc(-c5ccc(Cl)cc5)cc4)c(C(=O)O)c3)C2)ccc1NS(=O)(=O)CCc1ccc(Cl)cc1] |
| 18 | 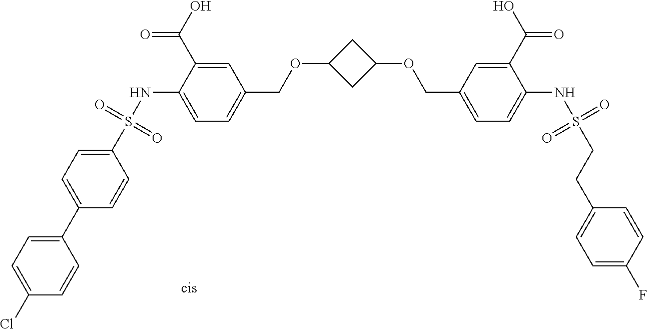 [SMILES: O=C(O)c1cc(COC2CC(OCc3ccc(NS(=O)(=O)c4ccc(-c5ccc(Cl)cc5)cc4)c(C(=O)O)c3)C2)ccc1NS(=O)(=O)CCc1ccc(F)cc1] |
| 19 | 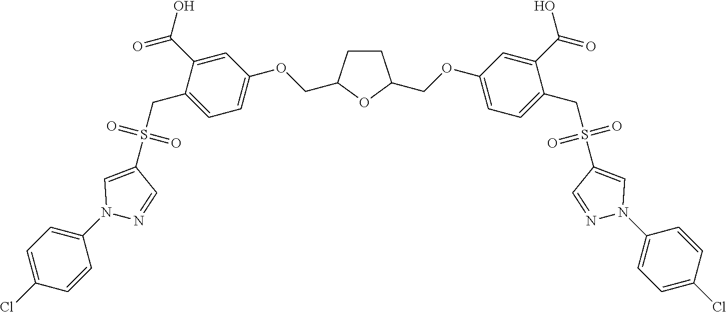 [SMILES: O=C(O)c1cc(OCC2CCC(COc3ccc(CS(=O)(=O)c4cnn(-c5ccc(Cl)cc5)c4)c(C(=O)O)c3)O2)ccc1CS(=O)(=O)c1cnn(-c2ccc(Cl)cc2)c1] |
| 20 | 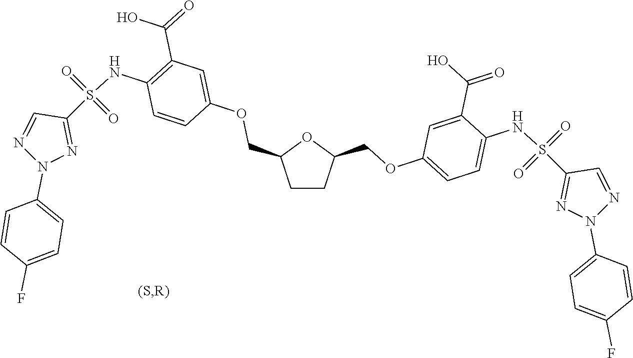 [SMILES: O=C(O)c1cc(OC[C@H]2CC[C@@H](COc3ccc(NS(=O)(=O)c4cnn(-c5ccc(F)cc5)n4)c(C(=O)O)c3)O2)ccc1NS(=O)(=O)c1cnn(-c2ccc(F)cc2)n1] |
| 21 | 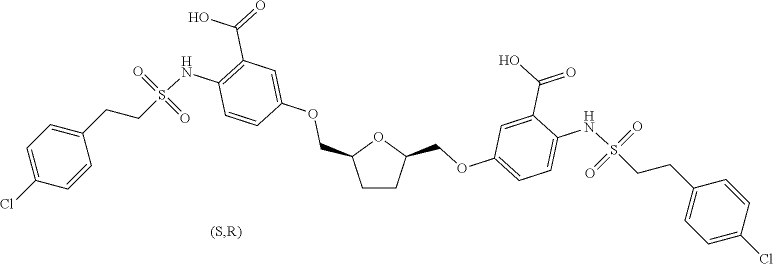 [SMILES: O=C(O)c1cc(OC[C@H]2CC[C@@H](COc3ccc(NS(=O)(=O)CCc4ccc(Cl)cc4)c(C(=O)O)c3)O2)ccc1NS(=O)(=O)CCc1ccc(Cl)cc1] |
| 22 | 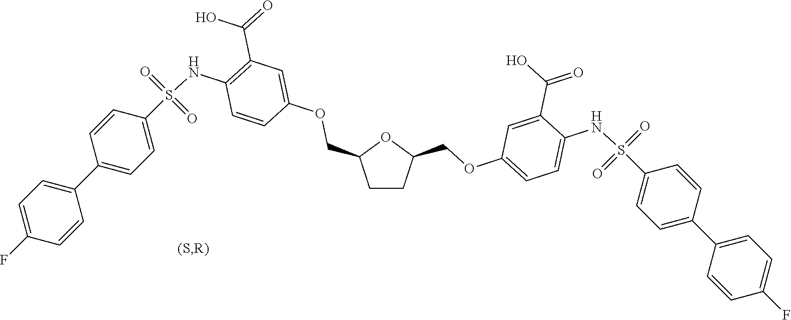 [SMILES: O=C(O)c1cc(OC[C@H]2CC[C@@H](COc3ccc(NS(=O)(=O)c4ccc(-c5ccc(F)cc5)cc4)c(C(=O)O)c3)O2)ccc1NS(=O)(=O)c1ccc(-c2ccc(F)cc2)cc1] |
| 23 | 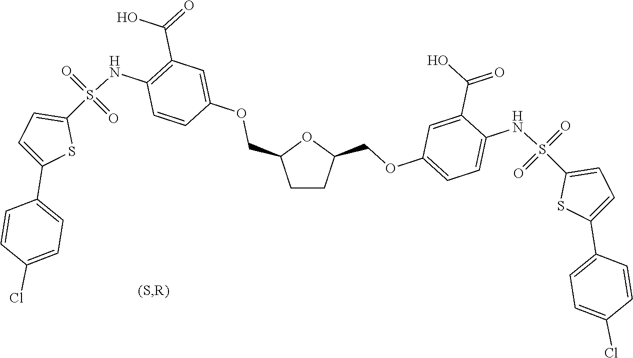 [SMILES: O=C(O)c1cc(OC[C@H]2CC[C@@H](COc3ccc(NS(=O)(=O)c4ccc(-c5ccc(Cl)cc5)s4)c(C(=O)O)c3)O2)ccc1NS(=O)(=O)c1ccc(-c2ccc(Cl)cc2)s1] |
| 24 | 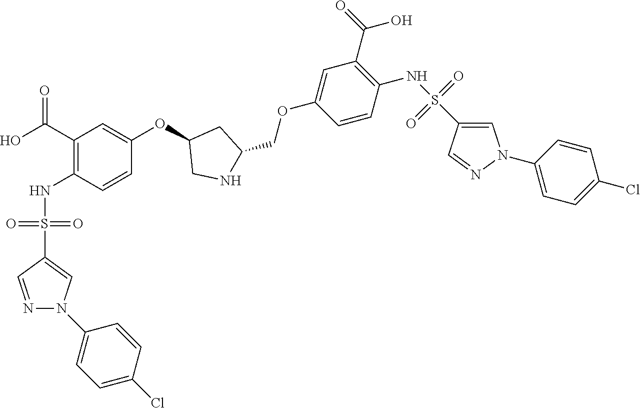 [SMILES: O=C(O)c1cc(OC[C@H]2C[C@H](Oc3ccc(NS(=O)(=O)c4cnn(-c5ccc(Cl)cc5)c4)c(C(=O)O)c3)CN2)ccc1NS(=O)(=O)c1cnn(-c2ccc(Cl)cc2)c1] |
| 25 | 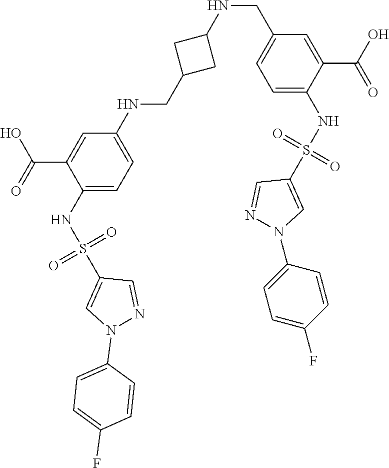 [SMILES: O=C(O)c1cc(CNC2CC(CNc3ccc(NS(=O)(=O)c4cnn(-c5ccc(F)cc5)c4)c(C(=O)O)c3)C2)ccc1NS(=O)(=O)c1cnn(-c2ccc(F)cc2)c1] |
| 26 | 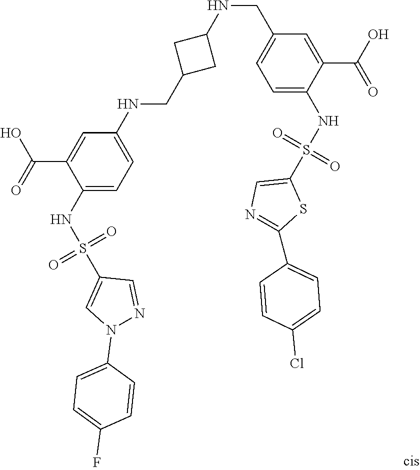 [SMILES: O=C(O)c1cc(NCC2CC(NCc3ccc(NS(=O)(=O)c4cnc(-c5ccc(Cl)cc5)s4)c(C(=O)O)c3)C2)ccc1NS(=O)(=O)c1cnn(-c2ccc(F)cc2)c1] |
| 27 | 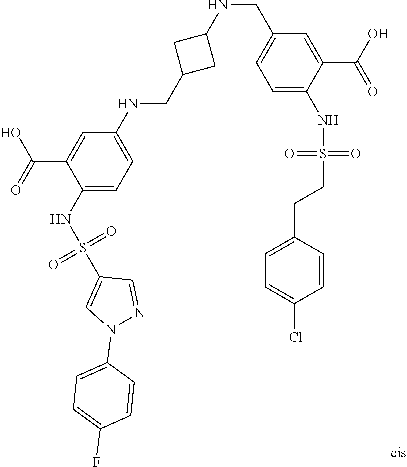 [SMILES: O=C(O)c1cc(CNC2CC(CNc3ccc(NS(=O)(=O)c4cnn(-c5ccc(F)cc5)c4)c(C(=O)O)c3)C2)ccc1NS(=O)(=O)CCc1ccc(Cl)cc1] |
| 28 | 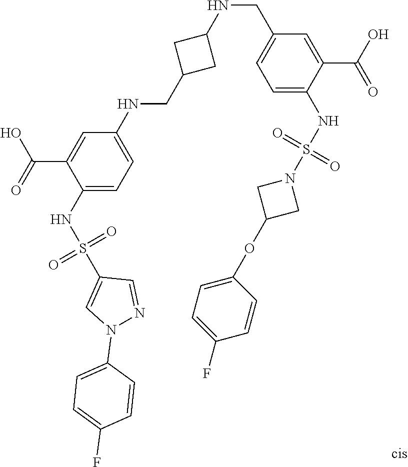 [SMILES: O=C(O)c1cc(NCC2CC(NCc3ccc(NS(=O)(=O)N4CC(Oc5ccc(F)cc5)C4)c(C(=O)O)c3)C2)ccc1NS(=O)(=O)c1cnn(-c2ccc(F)cc2)c1] |
| 29 | 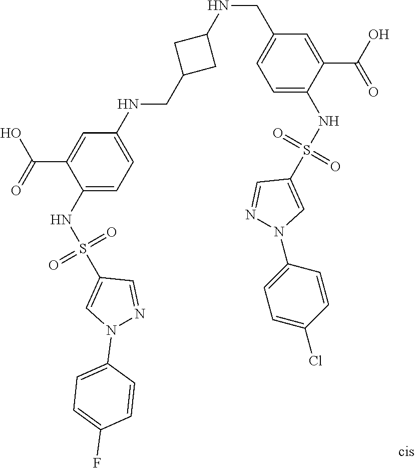 [SMILES: O=C(O)c1cc(CNC2CC(CNc3ccc(NS(=O)(=O)c4cnn(-c5ccc(F)cc5)c4)c(C(=O)O)c3)C2)ccc1NS(=O)(=O)c1cnn(-c2ccc(Cl)cc2)c1] |
| 30 | 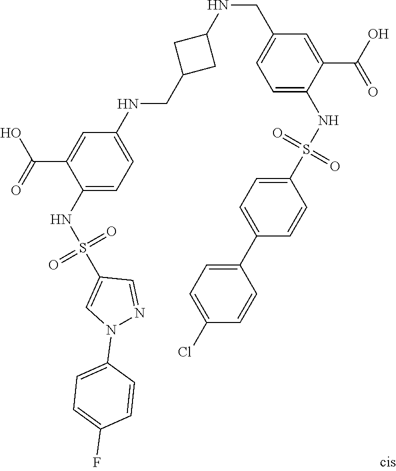 [SMILES: O=C(O)c1cc(CNC2CC(CNc3ccc(NS(=O)(=O)c4cnn(-c5ccc(F)cc5)c4)c(C(=O)O)c3)C2)ccc1NS(=O)(=O)c1ccc(-c2ccc(Cl)cc2)cc1] |
| 31 | 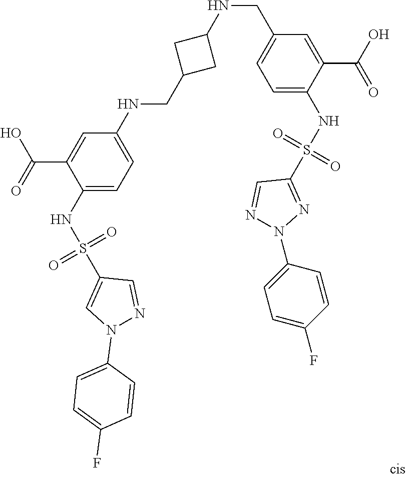 [SMILES: O=C(O)c1cc(NCC2CC(NCc3ccc(NS(=O)(=O)c4cnn(-c5ccc(F)cc5)n4)c(C(=O)O)c3)C2)ccc1NS(=O)(=O)c1cnn(-c2ccc(F)cc2)c1] |
| 32 | 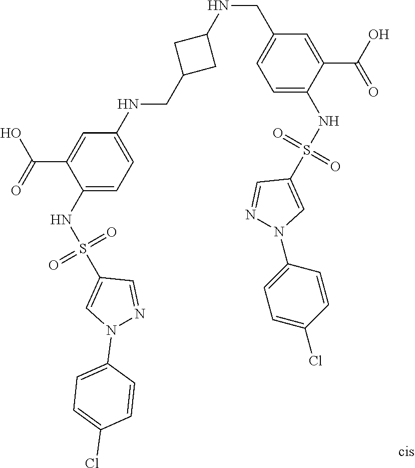 [SMILES: O=C(O)c1cc(CNC2CC(CNc3ccc(NS(=O)(=O)c4cnn(-c5ccc(Cl)cc5)c4)c(C(=O)O)c3)C2)ccc1NS(=O)(=O)c1cnn(-c2ccc(Cl)cc2)c1] |
| 33 | 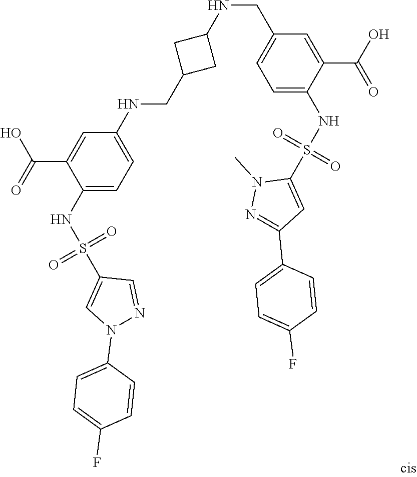 [SMILES: Cn1nc(-c2ccc(F)cc2)cc1S(=O)(=O)Nc1ccc(CNC2CC(CNc3ccc(NS(=O)(=O)c4cnn(-c5ccc(F)cc5)c4)c(C(=O)O)c3)C2)cc1C(=O)O] |
| 34 | 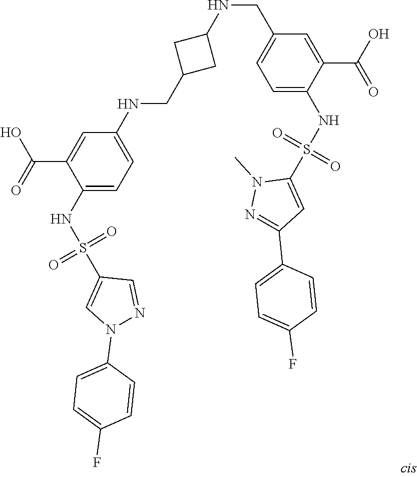 [SMILES: Cn1nc(-c2ccc(F)cc2)cc1S(=O)(=O)Nc1ccc(CNC2CC(CNc3ccc(NS(=O)(=O)c4cnn(-c5ccc(F)cc5)c4)c(C(=O)O)c3)C2)cc1C(=O)O] |
| 35 | 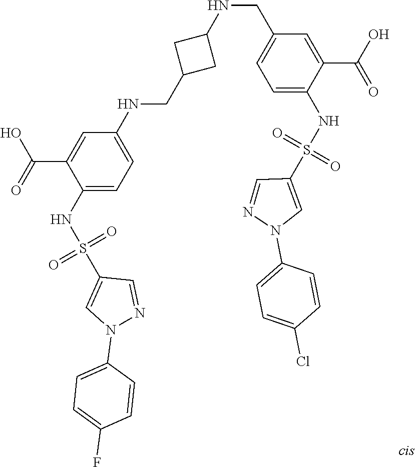 [SMILES: O=C(O)c1cc(CNC2CC(CNc3ccc(NS(=O)(=O)c4cnn(-c5ccc(F)cc5)c4)c(C(=O)O)c3)C2)ccc1NS(=O)(=O)c1cnn(-c2ccc(Cl)cc2)c1] |
| 36 | 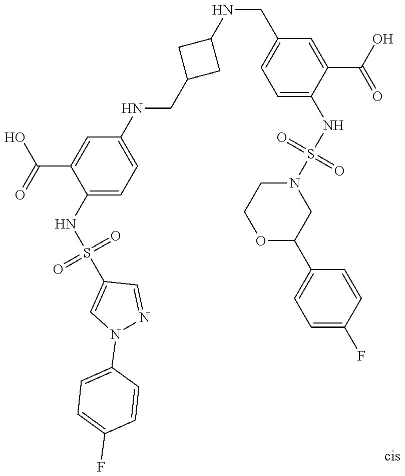 [SMILES: O=C(O)c1cc(NCC2CC(NCc3ccc(NS(=O)(=O)N4CCOC(c5ccc(F)cc5)C4)c(C(=O)O)c3)C2)ccc1NS(=O)(=O)c1cnn(-c2ccc(F)cc2)c1] |
| 37 | 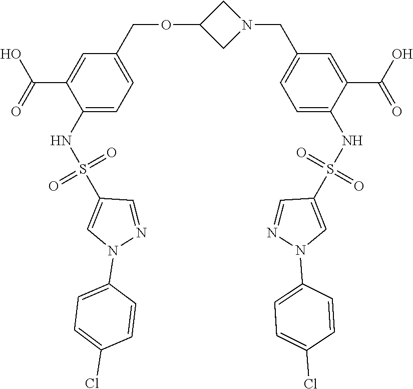 [SMILES: O=C(O)c1cc(COC2CN(Cc3ccc(NS(=O)(=O)c4cnn(-c5ccc(Cl)cc5)c4)c(C(=O)O)c3)C2)ccc1NS(=O)(=O)c1cnn(-c2ccc(Cl)cc2)c1] |
| 38 | 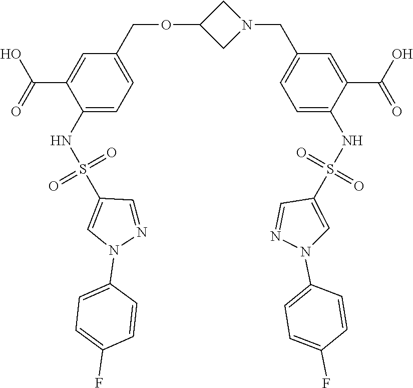 [SMILES: O=C(O)c1cc(COC2CN(Cc3ccc(NS(=O)(=O)c4cnn(-c5ccc(F)cc5)c4)c(C(=O)O)c3)C2)ccc1NS(=O)(=O)c1cnn(-c2ccc(F)cc2)c1] |
| 39 | 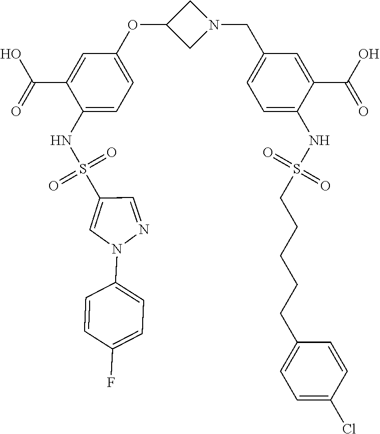 [SMILES: O=C(O)c1cc(CN2CC(Oc3ccc(NS(=O)(=O)c4cnn(-c5ccc(F)cc5)c4)c(C(=O)O)c3)C2)ccc1NS(=O)(=O)CCCCCc1ccc(Cl)cc1] |
| 40 | 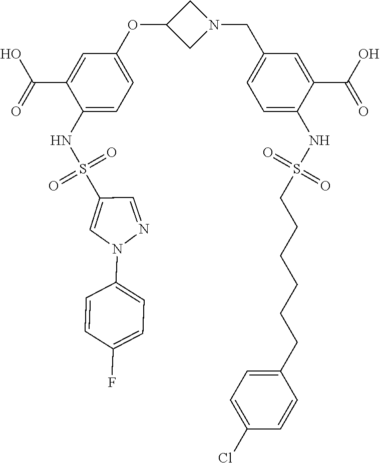 [SMILES: O=C(O)c1cc(CN2CC(Oc3ccc(NS(=O)(=O)c4cnn(-c5ccc(F)cc5)c4)c(C(=O)O)c3)C2)ccc1NS(=O)(=O)CCCCCCc1ccc(Cl)cc1] |
| 41 | 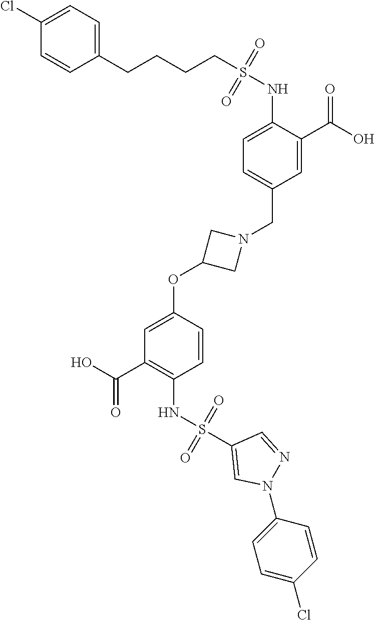 [SMILES: O=C(O)c1cc(CN2CC(Oc3ccc(NS(=O)(=O)c4cnn(-c5ccc(Cl)cc5)c4)c(C(=O)O)c3)C2)ccc1NS(=O)(=O)CCCCc1ccc(Cl)cc1] |
| 42 | 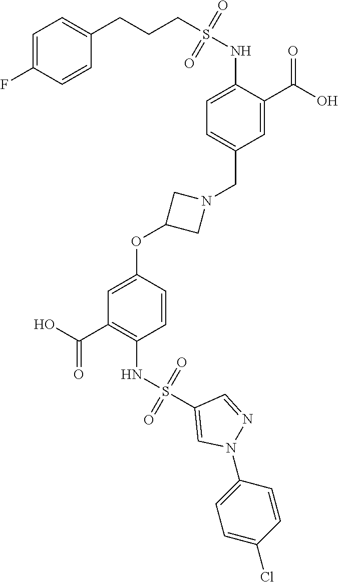 [SMILES: O=C(O)c1cc(CN2CC(Oc3ccc(NS(=O)(=O)c4cnn(-c5ccc(Cl)cc5)c4)c(C(=O)O)c3)C2)ccc1NS(=O)(=O)CCCc1ccc(F)cc1] |
| 43 | 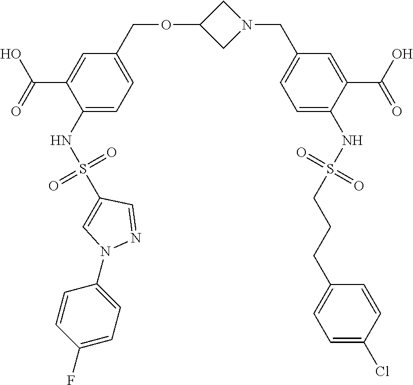 [SMILES: O=C(O)c1cc(CN2CC(OCc3ccc(NS(=O)(=O)c4cnn(-c5ccc(F)cc5)c4)c(C(=O)O)c3)C2)ccc1NS(=O)(=O)CCCc1ccc(Cl)cc1] |
| 44 | 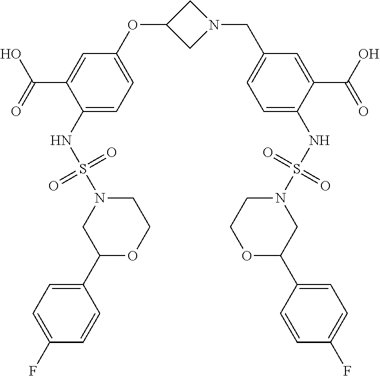 [SMILES: O=C(O)c1cc(CN2CC(Oc3ccc(NS(=O)(=O)N4CCOC(c5ccc(F)cc5)C4)c(C(=O)O)c3)C2)ccc1NS(=O)(=O)N1CCOC(c2ccc(F)cc2)C1] |
| 45 | 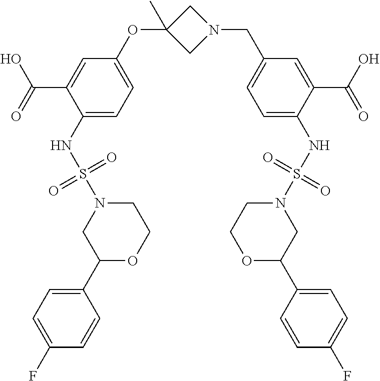 [SMILES: CC1(Oc2ccc(NS(=O)(=O)N3CCOC(c4ccc(F)cc4)C3)c(C(=O)O)c2)CN(Cc2ccc(NS(=O)(=O)N3CCOC(c4ccc(F)cc4)C3)c(C(=O)O)c2)C1] |
| 46 | 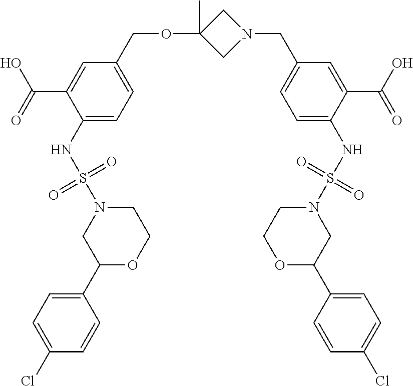 [SMILES: CC1(OCc2ccc(NS(=O)(=O)N3CCOC(c4ccc(Cl)cc4)C3)c(C(=O)O)c2)CN(Cc2ccc(NS(=O)(=O)N3CCOC(c4ccc(Cl)cc4)C3)c(C(=O)O)c2)C1] |
| 47 | 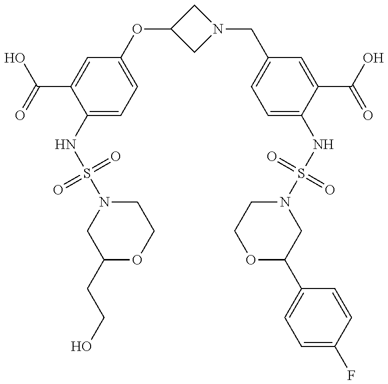 [SMILES: O=C(O)c1cc(OC2CN(Cc3ccc(NS(=O)(=O)N4CCOC(c5ccc(F)cc5)C4)c(C(=O)O)c3)C2)ccc1NS(=O)(=O)N1CCOC(CCO)C1] |
| 48 | 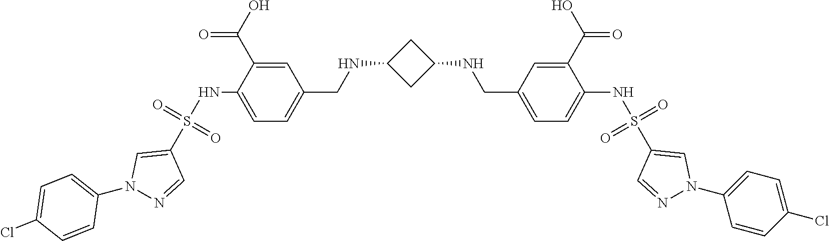 [SMILES: O=C(O)c1cc(CN[C@H]2C[C@@H](NCc3ccc(NS(=O)(=O)c4cnn(-c5ccc(Cl)cc5)c4)c(C(=O)O)c3)C2)ccc1NS(=O)(=O)c1cnn(-c2ccc(Cl)cc2)c1] |
| 49 | 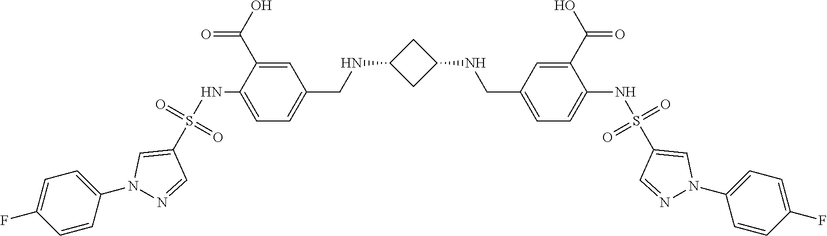 [SMILES: O=C(O)c1cc(CN[C@H]2C[C@@H](NCc3ccc(NS(=O)(=O)c4cnn(-c5ccc(F)cc5)c4)c(C(=O)O)c3)C2)ccc1NS(=O)(=O)c1cnn(-c2ccc(F)cc2)c1] |
| 50 | 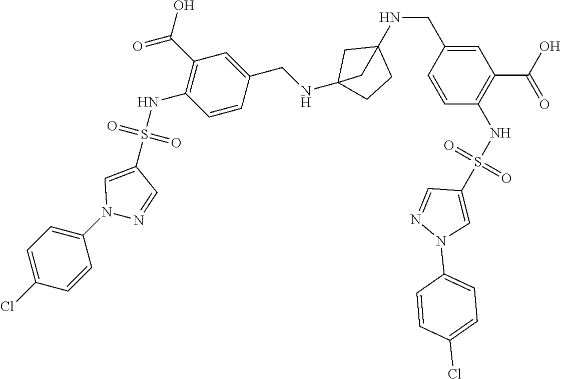 [SMILES: O=C(O)c1cc(CNC23CCC(NCc4ccc(NS(=O)(=O)c5cnn(-c6ccc(Cl)cc6)c5)c(C(=O)O)c4)(C2)C3)ccc1NS(=O)(=O)c1cnn(-c2ccc(Cl)cc2)c1] |
| 51 | 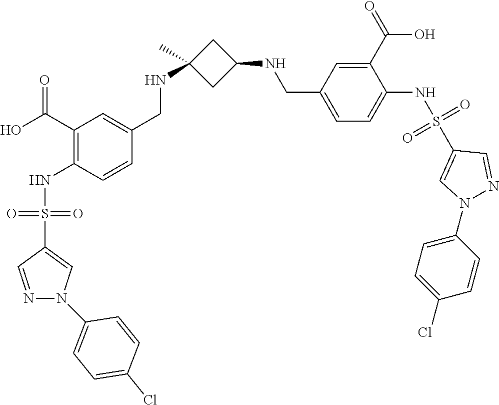 [SMILES: C[C@]1(NCc2ccc(NS(=O)(=O)c3cnn(-c4ccc(Cl)cc4)c3)c(C(=O)O)c2)C[C@@H](NCc2ccc(NS(=O)(=O)c3cnn(-c4ccc(Cl)cc4)c3)c(C(=O)O)c2)C1] |
| 52 | 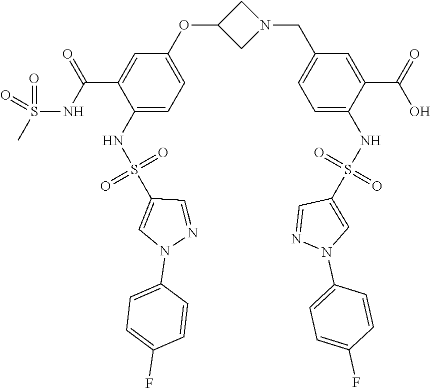 [SMILES: CS(=O)(=O)NC(=O)c1cc(OC2CN(Cc3ccc(NS(=O)(=O)c4cnn(-c5ccc(F)cc5)c4)c(C(=O)O)c3)C2)ccc1NS(=O)(=O)c1cnn(-c2ccc(F)cc2)c1] |
| 53 | 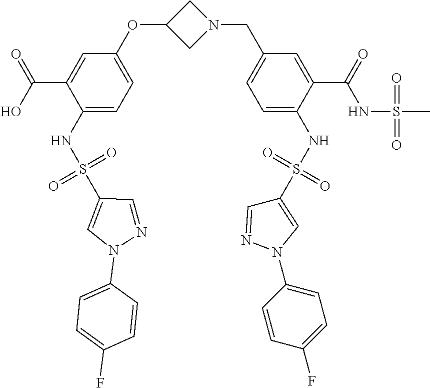 [SMILES: CS(=O)(=O)NC(=O)c1cc(CN2CC(Oc3ccc(NS(=O)(=O)c4cnn(-c5ccc(F)cc5)c4)c(C(=O)O)c3)C2)ccc1NS(=O)(=O)c1cnn(-c2ccc(F)cc2)c1] |
| 54 | 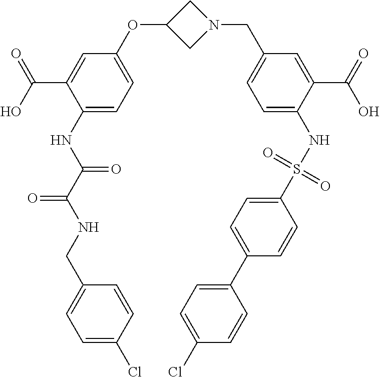 [SMILES: O=C(NCc1ccc(Cl)cc1)C(=O)Nc1ccc(OC2CN(Cc3ccc(NS(=O)(=O)c4ccc(-c5ccc(Cl)cc5)cc4)c(C(=O)O)c3)C2)cc1C(=O)O] |
| 56 | 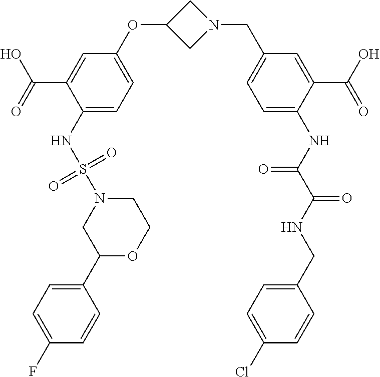 [SMILES: O=C(NCc1ccc(Cl)cc1)C(=O)Nc1ccc(CN2CC(Oc3ccc(NS(=O)(=O)N4CCOC(c5ccc(F)cc5)C4)c(C(=O)O)c3)C2)cc1C(=O)O] |
| 58 | 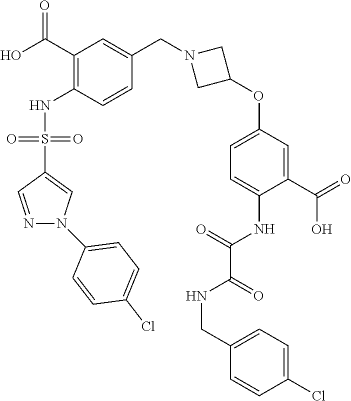 [SMILES: O=C(NCc1ccc(Cl)cc1)C(=O)Nc1ccc(OC2CN(Cc3ccc(NS(=O)(=O)c4cnn(-c5ccc(Cl)cc5)c4)c(C(=O)O)c3)C2)cc1C(=O)O] |
| 59 | 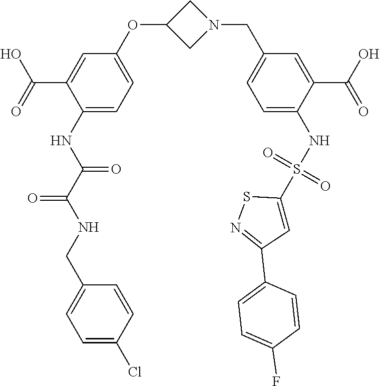 [SMILES: O=C(NCc1ccc(Cl)cc1)C(=O)Nc1ccc(OC2CN(Cc3ccc(NS(=O)(=O)c4cc(-c5ccc(F)cc5)ns4)c(C(=O)O)c3)C2)cc1C(=O)O] |
| 60 | 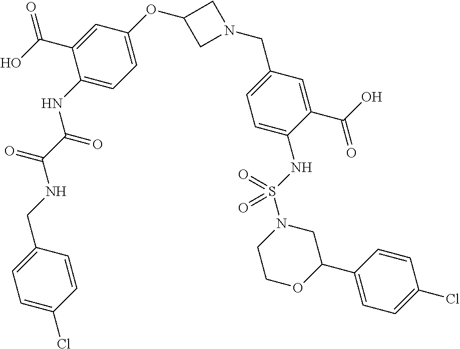 [SMILES: O=C(NCc1ccc(Cl)cc1)C(=O)Nc1ccc(OC2CN(Cc3ccc(NS(=O)(=O)N4CCOC(c5ccc(Cl)cc5)C4)c(C(=O)O)c3)C2)cc1C(=O)O] |
| 61 | 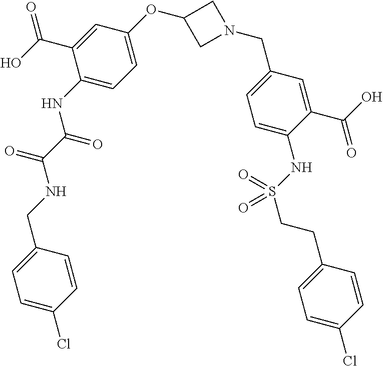 [SMILES: O=C(NCc1ccc(Cl)cc1)C(=O)Nc1ccc(OC2CN(Cc3ccc(NS(=O)(=O)CCc4ccc(Cl)cc4)c(C(=O)O)c3)C2)cc1C(=O)O] |
| 62 | 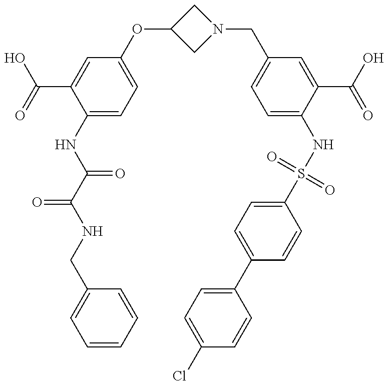 [SMILES: O=C(NCc1ccccc1)C(=O)Nc1ccc(OC2CN(Cc3ccc(NS(=O)(=O)c4ccc(-c5ccc(Cl)cc5)cc4)c(C(=O)O)c3)C2)cc1C(=O)O] |
| 63 | 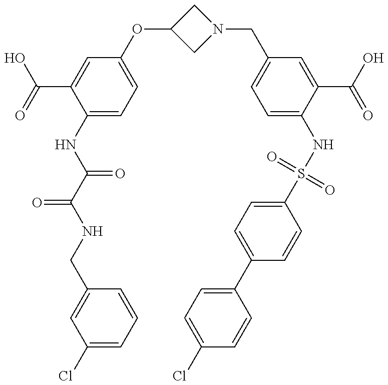 [SMILES: O=C(NCc1cccc(Cl)c1)C(=O)Nc1ccc(OC2CN(Cc3ccc(NS(=O)(=O)c4ccc(-c5ccc(Cl)cc5)cc4)c(C(=O)O)c3)C2)cc1C(=O)O] |
| 65 | 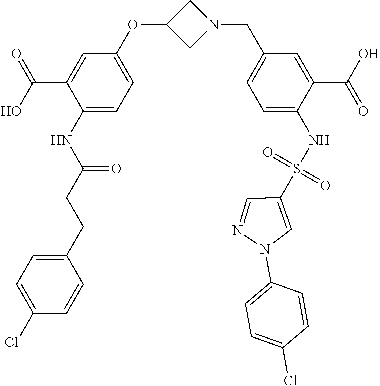 [SMILES: O=C(CCc1ccc(Cl)cc1)Nc1ccc(OC2CN(Cc3ccc(NS(=O)(=O)c4cnn(-c5ccc(Cl)cc5)c4)c(C(=O)O)c3)C2)cc1C(=O)O] |
| 66 | 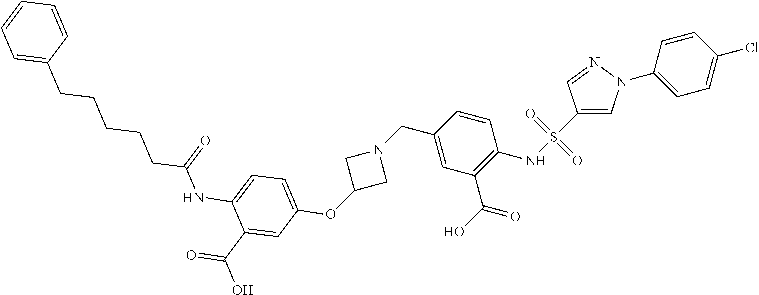 [SMILES: O=C(CCCCCc1ccccc1)Nc1ccc(OC2CN(Cc3ccc(NS(=O)(=O)c4cnn(-c5ccc(Cl)cc5)c4)c(C(=O)O)c3)C2)cc1C(=O)O] |
| 67 | 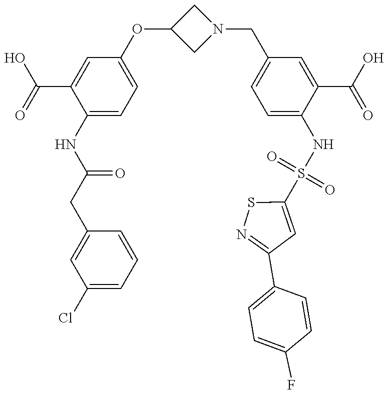 [SMILES: O=C(Cc1cccc(Cl)c1)Nc1ccc(OC2CN(Cc3ccc(NS(=O)(=O)c4cc(-c5ccc(F)cc5)ns4)c(C(=O)O)c3)C2)cc1C(=O)O] |
| 69 | 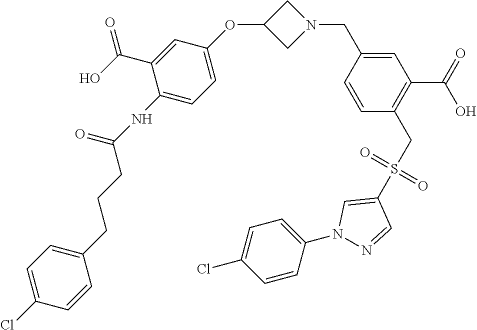 [SMILES: O=C(CCCc1ccc(Cl)cc1)Nc1ccc(OC2CN(Cc3ccc(CS(=O)(=O)c4cnn(-c5ccc(Cl)cc5)c4)c(C(=O)O)c3)C2)cc1C(=O)O] |
| 71 | 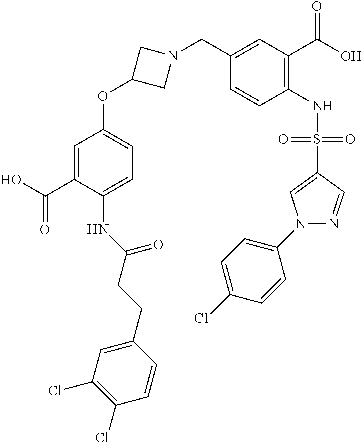 [SMILES: O=C(CCc1ccc(Cl)c(Cl)c1)Nc1ccc(OC2CN(Cc3ccc(NS(=O)(=O)c4cnn(-c5ccc(Cl)cc5)c4)c(C(=O)O)c3)C2)cc1C(=O)O] |
| 72 | 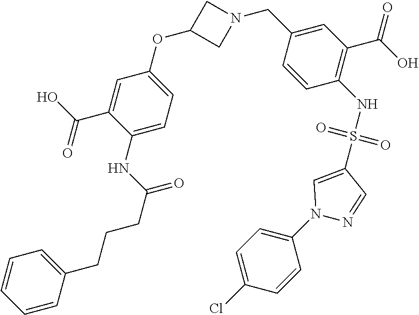 [SMILES: O=C(CCCc1ccccc1)Nc1ccc(OC2CN(Cc3ccc(NS(=O)(=O)c4cnn(-c5ccc(Cl)cc5)c4)c(C(=O)O)c3)C2)cc1C(=O)O] |
| 73 | 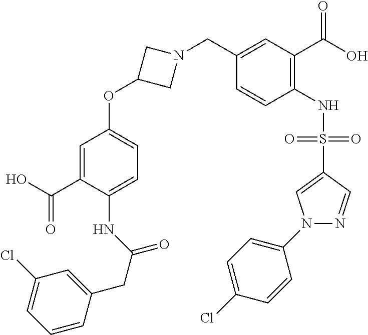 [SMILES: O=C(Cc1cccc(Cl)c1)Nc1ccc(OC2CN(Cc3ccc(NS(=O)(=O)c4cnn(-c5ccc(Cl)cc5)c4)c(C(=O)O)c3)C2)cc1C(=O)O] |
| 74 | 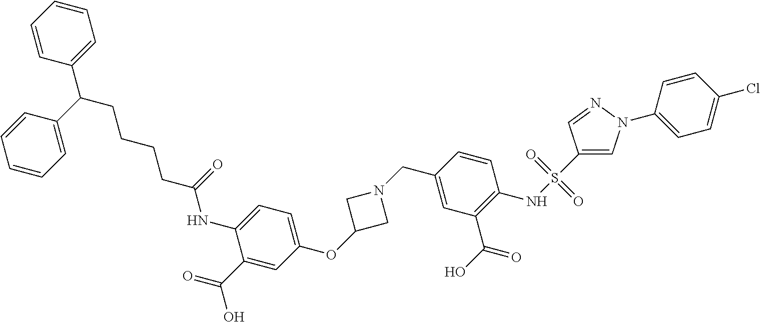 [SMILES: O=C(CCCCC(c1ccccc1)c1ccccc1)Nc1ccc(OC2CN(Cc3ccc(NS(=O)(=O)c4cnn(-c5ccc(Cl)cc5)c4)c(C(=O)O)c3)C2)cc1C(=O)O] |
| 75 | 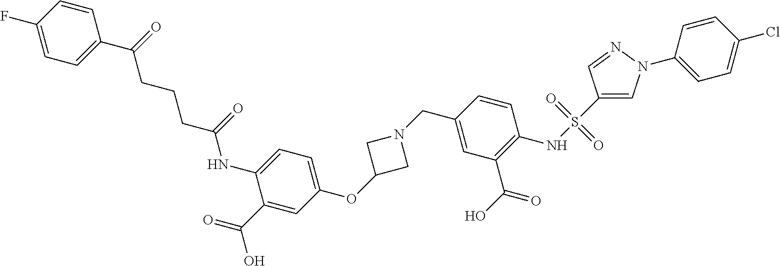 [SMILES: O=C(CCCC(=O)c1ccc(F)cc1)Nc1ccc(OC2CN(Cc3ccc(NS(=O)(=O)c4cnn(-c5ccc(Cl)cc5)c4)c(C(=O)O)c3)C2)cc1C(=O)O] |
| 78 | 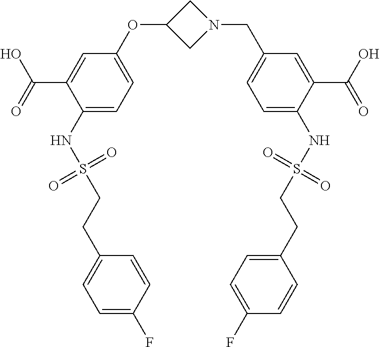 [SMILES: O=C(O)c1cc(CN2CC(Oc3ccc(NS(=O)(=O)CCc4ccc(F)cc4)c(C(=O)O)c3)C2)ccc1NS(=O)(=O)CCc1ccc(F)cc1] |
| 79 | 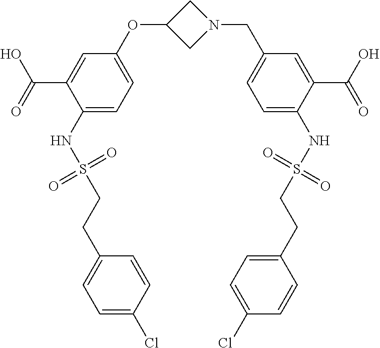 [SMILES: O=C(O)c1cc(CN2CC(Oc3ccc(NS(=O)(=O)CCc4ccc(Cl)cc4)c(C(=O)O)c3)C2)ccc1NS(=O)(=O)CCc1ccc(Cl)cc1] |
| 80 | 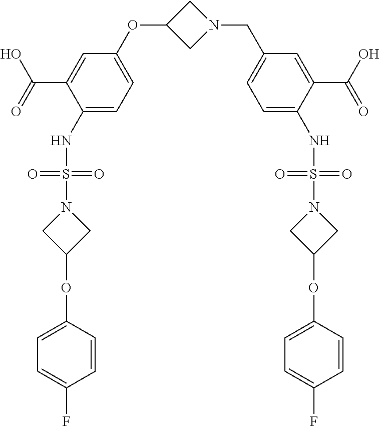 [SMILES: O=C(O)c1cc(CN2CC(Oc3ccc(NS(=O)(=O)N4CC(Oc5ccc(F)cc5)C4)c(C(=O)O)c3)C2)ccc1NS(=O)(=O)N1CC(Oc2ccc(F)cc2)C1] |
| 81 | 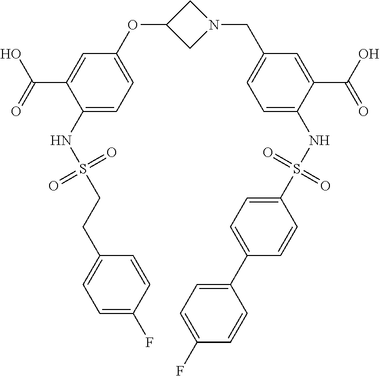 [SMILES: O=C(O)c1cc(OC2CN(Cc3ccc(NS(=O)(=O)c4ccc(-c5ccc(F)cc5)cc4)c(C(=O)O)c3)C2)ccc1NS(=O)(=O)CCc1ccc(F)cc1] |
| 82 | 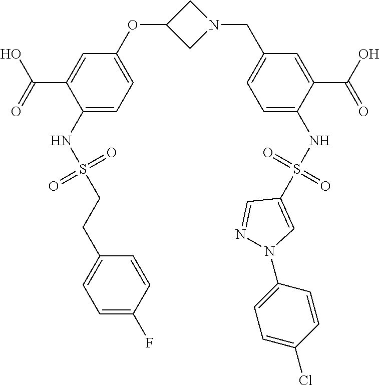 [SMILES: O=C(O)c1cc(OC2CN(Cc3ccc(NS(=O)(=O)c4cnn(-c5ccc(Cl)cc5)c4)c(C(=O)O)c3)C2)ccc1NS(=O)(=O)CCc1ccc(F)cc1] |
| 83 | 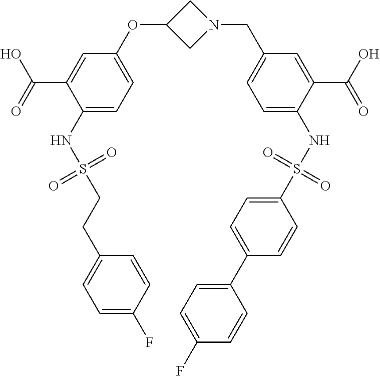 [SMILES: O=C(O)c1cc(OC2CN(Cc3ccc(NS(=O)(=O)c4ccc(-c5ccc(F)cc5)cc4)c(C(=O)O)c3)C2)ccc1NS(=O)(=O)CCc1ccc(F)cc1] |
| 84 | 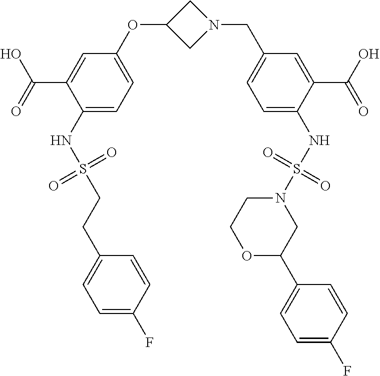 [SMILES: O=C(O)c1cc(OC2CN(Cc3ccc(NS(=O)(=O)N4CCOC(c5ccc(F)cc5)C4)c(C(=O)O)c3)C2)ccc1NS(=O)(=O)CCc1ccc(F)cc1] |
| 85 | 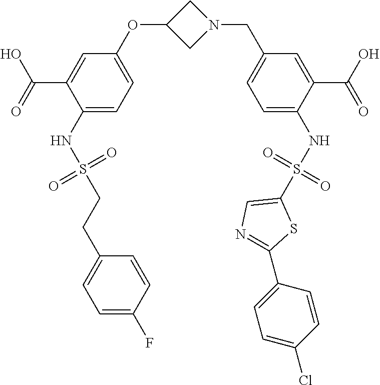 [SMILES: O=C(O)c1cc(OC2CN(Cc3ccc(NS(=O)(=O)c4cnc(-c5ccc(Cl)cc5)s4)c(C(=O)O)c3)C2)ccc1NS(=O)(=O)CCc1ccc(F)cc1] |
| 86 | 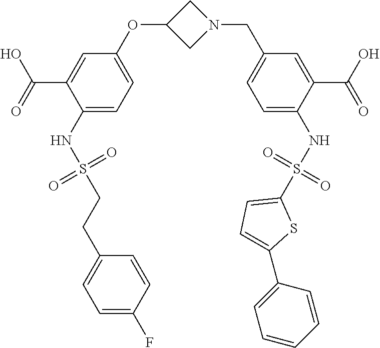 [SMILES: O=C(O)c1cc(OC2CN(Cc3ccc(NS(=O)(=O)c4ccc(-c5ccccc5)s4)c(C(=O)O)c3)C2)ccc1NS(=O)(=O)CCc1ccc(F)cc1] |
| 87 | 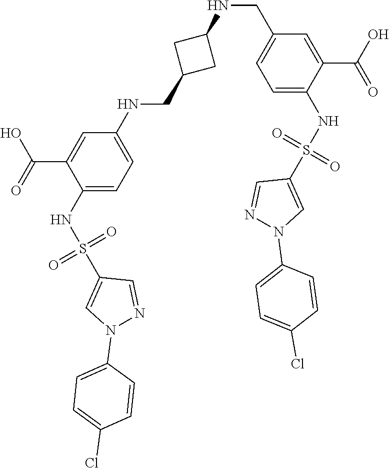 [SMILES: O=C(O)c1cc(CN[C@H]2C[C@@H](CNc3ccc(NS(=O)(=O)c4cnn(-c5ccc(Cl)cc5)c4)c(C(=O)O)c3)C2)ccc1NS(=O)(=O)c1cnn(-c2ccc(Cl)cc2)c1] |
| 88 | 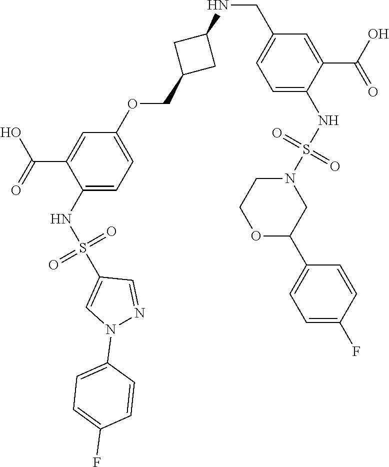 [SMILES: O=C(O)c1cc(OC[C@H]2C[C@@H](NCc3ccc(NS(=O)(=O)N4CCOC(c5ccc(F)cc5)C4)c(C(=O)O)c3)C2)ccc1NS(=O)(=O)c1cnn(-c2ccc(F)cc2)c1] |
| 89 | 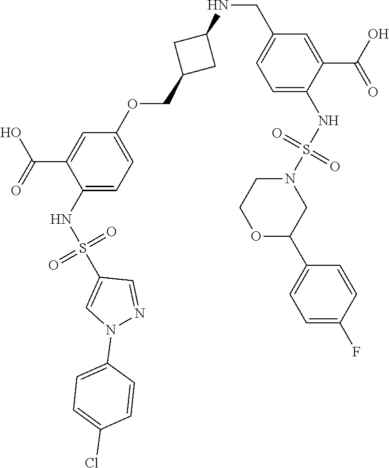 [SMILES: O=C(O)c1cc(OC[C@H]2C[C@@H](NCc3ccc(NS(=O)(=O)N4CCOC(c5ccc(F)cc5)C4)c(C(=O)O)c3)C2)ccc1NS(=O)(=O)c1cnn(-c2ccc(Cl)cc2)c1] |
| 90 | 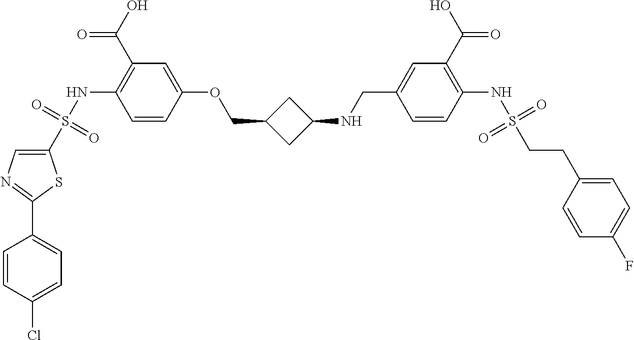 [SMILES: O=C(O)c1cc(CN[C@H]2C[C@@H](COc3ccc(NS(=O)(=O)c4cnc(-c5ccc(Cl)cc5)s4)c(C(=O)O)c3)C2)ccc1NS(=O)(=O)CCc1ccc(F)cc1] |
| 91 | 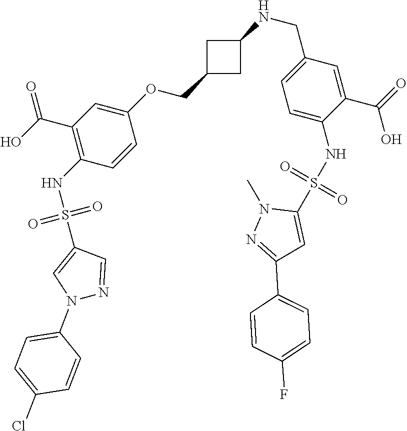 [SMILES: Cn1nc(-c2ccc(F)cc2)cc1S(=O)(=O)Nc1ccc(CN[C@H]2C[C@@H](COc3ccc(NS(=O)(=O)c4cnn(-c5ccc(Cl)cc5)c4)c(C(=O)O)c3)C2)cc1C(=O)O] |
| 92 | 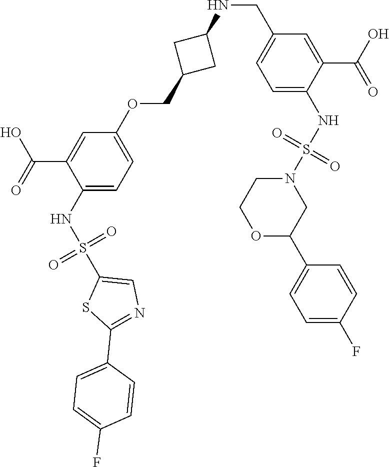 [SMILES: O=C(O)c1cc(OC[C@H]2C[C@@H](NCc3ccc(NS(=O)(=O)N4CCOC(c5ccc(F)cc5)C4)c(C(=O)O)c3)C2)ccc1NS(=O)(=O)c1cnc(-c2ccc(F)cc2)s1] |
| 93 | 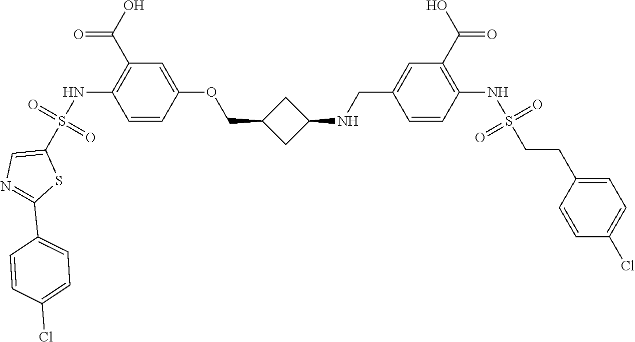 [SMILES: O=C(O)c1cc(CN[C@H]2C[C@@H](COc3ccc(NS(=O)(=O)c4cnc(-c5ccc(Cl)cc5)s4)c(C(=O)O)c3)C2)ccc1NS(=O)(=O)CCc1ccc(Cl)cc1] |
| 94 | 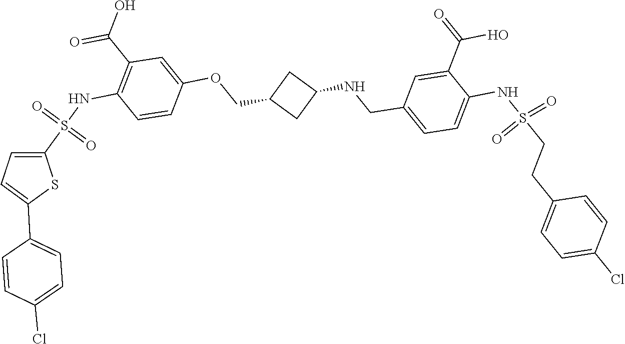 [SMILES: O=C(O)c1cc(CN[C@H]2C[C@@H](COc3ccc(NS(=O)(=O)c4ccc(-c5ccc(Cl)cc5)s4)c(C(=O)O)c3)C2)ccc1NS(=O)(=O)CCc1ccc(Cl)cc1] |
| 95 | 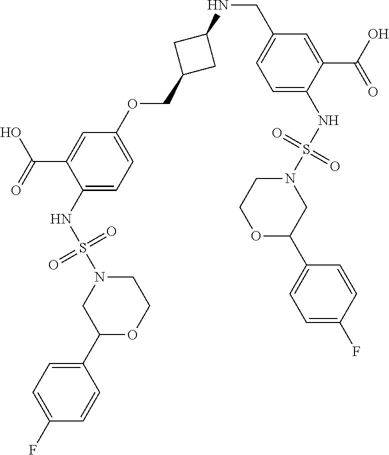 [SMILES: O=C(O)c1cc(CN[C@H]2C[C@@H](COc3ccc(NS(=O)(=O)N4CCOC(c5ccc(F)cc5)C4)c(C(=O)O)c3)C2)ccc1NS(=O)(=O)N1CCOC(c2ccc(F)cc2)C1] |
| 96 | 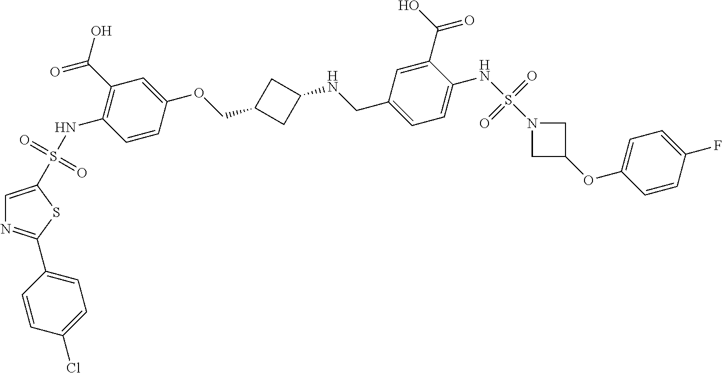 [SMILES: O=C(O)c1cc(OC[C@H]2C[C@@H](NCc3ccc(NS(=O)(=O)N4CC(Oc5ccc(F)cc5)C4)c(C(=O)O)c3)C2)ccc1NS(=O)(=O)c1cnc(-c2ccc(Cl)cc2)s1] |
| 97 | 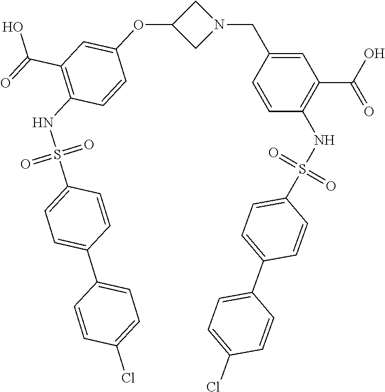 [SMILES: O=C(O)c1cc(CN2CC(Oc3ccc(NS(=O)(=O)c4ccc(-c5ccc(Cl)cc5)cc4)c(C(=O)O)c3)C2)ccc1NS(=O)(=O)c1ccc(-c2ccc(Cl)cc2)cc1] |
| 98 | 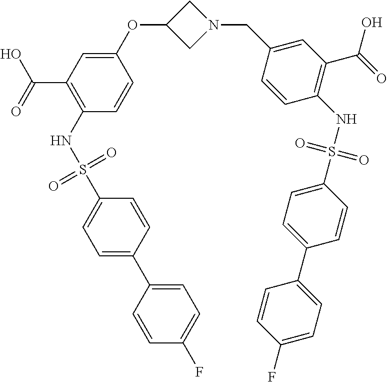 [SMILES: O=C(O)c1cc(CN2CC(Oc3ccc(NS(=O)(=O)c4ccc(-c5ccc(F)cc5)cc4)c(C(=O)O)c3)C2)ccc1NS(=O)(=O)c1ccc(-c2ccc(F)cc2)cc1] |
| 99 | 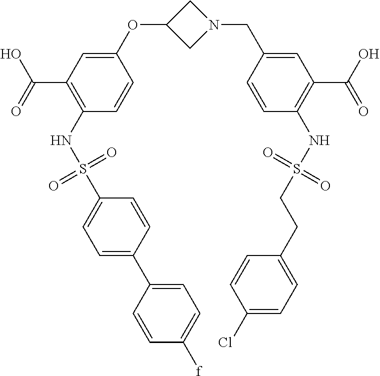 |
| 100 | 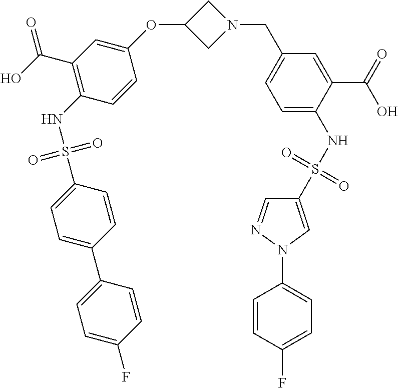 [SMILES: O=C(O)c1cc(CN2CC(Oc3ccc(NS(=O)(=O)c4ccc(-c5ccc(F)cc5)cc4)c(C(=O)O)c3)C2)ccc1NS(=O)(=O)c1cnn(-c2ccc(F)cc2)c1] |
| 101 | 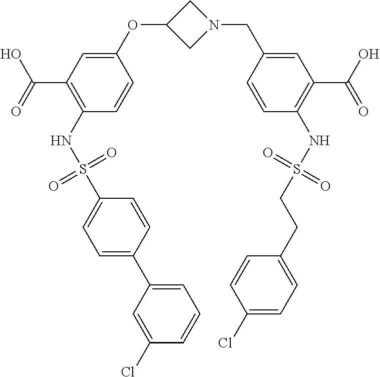 [SMILES: O=C(O)c1cc(CN2CC(Oc3ccc(NS(=O)(=O)c4ccc(-c5cccc(Cl)c5)cc4)c(C(=O)O)c3)C2)ccc1NS(=O)(=O)CCc1ccc(Cl)cc1] |
| 102 | 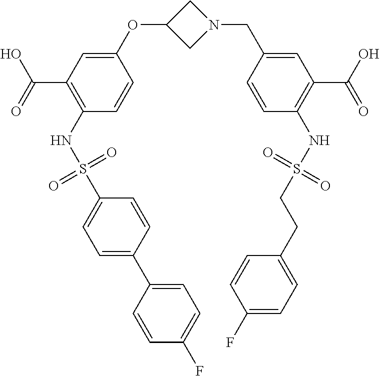 [SMILES: O=C(O)c1cc(CN2CC(Oc3ccc(NS(=O)(=O)c4ccc(-c5ccc(F)cc5)cc4)c(C(=O)O)c3)C2)ccc1NS(=O)(=O)CCc1ccc(F)cc1] |
| 103 | 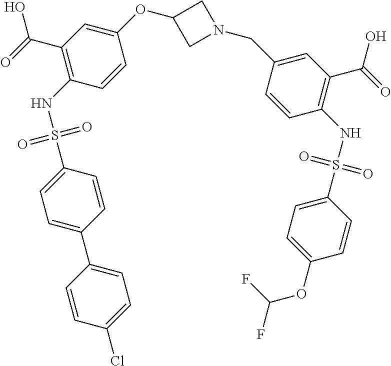 [SMILES: O=C(O)c1cc(CN2CC(Oc3ccc(NS(=O)(=O)c4ccc(-c5ccc(Cl)cc5)cc4)c(C(=O)O)c3)C2)ccc1NS(=O)(=O)c1ccc(OC(F)F)cc1] |
| 104 | 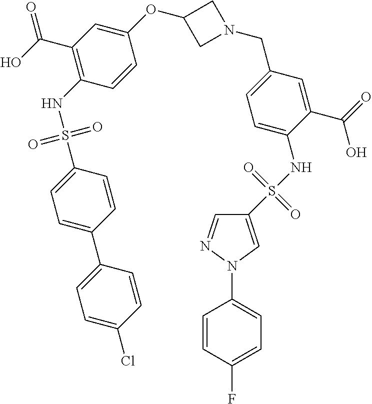 [SMILES: O=C(O)c1cc(CN2CC(Oc3ccc(NS(=O)(=O)c4ccc(-c5ccc(Cl)cc5)cc4)c(C(=O)O)c3)C2)ccc1NS(=O)(=O)c1cnn(-c2ccc(F)cc2)c1] |
| 105 | 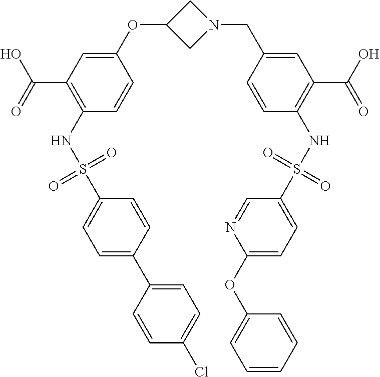 [SMILES: O=C(O)c1cc(CN2CC(Oc3ccc(NS(=O)(=O)c4ccc(-c5ccc(Cl)cc5)cc4)c(C(=O)O)c3)C2)ccc1NS(=O)(=O)c1ccc(Oc2ccccc2)nc1] |
| 106 | 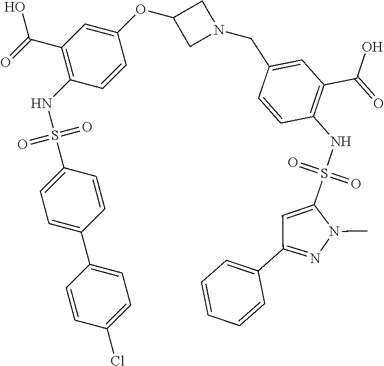 [SMILES: Cn1nc(-c2ccccc2)cc1S(=O)(=O)Nc1ccc(CN2CC(Oc3ccc(NS(=O)(=O)c4ccc(-c5ccc(Cl)cc5)cc4)c(C(=O)O)c3)C2)cc1C(=O)O] |
| 107 | 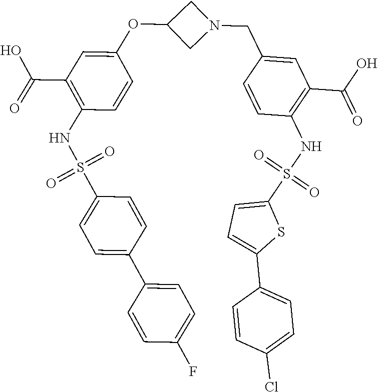 [SMILES: O=C(O)c1cc(OC2CN(Cc3ccc(NS(=O)(=O)c4ccc(-c5ccc(Cl)cc5)s4)c(C(=O)O)c3)C2)ccc1NS(=O)(=O)c1ccc(-c2ccc(F)cc2)cc1] |
| 108 | 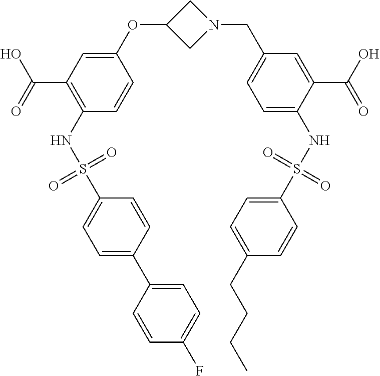 [SMILES: CCCCc1ccc(S(=O)(=O)Nc2ccc(CN3CC(Oc4ccc(NS(=O)(=O)c5ccc(-c6ccc(F)cc6)cc5)c(C(=O)O)c4)C3)cc2C(=O)O)cc1] |
| 114 | 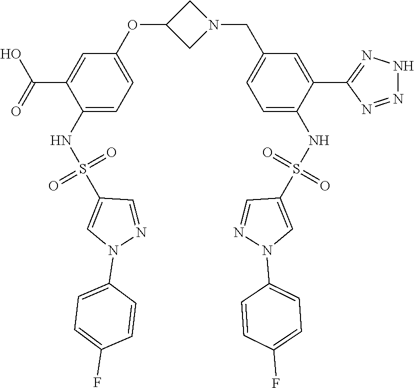 [SMILES: O=C(O)c1cc(OC2CN(Cc3ccc(NS(=O)(=O)c4cnn(-c5ccc(F)cc5)c4)c(-c4nn[nH]n4)c3)C2)ccc1NS(=O)(=O)c1cnn(-c2ccc(F)cc2)c1] |
| 115 | 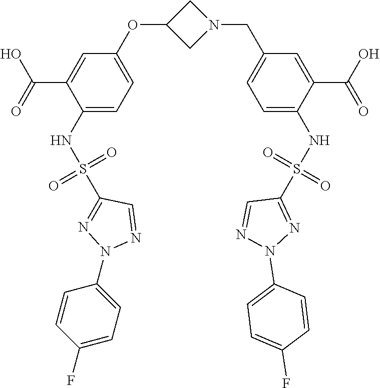 [SMILES: O=C(O)c1cc(CN2CC(Oc3ccc(NS(=O)(=O)c4cnn(-c5ccc(F)cc5)n4)c(C(=O)O)c3)C2)ccc1NS(=O)(=O)c1cnn(-c2ccc(F)cc2)n1] |
| 116 | 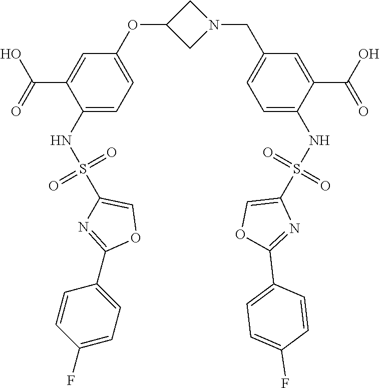 [SMILES: O=C(O)c1cc(CN2CC(Oc3ccc(NS(=O)(=O)c4coc(-c5ccc(F)cc5)n4)c(C(=O)O)c3)C2)ccc1NS(=O)(=O)c1coc(-c2ccc(F)cc2)n1] |
| 117 | 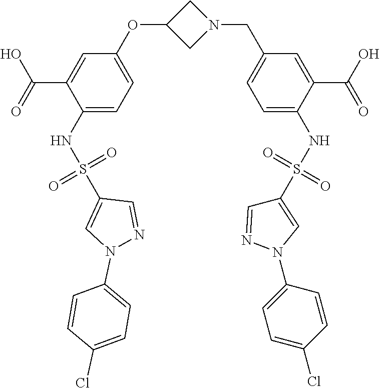 [SMILES: O=C(O)c1cc(CN2CC(Oc3ccc(NS(=O)(=O)c4cnn(-c5ccc(Cl)cc5)c4)c(C(=O)O)c3)C2)ccc1NS(=O)(=O)c1cnn(-c2ccc(Cl)cc2)c1] |
| 118 | 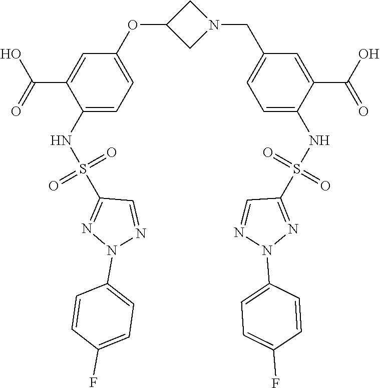 [SMILES: O=C(O)c1cc(CN2CC(Oc3ccc(NS(=O)(=O)c4cnn(-c5ccc(F)cc5)n4)c(C(=O)O)c3)C2)ccc1NS(=O)(=O)c1cnn(-c2ccc(F)cc2)n1] |
| 120 | 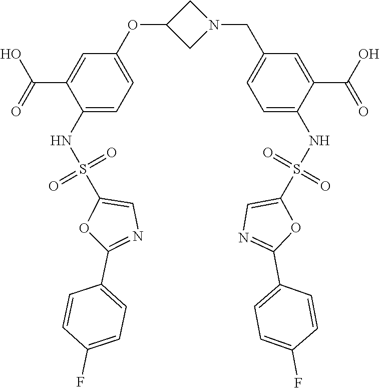 [SMILES: O=C(O)c1cc(CN2CC(Oc3ccc(NS(=O)(=O)c4cnc(-c5ccc(F)cc5)o4)c(C(=O)O)c3)C2)ccc1NS(=O)(=O)c1cnc(-c2ccc(F)cc2)o1] |
| 121 | 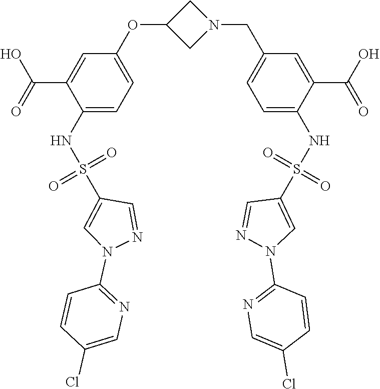 [SMILES: O=C(O)c1cc(CN2CC(Oc3ccc(NS(=O)(=O)c4cnn(-c5ccc(Cl)cn5)c4)c(C(=O)O)c3)C2)ccc1NS(=O)(=O)c1cnn(-c2ccc(Cl)cn2)c1] |

22. A pharmaceutical composition comprising a compound of claim 1, or a pharmaceutically acceptable salt thereof, and a pharmaceutically acceptable carrier.

23. (canceled)

24. A method of treating infections caused by any multi-drug resistant (MDR) Gram-negative bacteria comprising administering an effective amount of a compound of claim 1, or a pharmaceutically acceptable salt thereof, to a person in need thereof.

25. A method of treating bacterial infections in which MsbA is involved comprising administering an effective amount of a compound of claim 1, or a pharmaceutically acceptable salt thereof, to a person in need thereof.
